SEQUENCE LISTING (80 sequences)

SEQ ID NO: 1 - DNA, 495 bp, Homo sapiens
  Human IL-22
atgggatggtcatgtatcatcctttttctagtagcaactgcaactggagtacattcagcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcatt

SEQ ID NO: 2 - protein, 165 aa, Homo sapiens
  Human IL-22
MGWSCIILFLVATATGVHSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 3 - DNA, 438 bp, Homo sapiens
  IL-22 DNA (mature)
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcatt

SEQ ID NO: 4 - protein, 146 aa, Homo sapiens
  IL-22 (mature)
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 5 - DNA, 57 bp, Homo sapiens
  IL-22 leader sequence
atgggatggtcatgtatcatcctttttctagtagcaactgcaactggagtacattca

SEQ ID NO: 6 - protein, 19 aa, Homo sapiens
  IL-22 leader sequence
MGWSCIILFLVATATGVHS

SEQ ID NO: 7 - DNA, 1128 bp, Artificial Sequence
  Description of Artificial Sequence Synthetic polynucleotide
  IL-22 Fc fusion IgG4 (minus C-terminal Lys) N297G
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattcgcgttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcggaagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 8 - protein, 376 aa, Artificial Sequence
  Description of Artificial Sequence Synthetic polypeptide
  IL-22 Fc fusion IgG4 (minus C-terminal Lys) N297G
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIRVESKYGPPCPPCPAPEFLGGPSVFLFPPKPKDTLMISRTPEVTCVVVDVSQEDPEVQFNWYVDGVEVHNAKTKPREEQFGSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 9 - DNA, 1128 bp, Artificial Sequence
  Description of Artificial Sequence Synthetic polynucleotide
  IL-22 Fc fusion IgG4 (minus C-terminal Lys) N297A
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattcgcgttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcgctagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 10 - protein, 376 aa, Artificial Sequence
  Description of Artificial Sequence Synthetic polypeptide
  IL-22 Fc fusion IgG4 (minus C-terminal Lys) N297A
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIRVESKYGPPCPPCPAPEFLGGPSVFLFPPKPKDTLMISRTPEVTCVVVDVSQEDPEVQFNWYVDGVEVHNAKTKPREEQFASTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 11 - DNA, 1131 bp, Artificial Sequence
  Description of Artificial Sequence Synthetic polynucleotide
  IL-22 Fc fusion IgG1 (minus C-terminal Lys) N297G
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattgagcccaaatctagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 12 - protein, 377 aa, Artificial Sequence
  Description of Artificial Sequence Synthetic polypeptide
  IL-22 Fc fusion IgG1 (minus C-terminal Lys) N297G
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIEPKSSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - DNA, 1131 bp, Artificial Sequence
  Description of Artificial Sequence Synthetic polynucleotide
  IL-22 Fc fusion IgG1 (minus C-terminal Lys) N297A
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattgagcccaaatctagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 14 - protein, 377 aa, Artificial Sequence
  Description of Artificial Sequence Synthetic polypeptide
  IL-22 Fc fusion IgG1 (minus C-terminal Lys) N297A
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - DNA, 1131 bp, Artificial Sequence
  Description of Artificial Sequence Synthetic polynucleotide
  IL-22 Fc fusion IgG4 (full) N297G
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattcgcgttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcggaagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 16 - protein, 377 aa, Artificial Sequence
  Description of Artificial Sequence Synthetic polypeptide
  IL-22 Fc fusion IgG4 (full) N297G
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIRVESKYGPPCPPCPAPEFLGGPSVFLFPPKPKDTLMISRTPEVTCVVVDVSQEDPEVQFNWYVDGVEVHNAKTKPREEQFGSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - DNA, 1131 bp, Artificial Sequence
  Description of Artificial Sequence Synthetic polynucleotide
  IL-22 Fc fusion IgG4 (full) N297A
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattcgcgttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcgctagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 18 - protein, 377 aa, Artificial Sequence
  Description of Artificial Sequence Synthetic polypeptide
  IL-22 Fc fusion IgG4 (full) N297A
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIRVESKYGPPCPPCPAPEFLGGPSVFLFPPKPKDTLMISRTPEVTCVVVDVSQEDPEVQFNWYVDGVEVHNAKTKPREEQFASTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - DNA, 1134 bp, Artificial Sequence
  Description of Artificial Sequence Synthetic polynucleotide
  IL-22 Fc fusion IgG1 (full) N297G
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattgagcccaaatctagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 20 - protein, 378 aa, Artificial Sequence
  Description of Artificial Sequence Synthetic polypeptide
  IL-22 Fc fusion IgG1 (full) N297G
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIEPKSSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 1134 bp, Artificial Sequence
  Description of Artificial Sequence Synthetic polynucleotide
  IL-22 Fc fusion IgG1 (full) N297A
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattgagcccaaatctagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 22 - protein, 378 aa, Artificial Sequence
  Description of Artificial Sequence Synthetic polypeptide
  IL-22 Fc fusion IgG1 (full) N297A
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - DNA, 1128 bp, Artificial Sequence
  Description of Artificial Sequence Synthetic polynucleotide
  IL-22 Fc fusion IgG4 (wt N297, minus Lys)
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattcgcgttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 24 - protein, 376 aa, Artificial Sequence
  Description of Artificial Sequence Synthetic polypeptide
  IL-22 Fc fusion IgG4 (wt N297, minus Lys)
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 25 - DNA, 1131 bp, Artificial Sequence
  Description of Artificial Sequence Synthetic polynucleotide
  IL-22 Fc fusion IgG1 (wt N297, minus Lys)
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattgagcccaaatctagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 26 - protein, 377 aa, Artificial Sequence
  Description of Artificial Sequence Synthetic polypeptide
  IL-22 Fc fusion IgG1 (wt N297, minus Lys)
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - DNA, 1131 bp, Artificial Sequence
  Description of Artificial Sequence Synthetic polynucleotide
  IL-22 Fc fusion IgG4 (N297 wt)
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattcgcgttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 28 - protein, 377 aa, Artificial Sequence
  Description of Artificial Sequence Synthetic polypeptide
  IL-22 Fc fusion IgG4 (N297 wt)
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - DNA, 1134 bp, Artificial Sequence
  Description of Artificial Sequence Synthetic polynucleotide
  IL-22 Fc fusion IgG1 (N297 wt)
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattgagcccaaatctagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 30 - protein, 378 aa, Artificial Sequence
  Description of Artificial Sequence Synthetic polypeptide
  IL-22 Fc fusion IgG1 (N297 wt)
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 5 aa, Artificial Sequence
  Description of Artificial Sequence Synthetic hinge peptide
CPPCP

SEQ ID NO: 32 - protein, 5 aa, Artificial Sequence
  Description of Artificial Sequence Synthetic linker peptide
DKTHT

SEQ ID NO: 33 - protein, 10 aa, Artificial Sequence
  Description of Artificial Sequence Synthetic linker peptide
EPKSCDKTHT

SEQ ID NO: 34 - protein, 11 aa, Artificial Sequence
  Description of Artificial Sequence Synthetic linker peptide
VEPKSCDKTHT

SEQ ID NO: 35 - protein, 12 aa, Artificial Sequence
  Description of Artificial Sequence Synthetic linker peptide
KVEPKSCDKTHT

SEQ ID NO: 36 - protein, 13 aa, Artificial Sequence
  Description of Artificial Sequence Synthetic linker peptide
KKVEPKSCDKTHT

SEQ ID NO: 37 - protein, 14 aa, Artificial Sequence
  Description of Artificial Sequence Synthetic linker peptide
DKKVEPKSCDKTHT

SEQ ID NO: 38 - protein, 15 aa, Artificial Sequence
  Description of Artificial Sequence Synthetic linker peptide
VDKKVEPKSCDKTHT

SEQ ID NO: 39 - protein, 16 aa, Artificial Sequence
  Description of Artificial Sequence Synthetic linker peptide
KVDKKVEPKSCDKTHT

SEQ ID NO: 40 - protein, 10 aa, Artificial Sequence
  Description of Artificial Sequence Synthetic linker peptide
EPKSSDKTHT

SEQ ID NO: 41 - protein, 8 aa, Artificial Sequence
  Description of Artificial Sequence Synthetic linker peptide
GGGDKTHT

SEQ ID NO: 42 - protein, 12 aa, Artificial Sequence
  Description of Artificial Sequence Synthetic linker (IgG3) peptide
ELKTPLGDTTHT

SEQ ID NO: 43 - protein, 6 aa, Artificial Sequence
  Description of Artificial Sequence Synthetic linker peptide
SKYGPP

SEQ ID NO: 44 - protein, 9 aa, Artificial Sequence
  Description of Artificial Sequence Synthetic linker peptide
RVESKYGPP

SEQ ID NO: 45 - protein, 3 aa, Artificial Sequence
  Description of Artificial Sequence Synthetic linker peptide
GGS

SEQ ID NO: 46 - protein, 4 aa, Artificial Sequence
  Description of Artificial Sequence Synthetic linker peptide
GGGS

SEQ ID NO: 47 - protein, 5 aa, Artificial Sequence
  Description of Artificial Sequence Synthetic linker peptide
GGGGS

SEQ ID NO: 48 - protein, 146 aa, Pan troglodytes
APISSHCRLDKSSFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGENGEIKAIGELDLLFMSLRNACI

SEQ ID NO: 49 - protein, 146 aa, Pongo abelii
APISSHCRLDKSNFQQPYITNRTFMLAKEASLADNNTDVRLIGEKLFRGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 50 - protein, 146 aa, Mus musculus
LPVNTRCKLEVSNFQQPYIVNRTFMLAKEASLADNNTDVRLIGEKLFRGVSAKDQCYLMKQVLNFTLEDVLLPQSDRFQPYMQEVVPFLTKLSNQLSSCHISGDDQNIQKNVRRLKETVKKLGESGEIKAIGELDLLFMSLRNACV

SEQ ID NO: 51 - protein, 146 aa, Canis familiaris
LPISSHCRLDKSNFQQPYITNRTFMLAKEASLADNNTDVRLIGEKLFHGVNMGERCYLMKEVLNFTLEEVLLPQSDRFQPYMQEVVPFLARLSNKLSQCHIENDDQHIQRNVQKLKDTVQKLGENGEIKAIGELDLLFMALRNACV

SEQ ID NO: 52 - DNA, 94 bp, Artificial Sequence
  Description of Artificial Sequence Synthetic IL-22 Fc fusion protein IgG1 forward primer
ttgaattccaccatgggatggtcatgtatcatcctttttctagtagcaactgcaactggagtacattcagcgcccatcagctcccactgcaggc

SEQ ID NO: 53 - DNA, 33 bp, Artificial Sequence
  Description of Artificial Sequence Synthetic IL-22 Fc fusion IgG1 reverse primer
aggtcgactcatttacccggagacagggagagg

SEQ ID NO: 54 - DNA, 94 bp, Artificial Sequence; identical to SEQ ID NO: 52
  Description of Artificial Sequence Synthetic IL-22 Fc fusion IgG4 forward primer

SEQ ID NO: 55 - DNA, 33 bp, Artificial Sequence
  Description of Artificial Sequence Synthetic IL-22 Fc fusion IgG4 reverse primer
aggtcgacttatttacccagagacagggagagg

SEQ ID NO: 56 - DNA, 35 bp, Artificial Sequence
  Description of Artificial Sequence Synthetic IgG1 N297G forward primer
gcgggaggagcagtacggaagcacgtaccgtgtgg

SEQ ID NO: 57 - DNA, 35 bp, Artificial Sequence
  Description of Artificial Sequence Synthetic IgG1 N297G reverse primer
ccacacggtacgtgcttccgtactgctcctcccgc

SEQ ID NO: 58 - DNA, 51 bp, Artificial Sequence
  Description of Artificial Sequence Synthetic IgG4 N297G forward primer
acaaagccgcgggaggagcagttcggaagcacgtaccgtgtggtcagcgtc

SEQ ID NO: 59 - DNA, 51 bp, Artificial Sequence
  Description of Artificial Sequence Synthetic IgG4 N297G reverse primer
gacgctgaccacacggtacgtgcttccgaactgctcctcccgcggctttgt

SEQ ID NO: 60 - protein, 411 aa, Artificial Sequence
  Description of Artificial Sequence Synthetic polypeptide
  IL-22 Fc fusion IgG2a
MAVLQKSMSFSLMGTLAASCLLLIALWAQEANALPVNTRCKLEVSNFQQPYIVNRTFMLAKEASLADNNTDVRLIGEKLFRGVSAKDQCYLMKQVLNFTLEDVLLPQSDRFQPYMQEVVPFLTKLSNQLSSCHISGDDQNIQKNVRRLKETVKKLGESGEIKAIGELDLLFMSLRNACVARGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 61 - protein, 372 aa, Artificial Sequence
  Description of Artificial Sequence Synthetic polypeptide
  IL-22 IgG1 fusion knob (T366W) minus Lys
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 226 aa, Artificial Sequence
  Description of Artificial Sequence Synthetic polypeptide
  Monomeric Fc hole
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 63 - protein, 7 aa, Artificial Sequence
  Description of Artificial Sequence Synthetic linker peptide
GGGSTHT

SEQ ID NO: 64 - protein, 14 aa, Artificial Sequence
  Description of Artificial Sequence Synthetic linker peptide
DKKVEPKSSDKTHT

SEQ ID NO: 65 - protein, 16 aa, Artificial Sequence
  Description of Artificial Sequence Synthetic linker peptide
KVDKKVEPKSSDKTHT

SEQ ID NO: 66 - protein, 13 aa, Artificial Sequence
  Description of Artificial Sequence Synthetic linker peptide
KKVEPKSSDKTHT

SEQ ID NO: 67 - protein, 11 aa, Artificial Sequence
  Description of Artificial Sequence Synthetic linker peptide
VEPKSSDKTHT

SEQ ID NO: 68 - protein, 12 aa, Artificial Sequence
  Description of Artificial Sequence Synthetic linker peptide
KVEPKSSDKTHT

SEQ ID NO: 69 - protein, 15 aa, Artificial Sequence
  Description of Artificial Sequence Synthetic linker peptide
VDKKVEPKSSDKTHT

SEQ ID NO: 70 - DNA, 1152 bp, Homo sapiens
  CDS (58)..(597)
cttcagaacaggttctccttccccagtcaccagttgctcgagttagaattgtctgcaatggccgccctgcagaaatctgtgagctctttccttatggggaccctggccaccagctgcctccttctcttggccctcttggtacagggaggagcagctgcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcatttgaccagagcaaagctgaaaaatgaataactaaccccctttccctgctagaaataacaattagatgccccaaagcgattttttttaaccaaaaggaagatgggaagccaaactccatcatgatgggtggattccaaatgaacccctgcgttagttacaaaggaaaccaatgccacttttgtttataagaccagaaggtagactttctaagcatagatatttattgataacatttcattgtaactggtgttctatacacagaaaacaatttattttttaaataattgtctttttccataaaaaagattactttccattcctttaggggaaaaaacccctaaatagcttcatgtttccataatcagtactttatatttataaatgtatttattattattataagactgcattttatttatatcattttattaatatggatttatttatagaaacatcattcgatattgctacttgagtgtaaggctaatattgatatttatgacaataattatagagctataacatgtttatttgacctcaataaacacttggatatccc

SEQ ID NO: 71 - protein, 179 aa, Homo sapiens
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 72 - DNA, 1236 bp, Artificial Sequence
  Synthetic polypeptide
atggctgtcctgcagaaatctatgagtttttcccttatggggactttggccgccagctgcctgcttctcattgccctgtgggcccaggaggcaaatgcgctgcccgtcaacacccggtgcaagcttgaggtgtccaacttccagcagccatacatcgtcaaccgcacctttatgctggccaaggaggccagccttgcagataacaacacagatgtccggctcatcggggagaaactgttccgaggagtcagtgctaaggatcagtgctacctgatgaagcaggtgctcaacttcaccctggaagacgttctgctcccccagtcagacaggttccagccctacatgcaggaggtggtgcctttcctgaccaaactcagcaatcagctcagctcctgtcacatcagcggtgacgaccagaacatccagaagaatgtcagaaggctgaaggagacagtgaaaaagcttggagagagtggagagatcaaggcgattggggaactggacctgctgtttatgtctctgagaaatgcttgcgtcgctcgaggacccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctacgcgtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 73 - protein, 411 aa, Artificial Sequence; identical to SEQ ID NO: 60
  Synthetic polypeptide

SEQ ID NO: 74 - DNA, 20 bp, Artificial Sequence
  Synthetic primer
aggtccattcagatgctggt

SEQ ID NO: 75 - DNA, 20 bp, Artificial Sequence
  Synthetic primer
taggtgtggttgacgtggag

SEQ ID NO: 76 - DNA, 20 bp, Artificial Sequence
  Synthetic primer
ccaccccacactcacaccgg

SEQ ID NO: 77 - protein, 177 aa, Homo sapiens [sequence omitted]

SEQ ID NO: 78 - protein, 176 aa, Homo sapiens
MKASSLAFSLLSAAFYLLWTPSTGLKTLNLGSCVIATNLQEIRNGFSEIRGSVQAKDGNIDIRILRRTESLQDTKPANRCCLLRHLLRLYLDRVFKNYQTPDHYTLRKISSLANSFLTIKKDLRLCHAHMTCHCGEEAMKKYSQILSHFEKLEPQAAVVKALGELDILLQWMEETE

SEQ ID NO: 79 - protein, 207 aa, Homo sapiens
MNFQQRLQSLWTLASRPFCPPLLATASQMQMVVLPCLGFTLLLWSQVSGAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 80 - protein, 171 aa, Homo sapiens
MLVNFILRCGLLLVTLSLAIAKHKQSSFTKSCYPRGTLSQAVDALYIKAAWLKATIPEDRIKNIRLLKKKTKKQFMKNCQFQEQLLSFFMEDVFGQLQLQGCKKIRFVEDFHSLRQKLSHCISCASSAREMKSITRMKRIFYRIGNKGIYKAISELDILLSWIKKLLESSQ

(19) United States
(12) Patent Application Publication
(10) Pub. No.: US 2021/0139552 A1
(43) Pub. Date: May 13, 2021

(54) DOSING FOR TREATMENT WITH IL-22 Fc FUSION PROTEINS

(71) Applicant: Genentech, Inc., South San Francisco, CA (US)

(72) Inventors: Matthew KALO, San Francisco, CA (US); Timothy Then-Chioh LU, San Francisco, CA (US); Siddharth SUKUMARAN, Millbrae, CA (US); Yehong WANG, Fremont, CA (US); Peter William DAY, Danville, CA (US)

(21) Appl. No.: 16/998,597

(22) Filed: Aug. 20, 2020

Related U.S. Application Data
(63) Continuation of application No. PCT/US2019/019042, filed on Feb. 21, 2019.
(60) Provisional application No. 62/633,534, filed on Feb. 21, 2018.

(51) Int. Cl. C07K 14/54; A61K 45/06; A61P 1/04

(52) U.S. Cl. CPC C07K 14/54; A61K 38/00; A61P 1/04; A61K 45/06

(57) ABSTRACT

The invention relates to methods, uses, and compositions (e.g., articles of manufacture and kits) for the treatment of diseases associated with IL-22 (e.g., inflammatory bowel disease (IBD) (e.g., ulcerative colitis (UC (e.g., moderate to severe UC)) and Crohn's disease (CD)) and graft versus host disease (GVHD) (e.g., acute or chronic GVHD)).

### SEQUENCE LISTING

The instant application contains a Sequence Listing which has been submitted electronically in ASCII format and is hereby incorporated by reference in its entirety. Said ASCII copy, created on Aug. 18, 2020, is named 50474-173002 Sequence Listing 8.18.20 ST25 and is 106,582 bytes in size.

### FIELD OF THE INVENTION

The present invention relates to methods of treating disorders such as inflammatory bowel disorder (IBD) and graft versus host disease (GVHD).

### BACKGROUND OF THE INVENTION

Interleukin (IL)-22 is a member of the IL-10 family of cytokines that is produced, e.g., by Th22 cells, NK cells, lymphoid tissue inducer (LTi) cells, dendritic cells, and Th17 cells. IL-22 binds to the IL-22R1/IL-10R2 receptor complex, which is expressed in innate cells (e.g., epithelial cells, hepatocytes, and keratinocytes) and in barrier epithelial tissues of several organs (e.g., dermis, pancreas, intestine, and the respiratory system).

IL-22 plays an important role in mucosal immunity, mediating early host defense against attaching and effacing bacterial pathogens. IL-22 promotes the production of anti-microbial peptides and pro-inflammatory cytokines from epithelial cells and stimulates proliferation and migration of colonic epithelial cells in the gut. Upon bacterial infection, IL-22 knock-out mice displayed impaired gut epithelial regeneration, high bacterial load, and increased mortality. Similarly, infection of IL-22 knock-out mice with influenza virus resulted in severe weight loss and impaired regeneration of tracheal and bronchial epithelial cells. Thus, IL-22 plays a pro-inflammatory role in suppressing microbial infection as well as an anti-inflammatory protective role in epithelial regeneration in inflammatory responses.

There remains a need for improved methods for treatment of IBD, including ulcerative colitis (UC) and Crohn's disease (CD), as well as other disorders associated with IL-22, such as GVHD. For example, acute graft versus host disease (aGVHD) is a common and life-threatening complication of allogeneic hematopoietic stem cell transplantation (allo-HSCT) with a high unmet need for effective, non-immunosuppressive therapies for prevention and treatment.

### SUMMARY OF THE INVENTION

The present invention provides, inter alia, methods of treating IL-22 associated diseases such as IBD (e.g., UC (e.g., moderate to severe UC) and Crohn's disease), GVHD, hidradenitis suppurativa, chronic obstructive pulmonary disease (COPD), and nonalcoholic fatty acid liver disease (e.g., nonalcoholic steatohepatitis (NASH)), as well as related compositions, uses, and kits.

In one aspect, the invention features a method of treating a subject having an inflammatory bowel disease (IBD) comprising administering to the subject an IL-22 Fc fusion protein in a dosing regimen comprising at least a first dosing cycle, wherein the first dosing cycle comprises between two and ten doses, and wherein a total of about 60 μg/kg to about 900 μg/kg of the IL-22 Fc fusion protein is administered to the subject in the first dosing cycle. In some embodiments, the doses are administered to the subject every week (q1w), every two weeks (q2w), every four weeks (q4w), or every six weeks (q6w). In some embodiments, a total of about 90 μg/kg, about 180 μg/kg, about 270 μg/kg, about 360 μg/kg, or about 540 μg/kg of the IL-22 Fc fusion protein is administered to the subject in the first dosing cycle. In some embodiments, the length of the first dosing cycle is between about 5 weeks and about 15 weeks. In some embodiments, the length of the first dosing cycle is about 8 weeks. In some embodiments, the first dosing cycle comprises a first dose (C1D1), a second dose (C1D2), and a third dose (C1D3) of the IL-22 Fc fusion protein. In some embodiments, the first dosing cycle consists of the C1D1, the C1D2, and the C1D3. In some embodiments, the C1D1, the C1D2, and the C1D3 are each between about 15 μg/kg to about 90 μg/kg. In some embodiments, the C1D1, the C1D2, and the C1D3 are each about 30 μg/kg. In some embodiments, the C1D1, the C1D2, and the C1D3 are each about 60 μg/kg. In some embodiments, the method comprises administering the C1D1, the C1D2, and the C1D3 on or about Weeks 1, 4, and 8, respectively, of the first dosing cycle.

In another aspect, the invention features a method of treating a subject having an inflammatory bowel disease (IBD) comprising administering to the subject an IL-22 Fc fusion protein in a dosing regimen comprising a dosing cycle, wherein the dosing cycle comprises between two and six doses, and wherein a total of about 30 μg/kg to about 720 μg/kg of the IL-22 Fc fusion protein is administered to the subject in the dosing cycle. In some embodiments, the doses are administered to the subject every week (q1w), every two weeks (q2w), every four weeks (q4w), or every six weeks (q6w). In some embodiments, a total of about 540 μg/kg of the IL-22 Fc fusion protein is administered to the subject in the dosing cycle. In some embodiments, a total of about 360 μg/kg of the IL-22 Fc fusion protein is administered to the subject in the dosing cycle. In some embodiments, a total of about 270 μg/kg of the IL-22 Fc fusion protein is administered to the subject in the dosing cycle. In some embodiments, a total of about 180 μg/kg of the IL-22 Fc fusion protein is administered to the subject in the dosing cycle. In some embodiments, a total of about 90 μg/kg of the IL-22 Fc fusion protein is administered to the subject in the dosing cycle.

In another aspect, the invention features an IL-22 Fc fusion protein for use in a method of treating a subject having an IBD, wherein the IL-22 Fc fusion protein is for administration to the subject in a dosing regimen comprising at least a first dosing cycle, wherein the first dosing cycle comprises between two and ten doses, and wherein a total of about 60 μg/kg to about 900 μg/kg of the IL-22 Fc fusion protein is administered to the subject in the first dosing cycle. In some embodiments, the doses are to be administered to the subject every week (q1w), every two weeks (q2w), every four weeks (q4w), or every six weeks (q6w). In some embodiments, a total of about 90 μg/kg, about 180 μg/kg, about 270 μg/kg, about 360 μg/kg, or about 540 μg/kg of the IL-22 Fc fusion protein is to be administered to the subject in the first dosing cycle. In some embodiments, the length of the first dosing cycle is between about 5 weeks and about 15 weeks. In some embodiments, the length of the first dosing cycle is about 8 weeks.

In another aspect, the invention features the use of an IL-22 Fc fusion protein in the preparation of a medicament for use in a method of treating a subject having an IBD, wherein the IL-22 Fc fusion protein is for administration to the subject in a dosing regimen comprising at least a first dosing cycle, wherein the first dosing cycle comprises between two and ten doses, and wherein a total of about 60 μg/kg to about 900 μg/kg of the IL-22 Fc fusion protein is administered to the subject in the first dosing cycle. In some embodiments, the doses are to be administered to the subject every week (q1w), every two weeks (q2w), every four weeks (q4w), or every six weeks (q6w). In some embodiments, a total of about 90 μg/kg, about 180 μg/kg, about 270 μg/kg, about 360 μg/kg, or about 540 μg/kg of the IL-22 Fc fusion protein is to be administered to the subject in the first dosing cycle. In some embodiments, the length of the first dosing cycle is between about 5 weeks and about 15 weeks. In some embodiments, the length of the first dosing cycle is about 8 weeks.

In another aspect, the invention features an IL-22 Fc fusion protein for use in a method of treating a subject having an IBD, wherein the IL-22 Fc fusion protein is for administration to the subject in a dosing regimen comprising a dosing cycle, wherein the dosing cycle comprises between two and six doses, and wherein a total of about 30 μg/kg to about 720 μg/kg of the IL-22 Fc fusion protein is administered to the subject in the dosing cycle. In some embodiments, the doses are to be administered to the subject every week (q1w), every two weeks (q2w), every four weeks (q4w), or every six weeks (q6w). In some embodiments, a total of about 540 μg/kg of the IL-22 Fc fusion protein is to be administered to the subject in the dosing cycle. In some embodiments, a total of about 360 μg/kg of the IL-22 Fc fusion protein is to be administered to the subject in the dosing cycle. In some embodiments, a total of about 270 μg/kg of the IL-22 Fc fusion protein is to be administered to the subject in the dosing cycle. In some embodiments, a total of about 180 μg/kg of the IL-22 Fc fusion protein is to be administered to the subject in the dosing cycle. In some embodiments, a total of about 90 μg/kg of the IL-22 Fc fusion protein is to be administered to the subject in the dosing cycle.

In some embodiments of any of the preceding aspects, the length of the dosing cycle (e.g., the first dosing cycle) is between about 5 weeks and about 15 weeks. In some embodiments, the length of the dosing cycle (e.g., the first dosing cycle) is between 8 weeks and 12 weeks. In some embodiments, the length of the dosing cycle (e.g., the first dosing cycle) is about 8 weeks. In some embodiments, the length of the dosing cycle (e.g., the first dosing cycle) is about 10 weeks. In some embodiments, the length of the dosing cycle (e.g., the first dosing cycle) is up to about 10 weeks.

In some embodiments of any of the preceding aspects, the dosing cycle (e.g., the first dosing cycle) comprises a first dose (C1D1) and a second dose (C1D2) of the IL-22 Fc fusion protein. In some embodiments, the dosing cycle consists of the C1D1 and the C1D2. In some embodiments, the C1D1 and the C1D2 are each between about 30 μg/kg to about 135 μg/kg. In some embodiments, the C1D1 and the C1D2 are each about 90 μg/kg. In some embodiments, the method comprises administering to the subject the C1D1 and the C1D2 on or about Weeks 0 and 6, respectively, of the dosing cycle.

In some embodiments of any of the preceding aspects, the dosing cycle (e.g., the first dosing cycle) comprises a first dose (C1D1), a second dose (C1D2), and a third dose (C1D3) of the IL-22 Fc fusion protein. In some embodiments, the dosing cycle (e.g., the first dosing cycle) consists of the C1D1, the C1D2, and the C1D3. In some embodiments, the C1D1, the C1D2, and the C1D3 are each between about 15 μg/kg to about 90 μg/kg. In some embodiments, the C1D1, the C1D2, and the C1D3 are each between about 20 μg/kg to about 40 μg/kg. In some embodiments, the C1D1, the C1D2, and the C1D3 are each about 30 μg/kg. In some embodiments, the C1D1, the C1D2, and the C1D3 are each between about 50 μg/kg to about 70 μg/kg. In some embodiments, the C1D1, the C1D2, and the C1D3 are each about 60 μg/kg. In some embodiments, the C1D1, the C1D2, and the C1D3 are each about 90 μg/kg. In some embodiments, the method comprises administering the C1D1, the C1D2, and the C1D3 on or about Weeks 0, 4, and 8, respectively, of the dosing cycle (e.g., the first dosing cycle). In other embodiments, the method comprises administering the C1D1, the C1D2, and the C1D3 on or about Weeks 1, 4, and 8, respectively, of the dosing cycle (e.g., the first dosing cycle). In some embodiments, the C1D1 is administered on Day 0 of Week 1.

In some embodiments of any of the preceding aspects, the dosing cycle (e.g., the first dosing cycle) comprises a first dose (C1D1), a second dose (C1D2), a third dose (C1D3), a fourth dose (C1D4), a fifth dose (C1D5), and a sixth dose (C1D6) of the IL-22 Fc fusion protein. In some embodiments, the dosing cycle consists of the C1D1, the C1D2, the C1D3, the C1D4, the C1D5, and the C1D6. In some embodiments, the C1D1, the C1D2, the C1D3, the C1D4, the C1D5, and the C1D6 are each between about 30 μg/kg to about 90 μg/kg. In some embodiments, the C1D1, the C1D2, the C1D3, the C1D4, the C1D5, and the C1D6 are each between about 50 μg/kg to about 70 μg/kg. In some embodiments, the C1D1, the C1D2, the C1D3, the C1D4, the C1D5, and the C1D6 are each about 60 μg/kg. In some embodiments, the method comprises administering the C1D1, the C1D2, the C1D3, the C1D4, the C1D5, and the C1D6 on or about Weeks 0, 2, 4, 6, 8, and 10, respectively, of the dosing cycle.

In some embodiments of any of the preceding aspects, the dosing regimen further comprises a further (e.g., a second) dosing cycle. In some embodiments, the length of the further (e.g., second) dosing cycle is between about 10 weeks and about 40 weeks. In some embodiments, the length of the further (e.g., second) dosing cycle is between about 15 weeks and about 25 weeks. In some embodiments, the length of the further (e.g., second) dosing cycle is about 20 weeks. In some embodiments, the further (e.g., second) dosing cycle continues indefinitely or until clinical remission. In some embodiments, the further (e.g., second) dosing cycle is stopped following the clinical remission, and then restarted following a relapse of the IBD.

In some embodiments of any of the preceding aspects, the doses of the further (e.g., second) dosing cycle are administered to the subject every week (q1w), every two weeks (q2w), every four weeks (q4w), every six weeks (q6w), every eight weeks (q8w), every ten weeks (q10w), or every twelve weeks (q12w). In some embodiments, the doses of the further (e.g., second) dosing cycle are administered to the subject every eight weeks (q8w). In some embodiments, each dose of the further (e.g., second) dosing cycle is between about 30 μg/kg to about 90 μg/kg. In some embodiments, each dose of the further (e.g., second) dosing cycle is about 60 μg/kg.

In some embodiments of any of the preceding aspects, the further (e.g., second) dosing cycle comprises a first dose (C2D1), a second dose (C2D2), and a third dose (C2D3) of the IL-22 Fc fusion protein. In some embodiments, the C2D1, the C2D2, and the C2D3 are each between about 30 μg/kg to about 90 μg/kg. In some embodiments, the C2D1, the C2D2, and the C2D3 are each between about 50 μg/kg to about 70 μg/kg. In some embodiments, the C2D1, the C2D2, and the C2D3 are each about 60 μg/kg. In some embodiments, the method comprises administering the C2D1, the C2D2, and the C2D3 on or about Weeks 4, 12, and 20, respectively, of the further (e.g., second) dosing cycle.

In some embodiments of any of the preceding aspects, the first dose of the second dosing cycle is administered to the subject about 6 weeks to about 10 weeks after the last dose of the first dosing cycle.

In another aspect, the invention features a method of treating a subject having an IBD comprising a dosing regimen, the dosing regimen comprising administering to the subject an IL-22 Fc fusion protein every four weeks (q4w) until the subject has a clinical remission of the IBD. In some embodiments, each dose of the dosing regimen is between about 15 μg/kg to about 90 μg/kg. In some embodiments, each dose of the dosing regimen is about 30 μg/kg, about 60 μg/kg, or about 90 μg/kg. In some embodiments, each dose of the dosing regimen is about 30 μg/kg. In some embodiments, each dose of the dosing regimen is about 60 μg/kg. In some embodiments, each dose of the dosing regimen is about 90 μg/kg.

In another aspect, the invention features an IL-22 Fc fusion protein for use in a method of treating a subject having an IBD, wherein the IL-22 Fc fusion protein is for administration to the subject in a dosing regimen, the dosing regimen comprising administering to the subject an IL-22 Fc fusion protein every four weeks (q4w) until the subject has a clinical remission of the IBD. In some embodiments, each dose of the dosing regimen is between about 15 μg/kg to about 90 μg/kg. In some embodiments, each dose of the dosing regimen is about 30 μg/kg, about 60 μg/kg, or about 90 μg/kg. In some embodiments, each dose of the dosing regimen is about 30 μg/kg. In some embodiments, each dose of the dosing regimen is about 60 μg/kg. In some embodiments, each dose of the dosing regimen is about 90 μg/kg.

In another aspect, the invention features an IL-22 Fc fusion protein for use in the preparation of a medicament for use in a method of treating a subject having an IBD, wherein the IL-22 Fc fusion protein is for administration to the subject in a dosing regimen, the dosing regimen comprising administering to the subject an IL-22 Fc fusion protein every four weeks (q4w) until the subject has a clinical remission of the IBD. In some embodiments, each dose of the dosing regimen is between about 15 μg/kg to about 90 μg/kg. In some embodiments, each dose of the dosing regimen is about 30 μg/kg, about 60 μg/kg, or about 90 μg/kg. In some embodiments, each dose of the dosing regimen is about 30 μg/kg. In some embodiments, each dose of the dosing regimen is about 60 μg/kg. In some embodiments, each dose of the dosing regimen is about 90 μg/kg.

In another aspect, the invention features a method of treating a subject having an IBD comprising administering to the subject an IL-22 Fc fusion protein in a dosing regimen comprising a dosing cycle having a length of about 8 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 30 μg/kg, about 60 μg/kg, or about 90 μg/kg, and wherein the C1D1, the C1D2, and the C1D3 are administered to the subject on or about Weeks 1, 4, and 8, respectively, of the dosing cycle. In some embodiments, the C1D1 is administered on Day 0 of Week 1.

In another aspect, the invention features a method of treating a subject having an IBD comprising administering to the subject an IL-22 Fc fusion protein in a dosage regimen comprising at least a first dosing cycle and a second dosing cycle, wherein: (a) the first dosing cycle has a length of about 8 weeks and comprises a first dose (C1D1), a second dose (C1D2), and a third dose (C1D3) of the IL-22 Fc fusion protein, wherein the C1D1, the C1D2, and the C1D3 are each about 30 μg/kg, about 60 μg/kg, or about 90 μg/kg, and wherein the C1D1, the C1D2, and the C1D2 are administered to the subject on or about Weeks 1, 4, and 8, respectively, of the first dosing cycle; and (b) the second dosing cycle continues indefinitely or until clinical remission, and comprises administering about 60 μg/kg of the IL-22 Fc fusion protein to the subject every 8 weeks (q8w), wherein the first dose of the second dosing cycle is administered to the subject about 8 weeks after the last dose of the first dosing cycle. In some embodiments, the C1D1 is administered on Day 0 of Week 1.

In another aspect, the invention features a method of treating a subject having an IBD comprising administering to the subject an IL-22 Fc fusion protein in a dosing regimen comprising a dosing cycle having a length of about 10 weeks, wherein the dosing cycle comprises a first dose (C1D1) and a second dose (C1D2) of the IL-22 Fc fusion protein, wherein the C1D1 and the C1D2 are each about 90 μg/kg, and wherein the C1D1 and C1D2 are administered to the subject on or about Weeks 0 and 6, respectively, of the dosing cycle.

In another aspect, the invention features a method of treating a subject having an IBD comprising administering to the subject an IL-22 Fc fusion protein in a dosing regimen comprising a dosing cycle having a length of about 10 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 30 μg/kg, and wherein the C1D1, the C1D2, and the C1D3 are administered to the subject on or about Weeks 0, 4, and 8, respectively, of the dosing cycle.

In another aspect, the invention features a method of treating a subject having an IBD comprising administering to the subject an IL-22 Fc fusion protein in a dosing regimen comprising a dosing cycle having a length of about eight weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 90 μg/kg, and wherein the C1D1, the C1D2, and the C1D3 are administered to the subject on or about Weeks 0, 4, and 8, respectively, of the dosing cycle.

In another aspect, the invention features a method of treating a subject having an IBD comprising administering to the subject an IL-22 Fc fusion protein in a dosing regimen comprising a dosing cycle having a length of about 10 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 60 μg/kg, and wherein the C1D1, the C1D2, and the C1D3 are administered to the subject on or about Weeks 0, 4, and 8, respectively, of the dosing cycle.

In another aspect, the invention features a method of treating a subject having an IBD comprising administering to the subject an IL-22 Fc fusion protein in a dosing regimen comprising a dosing cycle having a length of about 10 weeks, wherein the dosing cycle comprises a first dose (C1D1), a second dose (C1D2), a third dose (C1D3), a fourth dose (C1D4), a fifth dose (C1D5), and a sixth dose (C1D6) of the IL-22 Fc fusion protein, wherein the C1D1, C1D2, the C1D3, the C1D4, the C1D5, and the C1D6 are each about 60 μg/kg, and wherein the C1D1, the C1D2, the C1D3, the C1D4, the C1D5, and the C1D6 are administered to the subject on or about Weeks 0, 2, 4, 6, 8, and 10, respectively, of the dosing cycle.

In another aspect, the invention features a method of treating a subject having an IBD comprising administering to the subject an IL-22 Fc fusion protein in a dosage regimen comprising at least a first dosing cycle and a second dosing cycle, wherein: (a) the first dosing cycle has a length of about 10 weeks and comprises a first dose (C1D1) and a second dose (C1D2) of the IL-22 Fc fusion protein, wherein the C1D1 and the C1D2 are each about 90 μg/kg, and wherein the C1D1 and the C1D2 are administered to the subject on or about Weeks 0 and 6, respectively, of the first dosing cycle; and (b) the second dosing cycle has a length of about 20 weeks and comprises a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are administered on or about Weeks 4, 12, and 20, respectively, of the second dosing cycle.

In another aspect, the invention features a method of treating a subject having an IBD comprising administering to the subject an IL-22 Fc fusion protein in a dosage regimen comprising at least a first dosing cycle and a second dosing cycle, wherein: (a) the first dosing cycle has a length of about 8 weeks and comprises a first dose (C1D1), a second dose (C1D2), and a third dose (C1D3) of the IL-22 Fc fusion protein, wherein the C1D1, the C1D2, and the C1D3 are each about 90 μg/kg, and wherein the C1D1, the C1D2, and the C1D2 are administered to the subject on or about Weeks 0, 4, and 8, respectively, of the first dosing cycle; and (b) the second dosing cycle has a length of about 20 weeks and comprises a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are administered on or about Weeks 4, 12, and 20, respectively, of the second dosing cycle.

In another aspect, the invention features a method of treating a subject having an IBD comprising administering to the subject an IL-22 Fc fusion protein in a dosage regimen comprising at least a first dosing cycle and a second dosing cycle, wherein: (a) the first dosing cycle has a length of about 10 weeks and comprises a first dose (C1D1), a second dose (C1D2), and a third dose (C1D3) of the IL-22 Fc fusion protein, wherein the C1D1, the C1D2, and the C1D3 are each about 30 μg/kg, and wherein the C1D1, the C1D2, and the C1D2 are administered to the subject on or about Weeks 0, 4, and 8, respectively, of the first dosing cycle; and (b) the second dosing cycle has a length of about 20 weeks and comprises a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are administered on or about Weeks 4, 12, and 20, respectively, of the second dosing cycle.

In another aspect, the invention features a method of treating a subject having an IBD comprising administering to the subject an IL-22 Fc fusion protein in a dosage regimen comprising at least a first dosing cycle and a second dosing cycle, wherein: (a) the first dosing cycle has a length of about 10 weeks and comprises a first dose (C1D1), a second dose (C1D2), and a third dose (C1D3) of the IL-22 Fc fusion protein, wherein the C1D1, the C1D2, and the C1D3 are each about 60 μg/kg, and wherein the C1D1, the C1D2, and the C1D2 are administered to the subject on or about Weeks 0, 4, and 8, respectively, of the first dosing cycle; and (b) the second dosing cycle has a length of about 20 weeks and comprises a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are administered on or about Weeks 4, 12, and 20, respectively, of the second dosing cycle.

In another aspect, the invention features a method of treating a subject having an IBD comprising administering to the subject an IL-22 Fc fusion protein in a dosage regimen comprising at least a first dosing cycle and a second dosing cycle, wherein: (a) the first dosing cycle has a length of about 10 weeks and comprises a first dose (C1D1), a second dose (C1D2), a third dose (C1D3), a fourth dose (C1D4), a fifth dose (C1D5), and a sixth dose (C1D6) of the IL-22 Fc fusion protein, wherein the C1D1, the C1D2, the C1D3, the C1D4, the C1D5, and the C1D6 are each about 60 μg/kg, and wherein the C1D1, the C1D2, the C1D3, the C1D4, the C1D5, and the C1D6 are administered to the subject on or about Weeks 0, 2, 4, 6, 8, and 10, respectively, of the dosing cycle; and (b) the second dosing cycle has a length of about 20 weeks and comprises a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are administered on or about Weeks 4, 12, and 20, respectively, of the second dosing cycle.

In another aspect, the invention features an IL-22 Fc fusion protein for use in a method of treating a subject having an IBD, wherein the IL-22 Fc fusion protein is for administration to the subject in a dosing regimen comprising a dosing cycle having a length of about 8 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 30 μg/kg, about 60 μg/kg, or about 90 μg/kg, and wherein the C1D1, the C1D2, and the C1D3 are administered to the subject on or about Weeks 1, 4, and 8, respectively, of the dosing cycle. In some embodiments, the C1D1 is administered on Day 0 of Week 1.

In another aspect, the invention features an IL-22 Fc fusion protein for use in a method of treating a subject having an IBD, wherein the IL-22 Fc fusion protein is for administration to the subject in a dosing regimen comprising at least a first dosing cycle and a second dosing cycle, wherein: (a) the first dosing cycle has a length of about 8 weeks and comprises a first dose (C1D1), a second dose (C1D2), and a third dose (C1D3) of the IL-22 Fc fusion protein, wherein the C1D1, the C1D2, and the C1D3 are each about 30 μg/kg, about 60 μg/kg, or about 90 μg/kg, and wherein the C1D1, the C1D2, and the C1D2 are administered to the subject on or about Weeks 1, 4, and 8, respectively, of the first dosing cycle; and (b) the second dosing cycle continues indefinitely or until clinical remission, and comprises administering about 60 μg/kg of the IL-22 Fc fusion protein to the subject every 8 weeks (q8w), wherein the first dose of the second dosing cycle is administered to the subject about 8 weeks after the last dose of the first dosing cycle. In some embodiments, the C1D1 is administered on Day 0 of Week 1.

In another aspect, the invention features an IL-22 Fc fusion protein for use in a method of treating a subject having an IBD, wherein the IL-22 Fc fusion protein is for administration to the subject in a dosing regimen comprising a dosing cycle having a length of about 10 weeks, wherein the dosing cycle comprises a first dose (C1D1) and a second dose (C1D2) of the IL-22 Fc fusion protein, wherein the C1D1 and the C1D2 are each about 90 μg/kg, and wherein the C1D1 and C1D2 are to be administered to the subject on or about Weeks 0 and 6, respectively, of the dosing cycle.

In another aspect, the invention features an IL-22 Fc fusion protein for use in a method of treating a subject having an IBD, wherein the IL-22 Fc fusion protein is for administration to the subject in a dosing regimen comprising a dosing cycle having a length of about 10 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 30 μg/kg, and wherein the C1D1, the C1D2, and the C1D3 are to be administered to the subject on or about Weeks 0, 4, and 8, respectively, of the dosing cycle.

In another aspect, the invention features an IL-22 Fc fusion protein for use in a method of treating a subject having an IBD, wherein the IL-22 Fc fusion protein is for administration to the subject in a dosing regimen comprising a dosing cycle having a length of about 10 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 60 μg/kg, and wherein the C1D1, the C1D2, and the C1D3 are to be administered to the subject on or about Weeks 0, 4, and 8, respectively, of the dosing cycle.

In another aspect, the invention features an IL-22 Fc fusion protein for use in a method of treating a subject having an IBD, wherein the IL-22 Fc fusion protein is for administration to the subject in a dosing regimen comprising a dosing cycle having a length of about eight weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 90 μg/kg, and wherein the C1D1, the C1D2, and the C1D3 are to be administered to the subject on or about Weeks 0, 4, and 8, respectively, of the dosing cycle.

In another aspect, the invention features an IL-22 Fc fusion protein for use in a method of treating a subject having an IBD, wherein the IL-22 Fc fusion protein is for administration to the subject in a dosing regimen comprising a dosing cycle having a length of about 10 weeks, wherein the dosing cycle comprises a first dose (C1D1), a second dose (C1D2), a third dose (C1D3), a fourth dose (C1D4), a fifth dose (C1D5), and a sixth dose (C1D6) of the IL-22 Fc fusion protein, wherein the C1D1, C1D2, the C1D3, the C1D4, the C1D5, and the C1D6 are each about 60 μg/kg, and wherein the C1D1, the C1D2, the C1D3, the C1D4, the C1D5, and the C1D6 are to be administered to the subject on or about Weeks 0, 2, 4, 6, 8, and 10, respectively, of the dosing cycle.

In another aspect, the invention features an IL-22 Fc fusion protein for use in a method of treating a subject having an IBD, wherein the IL-22 Fc fusion protein is for administration to the subject in a dosing regimen comprising at least a first dosing cycle and a second dosing cycle, wherein: (a) the first dosing cycle has a length of about 10 weeks and comprises a first dose (C1D1) and a second dose (C1D2) of the IL-22 Fc fusion protein, wherein the C1D1 and the C1D2 are each about 90 μg/kg, and wherein the C1D1 and the C1D2 are to be administered to the subject on or about Weeks 0 and 6, respectively, of the first dosing cycle; and (b) the second dosing cycle has a length of about 20 weeks and comprises a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are to be administered on or about Weeks 4, 12, and 20, respectively, of the second dosing cycle.

In another aspect, the invention features an IL-22 Fc fusion protein for use in a method of treating a subject having an IBD, wherein the IL-22 Fc fusion protein is for administration to the subject in a dosing regimen comprising at least a first dosing cycle and a second dosing cycle, wherein: (a) the first dosing cycle has a length of about 10 weeks and comprises a first dose (C1D1), a second dose (C1D2), and a third dose (C1D3) of the IL-22 Fc fusion protein, wherein the C1D1, the C1D2, and the C1D3 are each about 30 μg/kg, and wherein the C1D1, the C1D2, and the C1D2 are to be administered to the subject on or about Weeks 0, 4, and 8, respectively, of the first dosing cycle; and (b) the second dosing cycle has a length of about 20 weeks and comprises a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are to be administered on or about Weeks 4, 12, and 20, respectively, of the second dosing cycle.

In another aspect, the invention features an IL-22 Fc fusion protein for use in a method of treating a subject having an IBD, wherein the IL-22 Fc fusion protein is for administration to the subject in a dosing regimen comprising at least a first dosing cycle and a second dosing cycle, wherein: (a) the first dosing cycle has a length of about 8 weeks and comprises a first dose (C1D1), a second dose (C1D2), and a third dose (C1D3) of the IL-22 Fc fusion protein, wherein the C1D1, the C1D2, and the C1D3 are each about 90 μg/kg, and wherein the C1D1, the C1D2, and the C1D2 are to be administered to the subject on or about Weeks 0, 4, and 8, respectively, of the first dosing cycle; and (b) the second dosing cycle has a length of about 20 weeks and comprises a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are to be administered on or about Weeks 4, 12, and 20, respectively, of the second dosing cycle.

In another aspect, the invention features an IL-22 Fc fusion protein for use in a method of treating a subject having an IBD, wherein the IL-22 Fc fusion protein is for administration to the subject in a dosing regimen comprising at least a first dosing cycle and a second dosing cycle, wherein: (a) the first dosing cycle has a length of about 10 weeks and comprises a first dose (C1D1), a second dose (C1D2), and a third dose (C1D3) of the IL-22 Fc fusion protein, wherein the C1D1, the C1D2, and the C1D3 are each about 60 μg/kg, and wherein the C1D1, the C1D2, and the C1D2 are to be administered to the subject on or about Weeks 0, 4, and 8, respectively, of the first dosing cycle; and (b) the second dosing cycle has a length of about 20 weeks and comprises a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are to be administered on or about Weeks 4, 12, and 20, respectively, of the second dosing cycle.

In another aspect, the invention features an IL-22 Fc fusion protein for use in a method of treating a subject having an IBD, wherein the IL-22 Fc fusion protein is for administration to the subject in a dosing regimen comprising at least a first dosing cycle and a second dosing cycle, wherein: (a) the first dosing cycle has a length of about 10 weeks and comprises a first dose (C1D1), a second dose (C1D2), a third dose (C1D3), a fourth dose (C1D4), a fifth dose (C1D5), and a sixth dose (C1D6) of the IL-22 Fc fusion protein, wherein the C1D1, the C1D2, the C1D3, the C1D4, the C1D5, and the C1D6 are each about 60 μg/kg, and wherein the C1D1, the C1D2, the C1D3, the C1D4, the C1D5, and the C1D6 are to be administered to the subject on or about Weeks 0, 2, 4, 6, 8, and 10, respectively, of the dosing cycle; and (b) the second dosing cycle has a length of about 20 weeks and comprises a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are to be administered on or about Weeks 4, 12, and 20, respectively, of the second dosing cycle.

In another aspect, the invention features the use of an IL-22 Fc fusion protein in the preparation of a medicament for use in a method of treating a subject having an IBD, wherein the IL-22 Fc fusion protein is for administration to the subject in a dosing regimen comprising a dosing cycle having a length of about 8 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 30 μg/kg, about 60 μg/kg, or about 90 μg/kg, and wherein the C1D1, the C1D2, and the C1D3 are administered to the subject on or about Weeks 1, 4, and 8, respectively, of the dosing cycle. In some embodiments, the C1D1 is administered on Day 0 of Week 1.

In another aspect, the invention features the use of an IL-22 Fc fusion protein in the preparation of a medicament for use in a method of treating a subject having an IBD, wherein the IL-22 Fc fusion protein is for administration to the subject in a dosing regimen comprising at least a first dosing cycle and a second dosing cycle, wherein: (a) the first dosing cycle has a length of about 8 weeks and comprises a first dose (C1D1), a second dose (C1D2), and a third dose (C1D3) of the IL-22 Fc fusion protein, wherein the C1D1, the C1D2, and the C1D3 are each about 30 μg/kg, about 60 μg/kg, or about 90 μg/kg, and wherein the C1D1, the C1D2, and the C1D2 are administered to the subject on or about Weeks 1, 4, and 8, respectively, of the first dosing cycle; and (b) the second dosing cycle continues indefinitely or until clinical remission, and comprises administering about 60 μg/kg of the IL-22 Fc fusion protein to the subject every 8 weeks (q8w), wherein the first dose of the second dosing cycle is administered to the subject about 8 weeks after the last dose of the first dosing cycle. In some embodiments, the C1D1 is administered on Day 0 of Week 1.

In another aspect, the invention features the use of an IL-22 Fc fusion protein in the preparation of a medicament for use in a method of treating a subject having an IBD, wherein the IL-22 Fc fusion protein is for administration to the subject in a dosing regimen comprising a dosing cycle having a length of about 10 weeks, wherein the dosing cycle comprises a first dose (C1D1) and a second dose (C1D2) of the IL-22 Fc fusion protein, wherein the C1D1 and the C1D2 are each about 90 μg/kg, and wherein the C1D1 and C1D2 are to be administered to the subject on or about Weeks 0 and 6, respectively, of the dosing cycle.

In another aspect, the invention features the use of an IL-22 Fc fusion protein in the preparation of a medicament for use in a method of treating a subject having an IBD, wherein the IL-22 Fc fusion protein is for administration to the subject in a dosage regimen comprising a dosing cycle having a length of about 8 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 30 μg/kg, about 60 μg/kg, or about 90 μg/kg, and wherein the C1D1, the C1D2, and the C1D3 are administered to the subject on or about Weeks 0, 4, and 8, respectively, of the dosing cycle.

In another aspect, the invention features the use of an IL-22 Fc fusion protein in the preparation of a medicament for use in a method of treating a subject having an IBD, wherein the IL-22 Fc fusion protein is for administration to the subject in a dosing regimen comprising a dosing cycle having a length of about 10 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 30 μg/kg, and wherein the C1D1, the C1D2, and the C1D3 are to be administered to the subject on or about Weeks 0, 4, and 8, respectively, of the dosing cycle.

In another aspect, the invention features the use of an IL-22 Fc fusion protein in the preparation of a medicament for use in a method of treating a subject having an IBD, wherein the IL-22 Fc fusion protein is for administration to the subject in a dosing regimen comprising a dosing cycle having a length of about 10 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 60 μg/kg, and wherein the C1D1, the C1D2, and the C1D3 are to be administered to the subject on or about Weeks 0, 4, and 8, respectively, of the dosing cycle.

In another aspect, the invention features the use of an IL-22 Fc fusion protein in the preparation of a medicament for use in a method of treating a subject having an IBD, wherein the IL-22 Fc fusion protein is for administration to the subject in a dosing regimen comprising a dosing cycle having a length of about eight weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 90 μg/kg, and wherein the C1D1, the C1D2, and the C1D3 are to be administered to the subject on or about Weeks 0, 4, and 8, respectively, of the dosing cycle.

In another aspect, the invention features the use of an IL-22 Fc fusion protein in the preparation of a medicament for use in a method of treating a subject having an IBD, wherein the IL-22 Fc fusion protein is for administration to the subject in a dosing regimen comprising a dosing cycle having a length of about 10 weeks, wherein the dosing cycle comprises a first dose (C1D1), a second dose (C1D2), a third dose (C1D3), a fourth dose (C1D4), a fifth dose (C1D5), and a sixth dose (C1D6) of the IL-22 Fc fusion protein, wherein the C1D1, C1D2, the C1D3, the C1D4, the C1D5, and the C1D6 are each about 60 μg/kg, and wherein the C1D1, the C1D2, the C1D3, the C1D4, the C1D5, and the C1D6 are to be administered to the subject on or about Weeks 0, 2, 4, 6, 8, and 10, respectively, of the dosing cycle.

In another aspect, the invention features the use of an IL-22 Fc fusion protein in the preparation of a medicament for use in a method of treating a subject having an IBD, wherein the IL-22 Fc fusion protein is for administration to the subject in a dosing regimen comprising at least a first dosing cycle and a second dosing cycle, wherein: (a) the first dosing cycle has a length of about 10 weeks and comprises a first dose (C1D1) and a second dose (C1D2) of the IL-22 Fc fusion protein, wherein the C1D1 and the C1D2 are each about 90 μg/kg, and wherein the C1D1 and the C1D2 are to be administered to the subject on or about Weeks 0 and 6, respectively, of the first dosing cycle; and (b) the second dosing cycle has a length of about 20 weeks and comprises a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are to be administered on or about Weeks 4, 12, and 20, respectively, of the second dosing cycle.

In another aspect, the invention features the use of an IL-22 Fc fusion protein in the preparation of a medicament for use in a method of treating a subject having an IBD, wherein the IL-22 Fc fusion protein is formulated for administration to the subject in a dosing regimen comprising at least a first dosing cycle and a second dosing cycle, wherein: (a) the first dosing cycle has a length of about 10 weeks and comprises a first dose (C1D1), a second dose (C1D2), and a third dose (C1D3) of the IL-22 Fc fusion protein, wherein the C1D1, the C1D2, and the C1D3 are each about 30 μg/kg, and wherein the C1D1, the C1D2, and the C1D2 are to be administered to the subject on or about Weeks 0, 4, and 8, respectively, of the first dosing cycle; and (b) the second dosing cycle has a length of about 20 weeks and comprises a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are to be administered on or about Weeks 4, 12, and 20, respectively, of the second dosing cycle.

In another aspect, the invention features the use of an IL-22 Fc fusion protein in the preparation of a medicament for use in a method of treating a subject having an IBD, wherein the IL-22 Fc fusion protein is for administration to the subject in a dosing regimen comprising at least a first dosing cycle and a second dosing cycle, wherein: (a) the first dosing cycle has a length of about 8 weeks and comprises a first dose (C1D1), a second dose (C1D2), and a third dose (C1D3) of the IL-22 Fc fusion protein, wherein the C1D1, the C1D2, and the C1D3 are each about 90 μg/kg, and wherein the C1D1, the C1D2, and the C1D2 are to be administered to the subject on or about Weeks 0, 4, and 8, respectively, of the first dosing cycle; and (b) the second dosing cycle has a length of about 20 weeks and comprises a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are to be administered on or about Weeks 4, 12, and 20, respectively, of the second dosing cycle.

In another aspect, the invention features the use of an IL-22 Fc fusion protein in the preparation of a medicament for use in a method of treating a subject having an IBD, wherein the IL-22 Fc fusion protein is for administration to the subject in a dosing regimen comprising at least a first dosing cycle and a second dosing cycle, wherein: (a) the first dosing cycle has a length of about 10 weeks and comprises a first dose (C1D1), a second dose (C1D2), and a third dose (C1D3) of the IL-22 Fc fusion protein, wherein the C1D1, the C1D2, and the C1D3 are each about 60 μg/kg, and wherein the C1D1, the C1D2, and the C1D2 are to be administered to the subject on or about Weeks 0, 4, and 8, respectively, of the first dosing cycle; and (b) the second dosing cycle has a length of about 20 weeks and comprises a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are to be administered on or about Weeks 4, 12, and 20, respectively, of the second dosing cycle.

In another aspect, the invention features the use of an IL-22 Fc fusion protein in the preparation of a medicament for use in a method of treating a subject having an IBD, wherein the IL-22 Fc fusion protein is for administration to the subject in a dosing regimen comprising at least a first dosing cycle and a second dosing cycle, wherein: (a) the first dosing cycle has a length of about 10 weeks and comprises a first dose (C1D1), a second dose (C1D2), a third dose (C1D3), a fourth dose (C1D4), a fifth dose (C1D5), and a sixth dose (C1D6) of the IL-22 Fc fusion protein, wherein the C1D1, the C1D2, the C1D3, the C1D4, the C1D5, and the C1D6 are each about 60 μg/kg, and wherein the C1D1, the C1D2, the C1D3, the C1D4, the C1D5, and the C1D6 are to be administered to the subject on or about Weeks 0, 2, 4, 6, 8, and 10, respectively, of the dosing cycle; and (b) the second dosing cycle has a length of about 20 weeks and comprises a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are to be administered on or about Weeks 4, 12, and 20, respectively, of the second dosing cycle.

In some embodiments of any of the preceding aspects, the treating ameliorates one or more symptoms of the IBD. In some embodiments, the one or more symptoms of IBD include stool frequency, rectal bleeding, or mucosal appearance. In some embodiments, mucosal appearance comprises erythema, decreased or absent vascular pattern, friability, erosions, spontaneous bleeding, and/or ulceration.

In some embodiments of any of the preceding aspects, the treating results in a clinical remission. In some embodiments of any of the preceding aspects, the treating results in a clinical remission within about ten weeks from the first dose. In some embodiments, the treating results in a clinical remission within about six weeks from the first dose. In some embodiments, the clinical remission is a modified Mayo Clinic Score (MCS) of less than or equal to about 2 and a Mayo rectal bleeding subscore of 0 and other Mayo subscores of less than or equal to about 1. In some embodiments, the clinical remission is a sustained remission. In some embodiments, the sustained remission is a clinical remission at about ten weeks from the first dose and at about 30 weeks from the first dose. In some embodiments, the sustained remission has a length of at least about 30 weeks, or at least about 7, about 8, about 9, about 10, about 11, or about 12 months.

In some embodiments of any of the preceding aspects, the treating results in a clinical response. In some embodiments, the clinical response comprises a decrease in the subject's mMCS score relative to a baseline mMCS score. In some embodiments, the decrease in the subject's mMCS score is a decrease of at least about 1 point or higher relative to the baseline mMCS score. In some embodiments, the decrease in the subject's mMCS score is a decrease of at least about 3 points or higher relative to the baseline mMCS score. In some embodiments, the clinical response comprises a decrease in the subject's Mayo rectal bleeding subscore relative to a baseline Mayo rectal bleeding subscore or a Mayo rectal bleeding subscore of 0 or 1. In some embodiments, a decrease in the subject's Mayo rectal bleeding subscore is a decrease of about 1 point or higher relative to the baseline Mayo rectal bleeding subscore. In some embodiments, the clinical response is present about 6 weeks after the first dose. In some embodiments, the clinical response is present about 10 weeks after the first dose. In some embodiments, the clinical response is present about 30 weeks after the first dose.

In some embodiments of any of the preceding aspects, the treating results in endoscopic healing. In some embodiments, the endoscopic healing is a Mayo endoscopic subscore of less than or equal to about 1. In some embodiments, the endoscopic healing is present about 6 weeks after the first dose. In some embodiments, the endoscopic healing is present about 10 weeks after the first dose. In some embodiments, the endoscopic healing is present about 30 weeks after the first dose.

In some embodiments of any of the preceding aspects, the treating results in an endoscopic remission. In some embodiments, the endoscopic remission is a Mayo endoscopic subscore of zero. In some embodiments, the endoscopic remission is present about 6 weeks after the first dose. In some embodiments, the endoscopic remission is present about 10 weeks after the first dose. In some embodiments, the endoscopic remission is present about 30 weeks after the first dose.

In some embodiments of any of the preceding aspects, the treating results in a change from baseline in the subject's bowel movement signs and symptoms as assessed by the Ulcerative Colitis Patient-Reported Outcome Signs and Symptoms (UC-PRO/SS) score. In some embodiments, the change from baseline in the subject's bowel movement signs and symptoms as assessed by the UC-PRO/SS score is present about 10 weeks after the first dose. In some embodiments, the change from baseline in the subject's bowel movement signs and symptoms as assessed by the UC-PRO/SS score is present about 30 weeks after the first dose.

In some embodiments of any of the preceding aspects, the treating results in a change from baseline in the subject's abdominal signs and symptoms as assessed by the Ulcerative Colitis Patient-Reported Outcome Signs and Symptoms (UC-PRO/SS) score. In some embodiments, the change from baseline in the subject's abdominal signs and symptoms as assessed by the UC-PRO/SS score is present about 10 weeks after the first dose. In some embodiments, the change from baseline in the subject's abdominal signs and symptoms as assessed by the UC-PRO/SS score is present about 30 weeks after the first dose.

In some embodiments of any of the preceding aspects, the treating results in a change from baseline in the subject's patient-reported health-related quality of life (QOL) as assessed by an Inflammatory Bowel Disease Questionnaire (IBDQ) score. In some embodiments, the change from baseline in the subject's patient-reported health-related QOL as assessed by the IBDQ score is present about 10 weeks after the first dose. In some embodiments, the change from baseline in the subject's patient-reported health-related QOL as assessed by the IBDQ score is present about 30 weeks after the first dose.

In some embodiments of any of the preceding aspects, the treating results in mucosal healing. In some embodiments, the mucosal healing is endoscopic healing and histological remission of less than or equal to about 6, as assessed by Robarts Histological Index. In some embodiments, the mucosal healing is present about 6 weeks after the first dose. In some embodiments, the mucosal healing is present about 10 weeks after the first dose. In some embodiments, the mucosal healing is present about 30 weeks after the first dose.

In some embodiments of any of the preceding aspects, the treating results in a change from baseline in the subject's UC Endoscopic Index of Severity. In some embodiments, the change from baseline in the subject's UC Endoscopic Index of Severity is present about 6 weeks after the first dose.

In some embodiments, the change from baseline in the subject's UC Endoscopic Index of Severity is present about 10 weeks after the first dose. In some embodiments, the change from baseline in the subject's UC Endoscopic Index of Severity is present about 30 weeks after the first dose.

In some embodiments of any of the preceding aspects, the treating results in histological healing. In some embodiments, the histological healing is a Nancy score of 0 or 1 as assessed by the Nancy Histological Index. In some embodiments, the histological healing is present about 6 weeks after the first dose. In some embodiments, the histological healing is present about 10 weeks after the first dose. In some embodiments, the histological healing is present about 30 weeks after the first dose.

In some embodiments of any of the preceding aspects, the amelioration of one or more symptoms of IBD, clinical remission, and/or clinical response is maintained at least one month after the end of treatment. In some embodiments, the amelioration of symptoms, clinical remission, and/or clinical response is maintained at least three months after the end of treatment.

In some embodiments of any of the preceding aspects, the IBD is ulcerative colitis (UC) or Crohn's disease. In some embodiments, the IBD is UC. In some embodiments, the UC is moderate to severe UC. In some embodiments, the moderate to severe UC is defined as a mMCS of 5-9 with an endoscopic subscore of about 2 or higher, a rectal bleeding subscore of about 1 or higher, and a stool frequency subscore of about 1 or higher prior to the treating. In some embodiments, the subject has UC a minimum of about 20 cm from the anal verge as determined by baseline endoscopy. In some embodiments, the IBD is Crohn's disease. In some embodiments, the subject has left-sided colitis, extensive colitis, or pancolitis prior to the treating.

In some embodiments of any of the preceding aspects, the subject has had an inadequate response, loss of response, or intolerance to prior immunosuppressant treatment. In some embodiments, the prior immunosuppressant treatment is treatment with an aminosalicylate, an immunomodulatory agent, a tumor necrosis factor (TNF) antagonist, and/or a corticosteroid. In some embodiments, the subject has had persistent signs or symptoms of active disease despite treatment with at least one 12-week regimen of azathioprine (AZA), mercaptopurine (6-MP), and/or methotrexate (MTX) within five years prior to the treating. In some embodiments, the subject has a history of intolerance to AZA, 6-MP, or MTX within five years prior to the treating. In some embodiments, the subject has had persistent signs or symptoms of active disease despite treatment with at least two induction doses of infliximab, adalimumab, or golimumab within five years prior to the treating. In some embodiments, the subject has had recurrence of signs or symptoms of active disease during maintenance after initial response to induction therapy with infliximab, adalimumab, or golimumab. In some embodiments, the subject has had intolerance to a TNF antagonist. In some embodiments, (i) the subject has had persistent signs or symptoms of active disease despite treatment with at least one 4-week induction regimen that included 30 mg/day of oral prednisone (or equivalent) for at least 2 weeks or 30 mg/day of IV prednisone (or equivalent) for at least 1 week within five years prior to the treating; (ii) the subject has had two failed attempts to taper corticosteroids below 10 mg/day of oral prednisone (or equivalent); or (iii) the subject has a history of intolerance to corticosteroids within five years prior to the treating.

In another aspect, the invention features a method of treating or preventing graft versus host disease (GVHD) in a subject comprising administering to the subject an IL-22 Fc fusion protein in a dosing regimen comprising a dosing cycle, wherein the dosing cycle comprises between two and ten doses, and wherein a total of about 60 μg/kg to about 900 μg/kg of the IL-22 Fc fusion protein is administered to the subject in the dosing cycle.

In another aspect, the invention features an IL-22 Fc fusion protein for use in a method of treating or preventing graft versus host disease (GVHD) in a subject, wherein the IL-22 Fc fusion protein is for administration to the subject in a dosing regimen comprising a dosing cycle, wherein the dosing cycle comprises between two and ten doses, and wherein a total of about 60 μg/kg to about 900 μg/kg of the IL-22 Fc fusion protein is administered to the subject in the dosing cycle.

In another aspect, the invention features the use of an IL-22 Fc fusion protein in the preparation of a medicament for use in a method of treating or preventing graft versus host disease (GVHD) in a subject, wherein the IL-22 Fc fusion protein is for administration to the subject in a dosing regimen comprising a dosing cycle, wherein the dosing cycle comprises between two and ten doses, and wherein a total of about 60 μg/kg to about 900 μg/kg of the IL-22 Fc fusion protein is administered to the subject in the dosing cycle.

In some embodiments, the doses are administered to the subject every week (q1w), every two weeks (q2w), every four weeks (q4w), or every six weeks (q6w). In some embodiments, the doses are administered to the subject every two weeks (q2w). In some embodiments, the first dose of the dosing cycle is administered to the subject about 3 (±2) days prior to allogeneic hematopoietic stem cell transplantation (allo-HSCT). In some embodiments, the second dose is administered on or about Day 11 following the allo-HSCT. In some embodiments, a total of about 480 μg/kg of the IL-22 Fc fusion protein is administered to the subject in the dosing cycle. In some embodiments, the dosing cycle comprises a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein. In some embodiments, the dosing cycle consists of a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein. In some embodiments, the C1D1, the C1D2, the C1D3, the C1D4, the C1D5, the C1D6, the C1D7, and the C1D8 are each between about 15 μg/kg to about 90 μg/kg. In some embodiments, the C1D1, the C1D2, the C1D3, the C1D4, the C1D5, the C1D6, the C1D7, and the C1D8 are each about 60 μg/kg. In some embodiments, the length of the dosing cycle is between about 2 weeks and about 20 weeks. In some embodiments, the length of the dosing cycle is about 96 days.

In another aspect, the invention features a method of treating GVHD in a subject comprising a dosing regimen, the dosing regimen comprising administering to the subject an IL-22 Fc fusion protein every two weeks (q2w) until the subject has a clinical remission of the GVHD.

In another aspect, the invention features an IL-22 Fc fusion protein for use in a method of treating GVHD in a subject, wherein the IL-22 Fc fusion protein is for administration to the subject in a dosing regimen, the dosing regimen comprising administering to the subject an IL-22 Fc fusion protein every two weeks (q2w) until the subject has a clinical remission of the GVHD.

In another aspect, the invention features an IL-22 Fc fusion protein for use in the preparation of a medicament for use in a method of treating GVHD in a subject, wherein the IL-22 Fc fusion protein is for administration to the subject in a dosing regimen, the dosing regimen comprising administering to the subject an IL-22 Fc fusion protein every two weeks (q2w) until the subject has a clinical remission of the GVHD.

In some embodiments of any of the preceding aspects, the GVHD is chronic GVHD or acute GVHD. In some embodiments, the GVHD is acute GVHD. In some embodiments, the method is a method of preventing GVHD.

In another aspect, the invention features a method of preventing acute GVHD in a subject comprising administering to the subject an IL-22 Fc fusion protein in a dosing regimen comprising a dosing cycle having a length of about 96 days, wherein the dosing cycle comprises a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein, wherein the C1D1, the C1D2, the C1D3, the C1D4, the C1D5, the C1D6, the C1D7, and the C1D8 are each about 60 μg/kg, wherein the C1D1 is administered to the subject about 3 (±2) days prior to allo-HSCT, the C1D2 is administered about eleven days after the allo-HSCT, and the C1D3, C1D4, C1D5, C1D6, C1D7, and C1D8 are administered to the subject every two weeks (q2w) following administration of the C1D2.

In some embodiments of any of the preceding aspects, the GVHD is intestinal GVHD.

In some embodiments of any of the preceding aspects, the method prevents Grade II-IV acute GVHD. In some embodiments, the method prevents Grade II-IV acute GVHD at Day 100 after the allo-HSCT. In some embodiments, the Grade II-IV acute GVHD is assessed by the MAGIC GVHD Target Organ Staging.

In some embodiments of any of the preceding aspects, the method reduces the incidence of Stage 1, Stage 2, Stage 3, or Stage 4 acute GVHD of skin, gut, and liver. In some embodiments, the incidence of Stage 1, Stage 2, Stage 3, or Stage 4 acute GVHD of skin, gut, and liver is assessed by the MAGIC GVHD Target Organ Staging. In some embodiments, the method reduces the incidence of Grade I, Grade II, Grade III, or Grade IV acute GVHD. In some embodiments, the incidence of Grade I, Grade II, Grade III, or Grade IV acute GVHD is assessed by the MAGIC GVHD Target Organ Staging.

In some embodiments of any of the preceding aspects, the method (i) improves the gastrointestinal (GI) acute GVHD-free survival rate of the subject; (ii) improves the overall survival of the subject; (iii) improves the relapse-free survival rate of the subject, and/or (iv) reduces the incidence of chronic GVHD in the subject.

In some embodiments of any of the preceding aspects, the IL-22 Fc fusion protein comprises an IL-22 polypeptide linked to an Fc region by a linker. In some embodiments, the IL-22 polypeptide is glycosylated. In some embodiments, the IL-22 polypeptide is N-glycosylated. In some embodiments, the Fc region is not glycosylated. In some embodiments, the amino acid residue at position 297 as in the EU index of the Fc region is Gly. In some embodiments, the amino acid residue at position 297 as in the EU index of the Fc region is Ala. In some embodiments, the amino acid residue at position 299 as in the EU index of the Fc region is Ala, Gly, or Val. In some embodiments, the Fc region comprises the CH2 and CH3 domain of IgG1 or IgG4. In some embodiments, the Fc region comprises the CH2 and CH3 domain of IgG4.

In some embodiments of any of the preceding aspects, the IL-22 Fc fusion protein comprises an amino acid sequence having at least 95% sequence identity to the amino acid sequence of SEQ ID NO:8. In some embodiments, the IL-22 Fc fusion protein comprises an amino acid sequence having at least 96% sequence identity to the amino acid sequence of SEQ ID NO:8. In some embodiments, the IL-22 Fc fusion protein comprises an amino acid sequence having at least 97% sequence identity to the amino acid sequence of SEQ ID NO:8. In some embodiments, the IL-22 Fc fusion protein comprises an amino acid sequence having at least 98% sequence identity to the amino acid sequence of SEQ ID NO:8. In some embodiments, the IL-22 Fc fusion protein comprises an amino acid sequence having at least 99% sequence identity to the amino acid sequence of SEQ ID NO:8.

In some embodiments of any of the preceding aspects, the IL-22 Fc fusion protein comprises the amino acid sequence of SEQ ID NO:8, SEQ ID NO:10, or SEQ ID NO:16. In some embodiments, the IL-22 Fc fusion protein comprises the amino acid sequence of SEQ ID NO:8. In some embodiments, the IL-22 Fc fusion protein consists of the amino acid sequence of SEQ ID NO:8. In some embodiments, the IL-22 Fc fusion protein comprises the amino acid sequence of SEQ ID NO:10. In some embodiments, the IL-22 Fc fusion protein consists of the amino acid sequence of SEQ ID NO:10. In some embodiments, the IL-22 Fc fusion protein comprises the amino acid sequence of SEQ ID NO:16. In some embodiments, the IL-22 Fc fusion protein consists of the amino acid sequence of SEQ ID NO:16. In some embodiments, the Fc region is not N glycosylated.

In some embodiments of any of the preceding aspects, the IL-22 Fc fusion protein is a dimeric IL-22 Fc fusion protein. In other embodiments of any of the preceding aspects, the IL-22 Fc fusion protein is a monomeric IL-22 Fc fusion protein.

In some embodiments of any of the preceding aspects, the IL-22 polypeptide is a human IL-22 polypeptide. In some embodiments, the IL-22 polypeptide comprises the amino acid sequence of SEQ ID NO:4. In some embodiments, the linker comprises the amino acid sequence RVESKYGPP (SEQ ID NO: 44). In some embodiments, the linker consists of the amino acid sequence RVESKYGPP (SEQ ID NO: 44).

In some embodiments of any of the preceding aspects, the IL-22 Fc fusion protein binds to IL-22 receptor. In some embodiments, the IL-22 receptor is human IL-22 receptor. In some embodiments, the IL-22 Fc fusion protein binds to IL-22RA1 and/or IL-10R2. In some embodiments, the IL-22 Fc fusion protein binds to IL-22RA1.

In some embodiments of any of the preceding aspects, the IL-22 Fc fusion protein is administered to the subject in a pharmaceutical composition. In some embodiments, the pharmaceutical composition has an average sialic acid content in the range of 8 to 12 moles (e.g., about 8, about 9, about 10, about 11, or about 12 moles) of sialic acid per mole of the IL-22 Fc fusion protein. In some embodiments, the pharmaceutical composition has an average sialic acid content in the range of 8 to 9 moles of sialic acid per mole of the IL-22 Fc fusion protein. In some embodiments, the pharmaceutical composition has an average sialic acid content of 8 moles of sialic acid per mole of the IL-22 Fc fusion protein. In other embodiments, the pharmaceutical composition has an average sialic acid content of 9 moles of sialic acid per mole of the IL-22 Fc fusion protein. In some embodiments, the sialic acid comprises N-acetylneuraminic acid (NANA). In some embodiments, the pharmaceutical composition has an average NGNA content of less than 1 mole of NGNA per mole of the IL-22 Fc fusion protein.

In some embodiments, the IL-22 polypeptide is N-glycosylated. In some embodiments, the IL-22 polypeptide is glycosylated at one or more locations corresponding to amino acid residues Asn21, Asn35, Asn64, and/or Asn143 of SEQ ID NO: 4. In some embodiments, the IL-22 Fc fusion protein comprises a glycosylated IL-22 polypeptide linked to an Fc region by a linker, wherein the IL-22 polypeptide is glycosylated at one or more locations corresponding to amino acid residues Asn21, Asn35, Asn64, and/or Asn143 of SEQ ID NO: 4, and wherein: (a) the percent N-glycosylation site occupancy at residue Asn21 is in the range of 70 to 90; (b) the percent N-glycosylation site occupancy at residue Asn35 is in the range of 90 to 100; (c) the percent N-glycosylation site occupancy at residue Asn64 is in the range of 90 to 100; and/or (d) the percent N-glycosylation site occupancy at residue Asn143 is in the range of 25 to 35.

In some embodiments of any of the preceding aspects, the pharmaceutical composition is a liquid composition.

In some embodiments of any of the preceding aspects: (i) the IL-22 Fc fusion protein has a maximum observed concentration (Cₘₐₓ) of about 8,000 ng/mL to about 19,000 ng; (ii) the IL-22 Fc fusion protein has an area under the serum concentration-time curve from time 0 to the last measureable time point (AUCₗₐₛₜ) of about 7,000 day·ng/mL to about 25,000 day·ng/mL; and/or (iii) the IL-22 Fc fusion protein has a clearance (CL) of about 40 mL/kg/day to about 140 mL/kg/day. In some embodiments, the Cₘₐₓ, AUCₗₐₛₜ, and/or CL is assessed following intravenous administration of about 1,000 μg/kg of the IL-22 Fc fusion protein to a CD1 mouse.

In some embodiments of any of the preceding aspects, the IL-22 polypeptide comprises N-glycans having monoantennary, biantennary, triantennary, and/or tetraantennary structure. In some embodiments: (i) about 0.1% to about 2% of the N-glycans have monoantennary structure; (ii) about 10% to about 25% of the N-glycans have biantennary structure; (iii) about 25% to about 40% of the N-glycans have triantennary structure; and/or (iv) about 30% to about 51% of the N-glycans have tetraantennary structure. In some embodiments: (i) 0.1% to 2% of the N-glycans have monoantennary structure; (ii) 10% to 25% of the N-glycans have biantennary structure; (iii) 25% to 40% of the N-glycans have triantennary structure; and/or (iv) 30% to 51% of the N-glycans have tetraantennary structure.

In some embodiments of any of the preceding aspects, the IL-22 Fc fusion protein comprises N-glycans comprising zero, one, two, three, or four galactose moieties. In some embodiments: (i) about 9% to about 32% of the N-glycans comprise zero galactose moieties; (ii) about 10% to about 20% of the N-glycans comprise one galactose moiety; (iii) about 8% to about 25% of the N-glycans comprise two galactose moieties; (iv) about 12% to about 25% of the N-glycans comprise three galactose moieties; and/or (v) about 12% to about 30% of the N-glycans comprise four galactose moieties. In some embodiments: (i) 9% to 32% of the N-glycans comprise zero galactose moieties; (ii) 10% to 20% of the N-glycans comprise one galactose moiety; (iii) 8% to 25% of the N-glycans comprise two galactose moieties; (iv) 12% to 25% of the N-glycans comprise three galactose moieties; and/or (v) 12% to 30% of the N-glycans comprise four galactose moieties.

In some embodiments of any of the preceding aspects, the IL-22 Fc fusion protein comprises N-glycans comprising zero, one, two, three, or four sialic acid moieties. In some embodiments: (i) about 12% to about 35% of the N-glycans comprise zero sialic acid moieties; (ii) about 10% to about 30% of the N-glycans comprise one sialic acid moiety; (iii) about 10% to about 30% of the N-glycans comprise two sialic acid moieties; (iv) about 10% to about 30% of the N-glycans comprise three sialic acid moieties; and/or (v) about 1% to about 20% of the N-glycans comprise four sialic acid moieties. In some embodiments: (i) 12% to 35% of the N-glycans comprise zero sialic acid moieties; (ii) 10% to 30% of the N-glycans comprise one sialic acid moiety; (iii) 10% to 30% of the N-glycans comprise two sialic acid moieties; (iv) 10% to 30% of the N-glycans comprise three sialic acid moieties; and/or (v) 1% to 20% of the N-glycans comprise four sialic acid moieties.

In some embodiments of any of the preceding aspects, (i) the IL-22 polypeptide comprises about 0% to about 10% N-glycans comprising a terminal mannose moiety; and/or (ii) the IL-22 polypeptide comprises about 30% to about 55% N-glycans comprising a terminal N-acetylglucosamine (GlcNAc) moiety. In some embodiments, (i) the IL-22 polypeptide comprises 0% to 10% N-glycans comprising a terminal mannose moiety; and/or (ii) the IL-22 polypeptide comprises 30% to 55% N-glycans comprising a terminal GlcNAc moiety. In some embodiments, the IL-22 polypeptide comprises 0% to 10% N-glycans comprising a terminal mannose moiety. In some embodiments, the IL-22 polypeptide comprises 30% to 55% N-glycans comprising a terminal GlcNAc moiety.

In some embodiments of any of the preceding aspects, the N-glycans comprise one, two, three, or four terminal GlcNAc moieties. In some embodiments: (i) about 1% to about 20% of the N-glycans comprise one terminal GlcNAc moiety; (ii) about 1% to about 20% of the N-glycans comprise two terminal GlcNAc moieties; (iii) about 5% to about 25% of the N-glycans comprise three terminal GlcNAc moieties; and/or (iv) about 0% to about 15% of the N-glycans comprise four terminal GlcNAc moieties. In some embodiments: (i) 1% to 20% of the N-glycans comprise one terminal GlcNAc moiety; (ii) 1% to 20% of the N-glycans comprise two terminal GlcNAc moieties; (iii) 5% to 25% of the N-glycans comprise three terminal GlcNAc moieties; and/or (iv) 0% to 15% of the N-glycans comprise four terminal GlcNAc moieties.

In some embodiments of any of the preceding aspects, (i) the IL-22 polypeptide comprises about 20% to about 45% N-glycans comprising a terminal galactose (Gal) moiety; and/or (ii) the N-glycans comprise one, two, or three terminal Gal moieties. In some embodiments, (i) the IL-22 polypeptide comprises 20% to 45% N-glycans comprising a terminal Gal moiety; and/or (ii) the N-glycans comprise one, two, or three terminal Gal moieties.

In some embodiments of any of the preceding aspects: (i) about 15% to about 30% of the N-glycans comprise one terminal Gal moiety; (ii) about 1% to about 15% of the N-glycans comprise two terminal Gal moieties; and/or (iii) about 0.1% to about 6% of the N-glycans comprise three terminal Gal moieties. In some embodiments: (i) 15% to 30% of the N-glycans comprise one terminal Gal moiety; (ii) 1% to 15% of the N-glycans comprise two terminal Gal moieties; and/or (iii) 0.1% to 6% of the N-glycans comprise three terminal Gal moieties.

In some embodiments of any of the preceding aspects: (i) the IL-22 polypeptide comprises N-glycans comprises galactose N-acetylglucosamine (LacNAc) repeats; (ii) the IL-22 polypeptide comprises N-glycans comprising fucosylated N-glycans; and/or (iii) the IL-22 polypeptide comprises N-glycans comprising afucosylated N-glycans.

In some embodiments of any of the preceding aspects, the concentration of the IL-22 Fc fusion protein is about 0.5 mg/mL to about 20 mg/mL. In some embodiments, the concentration of the IL-22 Fc fusion protein is about 0.5 mg/mL to about 5 mg/mL. In some embodiments, the concentration of the IL-22 Fc fusion protein is about 1 mg/mL. In some embodiments, the concentration of the IL-22 Fc fusion protein is about 8 mg/mL to about 12 mg/mL. In some embodiments, the concentration of the IL-22 Fc fusion protein is about 10 mg/mL.

In some embodiments of any of the preceding aspects, the IL-22 Fc fusion is produced from a production culture having a volume of at least about 500 L. In some embodiments of any of the preceding aspects, the IL-22 Fc fusion protein has been produced from a production culture having a volume of about 500 L to about 5,000 L. In some embodiments, the IL-22 Fc fusion protein has been produced from a production culture having a volume of about 1,000 L to about 3,000 L. In some embodiments the IL-22 Fc fusion protein has been produced from a production culture having a volume of about 1,500 L to about 2,500 L. In some embodiments, the IL-22 Fc fusion protein has been produced from a production culture having a volume of about 2000 L.

In some embodiments of any of the preceding aspects, the IL-22 Fc fusion protein is administered to the subject as a monotherapy. In other embodiments of any of the preceding aspects, the IL-22 Fc fusion protein is administered to the subject as a combination therapy. In some embodiments, the IL-22 Fc fusion protein is administered to the subject concurrently with an additional therapeutic agent. In some embodiments, the IL-22 Fc fusion protein is administered to the subject prior to the administration of an additional therapeutic agent. In some embodiments, the IL-22 Fc fusion protein is administered in combination with an additional IBD therapy selected from an aminosalicylate, an immunomodulatory agent, a tumor necrosis factor (TNF) antagonist, an anti-integrin agent, a mucosal addressin cell adhesion molecule (MAdCAM) antagonist, an IL-23 antagonist, an IL-12 antagonist, an IL-12/IL-23 antagonist, an antibiotic, or a corticosteroid. In some embodiments, the additional IBD therapy is an aminosalicylate. In some embodiments, the aminosalicylate comprises 5-aminosalicylic acid (5-ASA). In some embodiments, the additional IBD therapy is an immunomodulatory agent. In some embodiments, the immunomodulatory agent is azathioprine, mercaptopurine, cyclosporine, tacrolimus, sirolimus, mycophenolic acid, or methotrexate. In some embodiments, the additional IBD therapy is a TNF antagonist. In some embodiments, the TNF antagonist is an anti-TNF antibody or a soluble TNF receptor. In some embodiments, the anti-TNF antibody is infliximab, adalimumab, golimumab, or certolizumab pegol. In some embodiments, the soluble TNF receptor is etanercept. In some embodiments, the additional IBD therapy is an anti-integrin agent. In some embodiments, the anti-integrin agent is an anti-integrin antibody. In some embodiments, the anti-integrin antibody is an anti-α4-integrin antibody. In some embodiments, the anti-α4-integrin antibody is natalizumab or vedolizumab. In some embodiments, the MAdCAM antagonist is an anti-MAdCAM antibody. In some embodiments, the anti-MAdCAM antibody is PF-00547659 or SHP647. In some embodiments, the IL-23 antagonist is an anti-IL-23 antibody. In some embodiments, the anti-IL-23 antibody is briakizumab, guselkumab, risankizumab, tilorakizumab, or ustekinumab. In some embodiments, the IL-12 antagonist is an anti-IL-12 antibody. In some embodiments, the anti-IL-12 antibody is ABT-874/J695. In some embodiments, the IL-12/IL-23 antagonist is an anti-IL-12/IL-23 antibody. In some embodiments, the anti-IL-12/IL-23 antibody is ustekinumab or briakinumab. In some embodiments, the IL-22 Fc fusion protein is administered in combination with an additional GVHD therapy selected from an immunosuppressive agent, a chemotherapy agent, a TNF antagonist, a steroid, light treatment, hydroxychloroquine, an anti-fibrotic agent, a monoclonal antibody, or a combination thereof. In some embodiments, the additional GVHD therapy is an immunosuppressive agent. In some embodiments, the immunosuppressive agent is cyclosporine or tacrolimus. In some embodiments, the additional GVHD therapy is standard of care for acute GVHD prophylaxis (e.g., calcineurin (CN) inhibitor (e.g., cyclosporine or tacrolimus)+methotrexate or mycophenolate mofetil (MMF)).

In some embodiments of any of the preceding aspects, the administering is by intravenous infusion. In other embodiments of any of the preceding aspects, the administering is by subcutaneous administration. In some embodiments of any of the preceding aspects, the subject is a human.

In another aspect, the invention features a kit comprising an IL-22 Fc fusion protein and instructions to administer the IL-22 Fc fusion protein to a subject suffering from an IBD in accordance with any of the methods described herein.

In another aspect, the invention features a kit comprising an IL-22 Fc fusion protein and instructions to administer the IL-22 Fc fusion protein to a subject suffering from or at risk of GVHD in accordance with any of the methods described herein.

Each and every embodiment can be combined unless the context clearly suggests otherwise. Each and every embodiment can be applied to each and every aspect of the invention unless the context clearly suggests otherwise.

Specific embodiments of the present invention will become evident from the following more detailed description of certain preferred embodiments and the claims.

### BRIEF DESCRIPTION OF THE DRAWINGS

FIGS. 1A and 1B are schematic diagrams showing a planned dose-escalation scheme (FIG. 1A) and changes to study (symbols and text) and a study flow diagram (FIG. 1B). *Four subjects (3 active: 1 placebo) were added to Cohort C (3 μg/kg SC). ^{†}The dose level for Cohort L was reduced from 240 μg/kg to 90 μg/kg; only sentinel dosing was carried out in Cohort K; Cohort M was canceled (see Example 2).

FIGS. 2A-1 and 2A-2 show dermatological adverse events. The frequency, severity, and duration of dry skin adverse events in IL-22 Fc fusion protein IV and SC cohorts is shown in FIG. 2A-1 and FIG. 2A-2, respectively.

FIG. 2B shows representative images of injection-site erythema and scaling from two subjects in Cohort G (30 μg/kg SC), from Day 5 though Day 14 post dose. Scale bars, 1 cm.

FIG. 2C shows hematoxylin- and eosin-stained sections of skin biopsies taken from IL-22 Fc fusion protein SC injection sites. The left panel of FIG. 2C shows a skin biopsy taken 17 days post dose from a subject in Cohort C (3 μg/kg SC). The left panel of FIG. 2C demonstrates mild/moderate epidermal hyperplasia accompanied by a mild perivascular inflammatory cellular infiltrate in the superficial dermis (arrows). The right panel of FIG. 2C shows a skin biopsy taken 17 days post dose from a subject in Cohort E (10 μg/kg SC), and demonstrates moderate psoriasiform epidermal hyperplasia characterized by the exaggerated downgrowth of rete ridges (asterisks) and parakeratosis (arrowhead). There is also a minimal perivascular inflammatory cellular infiltrate in the superficial dermis (arrow).

FIGS. 3A and 3B are a series of graphs showing mean serum concentration-time profiles for IL-22 Fc fusion protein following single dose administration by IV infusion (FIG. 3A) and SC injection (FIG. 3B).

FIGS. 4A-4D are a series of graphs showing visual evaluation of dose proportionality based on dose-normalized Cₘₐₓ (FIGS. 4A and 4C) and dose-normalized area under the concentration-time curve extrapolated to infinity (AUC_{inf}) (FIGS. 4B and 4D), after single doses of IL-22 Fc fusion protein administered as IV infusions (FIGS. 4A and 4B) or SC injections (FIGS. 4C and 4D). AUC_{inf} for low-dose cohorts (1, 3 μg/kg IV; 3 μg/kg SC) were not calculated due to limited terminal PK concentrations above the LLOQ (lower limit of quantification). Symbols represent observations from individual subjects. Lines represent median of each dose level.

FIGS. 5A-5C are a series of graphs showing mean percent change from baseline of REG3A, CRP, and SAA serum levels in IL-22 Fc fusion protein-treated IV (FIG. 5A) and SC (FIG. 5B) cohorts. One subject in Cohort D (10 μg/kg IV) who showed signs of a viral illness was excluded from plots in FIG. 5A. FIG. 5C shows a plot of the IV cohorts including this subject. Error bars, standard error (SE) for FIGS. 5A and 5B. LLOQs (lower limits of quantification) for REG3A and SAA were 0.15 ng/mL and 57 ng/mL, respectively.

FIGS. 6A-1 and 6A-2 are a series of graphs showing percent change from baseline of REG3A levels by individual subjects in IV cohorts.

FIGS. 6B-1 and 6B-2 are a series of graphs showing percent change from baseline of CRP levels by individual subjects in IV cohorts.

FIGS. 6C-1 and 6C-2 are a series of graphs showing percent change from baseline of SAA levels by individual subjects in IV cohorts.

FIG. 7 is a series of graph showing mean percent change from baseline of REG3A (top panel), CRP (middle panel), and SAA (bottom panel) in low-dose SC cohorts. Error bars, standard error (SE).

FIGS. 8A and 8B are a series of graphs showing serum cytokine levels in subjects treated with IL-22 Fc fusion protein (IV or SC) or placebo. One subject in Cohort D (10 μg/kg IV) cohort was above the lower limit of quantification (LLOQ) for IL-2, but no AEs were noted for this subject on Day 2. Black dashed lines, lower LLOQ.

FIG. 9 shows an amino acid sequence alignment of mature IL-22 from different mammalian species: human (GenBank Accession No. Q9GZX6, SEQ ID NO:4, chimpanzee (GenBank Accession No. XP 003313906, SEQ ID NO:48), orangutan (GenBank Accession No. XP 002823544, SEQ ID NO:49), mouse (GenBank Accession No. Q9JJY9, SEQ ID NO:50), and dog (GenBank Accession No. XP 538274, SEQ ID NO:51).

### DETAILED DESCRIPTION OF EMBODIMENTS OF THE INVENTION

### I. Definitions

Unless otherwise defined, all terms of art, notations, and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

The term “about” as used herein refers to the usual error range for the respective value readily known to the skilled person in this technical field. Reference to “about” a value or parameter herein includes (and describes) embodiments that are directed to that value or parameter per se.

As used herein, the singular forms “a,” “an,” and “the” include plural referents unless the context clearly dictates otherwise. For example, reference to “an isolated peptide” means one or more isolated peptides.

Throughout this specification and claims, the word “comprise,” or variations such as “comprises” or “comprising” will be understood to imply the inclusion of a stated integer or group of integers but not the exclusion of any other integer or group of integers.

The term “IL-22 Fc fusion protein” or “IL-22 fusion protein” or “IL-22 Ig fusion protein” as used herein refers to a fusion protein in which IL-22 protein or polypeptide is linked, directly or indirectly, to an IgG Fc region. In some embodiments, the IL-22 protein or polypeptide is glycosylated. In certain preferred embodiments, the IL-22 Fc fusion protein comprises a human IL-22 protein or polypeptide linked to a human IgG Fc region. In certain embodiments, the human IL-22 protein comprises the amino acid sequence of SEQ ID NO:4. However, it is understood that minor sequence variations such as insertions, deletions, substitutions, especially conservative amino acid substitutions of IL-22 or Fc that do not affect the function and/or activity of IL-22 or IL-22 Fc fusion protein are also contemplated by the invention. The IL-22 Fc fusion protein of the invention can bind to IL-22 receptor, which can lead to IL-22 receptor downstream signaling. In certain embodiments, the IL-22 Fc fusion protein is capable of binding to IL-22 receptor, and/or is capable of leading to IL-22 receptor downstream signaling. The functions and/or activities of the IL-22 Fc fusion protein can be assayed by methods known in the art, including without limitation, enzyme-linked immunosorbent assay (ELISA), ligand-receptor binding assay and Stat3 luciferase assay. In certain embodiments, the invention provides an IL-22 Fc fusion protein that binds to IL-22 receptor, in which the binding can lead to IL-22 receptor downstream signaling, the IL-22 Fc fusion protein comprising an amino acid sequence having at least 95% sequence identity to the amino acid sequence selected from the group consisting of SEQ ID NO:8, SEQ ID NO:10, SEQ ID NO:12, SEQ ID NO:14, and SEQ ID NO:16, and wherein the Fc region is not glycosylated. In certain particular embodiments, the Fc region of the IL-22 fusion protein does not possess effector activities (e.g., does not bind to FcγIIIR) or exhibits substantially lower effector activity than a whole (e.g., wild-type) IgG antibody. In certain other embodiments, the Fc region of the IL-22 Fc fusion protein does not trigger cytotoxicity such as antibody-dependent cellular cytotoxicity (ADCC) or complement dependent cytotoxicity (CDC). Unless otherwise specified, “IL-22 fusion protein,” “IL-22 Fc fusion,” “IL-22 Ig fusion protein,” “IL-22 Fc fusion protein,” or “IL-22 Fc” are used interchangeably throughout this application.

The term “IL-22” or “IL-22 polypeptide” or “IL-22 protein” as used herein, broadly refers to any native IL-22 from any mammalian source, including primates (e.g. humans) and rodents (e.g., mice and rats), unless otherwise indicated. The term encompasses “full-length,” unprocessed IL-22 as well as any forms of IL-22 that result from processing in the cell. For example, both full-length IL-22 containing the N-terminal leader sequence and the mature form IL-22 are encompassed by the current invention. The leader sequence (or signal peptide) can be the endogenous IL-22 leader sequence or an exogenous leader sequence of another mammalian secretary protein. In certain embodiments, the leader sequence can be from a eukaryotic or prokaryotic secretary protein. The term also encompasses naturally occurring variants of IL-22, e.g., splice variants or allelic variants. The amino acid sequence of an exemplary human IL-22 is shown in SEQ ID NO:4 (mature form, without a signal peptide). In certain embodiments, the amino acid sequence of full-length IL-22 protein with the endogenous leader sequence is provided in SEQ ID NO:71; while in other embodiments, the amino acid sequence of mature IL-22 protein with an exogenous leader sequence is provided in SEQ ID NO:2. Minor sequence variations, especially conservative amino acid substitutions of IL-22 that do not affect the IL-22's function and/or activity (e.g., binding to IL-22 receptor), are also contemplated by the invention. FIG. 1 shows an amino acid sequence alignment of mature IL-22 from several exemplary mammalian species. The asterisks indicate highly conserved amino acid residues across species that are likely important for the functions and/or activities of IL-22. Accordingly, in certain embodiments, the IL-22 Fc fusion protein comprises an IL-22 polypeptide comprising an amino acid sequence having at least 95%, at least 96%, at least 97%, at least 98%, or at least 99% sequence identity to SEQ ID NO:4. In certain other embodiments, the IL-22 protein has 95% or more sequence identity to SEQ ID NO:71; 96% or more sequence identity to SEQ ID NO:71; 97% or more sequence identity to SEQ ID NO:71; 98% or more sequence identity to SEQ ID NO:71; or 99% or more sequence identity to SEQ ID NO:71. The IL-22 polypeptides described herein can be isolated from a variety of sources, such as from human tissue or from another source, or prepared by recombinant or synthetic methods.

The term “IL-22 receptor” or “IL-22R” refers to a heterodimer consisting of IL-22R1 and IL-10R2 or naturally occurring allelic variants thereof. See, e.g., Ouyang et al., 2011, Annu. Rev. Immunol. 29:159-63. IL-10R2 is ubiquitously expressed by many cell types, and IL-22R1 is expressed only in innate cells such as epithelial cells, hepatocytes and keratinocytes. IL-22R1 is also known as IL-22Ra1 or IL-22Ra1. IL-22R1 may be paired with other polypeptides to form heterodimeric receptors for other IL-10 family members, for example IL-20 or IL-24. See, e.g., Ouyang et al., 2011, supra.

A “native sequence IL-22 polypeptide” or a “native sequence IL-22R polypeptide” refers to a polypeptide comprising the same amino acid sequence as a corresponding IL-22 or IL-22R polypeptide derived from nature. Such native sequence IL-22 or IL-22R polypeptides can be isolated from nature or can be produced by recombinant or synthetic means. The terms specifically encompass naturally-occurring truncated or secreted forms of the specific IL-22 or IL-22R polypeptide (e.g., an IL-22 lacking its associated signal peptide), naturally-occurring variant forms (e.g., alternatively spliced forms), and naturally-occurring allelic variants of the polypeptide. In various embodiments of the invention, the native sequence IL-22 or IL-22R polypeptides disclosed herein are mature or full-length native sequence polypeptides. An exemplary full length native human IL-22 is shown in SEQ ID NO:70 (DNA) and SEQ ID NO:71 (protein). While the IL-22 and IL-22R polypeptide sequences are shown to begin with methionine residues designated herein as amino acid position 1, it is conceivable and possible that other methionine residues located either upstream or downstream from the amino acid position 1 can be employed as the starting amino acid residue for the IL-22 or IL-22R polypeptides.

An “IL-22 variant,” an “IL-22R variant,” an “IL-22 variant polypeptide,” or an “IL-22R variant polypeptide” means an active IL-22 or IL-22R polypeptide as defined above having at least about 80% amino acid sequence identity with a full-length native sequence IL-22 or IL-22R polypeptide sequence. Ordinarily, an IL-22 or IL-22R polypeptide variant will have at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity, and alternatively at least about 99% amino acid sequence identity to a full-length or mature native sequence IL-22 or IL-22R polypeptide sequence.

The term “Fc region,” “Fc domain,” or “Fc” refers to a C-terminal non-antigen binding region of an immunoglobulin heavy chain that contains at least a portion of the constant region. The term includes native Fc regions and variant Fc regions. In certain embodiments, a human IgG heavy chain Fc region extends from Cys226 to the carboxyl-terminus of the heavy chain. However, the C-terminal lysine (Lys447) of the Fc region may or may not be present, without affecting the structure or stability of the Fc region. Unless otherwise specified herein, numbering of amino acid residues in the IgG or Fc region is according to the EU numbering system for antibodies, also called the EU index, as described in Kabat et al., Sequences of Proteins of Immunological Interest, 5th Ed. Public Health Service, National Institutes of Health, Bethesda, Md., 1991.

In certain embodiments, Fc region refers to an immunoglobulin IgG heavy chain constant region comprising a hinge region (starting at Cys226), an IgG CH2 domain, and CH3 domain. The term “hinge region” or “hinge sequence” as used herein refers to the amino acid sequence located between the linker and the CH2 domain. In certain embodiments, the hinge region comprises the amino acid sequence CPPCP (SEQ ID NO:31). In certain embodiments, the hinge region for IL-22 IgG4 Fc fusion protein comprises the CPPCP sequence (SEQ ID NO:31), a sequence found in the native IgG1 hinge region, to facilitate dimerization. In certain other embodiments, the Fc region starts at the hinge region and extends to the C-terminus of the IgG heavy chain. In certain particular embodiments, the Fc region comprises the Fc region of human IgG1, IgG2, IgG3 or IgG4. In certain particular embodiments, the Fc region comprises the CH2 and CH3 domain of IgG4. In certain other particular embodiments, the Fc region comprises the CH2 and CH3 domain of IgG1.

In certain embodiments, the IgG CH2 domain starts at Ala 231. In certain other embodiments, the CH3 domain starts at Gly 341. It is understood that the C-terminus Lys residue of human IgG can be optionally absent. It is also understood that conservative amino acid substitutions of the Fc region without affecting the desired structure and/or stability of Fc is contemplated within the scope of the invention.

In certain embodiments, the IL-22 is linked to the Fc region via a linker. In certain particular embodiments, the linker is a peptide that connects the C-terminus of IL-22 to the Fc region as described herein. In certain embodiments, native IgG sequences are present in the linker and/or hinge region to minimize and/or avoid the risk of immunogenicity. In other embodiments, minor sequence variations can be introduced to the native sequences to facilitate manufacturing. IL-22 Fc fusion constructs comprising exogenous linker or hinge sequences that exhibit high activity (as measured, e.g., by a luciferase assay) are also within the scope of the invention. In certain embodiments, the linker comprises an amino acid sequence that is 8-20 amino acids, 8-16, 8-15, 8-14, 8-13, 8-12, 8-11, 8-10, 8-9, 10-11, 10-12, 10-13, 10-14, 10-15, 10-16, 11-16, 8, 9, 10, 11, 12, 13, 14, 15, or 16 amino acids long. In certain other embodiments, the linker comprises the amino acid sequence DKTHT (SEQ ID NO:32). In certain particular embodiments, the linker does not comprise the sequence Gly-Gly-Ser (SEQ ID NO:45), Gly-Gly-Gly-Ser (SEQ ID NO:46), or Gly-Gly-Gly-Gly-Ser (SEQ ID NO:47).

In certain embodiments, the IL-22 Fc fusion protein comprises an IL-22 polypeptide linked to an Fc region by a linker. The term “linked to” or “fused to” refers to a covalent bond, e.g., a peptide bond, formed between two moieties.

The terms “glycosylation” and “glycosylated” as used herein refers to the presence of a carbohydrate (e.g., an oligosaccharide or a polysaccharide, also referred to as a “glycan”) attached to biological molecule (e.g., a protein or a lipid). In particular embodiments, glycosylation refers to the presence of a glycan (e.g., an N-glycan) attached to a protein (e.g., an IL-22 Fc fusion protein) or a portion of a protein of interest (e.g., an IL-22 polypeptide moiety of an IL-22 Fc fusion protein). N-linked glycosylation refers to the attachment of the carbohydrate moiety to the side-chain of an asparagine residue. The tripeptide sequences, asparagine-X-serine and asparagine-X-threonine, wherein X is any amino acid except proline, are recognition sequences for enzymatic attachment of the carbohydrate moiety to the asparagine side chain. O-linked glycosylation refers to the attachment of one of the sugars N-acetylgalactosamine, galactose, or xylose to a hydroxyamino acid, most commonly serine or threonine, although 5-hydroxyproline or 5-hydroxylysine can also be involved in O-linked glycosylation. For a review of glycosylation, see, e.g., Varki et al., Essentials of Glycobiology, 3^{rd} Edition, Cold Spring Harbor Laboratory Press, 2015-2017.

The terms “aglycosylated” and “not glycosylated,” as used interchangeably herein, refer to a protein or a portion of a protein of interest (e.g., the Fc region of an IL-22 Fc fusion protein) that is not glycosylated (e.g., not N-glycosylated). It is to be understood that in some embodiments, a portion of a protein of interest (e.g., an IL-22 Fc fusion protein) is glycosylated (e.g., the IL-22 polypeptide portion of an IL-22 Fc fusion protein), while another portion of the protein of interest is not glycosylated (e.g., the Fc region of the IL-22 Fc fusion protein).

In some embodiments, provided herein are IL-22 Fc fusion proteins in which the Fc region or CH2 domain is not glycosylated. In certain embodiments, the N-glycosylation site in the CH2 domain is mutated to prevent glycosylation. For example, an IL-22 Fc fusion protein with an aglycosylated Fc region can be made by mutagenizing the amino acid residue at position 297 as in the EU index in the CH2 domain of the Fc region (e.g., N297). In certain embodiments, the glycosylation in the CH2 domain of the Fc region can be eliminated by altering the glycosylation consensus site, i.e., Asn at position 297 followed by any amino acid residue (in the case of human IgG, Ser) and Thr. The glycosylation site can be altered by amino acid insertions, deletions, and/or substitutions. For example, one or more amino acid residues can be inserted between Asn and Ser or between Ser and Thr to alter the original glycosylation site, wherein the insertions do not regenerate an N-glycosylation site. In certain particular embodiments, the amino acid residue at position 297 as in the EU index (e.g., the N-glycosylated site in Fc) within the CH2 domain of human IgG Fc is mutated to abolish the glycosylation site. In certain particular embodiments, the amino acid residue at position 297 as in the EU index (e.g., N297) is changed to Gly, Ala, Gln, Asp, or Glu. In some particular embodiments, the amino acid residue at position 297 as in the EU index (e.g., N297) is changed to Gly or Ala. In other particular embodiments, the amino acid residue at position 297 as in the EU index (e.g., N297) is changed to Gly. In certain other embodiments, the amino acid residue at position 299 as in the EU index can be substituted with another amino acid, for example, Ala, Val, or Gly. In certain particular embodiments, the mutations that result in an aglycosylated Fc do not affect the structure and/or stability of the IL-22 Fc fusion protein.

In certain embodiments, the IL-22 Fc fusion protein comprises an Fc region in which the amino acid residue at position 297 as in the EU index in the CH2 domain is mutated. In certain embodiments, the amino acid residue at position 297 as in the EU index is changed to Gly or Ala, preferably to Gly. In certain other embodiments, the amino acid residue at position 297 as in the EU index is deleted. In certain embodiments, the IL-22 Fc fusion protein comprising an Fc having an amino acid substitution at the amino acid residue at position 297 as in the EU index is aglycosylated or not glycosylated.

In other embodiments, the N-glycan attached to the wild type amino acid residue at position 297 as in the EU index (e.g., N297) can be removed enzymatically, e.g., by deglycosylation. Suitable glycolytic enzymes include without limitation, peptide-N-glycosidase (PNGase).

The term “glycosylation occupancy” as used herein refers to the probability that a protein is glycosylated at a particular glycosylation site (e.g., an Asn residue of a consensus glycosylation site) or the percentage of proteins in a population of proteins that are glycosylated at a particular glycosylation site. For example, an IL-22 polypeptide may be glycosylated on amino acid residues Asn21, Asn35, Asn64, and/or Asn143 of SEQ ID NO: 4. In a further specific example, (a) the percent N-glycosylation site occupancy at residue Asn21 may be in the range of 70 to 90; (b) the percent N-glycosylation site occupancy at residue Asn35 may be in the range of 90 to 100; (c) the percent N-glycosylation site occupancy at residue Asn64 may be in the range of 90 to 100; and/or (d) the percent N-glycosylation site occupancy at residue Asn143 may be in the range of 25 to 35.

The terms “sialylation” and “sialylated” refers to the presence of sialic acid on a protein or a portion of a protein of interest, particularly as a component of a glycan (e.g., N-glycan) chain attached to a protein. Sialic acid (also referred to herein as a “sialic acid moiety”) refers generally to N- or O-substituted derivatives of neuraminic acid. N-acetylneuraminic acid (5-acetamido-2-keto-3,5-dideoxy-D-glycero-D-galactonononic acid; also known as NANA or Neu5Ac) is the most common sialic acid in mammals. Other exemplary sialic acids include, without limitation, 2-keto-3-deoxy-D-glycero-D-galactonononic acid (also known as Kdn), N-glycolylneuraminic acid (also known as Neu5Gc or NGNA), neuraminic acid (also known as Neu), and 2-deoxy-2,3-didehydro-Neu5Ac (also known as Neu2en5Ac). Free sialic acid (Sia) can be used for glycan synthesis after activation onto the nucleotide donor CMP-Sia. Transfer of Sia from CMP-Sias onto newly synthesized glycoconjugates (e.g., glycoproteins) in the Golgi system of eukaryotes is catalyzed by a family of linkage-specific sialyl-transferases (STs). Sialic acids are typically the terminating residues of glycan (e.g., N-glycan) branches. In some embodiments, sialic acids can occupy internal positions within glycans, most commonly when one sialic acid residue is attached to another. For a review of sialylation and sialic acid, see, e.g., Chapter 15 of Varki et al., Essentials of Glycobiology, 3^{rd} Edition, Cold Spring Harbor Laboratory Press, 2015-2017.

The term “sialic acid content” refers to the level or amount of sialylation of a glycosylated protein (e.g., an IL-22 Fc fusion protein) or a portion of a protein of interest. In some embodiments, an IL-22 Fc fusion protein has a sialic acid content of from about 4 to about 16 moles (e.g., about 4, about 5, about 6, about 7, about 8, about 9, about 10, about 11, about 12, about 13, about 14, about 15, or about 16 moles) of sialic acid per mole of the IL-22 Fc fusion protein. In some embodiments, an IL-22 Fc fusion protein has a sialic acid content of about 8, 9, 10, 11, or 12 moles of sialic acid per mole of the IL-22 Fc fusion protein.

The term “average sialic acid content” with respect to a composition containing an IL-22 Fc fusion protein (e.g., a pharmaceutical composition or a batch) according to the invention refers to the total number of moles of sialic acid in the composition per mole of IL-22 Fc fusion protein in the composition. Thus, for example, such a composition may contain a heterogeneous pool of IL-22 Fc fusion proteins with individual IL-22 Fc fusion proteins within the composition having varying levels of sialylation (e.g., in the range of 0-25 moles of sialic acid per mole of IL-22 Fc fusion protein). Unless indicated otherwise, all values for sialic acid content, including average sialic acid content, described herein refer to dimeric IL-22 Fc fusion proteins.

The term “afucosylation,” “afucosylated,” “defucosylation,” or “defucosylated” refers to the absence or removal of core-fucose from an N-glycan, e.g., an N-glycan attached to a protein or a portion of a protein (e.g., the CH2 domain of Fc).

The term “dimeric IL-22 Fc fusion protein” refers to a dimer in which each monomer comprises an IL-22 Fc fusion protein. The term “monomeric IL-22 Fc fusion protein” refers to a dimer in which one monomer comprises an IL-22 Fc fusion protein (the IL-22 Fc arm), while the other monomer comprises an Fc region without the IL-22 polypeptide (the Fc arm). Accordingly, the dimeric IL-22 Fc fusion protein is bivalent with respect to IL-22R binding, whereas the monomeric IL-22 Fc fusion protein is monovalent with respect to IL-22R binding. The heterodimerization of the monomeric IL-22 Fc fusion protein can be facilitated by methods known in the art, including without limitation, heterodimerization by the knob-into-hole technology. The structure and assembly method of the knob-into-hole technology can be found in, e.g., U.S. Pat. Nos. 5,821,333, 7,642,228, US 2011/0287009, and PCT/US2012/059810, hereby incorporated by reference in their entireties. This technology was developed by introducing a “knob” (or a protuberance) by replacing a small amino acid residue with a large one in the CH3 domain of one Fc, and introducing a “hole” (or a cavity) in the CH3 domain of the other Fc by replacing one or more large amino acid residues with smaller ones. In certain embodiments, the IL-22 Fc fusion arm comprises a knob, and the Fc only arm comprises a hole.

The preferred residues for the formation of a knob are generally naturally occurring amino acid residues and are preferably selected from arginine (R), phenylalanine (F), tyrosine (Y), and tryptophan (W). Most preferred are tryptophan and tyrosine. In one embodiment, the original residue for the formation of the knob has a small side chain volume, such as alanine, asparagine, aspartic acid, glycine, serine, threonine or valine. Exemplary amino acid substitutions in the CH3 domain for forming the knob include without limitation the T366W, T366Y, or F405W substitution.

The preferred residues for the formation of a hole are usually naturally occurring amino acid residues and are preferably selected from alanine (A), serine (S), threonine (T), and valine (V). In one embodiment, the original residue for the formation of the hole has a large side chain volume, such as tyrosine, arginine, phenylalanine, or tryptophan. Exemplary amino acid substitutions in the CH3 domain for generating the hole include without limitation the T366S, L368A, F405A, Y407A, Y407T, and Y407V substitutions. In certain embodiments, the knob comprises T366W substitution, and the hole comprises the T366S/L368A/Y407V substitutions. In certain particular embodiments, the Fc region of the monomeric IL-22 Fc fusion protein comprises an IgG1 Fc region. In certain particular embodiments, the monomeric IL-22 IgG1 Fc fusion comprises an IL-22 Fc knob arm and an Fc hole arm. In certain embodiments, the IL-22 Fc knob arm comprises a T366W substitution (SEQ ID NO:61), and the Fc hole arm comprises T366S, L368A, and Y407V (SEQ ID NO:62). In certain other embodiments, the Fc region of both arms further comprises an N297G or N297A mutation. In certain embodiments, the monomeric IL-22 Fc fusion protein is expressed in E. coli cells. It is understood that other modifications to the Fc region known in the art that facilitate heterodimerization are also contemplated and encompassed by the instant application.

“Affinity” refers to the strength of the sum total of non-covalent interactions between a single binding site of a molecule (e.g., a ligand or an antibody) and its binding partner (e.g., a receptor or an antigen). Unless indicated otherwise, as used herein, “binding affinity” refers to intrinsic binding affinity which reflects a 1:1 interaction between members of a binding pair (e.g., IL-22 Fc fusion protein and IL-22 receptor). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g. scFv), and multispecific antibodies formed from antibody fragments.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

“Effector functions” or “effector activities” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation. In certain embodiments, the IL-22 Fc fusion protein does not exhibit any effector function or any detectable effector function. In certain other embodiments, the IL-22 Fc fusion protein exhibits substantially reduced effector function, e.g., about 50%, 60%, 70% 80%, or 90% reduced effector function.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. The transformed cell includes transiently or stably transformed cell. Progeny may not be completely identical in nucleic acid content to a parent cell, but may contain mutations. Mutant progeny that have the same function or biological activity as screened or selected for in the originally transformed cell are included herein. In certain embodiments, the host cell is transiently transfected with the exogenous nucleic acid. In certain other embodiments, the host cell is stably transfected with the exogenous nucleic acid.

An “immunoconjugate” is an antibody or a fragment of an antibody conjugated to one or more heterologous molecule(s), including but not limited to a cytotoxic agent.

An “isolated” IL-22 Fc fusion protein is one which has been separated from the environment of a host cell that recombinantly produces the fusion protein. In some embodiments, an IL-22 Fc fusion protein is purified to greater than 80%, 85%, 90%, 95%, 96%, 97%, 98%, or 99% purity as determined by, for example, electrophoretic (e.g., SDS-PAGE, isoelectric focusing (IEF), capillary electrophoresis) or chromatographic (e.g., ion exchange or reverse phase HPLC) approaches.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “isolated nucleic acid encoding an IL-22 Fc fusion protein” refers to one or more nucleic acid molecules encoding an IL-22 Fc fusion protein, including such nucleic acid molecule(s) in a single vector or separate vectors, such nucleic acid molecule(s) transiently or stably transfected into a host cell, and such nucleic acid molecule(s) present at one or more locations in a host cell.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

A “native sequence Fc region” comprises an amino acid sequence identical to the amino acid sequence of an Fc region found in nature. Native sequence human Fc regions include, without limitation, a native sequence human IgG1 Fc region (non-A and A allotypes); native sequence human IgG2 Fc region; native sequence human IgG3 Fc region; and native sequence human IgG4 Fc region, as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith. In certain embodiments, the variant Fc region is not glycosylated.

A “disorder,” a “disease,” or a “condition,” as used interchangeably herein, is any condition that would benefit from treatment by a method described herein (e.g., a method that includes administering an IL-22 Fc fusion protein to the subject) or by a compound described herein (e.g., an IL-22 Fc fusion protein or a composition thereof (e.g., a pharmaceutical composition). This includes chronic and acute disorders or diseases including those pathological conditions which predispose the mammal to the disorder in question. In some embodiments, the disorder an IL-22 associated disorder. Exemplary disorders include, but are not limited to, IBD (e.g., UC or Crohn's disease), graft versus host disease (GVHD) (e.g., acute or chronic GVHD, including intestinal GVHD), microbial infection, acute kidney injury, acute pancreatitis, wounds, cardiovascular conditions, metabolic syndrome, acute endotoxemia, sepsis, hidradenitis suppurativa, chronic obstructive pulmonary disease (COPD), hidradenitis suppurativa, and nonalcoholic fatty acid liver disease (e.g., nonalcoholic steatohepatitis (NASH)). In particular embodiments, the disorder is IBD (e.g., UC (e.g., moderate to severe UC) or Crohn's disease). In other particular embodiments, the disorder is GVHD (e.g., acute or chronic GVHD, including intestinal GVHD).

The terms “inflammatory bowel disorder,” “inflammatory bowel disease,” or “IBD,” as used interchangeably herein, are used herein in the broadest sense and include all diseases and pathological conditions the pathogenesis of which involves recurrent inflammation in the intestine, including small intestine and colon. IBD includes, e.g., ulcerative colitis (UC) and Crohn's disease (CD). IBD is not limited to UC and CD. The manifestations of the disease include but are not limited to inflammation and a decrease in epithelial integrity in the intestine.

The term “ulcer” is a site of damage to the skin or mucous membrane that is often characterized by the formation of pus, death of tissue, and is frequently accompanied by an inflammatory reaction.

The terms “intestine” or “gut” as used interchangeably herein broadly encompasses the small intestine and large intestine.

The terms “graft versus host disease” and “GVHD,” as used interchangeably herein, refer to a complication of allogeneic stem cell transplantation. In GVHD, donor hematopoietic stem cells recognize the transplant recipient as foreign and attack the patient's tissues and organs, which can impair the tissue or organ's function or cause it to fail. As used herein, GVHD includes, for example, acute GVHD or chronic GVHD. In particular embodiments, the GVHD is acute GVHD. Further, non-limiting examples include intestinal GVHD.

By “reduce or inhibit” is meant the ability to cause an overall decrease preferably of 20% or greater, more preferably of 50% or greater, and most preferably of 75%, 85%, 90%, 95%, or greater. Reduce or inhibit can refer to the symptoms of the disorder being treated, e.g., the presence or amount of inflammation or ulcers.

A “subject,” “individual,” or “patient” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual, subject or patient is a human.

An “effective amount” or “therapeutically effective amount” of an agent, e.g., a pharmaceutical formulation, refers to an amount effective, at dosages and for periods of time necessary, to achieve the desired therapeutic or prophylactic result.

A “suboptimal amount” refers to the amount less than the optimal amount of a therapeutic agent typically used for a certain treatment. When two therapeutic agents are given to a subject, either concurrently or sequentially, each therapeutic agent can be given at a suboptimal amount as compared to the treatment when each therapeutic agent is given alone. For example, in certain embodiments, the subject in need of IBD treatment is administered with the pharmaceutical composition comprising the IL-22 Fc fusion protein of the invention and a dexamethasone at a suboptimal amount.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease (e.g., preventing GVHD (e.g., acute or chronic GVHD, including intestinal GVHD)), alleviation of symptoms, diminishment of any direct or indirect pathological consequences of the disease, preventing metastasis, decreasing the rate of disease progression, amelioration or palliation of the disease state, and remission or improved prognosis.

For example, with regard to IBD, “treatment” can refer to a decrease in the likelihood of developing IBD, a decrease in the rate of developing IBD, and a decrease in the severity of the disease. Those in need of treatment include those already with the disorder as well as those in which the disorder is to be prevented. Desirable effects of treatment include, but are not limited to, preventing occurrence or recurrence of disease, alleviating symptoms (e.g., diarrhea, fever, fatigue, abdominal pain, cramping, hematochezia, reduced appetite, and unintended weight loss), diminishing any direct or indirect pathological consequences of the disease, preventing the disease, decreasing the rate of disease progression, ameliorating or palliating the disease state, and causing remission or improved prognosis. In some embodiments, IL-22 Fc fusion protein of the invention are used to delay development of a disease or to slow the progression of a disease.

With regard to IBD, “remission” means a decrease or disappearance of the signs and/or symptoms of IBD. Remission includes clinical remission, endoscopic remission, radiographic remission, histological remission, surgical remission, and/or biochemical remission. In some embodiments, clinical remission is a modified Mayo Clinic Score (MCS) of less than or equal to about 2 (e.g., about 0, about 1, or about 2) and a Mayo rectal bleeding subscore of 0 and other Mayo subscores of less than or equal to about 1 (e.g., about 0 or about 1).

The terms “Mayo Clinic Score,” “MCS,” and “Mayo Score” are used interchangeably herein to refer to a scoring system for assessment of IBD (e.g., UC (e.g., moderate to severe UC) or Crohn's disease), for example, as described in Schroeder et al. N. Engl. J. Med. 317(26):1625-1629, 1987, which is incorporated herein by reference in its entirety. The MCS includes four components: stool frequency, rectal bleeding, endoscopy findings, and a physician's global assessment. The stool frequency subscore is determined according to the following criteria: 0=normal number of stools for the subject; 1=1-2 more stools than normal; 2=3-4 stools more than normal; and 3=5 or more stools more than normal. The rectal bleeding subscore is determined according to the following criteria: 0=no blood seen; 1=streaks of blood with stool less than half the time; 2=obvious blood with stool most of the time; and 3=blood alone is passed. The endoscopy findings (“endoscopic”) subscore is determined according to the following criteria: 0=normal or inactive disease; 1=mild disease (erythema, decreased vascular pattern, mild friability); 2=moderate disease (marked erythema, absent vascular pattern, friability, erosions); and 3=severe disease (spontaneous bleeding, ulceration). The physician's global assessment is determined according to the following criteria: 0=normal; 1=mild disease; 2=moderate disease; and 3=severe disease. In some embodiments, the subject's MCS is about 0, about 1, about 2, about 3, about 4, about 5, about 6, about 7, about 8, about 9, about 10, about 11, or about 12.

The terms “modified MCS” or “mMCS” refer to a composite of three MCS assessments: stool frequency, rectal bleeding, and endoscopy findings. In other words, the mMCS includes the stool frequency, rectal bleeding, and endoscopic subscores but omits the physician's global assessment subscore of the MCS. In some embodiments, the subject's mMCS is about 0, about 1, about 2, about 3, about 4, about 5, about 6, about 7, about 8, or about 9.

The terms “Inflammatory Bowel Disease Questionnaire” and “IBDQ” refer to a scoring system for assessment of IBD (e.g., UC (e.g., moderate to severe UC) or Crohn's disease) as described, for example, in Irvine et al J. Pediatr. Gastroenterol. Nutr. 28(4):523-527, 1999, which is incorporated herein by reference in its entirety. The IBDQ includes thirty-two questions scored on a 7-point scale from 1 (worst) to 7 (best) for a range of possible scores from 32 to 224.

The term “Robarts Histological Index” refers to a scoring system for assessment of IBD (e.g., UC (e.g., moderate to severe UC) or Crohn's disease) as described, for example, in Mosli et al. Gut 66(1):50-58, 2017, which is incorporated herein by reference in its entirety.

The terms “Ulcerative Colitis Endoscopic Index of Severity” and “UCEIS” refer to a scoring system for assessment of IBD (e.g., UC (e.g., moderate to severe UC) or Crohn's disease) as described, for example, in Travis et al. Gut 61:535-542, 2012, which is incorporated herein by reference in its entirety. The descriptors and definitions of the UCEIS are shown below in Table 1.

TABLE 1
UCEIS Descriptors and Definitions

| Descriptor | | |
|---|---|---|
| (score most | Likert scale | |
| severe lesions) | anchor points | Definition |
| Vascular | Normal (1) | Normal vascular pattern with arborization |
| pattern | | of capillaries clearly defined, or with |
| | | blurring or patchy loss of capillary margins |
| | Patchy | Patchy obliteration of vascular pattern |
| | obliteration (2) | |
| | Obliterated (3) | Complete obliteration of vascular pattern |
| Bleeding | None (1) | No visible blood |
| | Mucosal (2) | Some spots or streaks of coagulated blood |
| | | on the surface of the mucosa ahead of the |
| | | scope, which can be washed away |
| | Luminal mild (3) | Some free liquid blood in the lumen |
| | Luminal moderate | Frank blood in the lumen ahead of endoscope |
| | or severe (4) | or visible oozing from mucosa after washing |
| | | intraluminal blood, or visible oozing from |
| | | a haemorrhagic mucosa |
| Erosions | None (1) | Normal mucosa, no visible erosions or ulcers |
| and ulcers | Erosions (2) | Tiny (≤5 mm) defects in the mucosa, of a |
| | | white or yellow color with a flat edge |
| | Superficial | Larger (>5 mm) defects in the mucosa, |
| | ulcer (3) | which are discrete fibrin-covered ulcers |
| | | in comparison with erosions, but remain |
| | | superficial |
| | Deep ulcer (4) | Deeper excavated defects in the mucosa, |
| | | with a slightly raised edge |

The term “Nancy Histological Index” refers to a scoring system for assessment of IBD (e.g., UC (e.g., moderate to severe UC) or Crohn's disease) as described, for example, in Marchal-Bressenot et al. Gut 66(1):43-49, 2017, which is incorporated herein by reference in its entirety.

The “pathology” of a disease or condition includes all phenomena that compromise the well-being of the subject.

“Amelioration,” “ameliorating,” “alleviation,” “alleviating,” or equivalents thereof, refers to both therapeutic treatment and prophylactic or preventative measures, wherein the object is to ameliorate, prevent, slow down (lessen), decrease or inhibit a disease or condition, e.g., IBD (e.g., UC (e.g., moderate to severe UC) or Crohn's disease) or GVHD (e.g., acute or chronic GVHD, including intestinal GVHD). Those in need of treatment include those already with the disease or condition as well as those prone to having the disease or condition or those in whom the disease or condition is to be prevented.

“Chronic” administration refers to administration of an agent(s) in a continuous mode as opposed to an acute mode, so as to maintain the initial therapeutic effect for an extended period of time.

“Intermittent” administration is treatment that is not consecutively done without interruption, but rather is cyclic in nature.

The term “package insert” is used to refer to instructions customarily included in commercial packages of therapeutic products, that contain information about the indications, usage, dosage, administration, combination therapy, contraindications, and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Below are examples of how to calculate the % amino acid sequence identity of the amino acid sequence designated “Comparison Protein” or “Reference Protein” to the amino acid sequence designated “IL-22,” wherein “IL-22” represents the amino acid sequence of an IL-22 polypeptide of interest, “Comparison Protein” represents the amino acid sequence of a polypeptide against which the “IL-22” polypeptide of interest is being compared, and “X,” “Y,” and “Z” each represent different amino acid residues.

| | | |
|---|---|---|
| IL-22 | XXXXXXXXXXXXXXX | (Length = 15 amino acids) |
| Reference Protein | XXXXXYYYYYYY | (Length = 12 amino acids) |

% amino acid sequence identity =

(the number of identically matching amino acid residues between the two polypeptide sequences) divided by (the total number of amino acid residues of the IL-22 polypeptide)=5 divided by 15=33.3%

| | | |
|---|---|---|
| IL-22 | XXXXXXXXXX | (Length = 10 amino acids) |
| Reference Protein | XXXXXYYYYYYZZYZ | (Length = 15 amino acids) |

% amino acid sequence identity =
(the number of identically matching amino acid residues between the two
polypeptide sequences) divided by (the total number of amino acid
residues of the IL-22 polypeptide) = 5 divided by 10 = 50%

The term “agonist” is used in the broadest sense and includes any molecule that partially or fully mimics a biological activity of an IL-22 polypeptide. Also encompassed by “agonist” are molecules that stimulate the transcription or translation of mRNA encoding the polypeptide.

Suitable agonist molecules include, e.g., agonist antibodies or antibody fragments; a native polypeptide; fragments or amino acid sequence variants of a native polypeptide; peptides; antisense oligonucleotides; small organic molecules; and nucleic acids that encode polypeptides agonists or antibodies. Reference to “an” agonist encompasses a single agonist or a combination of two or more different agonists.

The term “IL-22 agonist” is used in the broadest sense, and includes any molecule that mimics a qualitative biological activity (as hereinabove defined) of a native sequence IL-22 polypeptide. IL-22 agonists specifically include IL-22-Fc or IL-22 Ig polypeptides (immunoadhesins), but also small molecules mimicking at least one IL-22 biological activity. Preferably, the biological activity is binding of the IL-22 receptor, interacting with IL-22BP, or facilitating an innate immune response pathway.

IL-22R1 pairs with other proteins to form heterodimers as the receptors for certain IL-10 family members. See Ouyang et al., 2011, supra. Thus, in certain embodiments, IL-22 agonists may include an IL-22 receptor agonist, including a cytokine (or a fusion protein or agonist thereof) that binds to and triggers downstream signaling of the IL-22R1. In certain embodiments, the IL-22 agonists include an IL-22R1 agonist, including without limitation an anti-IL-22R1 agonist antibody; an IL-20 agonist, including without limitation IL-20 polypeptide or IL-20 Fc fusion protein; and an IL-24 agonist, including without limitation IL-24 polypeptide or IL-24 fusion protein. In certain other embodiments, the IL-22R1 agonists include an IL-19 agonist, including without limitation IL-19 polypeptide or IL-19 Fc fusion protein; and an IL-26 agonist, including without limitation IL-26 polypeptide or IL-26 Fc fusion protein. Exemplary sequences for IL-19 (GenBank Accession No. AAG16755.1, SEQ ID NO:77), IL-20 (GenBank Accession No. AAH69311.1, SEQ ID NO:78), IL-24 (GenBank Accession No. AAH09681.1, SEQ ID NO:79) and IL-26 (GenBank Accession No. NP 060872.1, SEQ ID NO:80) are provided herein. In certain embodiments, an IL-19 polypeptide comprises the amino acid sequence of SEQ ID NO:77 or the mature protein without the signal peptide. In certain other embodiments, an IL-20 polypeptide comprises the amino acid sequence of SEQ ID NO:78 or the mature protein without the signal peptide. In yet other embodiments, an IL-24 polypeptide comprises the amino acid sequence of SEQ ID NO:79 or the mature protein without the signal peptide. In certain other embodiments, an IL-26 polypeptide comprises the amino acid sequence of SEQ ID NO:80 or the mature protein without the signal peptide.

A “small molecule” is defined herein to have a molecular weight below about 600, preferably below about 1000 daltons.

An “agonist antibody,” as used herein, is an antibody which partially or fully mimics a biological activity of an IL-22 polypeptide.

The terms “pharmaceutical formulation” or “pharmaceutical composition” are used interchangeably herein and refer to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, diluent, stabilizer, or preservative.

As used herein, “biological activity” of protein (e.g., an IL-22 Fc fusion protein) refers to the ability of the protein (e.g., an IL-22 Fc fusion protein) to bind its target, for example, the ability of an IL-22 Fc fusion protein to bind an IL-22 receptor. It can further include a biological response which can be measured in vitro or in vivo. Such activity may be antagonistic or agonistic. In particular embodiments, the activity is agonistic (e.g., receptor activation).

Within this application, unless otherwise stated, the techniques utilized may be found in any of several well-known references such as: Molecular Cloning: A Laboratory Manual (Sambrook, et al., 1989, Cold Spring Harbor Laboratory Press), PCR Protocols: A Guide to Methods and Applications (Innis, et al. 1990. Academic Press, San Diego, Calif.), and Harlow and Lane (1988) Antibodies: A Laboratory Manual ch.14 (Cold Spring Harbor Laboratory, Cold Spring Harbor, N.Y.).

As appropriate, procedures involving the use of commercially available kits and reagents are generally carried out in accordance with manufacturer defined protocols and/or parameters unless otherwise noted. Before the present methods and uses therefore are described, it is to be understood that this invention is not limited to the particular methodology, protocols, cell lines, animal species or genera, constructs, and reagents described as such can, of course, vary. It is also to be understood that the terminology used herein is for the purpose of describing particular embodiments only, and is not intended to limit the scope of the present invention which will be limited only by the appended claims.

### II. Methods of Treatment

Provided herein are methods of treating IL-22 associated diseases such as IBD (e.g., UC (e.g., moderate to severe UC) and Crohn's disease), GVHD, hidradenitis suppurativa, chronic obstructive pulmonary disease (COPD), and nonalcoholic fatty acid liver disease (e.g., nonalcoholic steatohepatitis (NASH)) in a subject that include administering to the subject an IL-22 Fc fusion protein. Also provided are related uses, compositions, articles of manufacture, and kits.

### A. Dosing Regimens and Administration

The methods and uses of the invention described herein include administering an IL-22 Fc fusion protein to a subject having an IL-22 associated disease such as IBD (e.g., UC (e.g., moderate to severe UC) or Crohn's disease), GVHD, hidradenitis suppurativa, COPD, and nonalcoholic fatty acid liver disease (e.g., NASH), thereby treating the subject. The appropriate doses and dosing regimen for the IL-22 Fc fusion protein (e.g., an IL-22 Fc fusion protein comprising or consisting of the amino acid sequence of SEQ ID NO:8, 10, or 16) may be determined based on the severity and course of the disease, the clinical condition of the subject, the subject's clinical history and response to the treatment, and the discretion of the attending physician.

IBD

The invention provides methods, dosing regimens, and dosing cycles for treating IBD (e.g., UC (e.g., moderate to severe UC) or Crohn's disease).

In one aspect, the invention provides a method of treating a subject having an IBD that includes administering to the subject an IL-22 Fc fusion protein in a dosing regimen comprising a dosing cycle, wherein the dosing cycle comprises between about one and about twenty doses (e.g., about one, about two, about three, about four, about five, about six, about seven, about eight, about nine, about ten, about eleven, about twelve, about thirteen, about fourteen, about fifteen, about sixteen, about seventeen, about eighteen, about nineteen, or about twenty doses), and wherein a total of about 1 μg/kg to about 2000 μg/kg of the IL-22 Fc fusion protein is administered to the subject in the dosing cycle.

For example, provided herein is a method of treating a subject having an IBD that includes administering to the subject an IL-22 Fc fusion protein in a dosing regimen comprising at least a first dosing cycle, wherein the first dosing cycle comprises between two and ten doses, and wherein a total of about 60 μg/kg to about 900 μg/kg of the IL-22 Fc fusion protein is administered to the subject in the first dosing cycle. In some embodiments, the doses are administered to the subject every week (q1w), every two weeks (q2w), every four weeks (q4w), or every six weeks (q6w). In some embodiments, a total of about 90 μg/kg, about 180 μg/kg, about 270 μg/kg, about 360 μg/kg, or about 540 μg/kg of the IL-22 Fc fusion protein is administered to the subject in the first dosing cycle. In some embodiments, the length of the first dosing cycle is between about 5 weeks and about 15 weeks. In some embodiments, the length of the first dosing cycle is about 8 weeks. In some embodiments, the first dosing cycle comprises a first dose (C1D1), a second dose (C1D2), and a third dose (C1D3) of the IL-22 Fc fusion protein. In some embodiments, the first dosing cycle consists of the C1D1, the C1D2, and the C1D3. In some embodiments, the C1D1, the C1D2, and the C1D3 are each between about 15 μg/kg to about 90 μg/kg. In some embodiments, the C1D1, the C1D2, and the C1D3 are each about 30 μg/kg. In some embodiments, the C1D1, the C1D2, and the C1D3 are each about 60 μg/kg. In some embodiments, the method comprises administering the C1D1, the C1D2, and the C1D3 on or about Weeks 1, 4, and 8, respectively, of the first dosing cycle.

In another example, provided herein is an IL-22 Fc fusion protein for use in a method of treating a subject having an IBD, wherein the IL-22 Fc fusion protein is for administration to the subject in a dosing regimen comprising at least a first dosing cycle, wherein the first dosing cycle comprises between two and ten doses, and wherein a total of about 60 μg/kg to about 900 μg/kg of the IL-22 Fc fusion protein is administered to the subject in the first dosing cycle. In some embodiments, the doses are administered to the subject every week (q1w), every two weeks (q2w), every four weeks (q4w), or every six weeks (q6w). In some embodiments, a total of about 90 μg/kg, about 180 μg/kg, about 270 μg/kg, about 360 μg/kg, or about 540 μg/kg of the IL-22 Fc fusion protein is administered to the subject in the first dosing cycle. In some embodiments, the length of the first dosing cycle is between about 5 weeks and about 15 weeks. In some embodiments, the length of the first dosing cycle is about 8 weeks. In some embodiments, the first dosing cycle comprises a first dose (C1D1), a second dose (C1D2), and a third dose (C1D3) of the IL-22 Fc fusion protein. In some embodiments, the first dosing cycle consists of the C1D1, the C1D2, and the C1D3. In some embodiments, the C1D1, the C1D2, and the C1D3 are each between about 15 μg/kg to about 90 μg/kg. In some embodiments, the C1D1, the C1D2, and the C1D3 are each about 30 μg/kg. In some embodiments, the C1D1, the C1D2, and the C1D3 are each about 60 μg/kg. In some embodiments, the method comprises administering the C1D1, the C1D2, and the C1D3 on or about Weeks 1, 4, and 8, respectively, of the first dosing cycle.

In yet another example, provided herein is the use of an IL-22 Fc fusion protein in the preparation of a medicament for use in a method of treating a subject having an IBD, wherein the IL-22 Fc fusion protein is for administration to the subject in a dosing regimen comprising at least a first dosing cycle, wherein the first dosing cycle comprises between two and ten doses, and wherein a total of about 60 μg/kg to about 900 μg/kg of the IL-22 Fc fusion protein is administered to the subject in the first dosing cycle. In some embodiments, the doses are administered to the subject every week (q1w), every two weeks (q2w), every four weeks (q4w), or every six weeks (q6w). In some embodiments, a total of about 90 μg/kg, about 180 μg/kg, about 270 μg/kg, about 360 μg/kg, or about 540 μg/kg of the IL-22 Fc fusion protein is administered to the subject in the first dosing cycle. In some embodiments, the length of the first dosing cycle is between about 5 weeks and about 15 weeks. In some embodiments, the length of the first dosing cycle is about 8 weeks. In some embodiments, the first dosing cycle comprises a first dose (C1D1), a second dose (C1D2), and a third dose (C1D3) of the IL-22 Fc fusion protein. In some embodiments, the first dosing cycle consists of the C1D1, the C1D2, and the C1D3. In some embodiments, the C1D1, the C1D2, and the C1D3 are each between about 15 μg/kg to about 90 μg/kg. In some embodiments, the C1D1, the C1D2, and the C1D3 are each about 30 μg/kg. In some embodiments, the C1D1, the C1D2, and the C1D3 are each about 60 μg/kg. In some embodiments, the method comprises administering the C1D1, the C1D2, and the C1D3 on or about Weeks 1, 4, and 8, respectively, of the first dosing cycle.

In some embodiments, the dose(s) are administered to the subject every week (q1w), every two weeks (q2w), every three weeks (q3w), every four weeks, (q4w), every five weeks (q5w), every six weeks (q6w), every seven weeks (q7w), every eight weeks (q8w), every nine weeks (q9w), every ten weeks (q10w), every 12 weeks (q12w), every fourteen weeks (q14w), every sixteen weeks (q16w), every eighteen weeks (q18w), or every twenty weeks (q20w). For example, in some embodiments, the doses are administered to the subject every week (q1w), every two weeks (q2w), every four weeks (q4w), or every six weeks (q6w). In some embodiments, a total of about 540 μg/kg of the IL-22 Fc fusion protein is administered to the subject in the dosing cycle (e.g., about 54 μg/kg q1w, about 90 μg/kg q2w, about 180 μg/kg q4w, or about 270 μg/kg q6w in a ten-week dosing cycle). In some embodiments, a total of about 270 μg/kg of the IL-22 Fc fusion protein is administered to the subject in the dosing cycle (e.g., about 33.8 μg/kg q1w, about 54 μg/kg q2w, about 90 μg/kg q4w, or about 135 μg/kg q6w in a eight-week dosing cycle). In some embodiments, a total of about 360 μg/kg of the IL-22 Fc fusion protein is administered to the subject in the dosing cycle (e.g., about 36 μg/kg q1w, about 60 μg/kg q2w, about 120 μg/kg q4w, or about 180 μg/kg q6w in a ten-week dosing cycle). In some embodiments, a total of about 180 μg/kg of the IL-22 Fc fusion protein is administered to the subject in the dosing cycle (e.g., about 18 μg/kg q1w, about 30 μg/kg q2w, about 60 μg/kg q4w, or about 90 μg/kg q6w in a ten-week dosing cycle). In some embodiments, a total of about 90 μg/kg of the IL-22 Fc fusion protein is administered to the subject in the dosing cycle (e.g., about 9 μg/kg q1w, about 15 μg/kg q2w, about 30 μg/kg q4w, or about 45 μg/kg q6w in a ten-week dosing cycle).

For example, in some embodiments of any of the preceding methods, the dosing cycle (e.g., the first dosing cycle) includes about one to about twenty doses, about one to about nineteen doses, about one to about eighteen doses, about one to about seventeen doses, about one to about sixteen doses, about one to about fifteen doses, about one to about fourteen doses, about one to about thirteen doses, about one to about twelve doses, about one to about eleven doses, about one to about ten doses, about one to about nine doses, about one to about eight doses, about one to about seven doses, about one to about six doses, about one to about five doses, about one to about four doses, about one to about three doses, about one to about two doses, about two to about twenty doses, about two to about nineteen doses, about two to about eighteen doses, about two to about seventeen doses, about two to about sixteen doses, about two to about fifteen doses, about two to about fourteen doses, about two to about thirteen doses, about two to about twelve doses, about two to about eleven doses, about two to about ten doses, about two to about nine doses, about two to about eight doses, about two to about seven doses, about two to about six doses, about two to about five doses, about two to about four doses, about two to about three doses, about three to about twenty doses, about three to about nineteen doses, about three to about eighteen doses, about three to about seventeen doses, about three to about sixteen doses, about three to about fifteen doses, about three to about fourteen doses, about three to about thirteen doses, about three to about twelve doses, about three to about eleven doses, about three to about ten doses, about three to about nine doses, about three to about eight doses, about three to about seven doses, about three to about six doses, about three to about five doses, about three to about four doses, about four to about twenty doses, about four to about nineteen doses, about four to about eighteen doses, about four to about seventeen doses, about four to about sixteen doses, about four to about fifteen doses, about four to about fourteen doses, about four to about thirteen doses, about four to about twelve doses, about four to about eleven doses, about four to about ten doses, about four to about nine doses, about four to about eight doses, about four to about seven doses, about four to about six doses, about four to about five doses, about five to about twenty doses, about five to about nineteen doses, about five to about eighteen doses, about five to about seventeen doses, about five to about sixteen doses, about five to about fifteen doses, about five to about fourteen doses, about five to about thirteen doses, about five to about twelve doses, about five to about eleven doses, about five to about ten doses, about five to about nine doses, about five to about eight doses, about five to about seven doses, about five to about six doses, about six to about twenty doses, about six to about nineteen doses, about six to about eighteen doses, about six to about seventeen doses, about six to about sixteen doses, about six to about fifteen doses, about six to about fourteen doses, about six to about thirteen doses, about six to about twelve doses, about six to about eleven doses, about six to about ten doses, about six to about nine doses, about six to about eight doses, about six to about seven doses, about seven to about twenty doses, about seven to about nineteen doses, about seven to about eighteen doses, about seven to about seventeen doses, about seven to about sixteen doses, about seven to about fifteen doses, about seven to about fourteen doses, about seven to about thirteen doses, about seven to about twelve doses, about seven to about eleven doses, about seven to about ten doses, about seven to about nine doses, about seven to about eight doses, about eight to about twenty doses, about eight to about nineteen doses, about eight to about eighteen doses, about eight to about seventeen doses, about eight to about sixteen doses, about eight to about fifteen doses, about eight to about fourteen doses, about eight to about thirteen doses, about eight to about twelve doses, about eight to about eleven doses, about eight to about ten doses, about eight to about nine doses, about nine to about twenty doses, about nine to about nineteen doses, about nine to about eighteen doses, about nine to about seventeen doses, about nine to about sixteen doses, about nine to about fifteen doses, about nine to about fourteen doses, about nine to about thirteen doses, about nine to about twelve doses, about nine to about eleven doses, about nine to about ten doses, about ten to about twenty doses, about ten to about nineteen doses, about ten to about eighteen doses, about ten to about seventeen doses, about ten to about sixteen doses, about ten to about fifteen doses, about ten to about fourteen doses, about ten to about thirteen doses, about ten to about twelve doses, about ten to about eleven doses, about eleven to about twenty doses, about eleven to about nineteen doses, about eleven to about eighteen doses, about eleven to about seventeen doses, about eleven to about sixteen doses, about eleven to about fifteen doses, about eleven to about fourteen doses, about eleven to about thirteen doses, about eleven to about twelve doses, about twelve to about twenty doses, about twelve to about nineteen doses, about twelve to about eighteen doses, about twelve to about seventeen doses, about twelve to about sixteen doses, about twelve to about fifteen doses, about twelve to about fourteen doses, about twelve to about thirteen doses, about thirteen to about twenty doses, about thirteen to about nineteen doses, about thirteen to about eighteen doses, about thirteen to about seventeen doses, about thirteen to about sixteen doses, about thirteen to about fifteen doses, about thirteen to about fourteen doses, about fourteen to about twenty doses, about fourteen to about nineteen doses, about fourteen to about eighteen doses, about fourteen to about seventeen doses, about fourteen to about sixteen doses, about fourteen to about fifteen doses, about fifteen to about twenty doses, about fifteen to about nineteen doses, about fifteen to about eighteen doses, about fifteen to about seventeen doses, about fifteen to about sixteen doses, about sixteen to about twenty doses, about sixteen to about nineteen doses, about sixteen to about eighteen doses, about sixteen to about seventeen doses, about seventeen to about twenty doses, about seventeen to about nineteen doses, about seventeen to about eighteen doses, about eighteen to about twenty doses, about eighteen to about nineteen doses, or about nineteen to about twenty doses.

For example, in some embodiments of any of the preceding methods, a total of about 1 μg/kg to about 2000 μg/kg of the IL-22 Fc fusion protein is administered to the subject in the dosing cycle (e.g., the first dosing cycle), e.g., about 1 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 110 μg/kg, about 120 μg/kg, about 130 μg/kg, about 140 μg/kg, about 150 μg/kg, about 160 μg/kg, about 170 μg/kg, about 180 μg/kg, about 190 μg/kg, about 200 μg/kg, about 210 μg/kg, about 220 μg/kg, about 230 μg/kg, about 240 μg/kg, about 250 μg/kg, about 260 μg/kg, about 270 μg/kg, about 280 μg/kg, about 290 μg/kg, about 300 μg/kg, about 310 μg/kg, about 320 μg/kg, about 330 μg/kg, about 340 μg/kg, about 350 μg/kg, about 360 μg/kg, about 370 μg/kg, about 380 μg/kg, about 390 μg/kg, about 400 μg/kg, about 410 μg/kg, about 420 μg/kg, about 430 μg/kg, about 440 μg/kg, about 450 μg/kg, about 460 μg/kg, about 470 μg/kg, about 480 μg/kg, about 490 μg/kg, about 500 μg/kg, about 510 μg/kg, about 520 μg/kg, about 530 μg/kg, about 540 μg/kg, about 550 μg/kg, about 560 μg/kg, about 570 μg/kg, about 580 μg/kg, about 590 μg/kg, about 600 μg/kg, about 610 μg/kg, about 620 μg/kg, about 630 μg/kg, about 640 μg/kg, about 650 μg/kg, about 660 μg/kg, about 670 μg/kg, about 680 μg/kg, about 690 μg/kg, about 700 μg/kg, about 710 μg/kg, about 720 μg/kg, about 730 μg/kg, about 740 μg/kg, about 750 μg/kg, about 760 μg/kg, about 770 μg/kg, about 780 μg/kg, about 790 μg/kg, about 800 μg/kg, about 810 μg/kg, about 820 μg/kg, about 830 μg/kg, about 840 μg/kg, about 850 μg/kg, about 860 μg/kg, about 870 μg/kg, about 880 μg/kg, about 890 μg/kg, about 900 μg/kg, about 910 μg/kg, about 920 μg/kg, about 930 μg/kg, about 940 μg/kg, about 950 μg/kg, about 960 μg/kg, about 970 μg/kg, about 980 μg/kg, about 990 μg/kg, about 1000 μg/kg, about 1010 μg/kg, about 1020 μg/kg, about 1030 μg/kg, about 1040 μg/kg, about 1050 μg/kg, about 1060 μg/kg, about 1070 μg/kg, about 1080 μg/kg, about 1090 μg/kg, about 1100 μg/kg, about 1110 μg/kg, about 1120 μg/kg, about 1130 μg/kg, about 1140 μg/kg, about 1150 μg/kg, about 1160 μg/kg, about 1170 μg/kg, about 1180 μg/kg, about 1190 μg/kg, about 1200 μg/kg, about 1210 μg/kg, about 1220 μg/kg, about 1230 μg/kg, about 1240 μg/kg, about 1250 μg/kg, about 1260 μg/kg, about 1270 μg/kg, about 1280 μg/kg, about 1290 μg/kg, about 1300 μg/kg, about 1310 μg/kg, about 1320 μg/kg, about 1330 μg/kg, about 1340 μg/kg, about 1350 μg/kg, about 1360 μg/kg, about 1370 μg/kg, about 1380 μg/kg, about 1390 μg/kg, about 1400 μg/kg, about 1410 μg/kg, about 1420 μg/kg, about 1430 μg/kg, about 1440 μg/kg, about 1450 μg/kg, about 1460 μg/kg, about 1470 μg/kg, about 1480 μg/kg, about 1490 μg/kg, about 1500 μg/kg, about 1510 μg/kg, about 1520 μg/kg, about 1530 μg/kg, about 1540 μg/kg, about 1550 μg/kg, about 1560 μg/kg, about 1570 μg/kg, about 1580 μg/kg, about 1590 μg/kg, about 1600 μg/kg, about 1610 μg/kg, about 1620 μg/kg, about 1630 μg/kg, about 1640 μg/kg, about 1650 μg/kg, about 1660 μg/kg, about 1670 μg/kg, about 1680 μg/kg, about 1690 μg/kg, about 1700 μg/kg, about 1710 μg/kg, about 1720 μg/kg, about 1730 μg/kg, about 1740 μg/kg, about 1750 μg/kg, about 1760 μg/kg, about 1770 μg/kg, about 1780 μg/kg, about 1790 μg/kg, about 1800 μg/kg, about 1810 μg/kg, about 1820 μg/kg, about 1830 μg/kg, about 1840 μg/kg, about 1850 μg/kg, about 1860 μg/kg, about 1870 μg/kg, about 1880 μg/kg, about 1890 μg/kg, about 1900 μg/kg, about 1910 μg/kg, about 1920 μg/kg, about 1930 μg/kg, about 1940 μg/kg, about 1950 μg/kg, about 1960 μg/kg, about 1970 μg/kg, about 1980 μg/kg, about 1990 μg/kg, or about 2000 μg/kg.

For example, in some embodiments of any of the preceding methods, a total of about 1 μg/kg to about 2000 μg/kg, about 1 μg/kg to about 1900 μg/kg, about 1 μg/kg to about 1800 μg/kg, about 1 μg/kg to about 1700 μg/kg, about 1 μg/kg to about 1600 μg/kg, about 1 μg/kg to about 1500 μg/kg, about 1 μg/kg to about 1400 μg/kg, about 1 μg/kg to about 1300 μg/kg, about 1 μg/kg to about 1200 μg/kg, about 1 μg/kg to about 1100 μg/kg, about 1 μg/kg to about 1000 μg/kg, about 1 μg/kg to about 900 μg/kg, about 1 μg/kg to about 800 μg/kg, about 1 μg/kg to about 750 μg/kg, about 1 μg/kg to about 700 μg/kg, about 1 μg/kg to about 650 μg/kg, about 1 μg/kg to about 600 μg/kg, about 1 μg/kg to about 550 μg/kg, about 1 μg/kg to about 500 μg/kg, about 1 μg/kg to about 450 μg/kg, about 1 μg/kg to about 400 μg/kg, about 1 μg/kg to about 350 μg/kg, about 1 μg/kg to about 300 μg/kg, about 1 μg/kg to about 250 μg/kg, about 1 μg/kg to about 200 μg/kg, about 1 μg/kg to about 150 μg/kg, about 1 μg/kg to about 100 μg/kg, about 1 μg/kg to about 50 μg/kg, 50 μg/kg to about 2000 μg/kg, about 50 μg/kg to about 1900 μg/kg, about 50 μg/kg to about 1800 μg/kg, about 50 μg/kg to about 1700 μg/kg, about 50 μg/kg to about 1600 μg/kg, about 50 μg/kg to about 1500 μg/kg, about 50 μg/kg to about 1400 μg/kg, about 50 μg/kg to about 1300 μg/kg, about 50 μg/kg to about 1200 μg/kg, about 50 μg/kg to about 1100 μg/kg, about 50 μg/kg to about 1000 μg/kg, about 50 μg/kg to about 900 μg/kg, about 50 μg/kg to about 800 μg/kg, about 50 μg/kg to about 750 μg/kg, about 50 μg/kg to about 700 μg/kg, about 50 μg/kg to about 650 μg/kg, about 50 μg/kg to about 600 μg/kg, about 50 μg/kg to about 550 μg/kg, about 50 μg/kg to about 500 μg/kg, about 50 μg/kg to about 450 μg/kg, about 50 μg/kg to about 400 μg/kg, about 50 μg/kg to about 350 μg/kg, about 50 μg/kg to about 300 μg/kg, about 50 μg/kg to about 250 μg/kg, about 50 μg/kg to about 200 μg/kg, about 50 μg/kg to about 150 μg/kg, about 50 μg/kg to about 100 μg/kg, about 100 μg/kg to about 2000 μg/kg, about 100 μg/kg to about 1900 μg/kg, about 100 μg/kg to about 1800 μg/kg, about 100 μg/kg to about 1700 μg/kg, about 100 μg/kg to about 1600 μg/kg, about 100 μg/kg to about 1500 μg/kg, about 100 μg/kg to about 1400 μg/kg, about 100 μg/kg to about 1300 μg/kg, about 100 μg/kg to about 1200 μg/kg, about 100 μg/kg to about 1100 μg/kg, about 100 μg/kg to about 1000 μg/kg, about 100 μg/kg to about 900 μg/kg, about 100 μg/kg to about 800 μg/kg, about 100 μg/kg to about 750 μg/kg, about 100 μg/kg to about 700 μg/kg, about 100 μg/kg to about 650 μg/kg, about 100 μg/kg to about 600 μg/kg, about 100 μg/kg to about 550 μg/kg, about 100 μg/kg to about 500 μg/kg, about 100 μg/kg to about 450 μg/kg, about 100 μg/kg to about 400 μg/kg, about 100 μg/kg to about 350 μg/kg, about 100 μg/kg to about 300 μg/kg, about 100 μg/kg to about 250 μg/kg, about 100 μg/kg to about 200 μg/kg, about 100 μg/kg to about 150 μg/kg, about 150 μg/kg to about 2000 μg/kg, about 150 μg/kg to about 1900 μg/kg, about 150 μg/kg to about 1800 μg/kg, about 150 μg/kg to about 1700 μg/kg, about 150 μg/kg to about 1600 μg/kg, about 150 μg/kg to about 1500 μg/kg, about 150 μg/kg to about 1400 μg/kg, about 150 μg/kg to about 1300 μg/kg, about 150 μg/kg to about 1200 μg/kg, about 150 μg/kg to about 1100 μg/kg, about 150 μg/kg to about 1000 μg/kg, about 150 μg/kg to about 900 μg/kg, about 150 μg/kg to about 800 μg/kg, about 150 μg/kg to about 750 μg/kg, about 150 μg/kg to about 700 μg/kg, about 150 μg/kg to about 650 μg/kg, about 150 μg/kg to about 600 μg/kg, about 150 μg/kg to about 550 μg/kg, about 150 μg/kg to about 500 μg/kg, about 150 μg/kg to about 450 μg/kg, about 150 μg/kg to about 400 μg/kg, about 150 μg/kg to about 350 μg/kg, about 150 μg/kg to about 300 μg/kg, about 150 μg/kg to about 250 μg/kg, about 150 μg/kg to about 200 μg/kg, about 200 μg/kg to about 2000 μg/kg, about 200 μg/kg to about 1900 μg/kg, about 200 μg/kg to about 1800 μg/kg, about 200 μg/kg to about 1700 μg/kg, about 200 μg/kg to about 1600 μg/kg, about 200 μg/kg to about 1500 μg/kg, about 200 μg/kg to about 1400 μg/kg, about 200 μg/kg to about 1300 μg/kg, about 200 μg/kg to about 1200 μg/kg, about 200 μg/kg to about 1100 μg/kg, about 200 μg/kg to about 1000 μg/kg, about 200 μg/kg to about 900 μg/kg, about 200 μg/kg to about 800 μg/kg, about 200 μg/kg to about 750 μg/kg, about 200 μg/kg to about 700 μg/kg, about 200 μg/kg to about 650 μg/kg, about 200 μg/kg to about 600 μg/kg, about 200 μg/kg to about 550 μg/kg, about 200 μg/kg to about 500 μg/kg, about 200 μg/kg to about 450 μg/kg, about 200 μg/kg to about 400 μg/kg, about 200 μg/kg to about 350 μg/kg, about 200 μg/kg to about 300 μg/kg, about 200 μg/kg to about 250 μg/kg, about 250 μg/kg to about 2000 μg/kg, about 250 μg/kg to about 1900 μg/kg, about 250 μg/kg to about 1800 μg/kg, about 250 μg/kg to about 1700 μg/kg, about 250 μg/kg to about 1600 μg/kg, about 250 μg/kg to about 1500 μg/kg, about 250 μg/kg to about 1400 μg/kg, about 250 μg/kg to about 1300 μg/kg, about 250 μg/kg to about 1200 μg/kg, about 250 μg/kg to about 1100 μg/kg, about 250 μg/kg to about 1000 μg/kg, about 250 μg/kg to about 900 μg/kg, about 250 μg/kg to about 800 μg/kg, about 250 μg/kg to about 750 μg/kg, about 250 μg/kg to about 700 μg/kg, about 250 μg/kg to about 650 μg/kg, about 250 μg/kg to about 600 μg/kg, about 250 μg/kg to about 550 μg/kg, about 250 μg/kg to about 500 μg/kg, about 250 μg/kg to about 450 μg/kg, about 250 μg/kg to about 400 μg/kg, about 250 μg/kg to about 350 μg/kg, about 250 μg/kg to about 300 μg/kg, about 300 μg/kg to about 2000 μg/kg, about 300 μg/kg to about 1900 μg/kg, about 300 μg/kg to about 1800 μg/kg, about 300 μg/kg to about 1700 μg/kg, about 300 μg/kg to about 1600 μg/kg, about 300 μg/kg to about 1500 μg/kg, about 300 μg/kg to about 1400 μg/kg, about 300 μg/kg to about 1300 μg/kg, about 300 μg/kg to about 1200 μg/kg, about 300 μg/kg to about 1100 μg/kg, about 300 μg/kg to about 1000 μg/kg, about 300 μg/kg to about 900 μg/kg, about 300 μg/kg to about 800 μg/kg, about 300 μg/kg to about 750 μg/kg, about 300 μg/kg to about 700 μg/kg, about 300 μg/kg to about 650 μg/kg, about 300 μg/kg to about 600 μg/kg, about 300 μg/kg to about 550 μg/kg, about 300 μg/kg to about 500 μg/kg, about 300 μg/kg to about 450 μg/kg, about 300 μg/kg to about 400 μg/kg, about 300 μg/kg to about 350 μg/kg, about 350 μg/kg to about 2000 μg/kg, about 350 μg/kg to about 1900 μg/kg, about 350 μg/kg to about 1800 μg/kg, about 350 μg/kg to about 1700 μg/kg, about 350 μg/kg to about 1600 μg/kg, about 350 μg/kg to about 1500 μg/kg, about 350 μg/kg to about 1400 μg/kg, about 350 μg/kg to about 1300 μg/kg, about 350 μg/kg to about 1200 μg/kg, about 350 μg/kg to about 1100 μg/kg, about 350 μg/kg to about 1000 μg/kg, about 350 μg/kg to about 900 μg/kg, about 350 μg/kg to about 800 μg/kg, about 350 μg/kg to about 750 μg/kg, about 350 μg/kg to about 700 μg/kg, about 350 μg/kg to about 650 μg/kg, about 350 μg/kg to about 600 μg/kg, about 350 μg/kg to about 550 μg/kg, about 350 μg/kg to about 500 μg/kg, about 350 μg/kg to about 450 μg/kg, about 350 μg/kg to about 400 μg/kg, about 400 μg/kg to about 2000 μg/kg, about 400 μg/kg to about 1900 μg/kg, about 400 μg/kg to about 1800 μg/kg, about 400 μg/kg to about 1700 μg/kg, about 400 μg/kg to about 1600 μg/kg, about 400 μg/kg to about 1500 μg/kg, about 400 μg/kg to about 1400 μg/kg, about 400 μg/kg to about 1300 μg/kg, about 400 μg/kg to about 1200 μg/kg, about 400 μg/kg to about 1100 μg/kg, about 400 μg/kg to about 1000 μg/kg, about 400 μg/kg to about 900 μg/kg, about 400 μg/kg to about 800 μg/kg, about 400 μg/kg to about 750 μg/kg, about 400 μg/kg to about 700 μg/kg, about 400 μg/kg to about 650 μg/kg, about 400 μg/kg to about 600 μg/kg, about 400 μg/kg to about 550 μg/kg, about 400 μg/kg to about 500 μg/kg, about 400 μg/kg to about 450 μg/kg, about 450 μg/kg to about 2000 μg/kg, about 450 μg/kg to about 1900 μg/kg, about 450 μg/kg to about 1800 μg/kg, about 450 μg/kg to about 1700 μg/kg, about 450 μg/kg to about 1600 μg/kg, about 450 μg/kg to about 1500 μg/kg, about 450 μg/kg to about 1400 μg/kg, about 450 μg/kg to about 1300 μg/kg, about 450 μg/kg to about 1200 μg/kg, about 450 μg/kg to about 1100 μg/kg, about 450 μg/kg to about 1000 μg/kg, about 450 μg/kg to about 900 μg/kg, about 450 μg/kg to about 800 μg/kg, about 450 μg/kg to about 750 μg/kg, about 450 μg/kg to about 700 μg/kg, about 450 μg/kg to about 650 μg/kg, about 450 μg/kg to about 600 μg/kg, about 450 μg/kg to about 550 μg/kg, about 450 μg/kg to about 500 μg/kg, about 500 μg/kg to about 2000 μg/kg, about 500 μg/kg to about 1900 μg/kg, about 500 μg/kg to about 1800 μg/kg, about 500 μg/kg to about 1700 μg/kg, about 500 μg/kg to about 1600 μg/kg, about 500 μg/kg to about 1500 μg/kg, about 500 μg/kg to about 1400 μg/kg, about 500 μg/kg to about 1300 μg/kg, about 500 μg/kg to about 1200 μg/kg, about 500 μg/kg to about 1100 μg/kg, about 500 μg/kg to about 1000 μg/kg, about 500 μg/kg to about 900 μg/kg, about 500 μg/kg to about 800 μg/kg, about 500 μg/kg to about 750 μg/kg, about 500 μg/kg to about 700 μg/kg, about 500 μg/kg to about 650 μg/kg, about 500 μg/kg to about 600 μg/kg, about 500 μg/kg to about 550 μg/kg, about 550 μg/kg to about 2000 μg/kg, about 550 μg/kg to about 1900 μg/kg, about 550 μg/kg to about 1800 μg/kg, about 550 μg/kg to about 1700 μg/kg, about 550 μg/kg to about 1600 μg/kg, about 550 μg/kg to about 1500 μg/kg, about 550 μg/kg to about 1400 μg/kg, about 550 μg/kg to about 1300 μg/kg, about 550 μg/kg to about 1200 μg/kg, about 550 μg/kg to about 1100 μg/kg, about 550 μg/kg to about 1000 μg/kg, about 550 μg/kg to about 900 μg/kg, about 550 μg/kg to about 800 μg/kg, about 550 μg/kg to about 750 μg/kg, about 550 μg/kg to about 700 μg/kg, about 550 μg/kg to about 650 μg/kg, about 550 μg/kg to about 600 μg/kg, about 600 μg/kg to about 2000 μg/kg, about 600 μg/kg to about 1900 μg/kg, about 600 μg/kg to about 1800 μg/kg, about 600 μg/kg to about 1700 μg/kg, about 600 μg/kg to about 1600 μg/kg, about 600 μg/kg to about 1500 μg/kg, about 600 μg/kg to about 1400 μg/kg, about 600 μg/kg to about 1300 μg/kg, about 600 μg/kg to about 1200 μg/kg, about 600 μg/kg to about 1100 μg/kg, about 600 μg/kg to about 1000 μg/kg, about 600 μg/kg to about 900 μg/kg, about 600 μg/kg to about 800 μg/kg, about 600 μg/kg to about 750 μg/kg, about 600 μg/kg to about 700 μg/kg, about 600 μg/kg to about 650 μg/kg, about 650 μg/kg to about 2000 μg/kg, about 650 μg/kg to about 1900 μg/kg, about 650 μg/kg to about 1800 μg/kg, about 650 μg/kg to about 1700 μg/kg, about 650 μg/kg to about 1600 μg/kg, about 650 μg/kg to about 1500 μg/kg, about 650 μg/kg to about 1400 μg/kg, about 650 μg/kg to about 1300 μg/kg, about 650 μg/kg to about 1200 μg/kg, about 650 μg/kg to about 1100 μg/kg, about 650 μg/kg to about 1000 μg/kg, about 650 μg/kg to about 900 μg/kg, about 650 μg/kg to about 800 μg/kg, about 650 μg/kg to about 750 μg/kg, about 650 μg/kg to about 700 μg/kg, about 700 μg/kg to about 2000 μg/kg, about 700 μg/kg to about 1900 μg/kg, about 700 μg/kg to about 1800 μg/kg, about 700 μg/kg to about 1700 μg/kg, about 700 μg/kg to about 1600 μg/kg, about 700 μg/kg to about 1500 μg/kg, about 700 μg/kg to about 1400 μg/kg, about 700 μg/kg to about 1300 μg/kg, about 700 μg/kg to about 1200 μg/kg, about 700 μg/kg to about 1100 μg/kg, about 700 μg/kg to about 1000 μg/kg, about 700 μg/kg to about 900 μg/kg, about 700 μg/kg to about 800 μg/kg, about 700 μg/kg to about 750 μg/kg, about 750 μg/kg to about 2000 μg/kg, about 750 μg/kg to about 1900 μg/kg, about 750 μg/kg to about 1800 μg/kg, about 750 μg/kg to about 1700 μg/kg, about 750 μg/kg to about 1600 μg/kg, about 750 μg/kg to about 1500 μg/kg, about 750 μg/kg to about 1400 μg/kg, about 750 μg/kg to about 1300 μg/kg, about 750 μg/kg to about 1200 μg/kg, about 750 μg/kg to about 1100 μg/kg, about 750 μg/kg to about 1000 μg/kg, about 750 μg/kg to about 900 μg/kg, about 750 μg/kg to about 800 μg/kg, about 800 μg/kg to about 2000 μg/kg, about 800 μg/kg to about 1900 μg/kg, about 800 μg/kg to about 1800 μg/kg, about 800 μg/kg to about 1700 μg/kg, about 800 μg/kg to about 1600 μg/kg, about 800 μg/kg to about 1500 μg/kg, about 800 μg/kg to about 1400 μg/kg, about 800 μg/kg to about 1300 μg/kg, about 800 μg/kg to about 1200 μg/kg, about 800 μg/kg to about 1100 μg/kg, about 800 μg/kg to about 1000 μg/kg, about 800 μg/kg to about 900 μg/kg, about 850 μg/kg to about 2000 μg/kg, about 850 μg/kg to about 1900 μg/kg, about 850 μg/kg to about 1800 μg/kg, about 850 μg/kg to about 1700 μg/kg, about 850 μg/kg to about 1600 μg/kg, about 850 μg/kg to about 1500 μg/kg, about 850 μg/kg to about 1400 μg/kg, about 850 μg/kg to about 1300 μg/kg, about 850 μg/kg to about 1200 μg/kg, about 850 μg/kg to about 1100 μg/kg, about 850 μg/kg to about 1000 μg/kg, about 850 μg/kg to about 900 μg/kg, about 900 μg/kg to about 2000 μg/kg, about 900 μg/kg to about 1900 μg/kg, about 900 μg/kg to about 1800 μg/kg, about 900 μg/kg to about 1700 μg/kg, about 900 μg/kg to about 1600 μg/kg, about 900 μg/kg to about 1500 μg/kg, about 900 μg/kg to about 1400 μg/kg, about 900 μg/kg to about 1300 μg/kg, about 900 μg/kg to about 1200 μg/kg, about 900 μg/kg to about 1100 μg/kg, about 900 μg/kg to about 1000 μg/kg, about 950 μg/kg to about 2000 μg/kg, about 950 μg/kg to about 1900 μg/kg, about 950 μg/kg to about 1800 μg/kg, about 950 μg/kg to about 1700 μg/kg, about 950 μg/kg to about 1600 μg/kg, about 950 μg/kg to about 1500 μg/kg, about 950 μg/kg to about 1400 μg/kg, about 950 μg/kg to about 1300 μg/kg, about 950 μg/kg to about 1200 μg/kg, about 950 μg/kg to about 1100 μg/kg, about 950 μg/kg to about 1000 μg/kg, about 1000 μg/kg to about 2000 μg/kg, about 1000 μg/kg to about 1900 μg/kg, about 1000 μg/kg to about 1800 μg/kg, about 1000 μg/kg to about 1700 μg/kg, about 1000 μg/kg to about 1600 μg/kg, about 1000 μg/kg to about 1500 μg/kg, about 1000 μg/kg to about 1400 μg/kg, about 1000 μg/kg to about 1300 μg/kg, about 1000 μg/kg to about 1200 μg/kg, about 1000 μg/kg to about 1100 μg/kg, about 1100 μg/kg to about 2000 μg/kg, about 1100 μg/kg to about 1900 μg/kg, about 1100 μg/kg to about 1800 μg/kg, about 1100 μg/kg to about 1700 μg/kg, about 1100 μg/kg to about 1600 μg/kg, about 1100 μg/kg to about 1500 μg/kg, about 1100 μg/kg to about 1400 μg/kg, about 1100 μg/kg to about 1300 μg/kg, about 1100 μg/kg to about 1200 μg/kg, about 1200 μg/kg to about 2000 μg/kg, about 1200 μg/kg to about 1900 μg/kg, about 1200 μg/kg to about 1800 μg/kg, about 1200 μg/kg to about 1700 μg/kg, about 1200 μg/kg to about 1600 μg/kg, about 1200 μg/kg to about 1500 μg/kg, about 1200 μg/kg to about 1400 μg/kg, about 1200 μg/kg to about 1300 μg/kg, about 1300 μg/kg to about 2000 μg/kg, about 1300 μg/kg to about 1900 μg/kg, about 1300 μg/kg to about 1800 μg/kg, about 1300 μg/kg to about 1700 μg/kg, about 1300 μg/kg to about 1600 μg/kg, about 1300 μg/kg to about 1500 μg/kg, about 1300 μg/kg to about 1400 μg/kg, about 1400 μg/kg to about 2000 μg/kg, about 1400 μg/kg to about 1900 μg/kg, about 1400 μg/kg to about 1800 μg/kg, about 1400 μg/kg to about 1700 μg/kg, about 1400 μg/kg to about 1600 μg/kg, about 1400 μg/kg to about 1500 μg/kg, about 1500 μg/kg to about 2000 μg/kg, about 1500 μg/kg to about 1900 μg/kg, about 1500 μg/kg to about 1800 μg/kg, about 1500 μg/kg to about 1700 μg/kg, about 1500 μg/kg to about 1600 μg/kg, about 1600 μg/kg to about 2000 μg/kg, about 1600 μg/kg to about 1900 μg/kg, about 1600 μg/kg to about 1800 μg/kg, about 1600 μg/kg to about 1700 μg/kg, about 1700 μg/kg to about 2000 μg/kg, about 1700 μg/kg to about 1900 μg/kg, about 1700 μg/kg to about 1800 μg/kg, about 1800 μg/kg to about 2000 μg/kg, about 1800 μg/kg to about 1900 μg/kg, or about 1900 μg/kg to about 2000 μg/kg of the IL-22 Fc fusion protein is administered to the subject in the dosing cycle (e.g., the first dosing cycle).

For example, in some embodiments, the invention provides a method of treating a subject having an IBD comprising administering to the subject an IL-22 Fc fusion protein in a dosing regimen comprising a dosing cycle, wherein the dosing cycle comprises between two and six doses, and wherein a total of about 30 μg/kg to about 720 μg/kg of the IL-22 Fc fusion protein is administered to the subject in the dosing cycle. In some embodiments, a total of about 30 μg/kg to about 540 μg/kg of the IL-22 Fc fusion protein is administered to the subject in the dosing cycle.

In some embodiments, a total of about 90 μg/kg to about 360 μg/kg of the IL-22 Fc fusion protein is administered to the subject in the dosing cycle.

In some embodiments of any of the preceding methods, the length of the dosing cycle (e.g., the first dosing cycle) is between about 1 week and about 30 weeks, e.g., about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, or about 30 weeks.

For example, in some embodiments, the length of the dosing cycle (e.g., the first dosing cycle) is between about 1 week and about 30 weeks, between about 1 week about 25 weeks, between about 1 week and about 20 weeks, between about 1 week and about 15 weeks, between about 1 week and about 11 weeks, between about 1 week and about 10 weeks, between about 1 week and about 9 weeks, between about 1 week and about 8 weeks, between about 1 week and about 7 weeks, between about 1 week and about 6 weeks, between about 1 week and about 5 weeks, between about 1 week and about 4 weeks, between about 1 week and about 3 weeks, between about 1 week and about 2 weeks, about 2 weeks and about 30 weeks, between about 2 weeks about 25 weeks, between about 2 weeks and about 20 weeks, between about 2 weeks and about 15 weeks, between about 2 weeks and about 11 weeks, between about 2 weeks and about 10 weeks, between about 2 weeks and about 9 weeks, between about 2 weeks and about 8 weeks, between about 2 weeks and about 7 weeks, between about 2 weeks and about 6 weeks, between about 2 weeks and about 5 weeks, between about 2 weeks and about 4 weeks, between about 2 weeks and about 3 weeks, between about 3 weeks and about 30 weeks, between about 3 weeks about 25 weeks, between about 3 weeks and about 20 weeks, between about 3 weeks and about 15 weeks, between about 3 weeks and about 11 weeks, between about 3 weeks and about 10 weeks, between about 3 weeks and about 9 weeks, between about 3 weeks and about 8 weeks, between about 3 weeks and about 7 weeks, between about 3 weeks and about 6 weeks, between about 3 weeks and about 5 weeks, between about 3 weeks and about 4 weeks, between about 4 weeks and about 30 weeks, between about 4 weeks about 25 weeks, between about 4 weeks and about 20 weeks, between about 4 weeks and about 15 weeks, between about 4 weeks and about 11 weeks, between about 4 weeks and about 10 weeks, between about 4 weeks and about 9 weeks, between about 4 weeks and about 8 weeks, between about 4 weeks and about 7 weeks, between about 4 weeks and about 6 weeks, between about 4 weeks and about 5 weeks, between about 5 weeks and about 30 weeks, between about 5 weeks about 25 weeks, between about 5 weeks and about 20 weeks, between about 5 weeks and about 15 weeks, between about 5 weeks and about 11 weeks, between about 5 weeks and about 10 weeks, between about 5 weeks and about 9 weeks, between about 5 weeks and about 8 weeks, between about 5 weeks and about 7 weeks, between about 5 weeks and about 6 weeks, between about 6 weeks and about 30 weeks, between about 6 weeks about 25 weeks, between about 6 weeks and about 20 weeks, between about 6 weeks and about 15 weeks, between about 6 weeks and about 11 weeks, between about 6 weeks and about 10 weeks, between about 6 weeks and about 9 weeks, between about 6 weeks and about 8 weeks, between about 6 weeks and about 7 weeks, between about 7 weeks and about 30 weeks, between about 7 weeks about 25 weeks, between about 7 weeks and about 20 weeks, between about 7 weeks and about 15 weeks, between about 7 weeks and about 11 weeks, between about 7 weeks and about 10 weeks, between about 7 weeks and about 9 weeks, between about 7 weeks and about 8 weeks, between about 8 weeks and about 30 weeks, between about 8 weeks about 25 weeks, between about 8 weeks and about 20 weeks, between about 8 weeks and about 15 weeks, between about 8 weeks and about 11 weeks, between about 8 weeks and about 10 weeks, between about 8 weeks and about 9 weeks, between about 9 weeks and about 30 weeks, between about 9 weeks about 25 weeks, between about 9 weeks and about 20 weeks, between about 9 weeks and about 15 weeks, between about 9 weeks and about 11 weeks, between about 9 weeks and about 10 weeks, between about 10 weeks and about 30 weeks, between about 10 weeks about 25 weeks, between about 10 weeks and about 20 weeks, between about 10 weeks and about 15 weeks, between about 10 weeks and about 11 weeks, between about 11 weeks and about 30 weeks, between about 11 weeks about 25 weeks, between about 11 weeks and about 20 weeks, between about 11 weeks and about 15 weeks, between about 12 weeks and about 30 weeks, between about 12 weeks about 25 weeks, between about 12 weeks and about 20 weeks, between about 12 weeks and about 15 weeks, between about 13 weeks and about 30 weeks, between about 13 weeks about 25 weeks, between about 13 weeks and about 20 weeks, between about 13 weeks and about 15 weeks, between about 14 weeks and about 30 weeks, between about 14 weeks about 25 weeks, between about 14 weeks and about 20 weeks, between about 14 weeks and about 15 weeks, between about 15 weeks and about 30 weeks, between about 15 weeks about 25 weeks, between about 15 weeks and about 20 weeks, between about 20 weeks and about 30 weeks, between about 20 weeks about 25 weeks, or between about 25 weeks and about 30 weeks. In some embodiments, the length of the dosing cycle (e.g., the first dosing cycle) is between 5 weeks and 15 weeks. In some embodiments, the length of the dosing cycle (e.g., the first dosing cycle) is between 8 weeks and 12 weeks. In particular embodiments, the length of the dosing cycle is about 8 weeks. In other particular embodiments, the length of the dosing cycle (e.g., the first dosing cycle) is about 10 weeks. In other particular embodiments, the length of the dosing cycle (e.g., the first dosing cycle) is up to about 10 weeks.

In some embodiments of any of the preceding methods, the dosing cycle (e.g., the first dosing cycle) includes a first dose (C1D1) and a second dose (C1D2) of the IL-22 Fc fusion protein. In some embodiments, the dosing cycle consists of the C1D1 and the C1D2. The C1D1 and the C1D2 may be administered, for example, q6w in a dosing cycle of about 10 weeks. The C1D1 and the C1D2 may be the same amount or different amounts of the IL-22 Fc fusion protein. In some embodiments, the C1D1 and the C1D2 are each between about 1 μg/kg to about 500 μg/kg, e.g., about 1 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 110 μg/kg, about 120 μg/kg, about 130 μg/kg, about 140 μg/kg, about 150 μg/kg, about 160 μg/kg, about 170 μg/kg, about 180 μg/kg, about 190 μg/kg, about 200 μg/kg, about 210 μg/kg, about 220 μg/kg, about 230 μg/kg, about 240 μg/kg, about 250 μg/kg, about 260 μg/kg, about 270 μg/kg, about 280 μg/kg, about 290 μg/kg, about 300 μg/kg, about 310 μg/kg, about 320 μg/kg, about 330 μg/kg, about 340 μg/kg, about 350 μg/kg, about 360 μg/kg, about 370 μg/kg, about 380 μg/kg, about 390 μg/kg, about 400 μg/kg, about 410 μg/kg, about 420 μg/kg, about 430 μg/kg, about 440 μg/kg, about 450 μg/kg, about 460 μg/kg, about 470 μg/kg, about 480 μg/kg, about 490 μg/kg, or about 500 μg/kg. In some embodiments, the C1D1 and the C1D2 are each between about 1 μg/kg to about 500 μg/kg, between about 1 μg/kg to about 450 μg/kg, between about 1 μg/kg to about 400 μg/kg, between about 1 μg/kg to about 350 μg/kg, between about 1 μg/kg to about 300 μg/kg, between about 1 μg/kg to about 250 μg/kg, between about 1 μg/kg to about 200 μg/kg, between about 1 μg/kg to about 150 μg/kg, between about 1 μg/kg to about 135 μg/kg, between about 1 μg/kg to about 100 μg/kg, between about 1 μg/kg to about 90 μg/kg, between about 1 μg/kg to about 75 μg/kg, between about 1 μg/kg to about 50 μg/kg, between about 1 μg/kg to about 25 μg/kg, between about 25 μg/kg to about 500 μg/kg, between about 25 μg/kg to about 450 μg/kg, between about 25 μg/kg to about 400 μg/kg, between about 25 μg/kg to about 350 μg/kg, between about 25 μg/kg to about 300 μg/kg, between about 25 μg/kg to about 250 μg/kg, between about 25 μg/kg to about 200 μg/kg, between about 25 μg/kg to about 150 μg/kg, between about 25 μg/kg to about 135 μg/kg, between about 25 μg/kg to about 100 μg/kg, between about 25 μg/kg to about 75 μg/kg, between about 25 μg/kg to about 50 μg/kg, between about 50 μg/kg to about 500 μg/kg, between about 50 μg/kg to about 450 μg/kg, between about 50 μg/kg to about 400 μg/kg, between about 50 μg/kg to about 350 μg/kg, between about 50 μg/kg to about 300 μg/kg, between about 50 μg/kg to about 250 μg/kg, between about 50 μg/kg to about 200 μg/kg, between about 50 μg/kg to about 150 μg/kg, between about 50 μg/kg to about 135 μg/kg, between about 50 μg/kg to about 100 μg/kg, between about 50 μg/kg to about 75 μg/kg, between about 75 μg/kg to about 500 μg/kg, between about 75 μg/kg to about 450 μg/kg, between about 75 μg/kg to about 400 μg/kg, between about 75 μg/kg to about 350 μg/kg, between about 75 μg/kg to about 300 μg/kg, between about 75 μg/kg to about 250 μg/kg, between about 75 μg/kg to about 200 μg/kg, between about 75 μg/kg to about 150 μg/kg, between about 75 μg/kg to about 135 μg/kg, between about 75 μg/kg to about 100 μg/kg, between about 80 μg/kg to about 500 μg/kg, between about 80 μg/kg to about 450 μg/kg, between about 80 μg/kg to about 400 μg/kg, between about 80 μg/kg to about 350 μg/kg, between about 80 μg/kg to about 300 μg/kg, between about 80 μg/kg to about 250 μg/kg, between about 80 μg/kg to about 200 μg/kg, between about 80 μg/kg to about 150 μg/kg, between about 80 μg/kg to about 135 μg/kg, between about 80 μg/kg to about 100 μg/kg, between about 90 μg/kg to about 500 μg/kg, between about 90 μg/kg to about 450 μg/kg, between about 90 μg/kg to about 400 μg/kg, between about 90 μg/kg to about 350 μg/kg, between about 90 μg/kg to about 300 μg/kg, between about 90 μg/kg to about 250 μg/kg, between about 90 μg/kg to about 200 μg/kg, between about 90 μg/kg to about 150 μg/kg, between about 90 μg/kg to about 135 μg/kg, between about 90 μg/kg to about 100 μg/kg, between about 100 μg/kg to about 500 μg/kg, between about 100 μg/kg to about 450 μg/kg, between about 100 μg/kg to about 400 μg/kg, between about 100 μg/kg to about 350 μg/kg, between about 100 μg/kg to about 300 μg/kg, between about 100 μg/kg to about 250 μg/kg, between about 100 μg/kg to about 200 μg/kg, between about 100 μg/kg to about 150 μg/kg, between about 100 μg/kg to about 135 μg/kg, between about 125 μg/kg to about 500 μg/kg, between about 125 μg/kg to about 450 μg/kg, between about 125 μg/kg to about 400 μg/kg, between about 125 μg/kg to about 350 μg/kg, between about 125 μg/kg to about 300 μg/kg, between about 125 μg/kg to about 250 μg/kg, between about 125 μg/kg to about 200 μg/kg, between about 125 μg/kg to about 150 μg/kg, between about 125 μg/kg to about 135 μg/kg, between about 135 μg/kg to about 500 μg/kg, between about 135 μg/kg to about 450 μg/kg, between about 135 μg/kg to about 400 μg/kg, between about 135 μg/kg to about 350 μg/kg, between about 135 μg/kg to about 300 μg/kg, between about 135 μg/kg to about 250 μg/kg, between about 135 μg/kg to about 200 μg/kg, between about 135 μg/kg to about 150 μg/kg, between about 150 μg/kg to about 500 μg/kg, between about 150 μg/kg to about 450 μg/kg, between about 150 μg/kg to about 400 μg/kg, between about 150 μg/kg to about 350 μg/kg, between about 150 μg/kg to about 300 μg/kg, between about 150 μg/kg to about 250 μg/kg, between about 150 μg/kg to about 200 μg/kg, between about 200 μg/kg to about 500 μg/kg, between about 200 μg/kg to about 450 μg/kg, between about 200 μg/kg to about 400 μg/kg, between about 200 μg/kg to about 350 μg/kg, between about 200 μg/kg to about 300 μg/kg, between about 200 μg/kg to about 250 μg/kg, between about 250 μg/kg to about 500 μg/kg, between about 250 μg/kg to about 450 μg/kg, between about 250 μg/kg to about 400 μg/kg, between about 250 μg/kg to about 350 μg/kg, between about 250 μg/kg to about 300 μg/kg, between about 300 μg/kg to about 500 μg/kg, between about 300 μg/kg to about 450 μg/kg, between about 300 μg/kg to about 400 μg/kg, between about 300 μg/kg to about 350 μg/kg, between about 350 μg/kg to about 500 μg/kg, between about 350 μg/kg to about 450 μg/kg, between about 350 μg/kg to about 400 μg/kg, between about 400 μg/kg to about 500 μg/kg, between about 400 μg/kg to about 450 μg/kg, or between about 450 μg/kg to about 500 μg/kg. In some embodiments, the C1D1 and the C1D2 are each between about 45 μg/kg to about 135 μg/kg. In particular embodiments, the C1D1 and the C1D2 are each about 90 μg/kg. For example, in some embodiments, about 90 μg/kg of the IL-22 Fc fusion protein is administered to the subject q6w. In other particular embodiments, the C1D1 and the C1D2 are each about 45 μg/kg. For example, in some embodiments, about 45 μg/kg of the IL-22 Fc fusion protein is administered to the subject q6w.

The C1D1 and the C1D2 can be administered on any suitable week of the dosing cycle (e.g., the first dosing cycle) in any of the preceding methods. For example, in some embodiments, the C1D1 is administered on Week 0, on Week 1, on Week 2, on Week 3, on Week 4, on Week 5, on Week 6, on Week 7, on Week 8, on Week 9, on Week 10, on Week 11, on Week 12, on Week 13, on Week 14, on Week 15, on Week 16, on Week 17, on Week 18, on Week 19, on Week 20, on Week 21, on Week 22, on Week 23, on Week 24, on Week 25, on Week 26, on Week 27, on Week 28, on Week 29, or on Week 30 of the dosing cycle. For example, in some embodiments, the C1D2 is administered on Week 0, on Week 1, on Week 2, on Week 3, on Week 4, on Week 5, on Week 6, on Week 7, on Week 8, on Week 9, on Week 10, on Week 11, on Week 12, on Week 13, on Week 14, on Week 15, on Week 16, on Week 17, on Week 18, on Week 19, on Week 20, on Week 21, on Week 22, on Week 23, on Week 24, on Week 25, on Week 26, on Week 27, on Week 28, on Week 29, or on Week 30 of the dosing cycle. In some embodiments, the method includes administering to the subject the C1D1 and the C1D2 on or about Weeks 0 and 6, respectively, of the dosing cycle.

In some embodiments of any of the preceding methods, the dosing cycle (e.g., the first dosing cycle) includes a first dose (C1D1), a second dose (C1D2), and a third dose (C1D3) of the IL-22 Fc fusion protein. In some embodiments, the dosing cycle consists of the C1D1, the C1D2, and the C1D3. The C1D1, the C1D2, and the C1D3 may be administered, for example, q4w in a dosing cycle of about 10 weeks. In other examples, the C1D1, the C1D2, and the C1D3 may be administered, for example, q4w in a dosing cycle of about 8 weeks. The C1D1, the C1D2, and the C1D3 may be the same amount or different amounts of the IL-22 Fc fusion protein. In some embodiments, the C1D1, the C1D2, and the C1D3 are each between about 1 μg/kg to about 500 μg/kg, e.g., about 1 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 110 μg/kg, about 120 μg/kg, about 130 μg/kg, about 140 μg/kg, about 150 μg/kg, about 160 μg/kg, about 170 μg/kg, about 180 μg/kg, about 190 μg/kg, about 200 μg/kg, about 210 μg/kg, about 220 μg/kg, about 230 μg/kg, about 240 μg/kg, about 250 μg/kg, about 260 μg/kg, about 270 μg/kg, about 280 μg/kg, about 290 μg/kg, about 300 μg/kg, about 310 μg/kg, about 320 μg/kg, about 330 μg/kg, about 340 μg/kg, about 350 μg/kg, about 360 μg/kg, about 370 μg/kg, about 380 μg/kg, about 390 μg/kg, about 400 μg/kg, about 410 μg/kg, about 420 μg/kg, about 430 μg/kg, about 440 μg/kg, about 450 μg/kg, about 460 μg/kg, about 470 μg/kg, about 480 μg/kg, about 490 μg/kg, or about 500 μg/kg. In some embodiments, the C1D1, the C1D2, and the C1D3 are each between about 1 μg/kg to about 500 μg/kg, between about 1 μg/kg to about 450 μg/kg, between about 1 μg/kg to about 400 μg/kg, between about 1 μg/kg to about 350 μg/kg, between about 1 μg/kg to about 300 μg/kg, between about 1 μg/kg to about 250 μg/kg, between about 1 μg/kg to about 200 μg/kg, between about 1 μg/kg to about 150 μg/kg, between about 1 μg/kg to about 135 μg/kg, between about 1 μg/kg to about 100 μg/kg, between about 1 μg/kg to about 90 μg/kg, between about 1 μg/kg to about 75 μg/kg, between about 1 μg/kg to about 50 μg/kg, between about 1 μg/kg to about 25 μg/kg, between about 25 μg/kg to about 500 μg/kg, between about 25 μg/kg to about 450 μg/kg, between about 25 μg/kg to about 400 μg/kg, between about 25 μg/kg to about 350 μg/kg, between about 25 μg/kg to about 300 μg/kg, between about 25 μg/kg to about 250 μg/kg, between about 25 μg/kg to about 200 μg/kg, between about 25 μg/kg to about 150 μg/kg, between about 25 μg/kg to about 135 μg/kg, between about 25 μg/kg to about 100 μg/kg, between about 25 μg/kg to about 75 μg/kg, between about 25 μg/kg to about 50 μg/kg, between about 50 μg/kg to about 500 μg/kg, between about 50 μg/kg to about 450 μg/kg, between about 50 μg/kg to about 400 μg/kg, between about 50 μg/kg to about 350 μg/kg, between about 50 μg/kg to about 300 μg/kg, between about 50 μg/kg to about 250 μg/kg, between about 50 μg/kg to about 200 μg/kg, between about 50 μg/kg to about 150 μg/kg, between about 50 μg/kg to about 135 μg/kg, between about 50 μg/kg to about 100 μg/kg, between about 50 μg/kg to about 75 μg/kg, between about 75 μg/kg to about 500 μg/kg, between about 75 μg/kg to about 450 μg/kg, between about 75 μg/kg to about 400 μg/kg, between about 75 μg/kg to about 350 μg/kg, between about 75 μg/kg to about 300 μg/kg, between about 75 μg/kg to about 250 μg/kg, between about 75 μg/kg to about 200 μg/kg, between about 75 μg/kg to about 150 μg/kg, between about 75 μg/kg to about 135 μg/kg, between about 75 μg/kg to about 100 μg/kg, between about 80 μg/kg to about 500 μg/kg, between about 80 μg/kg to about 450 μg/kg, between about 80 μg/kg to about 400 μg/kg, between about 80 μg/kg to about 350 μg/kg, between about 80 μg/kg to about 300 μg/kg, between about 80 μg/kg to about 250 μg/kg, between about 80 μg/kg to about 200 μg/kg, between about 80 μg/kg to about 150 μg/kg, between about 80 μg/kg to about 135 μg/kg, between about 80 μg/kg to about 100 μg/kg, between about 90 μg/kg to about 500 μg/kg, between about 90 μg/kg to about 450 μg/kg, between about 90 μg/kg to about 400 μg/kg, between about 90 μg/kg to about 350 μg/kg, between about 90 μg/kg to about 300 μg/kg, between about 90 μg/kg to about 250 μg/kg, between about 90 μg/kg to about 200 μg/kg, between about 90 μg/kg to about 150 μg/kg, between about 90 μg/kg to about 135 μg/kg, between about 90 μg/kg to about 100 μg/kg, between about 100 μg/kg to about 500 μg/kg, between about 100 μg/kg to about 450 μg/kg, between about 100 μg/kg to about 400 μg/kg, between about 100 μg/kg to about 350 μg/kg, between about 100 μg/kg to about 300 μg/kg, between about 100 μg/kg to about 250 μg/kg, between about 100 μg/kg to about 200 μg/kg, between about 100 μg/kg to about 150 μg/kg, between about 100 μg/kg to about 135 μg/kg, between about 125 μg/kg to about 500 μg/kg, between about 125 μg/kg to about 450 μg/kg, between about 125 μg/kg to about 400 μg/kg, between about 125 μg/kg to about 350 μg/kg, between about 125 μg/kg to about 300 μg/kg, between about 125 μg/kg to about 250 μg/kg, between about 125 μg/kg to about 200 μg/kg, between about 125 μg/kg to about 150 μg/kg, between about 125 μg/kg to about 135 μg/kg, between about 135 μg/kg to about 500 μg/kg, between about 135 μg/kg to about 450 μg/kg, between about 135 μg/kg to about 400 μg/kg, between about 135 μg/kg to about 350 μg/kg, between about 135 μg/kg to about 300 μg/kg, between about 135 μg/kg to about 250 μg/kg, between about 135 μg/kg to about 200 μg/kg, between about 135 μg/kg to about 150 μg/kg, between about 150 μg/kg to about 500 μg/kg, between about 150 μg/kg to about 450 μg/kg, between about 150 μg/kg to about 400 μg/kg, between about 150 μg/kg to about 350 μg/kg, between about 150 μg/kg to about 300 μg/kg, between about 150 μg/kg to about 250 μg/kg, between about 150 μg/kg to about 200 μg/kg, between about 200 μg/kg to about 500 μg/kg, between about 200 μg/kg to about 450 μg/kg, between about 200 μg/kg to about 400 μg/kg, between about 200 μg/kg to about 350 μg/kg, between about 200 μg/kg to about 300 μg/kg, between about 200 μg/kg to about 250 μg/kg, between about 250 μg/kg to about 500 μg/kg, between about 250 μg/kg to about 450 μg/kg, between about 250 μg/kg to about 400 μg/kg, between about 250 μg/kg to about 350 μg/kg, between about 250 μg/kg to about 300 μg/kg, between about 300 μg/kg to about 500 μg/kg, between about 300 μg/kg to about 450 μg/kg, between about 300 μg/kg to about 400 μg/kg, between about 300 μg/kg to about 350 μg/kg, between about 350 μg/kg to about 500 μg/kg, between about 350 μg/kg to about 450 μg/kg, between about 350 μg/kg to about 400 μg/kg, between about 400 μg/kg to about 500 μg/kg, between about 400 μg/kg to about 450 μg/kg, or between about 450 μg/kg to about 500 μg/kg. In some embodiments, the C1D1, the C1D2, and the C1D3 are each between about 15 μg/kg to about 90 μg/kg. In some embodiments, the C1D1, the C1D2, and the C1D3 are each between about 20 μg/kg to about 40 μg/kg. In some embodiments, the C1D1, the C1D2, and the C1D3 are each between about 50 μg/kg to about 70 μg/kg. In particular embodiments, the C1D1, the C1D2, and the C1D3 are each about 30 μg/kg. For example, in some embodiments, about 30 μg/kg of the IL-22 Fc fusion protein is administered to the subject q4w. In other particular embodiments, the C1D1, the C1D2, and the C1D3 are each about 60 μg/kg. For example, in some embodiments, about 60 μg/kg of the IL-22 Fc fusion protein is administered to the subject q4w.

The C1D1, the C1D2, and the C1D3 can be administered on any suitable day of the dosing cycle (e.g., the first dosing cycle) in any of the preceding methods. For example, in some embodiments, the C1D1 is administered on Week 0, on Week 1, on Week 2, on Week 3, on Week 4, on Week 5, on Week 6, on Week 7, on Week 8, on Week 9, on Week 10, on Week 11, on Week 12, on Week 13, on Week 14, on Week 15, on Week 16, on Week 17, on Week 18, on Week 19, on Week 20, on Week 21, on Week 22, on Week 23, on Week 24, on Week 25, on Week 26, on Week 27, on Week 28, on Week 29, or on Week 30 of the dosing cycle. For example, in some embodiments, the C1D2 is administered on on Week 0, on Week 1, on Week 2, on Week 3, on Week 4, on Week 5, on Week 6, on Week 7, on Week 8, on Week 9, on Week 10, on Week 11, on Week 12, on Week 13, on Week 14, on Week 15, on Week 16, on Week 17, on Week 18, on Week 19, on Week 20, on Week 21, on Week 22, on Week 23, on Week 24, on Week 25, on Week 26, on Week 27, on Week 28, on Week 29, or on Week 30 of the dosing cycle. For example, in some embodiments, the C1D3 is administered on Week 0, on Week 1, on Week 2, on Week 3, on Week 4, on Week 5, on Week 6, on Week 7, on Week 8, on Week 9, on Week 10, on Week 11, on Week 12, on Week 13, on Week 14, on Week 15, on Week 16, on Week 17, on Week 18, on Week 19, on Week 20, on Week 21, on Week 22, on Week 23, on Week 24, on Week 25, on Week 26, on Week 27, on Week 28, on Week 29, or on Week 30 of the dosing cycle. In particular embodiments, the method includes administering the C1D1, the C1D2, and the C1D3 on or about Weeks 0, 4, and 8, respectively, of the dosing cycle. In other particular embodiments, the method includes administering the C1D1, the C1D2, and the C1D3 on or about Weeks 1, 4, and 8, respectively, of the dosing cycle. In some embodiments, the C1D1 is administered on Day 0 of Week 1.

For example, in some embodiments, the dosing cycle (e.g., the first dosing cycle) comprises a first dose (C1D1), a second dose (C1D2), and a third dose (C1D3) of the IL-22 Fc fusion protein. In some embodiments, the dosing cycle (e.g., the first dosing cycle) consists of the C1D1, the C1D2, and the C1D3. In some embodiments, the C1D1, the C1D2, and the C1D3 are each between about 15 μg/kg to about 90 μg/kg. In some embodiments, the C1D1, the C1D2, and the C1D3 are each between about 20 μg/kg to about 40 μg/kg. In some embodiments, the C1D1, the C1D2, and the C1D3 are each about 30 μg/kg. In some embodiments, the C1D1, the C1D2, and the C1D3 are each between about 50 μg/kg to about 70 μg/kg. In some embodiments, the C1D1, the C1D2, and the C1D3 are each about 60 μg/kg. In some embodiments, the C1D1, the C1D2, and the C1D3 are each about 90 μg/kg. In some embodiments, the method comprises administering the C1D1, the C1D2, and the C1D3 on or about Weeks 0, 4, and 8, respectively, of the dosing cycle (e.g., the first dosing cycle). In other embodiments, the method comprises administering the C1D1, the C1D2, and the C1D3 on or about Weeks 1, 4, and 8, respectively, of the dosing cycle (e.g., the first dosing cycle). In some embodiments, the C1D1 is administered on Day 0 of Week 1.

In some embodiments of any of the preceding methods, the dosing cycle (e.g., the first dosing cycle) includes comprises a first dose (C1D1), a second dose (C1D2), a third dose (C1D3), a fourth dose (C1D4), a fifth dose (C1D5), and a sixth dose (C1D6) of the IL-22 Fc fusion protein. In some embodiments, the dosing cycle consists of the C1D1, the C1D2, the C1D3, the C1D4, the C1D5, and the C1D6. The C1D1, the C1D2, the C1D3, the C1D4, the C1D5, and the C1D6 may be administered, for example, q2w in a dosing cycle of about 10 weeks. The C1D1, the C1D2, the C1D3, the C1D4, the C1D5, and the C1D6 may be the same amount or different amounts of the IL-22 Fc fusion protein. In some embodiments, the C1D1, the C1D2, the C1D3, the C1D4, the C1D5, and the C1D6 are each between about 1 μg/kg to about 500 μg/kg, e.g., about 1 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 110 μg/kg, about 120 μg/kg, about 130 μg/kg, about 140 μg/kg, about 150 μg/kg, about 160 μg/kg, about 170 μg/kg, about 180 μg/kg, about 190 μg/kg, about 200 μg/kg, about 210 μg/kg, about 220 μg/kg, about 230 μg/kg, about 240 μg/kg, about 250 μg/kg, about 260 μg/kg, about 270 μg/kg, about 280 μg/kg, about 290 μg/kg, about 300 μg/kg, about 310 μg/kg, about 320 μg/kg, about 330 μg/kg, about 340 μg/kg, about 350 μg/kg, about 360 μg/kg, about 370 μg/kg, about 380 μg/kg, about 390 μg/kg, about 400 μg/kg, about 410 μg/kg, about 420 μg/kg, about 430 μg/kg, about 440 μg/kg, about 450 μg/kg, about 460 μg/kg, about 470 μg/kg, about 480 μg/kg, about 490 μg/kg, or about 500 μg/kg. In some embodiments, the C1D1, the C1D2, the C1D3, the C1D4, the C1D5, and the C1D6 are each between about 1 μg/kg to about 500 μg/kg, between about 1 μg/kg to about 450 μg/kg, between about 1 μg/kg to about 400 μg/kg, between about 1 μg/kg to about 350 μg/kg, between about 1 μg/kg to about 300 μg/kg, between about 1 μg/kg to about 250 μg/kg, between about 1 μg/kg to about 200 μg/kg, between about 1 μg/kg to about 150 μg/kg, between about 1 μg/kg to about 135 μg/kg, between about 1 μg/kg to about 100 μg/kg, between about 1 μg/kg to about 90 μg/kg, between about 1 μg/kg to about 75 μg/kg, between about 1 μg/kg to about 50 μg/kg, between about 1 μg/kg to about 25 μg/kg, between about 25 μg/kg to about 500 μg/kg, between about 25 μg/kg to about 450 μg/kg, between about 25 μg/kg to about 400 μg/kg, between about 25 μg/kg to about 350 μg/kg, between about 25 μg/kg to about 300 μg/kg, between about 25 μg/kg to about 250 μg/kg, between about 25 μg/kg to about 200 μg/kg, between about 25 μg/kg to about 150 μg/kg, between about 25 μg/kg to about 135 μg/kg, between about 25 μg/kg to about 100 μg/kg, between about 25 μg/kg to about 75 μg/kg, between about 25 μg/kg to about 50 μg/kg, between about 50 μg/kg to about 500 μg/kg, between about 50 μg/kg to about 450 μg/kg, between about 50 μg/kg to about 400 μg/kg, between about 50 μg/kg to about 350 μg/kg, between about 50 μg/kg to about 300 μg/kg, between about 50 μg/kg to about 250 μg/kg, between about 50 μg/kg to about 200 μg/kg, between about 50 μg/kg to about 150 μg/kg, between about 50 μg/kg to about 135 μg/kg, between about 50 μg/kg to about 100 μg/kg, between about 50 μg/kg to about 75 μg/kg, between about 75 μg/kg to about 500 μg/kg, between about 75 μg/kg to about 450 μg/kg, between about 75 μg/kg to about 400 μg/kg, between about 75 μg/kg to about 350 μg/kg, between about 75 μg/kg to about 300 μg/kg, between about 75 μg/kg to about 250 μg/kg, between about 75 μg/kg to about 200 μg/kg, between about 75 μg/kg to about 150 μg/kg, between about 75 μg/kg to about 135 μg/kg, between about 75 μg/kg to about 100 μg/kg, between about 80 μg/kg to about 500 μg/kg, between about 80 μg/kg to about 450 μg/kg, between about 80 μg/kg to about 400 μg/kg, between about 80 μg/kg to about 350 μg/kg, between about 80 μg/kg to about 300 μg/kg, between about 80 μg/kg to about 250 μg/kg, between about 80 μg/kg to about 200 μg/kg, between about 80 μg/kg to about 150 μg/kg, between about 80 μg/kg to about 135 μg/kg, between about 80 μg/kg to about 100 μg/kg, between about 90 μg/kg to about 500 μg/kg, between about 90 μg/kg to about 450 μg/kg, between about 90 μg/kg to about 400 μg/kg, between about 90 μg/kg to about 350 μg/kg, between about 90 μg/kg to about 300 μg/kg, between about 90 μg/kg to about 250 μg/kg, between about 90 μg/kg to about 200 μg/kg, between about 90 μg/kg to about 150 μg/kg, between about 90 μg/kg to about 135 μg/kg, between about 90 μg/kg to about 100 μg/kg, between about 100 μg/kg to about 500 μg/kg, between about 100 μg/kg to about 450 μg/kg, between about 100 μg/kg to about 400 μg/kg, between about 100 μg/kg to about 350 μg/kg, between about 100 μg/kg to about 300 μg/kg, between about 100 μg/kg to about 250 μg/kg, between about 100 μg/kg to about 200 μg/kg, between about 100 μg/kg to about 150 μg/kg, between about 100 μg/kg to about 135 μg/kg, between about 125 μg/kg to about 500 μg/kg, between about 125 μg/kg to about 450 μg/kg, between about 125 μg/kg to about 400 μg/kg, between about 125 μg/kg to about 350 μg/kg, between about 125 μg/kg to about 300 μg/kg, between about 125 μg/kg to about 250 μg/kg, between about 125 μg/kg to about 200 μg/kg, between about 125 μg/kg to about 150 μg/kg, between about 125 μg/kg to about 135 μg/kg, between about 135 μg/kg to about 500 μg/kg, between about 135 μg/kg to about 450 μg/kg, between about 135 μg/kg to about 400 μg/kg, between about 135 μg/kg to about 350 μg/kg, between about 135 μg/kg to about 300 μg/kg, between about 135 μg/kg to about 250 μg/kg, between about 135 μg/kg to about 200 μg/kg, between about 135 μg/kg to about 150 μg/kg, between about 150 μg/kg to about 500 μg/kg, between about 150 μg/kg to about 450 μg/kg, between about 150 μg/kg to about 400 μg/kg, between about 150 μg/kg to about 350 μg/kg, between about 150 μg/kg to about 300 μg/kg, between about 150 μg/kg to about 250 μg/kg, between about 150 μg/kg to about 200 μg/kg, between about 200 μg/kg to about 500 μg/kg, between about 200 μg/kg to about 450 μg/kg, between about 200 μg/kg to about 400 μg/kg, between about 200 μg/kg to about 350 μg/kg, between about 200 μg/kg to about 300 μg/kg, between about 200 μg/kg to about 250 μg/kg, between about 250 μg/kg to about 500 μg/kg, between about 250 μg/kg to about 450 μg/kg, between about 250 μg/kg to about 400 μg/kg, between about 250 μg/kg to about 350 μg/kg, between about 250 μg/kg to about 300 μg/kg, between about 300 μg/kg to about 500 μg/kg, between about 300 μg/kg to about 450 μg/kg, between about 300 μg/kg to about 400 μg/kg, between about 300 μg/kg to about 350 μg/kg, between about 350 μg/kg to about 500 μg/kg, between about 350 μg/kg to about 450 μg/kg, between about 350 μg/kg to about 400 μg/kg, between about 400 μg/kg to about 500 μg/kg, between about 400 μg/kg to about 450 μg/kg, or between about 450 μg/kg to about 500 μg/kg. In some embodiments, the C1D1, the C1D2, the C1D3, the C1D4, the C1D5, and the C1D6 are each between about 30 μg/kg to about 90 μg/kg. In some embodiments, the C1D1, the C1D2, the C1D3, the C1D4, the C1D5, and the C1D6 are each between about 50 μg/kg to about 70 μg/kg. In particular embodiments, the C1D1, the C1D2, the C1D3, the C1D4, the C1D5, and the C1D6 are each about 60 μg/kg. For example, in some embodiments, about 60 μg/kg of the IL-22 Fc fusion protein is administered to the subject q2w. In other particular embodiments, the C1D1, the C1D2, the C1D3, the C1D4, the C1D5, and the C1D6 are each about 15 μg/kg. For example, in some embodiments, about 15 μg/kg of the IL-22 Fc fusion protein is administered to the subject q2w. In other particular embodiments, the C1D1, the C1D2, the C1D3, the C1D4, the C1D5, and the C1D6 are each about 30 μg/kg. For example, in some embodiments, about 30 μg/kg of the IL-22 Fc fusion protein is administered to the subject q2w. In other particular embodiments, the C1D1, the C1D2, the C1D3, the C1D4, the C1D5, and the C1D6 are each about 90 μg/kg. For example, in some embodiments, about 90 μg/kg of the IL-22 Fc fusion protein is administered to the subject q2w.

The C1D1, the C1D2, the C1D3, the C1D4, the C1D5, and the C1D6 can be administered on any suitable day of the dosing cycle (e.g., the first dosing cycle) in any of the preceding methods. For example, in some embodiments, the C1D1 is administered on Week 0, on Week 1, on Week 2, on Week 3, on Week 4, on Week 5, on Week 6, on Week 7, on Week 8, on Week 9, on Week 10, on Week 11, on Week 12, on Week 13, on Week 14, on Week 15, on Week 16, on Week 17, on Week 18, on Week 19, on Week 20, on Week 21, on Week 22, on Week 23, on Week 24, on Week 25, on Week 26, on Week 27, on Week 28, on Week 29, or on Week 30 of the dosing cycle. For example, in some embodiments, the C1D2 is administered on Week 0, on Week 1, on Week 2, on Week 3, on Week 4, on Week 5, on Week 6, on Week 7, on Week 8, on Week 9, on Week 10, on Week 11, on Week 12, on Week 13, on Week 14, on Week 15, on Week 16, on Week 17, on Week 18, on Week 19, on Week 20, on Week 21, on Week 22, on Week 23, on Week 24, on Week 25, on Week 26, on Week 27, on Week 28, on Week 29, or on Week 30 of the dosing cycle. For example, in some embodiments, the C1D3 is administered on Week 0, on Week 1, on Week 2, on Week 3, on Week 4, on Week 5, on Week 6, on Week 7, on Week 8, on Week 9, on Week 10, on Week 11, on Week 12, on Week 13, on Week 14, on Week 15, on Week 16, on Week 17, on Week 18, on Week 19, on Week 20, on Week 21, on Week 22, on Week 23, on Week 24, on Week 25, on Week 26, on Week 27, on Week 28, on Week 29, or on Week 30 of the dosing cycle. For example, in some embodiments, the C1D4 is administered on Week 0, on Week 1, on Week 2, on Week 3, on Week 4, on Week 5, on Week 6, on Week 7, on Week 8, on Week 9, on Week 10, on Week 11, on Week 12, on Week 13, on Week 14, on Week 15, on Week 16, on Week 17, on Week 18, on Week 19, on Week 20, on Week 21, on Week 22, on Week 23, on Week 24, on Week 25, on Week 26, on Week 27, on Week 28, on Week 29, or on Week 30 of the dosing cycle. For example, in some embodiments, the C1D5 is administered on Week 0, on Week 1, on Week 2, on Week 3, on Week 4, on Week 5, on Week 6, on Week 7, on Week 8, on Week 9, on Week 10, on Week 11, on Week 12, on Week 13, on Week 14, on Week 15, on Week 16, on Week 17, on Week 18, on Week 19, on Week 20, on Week 21, on Week 22, on Week 23, on Week 24, on Week 25, on Week 26, on Week 27, on Week 28, on Week 29, or on Week 30 of the dosing cycle. For example, in some embodiments, the C1D6 is administered on Week 0, on Week 1, on Week 2, on Week 3, on Week 4, on Week 5, on Week 6, on Week 7, on Week 8, on Week 9, on Week 10, on Week 11, on Week 12, on Week 13, on Week 14, on Week 15, on Week 16, on Week 17, on Week 18, on Week 19, on Week 20, on Week 21, on Week 22, on Week 23, on Week 24, on Week 25, on Week 26, on Week 27, on Week 28, on Week 29, or on Week 30 of the dosing cycle. In particular embodiments, the method includes administering C1D1, the C1D2, the C1D3, the C1D4, the C1D5, and the C1D6 on or about Weeks 0, 2, 4, 6, 8, and 10, respectively, of the dosing cycle.

In any of the preceding methods, in some embodiments, the dosing regimen further includes one or more further dosing cycles. For example, in some embodiments, the dosing regimen further includes one, two, three, four, five, six, seven, eight, nine, ten, eleven, twelve, thirteen, fourteen, fifteen, sixteen, seventeen, eighteen, nineteen, or twenty further dosing cycles. In particular embodiments, the dosing regimen further comprises a second dosing cycle.

In some embodiments, the dose(s) in the further (e.g., second) dosing cycle(s) are administered to the subject every week (q1w), every two weeks (q2w), every three weeks (q3w), every four weeks, (q4w), every five weeks (q5w), every six weeks (q6w), every seven weeks (q7w), every eight weeks (q8w), every nine weeks (q9w), every ten weeks (q10w), every 12 weeks (q12w), every fourteen weeks (q14w), every sixteen weeks (q16w), every eighteen weeks (q18w), or every twenty weeks (q20w).

In some embodiments of any of the preceding methods, the length of the further (e.g., second) dosing cycle is between about 5 weeks and about 80 weeks, e.g., about 5 weeks, about 6 weeks, about 7 weeks, about 9 weeks, about 10 weeks, about 11 weeks, about 12 weeks, about 14 weeks, about 16 weeks, about 18 weeks, about 20 weeks, about 22 weeks, about 24 weeks, about 26 weeks, about 28 weeks, about 30 weeks, about 32 weeks, about 34 weeks, about 36 weeks, about 38 weeks, about 40 weeks, about 42 weeks, about 44 weeks, about 46 weeks, about 48 weeks, about 50 weeks, about 52 weeks, about 54 weeks, about 56 weeks, about 58 weeks, about 60 weeks, about 62 weeks, about 64 weeks, about 66 weeks, about 68 weeks, about 70 weeks, about 72 weeks, about 74 weeks, about 76 weeks, about 78 weeks, or about 80 weeks.

For example, in some embodiments of any of the preceding methods, the length of the further (e.g., second) dosing cycle is between about 5 weeks and about 80 weeks, between about 5 weeks and about 75 weeks, between about 5 weeks and about 70 weeks, between about 5 weeks and about 65 weeks, between about 5 weeks and about 60 weeks, between about 5 weeks and about 55 weeks, between about 5 weeks and about 50 weeks, between about 5 weeks and about 45 weeks, between about 5 weeks and about 40 weeks, between about 5 weeks and about 35 weeks, between about 5 weeks and about 30 weeks, between about 5 weeks and about 25 weeks, between about 5 weeks and about 20 weeks, between about 5 weeks and about 15 weeks, between about 5 weeks and about 10 weeks, between about 10 weeks and about 80 weeks, between about 10 weeks and about 75 weeks, between about 10 weeks and about 70 weeks, between about 10 weeks and about 65 weeks, between about 10 weeks and about 60 weeks, between about 10 weeks and about 55 weeks, between about 10 weeks and about 50 weeks, between about 10 weeks and about 45 weeks, between about 10 weeks and about 40 weeks, between about 10 weeks and about 35 weeks, between about 10 weeks and about 30 weeks, between about 10 weeks and about 25 weeks, between about 10 weeks and about 20 weeks, between about 10 weeks and about 15 weeks, between about 15 weeks and about 80 weeks, between about 15 weeks and about 75 weeks, between about 15 weeks and about 70 weeks, between about 15 weeks and about 65 weeks, between about 15 weeks and about 60 weeks, between about 15 weeks and about 55 weeks, between about 15 weeks and about 50 weeks, between about 15 weeks and about 45 weeks, between about 15 weeks and about 40 weeks, between about 15 weeks and about 35 weeks, between about 15 weeks and about 30 weeks, between about 15 weeks and about 25 weeks, between about 15 weeks and about 20 weeks, between about 20 weeks and about 80 weeks, between about 20 weeks and about 75 weeks, between about 20 weeks and about 70 weeks, between about 20 weeks and about 65 weeks, between about 20 weeks and about 60 weeks, between about 20 weeks and about 55 weeks, between about 20 weeks and about 50 weeks, between about 20 weeks and about 45 weeks, between about 20 weeks and about 40 weeks, between about 20 weeks and about 35 weeks, between about 20 weeks and about 30 weeks, between about 20 weeks and about 25 weeks, between about 25 weeks and about 80 weeks, between about 25 weeks and about 75 weeks, between about 25 weeks and about 70 weeks, between about 25 weeks and about 65 weeks, between about 25 weeks and about 60 weeks, between about 25 weeks and about 55 weeks, between about 25 weeks and about 50 weeks, between about 25 weeks and about 45 weeks, between about 25 weeks and about 40 weeks, between about 25 weeks and about 35 weeks, between about 25 weeks and about 30 weeks, between about 30 weeks and about 80 weeks, between about 30 weeks and about 75 weeks, between about 30 weeks and about 70 weeks, between about 30 weeks and about 65 weeks, between about 30 weeks and about 60 weeks, between about 30 weeks and about 55 weeks, between about 30 weeks and about 50 weeks, between about 30 weeks and about 45 weeks, between about 30 weeks and about 40 weeks, between about 30 weeks and about 35 weeks, between about 35 weeks and about 80 weeks, between about 35 weeks and about 75 weeks, between about 35 weeks and about 70 weeks, between about 35 weeks and about 65 weeks, between about 35 weeks and about 60 weeks, between about 35 weeks and about 55 weeks, between about 35 weeks and about 50 weeks, between about 35 weeks and about 45 weeks, between about 35 weeks and about 40 weeks, between about 40 weeks and about 80 weeks, between about 40 weeks and about 75 weeks, between about 40 weeks and about 70 weeks, between about 40 weeks and about 65 weeks, between about 40 weeks and about 60 weeks, between about 40 weeks and about 55 weeks, between about 40 weeks and about 50 weeks, between about 40 weeks and about 45 weeks, between about 45 weeks and about 80 weeks, between about 45 weeks and about 75 weeks, between about 45 weeks and about 70 weeks, between about 45 weeks and about 65 weeks, between about 45 weeks and about 60 weeks, between about 45 weeks and about 55 weeks, between about 45 weeks and about 50 weeks, between about 50 weeks and about 80 weeks, between about 50 weeks and about 75 weeks, between about 50 weeks and about 70 weeks, between about 50 weeks and about 65 weeks, between about 50 weeks and about 60 weeks, between about 50 weeks and about 55 weeks, between about 55 weeks and about 80 weeks, between about 55 weeks and about 75 weeks, between about 55 weeks and about 70 weeks, between about 55 weeks and about 65 weeks, between about 55 weeks and about 60 weeks, between about 60 weeks and about 80 weeks, between about 60 weeks and about 75 weeks, between about 60 weeks and about 70 weeks, between about 60 weeks and about 65 weeks, between about 65 weeks and about 80 weeks, between about 65 weeks and about 75 weeks, between about 65 weeks and about 70 weeks, between about 70 weeks and about 80 weeks, between about 70 weeks and about 75 weeks, or between about 75 weeks and about 80 weeks. In some embodiments, the length of the further (e.g., second) dosing cycle is between about 10 weeks and about 40 weeks. In some embodiments, the length of the further (e.g., second) dosing cycle is between about 15 weeks and about 25 weeks. In particular embodiments, the length of the further (e.g., second) dosing cycle is about 20 weeks.

In other embodiments of any of the preceding methods, the further (e.g., second) dosing cycle continues indefinitely or until clinical remission. In some embodiments, the further (e.g., second) dosing cycle is stopped following the clinical remission, and then restarted following a relapse of the IBD.

The further (e.g., second) dosing cycle may include any suitable number of doses, e.g., about one, about two, about three, about four, about five, about six, about seven, about eight, about nine, about ten, about eleven, about twelve, about thirteen, about fourteen, about fifteen, about sixteen, about seventeen, about eighteen, about nineteen, or about twenty doses. For example, in some embodiments of any of the preceding methods, the further (e.g., second) dosing cycle includes about one to about twenty doses, about one to about nineteen doses, about one to about eighteen doses, about one to about seventeen doses, about one to about sixteen doses, about one to about fifteen doses, about one to about fourteen doses, about one to about thirteen doses, about one to about twelve doses, about one to about eleven doses, about one to about ten doses, about one to about nine doses, about one to about eight doses, about one to about seven doses, about one to about six doses, about one to about five doses, about one to about four doses, about one to about three doses, about one to about two doses, about two to about twenty doses, about two to about nineteen doses, about two to about eighteen doses, about two to about seventeen doses, about two to about sixteen doses, about two to about fifteen doses, about two to about fourteen doses, about two to about thirteen doses, about two to about twelve doses, about two to about eleven doses, about two to about ten doses, about two to about nine doses, about two to about eight doses, about two to about seven doses, about two to about six doses, about two to about five doses, about two to about four doses, about two to about three doses, about three to about twenty doses, about three to about nineteen doses, about three to about eighteen doses, about three to about seventeen doses, about three to about sixteen doses, about three to about fifteen doses, about three to about fourteen doses, about three to about thirteen doses, about three to about twelve doses, about three to about eleven doses, about three to about ten doses, about three to about nine doses, about three to about eight doses, about three to about seven doses, about three to about six doses, about three to about five doses, about three to about four doses, about four to about twenty doses, about four to about nineteen doses, about four to about eighteen doses, about four to about seventeen doses, about four to about sixteen doses, about four to about fifteen doses, about four to about fourteen doses, about four to about thirteen doses, about four to about twelve doses, about four to about eleven doses, about four to about ten doses, about four to about nine doses, about four to about eight doses, about four to about seven doses, about four to about six doses, about four to about five doses, about five to about twenty doses, about five to about nineteen doses, about five to about eighteen doses, about five to about seventeen doses, about five to about sixteen doses, about five to about fifteen doses, about five to about fourteen doses, about five to about thirteen doses, about five to about twelve doses, about five to about eleven doses, about five to about ten doses, about five to about nine doses, about five to about eight doses, about five to about seven doses, about five to about six doses, about six to about twenty doses, about six to about nineteen doses, about six to about eighteen doses, about six to about seventeen doses, about six to about sixteen doses, about six to about fifteen doses, about six to about fourteen doses, about six to about thirteen doses, about six to about twelve doses, about six to about eleven doses, about six to about ten doses, about six to about nine doses, about six to about eight doses, about six to about seven doses, about seven to about twenty doses, about seven to about nineteen doses, about seven to about eighteen doses, about seven to about seventeen doses, about seven to about sixteen doses, about seven to about fifteen doses, about seven to about fourteen doses, about seven to about thirteen doses, about seven to about twelve doses, about seven to about eleven doses, about seven to about ten doses, about seven to about nine doses, about seven to about eight doses, about eight to about twenty doses, about eight to about nineteen doses, about eight to about eighteen doses, about eight to about seventeen doses, about eight to about sixteen doses, about eight to about fifteen doses, about eight to about fourteen doses, about eight to about thirteen doses, about eight to about twelve doses, about eight to about eleven doses, about eight to about ten doses, about eight to about nine doses, about nine to about twenty doses, about nine to about nineteen doses, about nine to about eighteen doses, about nine to about seventeen doses, about nine to about sixteen doses, about nine to about fifteen doses, about nine to about fourteen doses, about nine to about thirteen doses, about nine to about twelve doses, about nine to about eleven doses, about nine to about ten doses, about ten to about twenty doses, about ten to about nineteen doses, about ten to about eighteen doses, about ten to about seventeen doses, about ten to about sixteen doses, about ten to about fifteen doses, about ten to about fourteen doses, about ten to about thirteen doses, about ten to about twelve doses, about ten to about eleven doses, about eleven to about twenty doses, about eleven to about nineteen doses, about eleven to about eighteen doses, about eleven to about seventeen doses, about eleven to about sixteen doses, about eleven to about fifteen doses, about eleven to about fourteen doses, about eleven to about thirteen doses, about eleven to about twelve doses, about twelve to about twenty doses, about twelve to about nineteen doses, about twelve to about eighteen doses, about twelve to about seventeen doses, about twelve to about sixteen doses, about twelve to about fifteen doses, about twelve to about fourteen doses, about twelve to about thirteen doses, about thirteen to about twenty doses, about thirteen to about nineteen doses, about thirteen to about eighteen doses, about thirteen to about seventeen doses, about thirteen to about sixteen doses, about thirteen to about fifteen doses, about thirteen to about fourteen doses, about fourteen to about twenty doses, about fourteen to about nineteen doses, about fourteen to about eighteen doses, about fourteen to about seventeen doses, about fourteen to about sixteen doses, about fourteen to about fifteen doses, about fifteen to about twenty doses, about fifteen to about nineteen doses, about fifteen to about eighteen doses, about fifteen to about seventeen doses, about fifteen to about sixteen doses, about sixteen to about twenty doses, about sixteen to about nineteen doses, about sixteen to about eighteen doses, about sixteen to about seventeen doses, about seventeen to about twenty doses, about seventeen to about nineteen doses, about seventeen to about eighteen doses, about eighteen to about twenty doses, about eighteen to about nineteen doses, or about nineteen to about twenty doses.

For example, in some embodiments of any of the preceding methods, the further (e.g., second) dosing cycle comprises a first dose (C2D1), a second dose (C2D2), and a third dose (C2D3) of the IL-22 Fc fusion protein. In some embodiments, the C2D1, the C2D2, and the C2D3 are administered to the subject q8w. In other embodiments, the C2D1, the C2D2, and the C2D3 are administered to the subject q4w.

In some embodiments, the further (e.g., second) dosing cycle consists of the C2D1, the C2D2, and the C2D3. The C2D1, the C2D2, and the C2D3 may be the same amount or different amounts of the IL-22 Fc fusion protein. In some embodiments, the C2D1, the C2D2, and the C2D3 are each between about 1 μg/kg to about 500 μg/kg, e.g., about 1 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 110 μg/kg, about 120 μg/kg, about 130 μg/kg, about 140 μg/kg, about 150 μg/kg, about 160 μg/kg, about 170 μg/kg, about 180 μg/kg, about 190 μg/kg, about 200 μg/kg, about 210 μg/kg, about 220 μg/kg, about 230 μg/kg, about 240 μg/kg, about 250 μg/kg, about 260 μg/kg, about 270 μg/kg, about 280 μg/kg, about 290 μg/kg, about 300 μg/kg, about 310 μg/kg, about 320 μg/kg, about 330 μg/kg, about 340 μg/kg, about 350 μg/kg, about 360 μg/kg, about 370 μg/kg, about 380 μg/kg, about 390 μg/kg, about 400 μg/kg, about 410 μg/kg, about 420 μg/kg, about 430 μg/kg, about 440 μg/kg, about 450 μg/kg, about 460 μg/kg, about 470 μg/kg, about 480 μg/kg, about 490 μg/kg, or about 500 μg/kg. In some embodiments, the C2D1, the C2D2, and the C2D3 are each between about 1 μg/kg to about 500 μg/kg, between about 1 μg/kg to about 450 μg/kg, between about 1 μg/kg to about 400 μg/kg, between about 1 μg/kg to about 350 μg/kg, between about 1 μg/kg to about 300 μg/kg, between about 1 μg/kg to about 250 μg/kg, between about 1 μg/kg to about 200 μg/kg, between about 1 μg/kg to about 150 μg/kg, between about 1 μg/kg to about 135 μg/kg, between about 1 μg/kg to about 100 μg/kg, between about 1 μg/kg to about 90 μg/kg, between about 1 μg/kg to about 75 μg/kg, between about 1 μg/kg to about 50 μg/kg, between about 1 μg/kg to about 25 μg/kg, between about 25 μg/kg to about 500 μg/kg, between about 25 μg/kg to about 450 μg/kg, between about 25 μg/kg to about 400 μg/kg, between about 25 μg/kg to about 350 μg/kg, between about 25 μg/kg to about 300 μg/kg, between about 25 μg/kg to about 250 μg/kg, between about 25 μg/kg to about 200 μg/kg, between about 25 μg/kg to about 150 μg/kg, between about 25 μg/kg to about 135 μg/kg, between about 25 μg/kg to about 100 μg/kg, between about 25 μg/kg to about 75 μg/kg, between about 25 μg/kg to about 50 μg/kg, between about 50 μg/kg to about 500 μg/kg, between about 50 μg/kg to about 450 μg/kg, between about 50 μg/kg to about 400 μg/kg, between about 50 μg/kg to about 350 μg/kg, between about 50 μg/kg to about 300 μg/kg, between about 50 μg/kg to about 250 μg/kg, between about 50 μg/kg to about 200 μg/kg, between about 50 μg/kg to about 150 μg/kg, between about 50 μg/kg to about 135 μg/kg, between about 50 μg/kg to about 100 μg/kg, between about 50 μg/kg to about 75 μg/kg, between about 75 μg/kg to about 500 μg/kg, between about 75 μg/kg to about 450 μg/kg, between about 75 μg/kg to about 400 μg/kg, between about 75 μg/kg to about 350 μg/kg, between about 75 μg/kg to about 300 μg/kg, between about 75 μg/kg to about 250 μg/kg, between about 75 μg/kg to about 200 μg/kg, between about 75 μg/kg to about 150 μg/kg, between about 75 μg/kg to about 135 μg/kg, between about 75 μg/kg to about 100 μg/kg, between about 80 μg/kg to about 500 μg/kg, between about 80 μg/kg to about 450 μg/kg, between about 80 μg/kg to about 400 μg/kg, between about 80 μg/kg to about 350 μg/kg, between about 80 μg/kg to about 300 μg/kg, between about 80 μg/kg to about 250 μg/kg, between about 80 μg/kg to about 200 μg/kg, between about 80 μg/kg to about 150 μg/kg, between about 80 μg/kg to about 135 μg/kg, between about 80 μg/kg to about 100 μg/kg, between about 90 μg/kg to about 500 μg/kg, between about 90 μg/kg to about 450 μg/kg, between about 90 μg/kg to about 400 μg/kg, between about 90 μg/kg to about 350 μg/kg, between about 90 μg/kg to about 300 μg/kg, between about 90 μg/kg to about 250 μg/kg, between about 90 μg/kg to about 200 μg/kg, between about 90 μg/kg to about 150 μg/kg, between about 90 μg/kg to about 135 μg/kg, between about 90 μg/kg to about 100 μg/kg, between about 100 μg/kg to about 500 μg/kg, between about 100 μg/kg to about 450 μg/kg, between about 100 μg/kg to about 400 μg/kg, between about 100 μg/kg to about 350 μg/kg, between about 100 μg/kg to about 300 μg/kg, between about 100 μg/kg to about 250 μg/kg, between about 100 μg/kg to about 200 μg/kg, between about 100 μg/kg to about 150 μg/kg, between about 100 μg/kg to about 135 μg/kg, between about 125 μg/kg to about 500 μg/kg, between about 125 μg/kg to about 450 μg/kg, between about 125 μg/kg to about 400 μg/kg, between about 125 μg/kg to about 350 μg/kg, between about 125 μg/kg to about 300 μg/kg, between about 125 μg/kg to about 250 μg/kg, between about 125 μg/kg to about 200 μg/kg, between about 125 μg/kg to about 150 μg/kg, between about 125 μg/kg to about 135 μg/kg, between about 135 μg/kg to about 500 μg/kg, between about 135 μg/kg to about 450 μg/kg, between about 135 μg/kg to about 400 μg/kg, between about 135 μg/kg to about 350 μg/kg, between about 135 μg/kg to about 300 μg/kg, between about 135 μg/kg to about 250 μg/kg, between about 135 μg/kg to about 200 μg/kg, between about 135 μg/kg to about 150 μg/kg, between about 150 μg/kg to about 500 μg/kg, between about 150 μg/kg to about 450 μg/kg, between about 150 μg/kg to about 400 μg/kg, between about 150 μg/kg to about 350 μg/kg, between about 150 μg/kg to about 300 μg/kg, between about 150 μg/kg to about 250 μg/kg, between about 150 μg/kg to about 200 μg/kg, between about 200 μg/kg to about 500 μg/kg, between about 200 μg/kg to about 450 μg/kg, between about 200 μg/kg to about 400 μg/kg, between about 200 μg/kg to about 350 μg/kg, between about 200 μg/kg to about 300 μg/kg, between about 200 μg/kg to about 250 μg/kg, between about 250 μg/kg to about 500 μg/kg, between about 250 μg/kg to about 450 μg/kg, between about 250 μg/kg to about 400 μg/kg, between about 250 μg/kg to about 350 μg/kg, between about 250 μg/kg to about 300 μg/kg, between about 300 μg/kg to about 500 μg/kg, between about 300 μg/kg to about 450 μg/kg, between about 300 μg/kg to about 400 μg/kg, between about 300 μg/kg to about 350 μg/kg, between about 350 μg/kg to about 500 μg/kg, between about 350 μg/kg to about 450 μg/kg, between about 350 μg/kg to about 400 μg/kg, between about 400 μg/kg to about 500 μg/kg, between about 400 μg/kg to about 450 μg/kg, or between about 450 μg/kg to about 500 μg/kg. In some embodiments, the C2D1, the C2D2, and the C2D3 are each between about 15 μg/kg to about 90 μg/kg. In some embodiments, the C2D1, the C2D2, and the C2D3 are each between about 20 μg/kg to about 40 μg/kg. In some embodiments, the C2D1, the C2D2, and the C2D3 are each between about 50 μg/kg to about 70 μg/kg. In particular embodiments, the C2D1, the C2D2, and the C2D3 are each about 60 μg/kg.

The C2D1, the C2D2, and the C2D3 can be administered on any suitable day of the further (e.g., second) dosing cycle in any of the preceding methods. For example, in some embodiments, the C2D1 is administered on Week 0, on Week 1, on Week 2, on Week 3, on Week 4, on Week 5, on Week 6, on Week 7, on Week 8, on Week 9, on Week 10, on Week 11, on Week 12, on Week 13, on Week 14, on Week 15, on Week 16, on Week 17, on Week 18, on Week 19, on Week 20, on Week 21, on Week 22, on Week 23, on Week 24, on Week 25, on Week 26, on Week 27, on Week 28, on Week 29, or on Week 30 of the further dosing cycle. For example, in some embodiments, the C2D2 is administered on Week 0, on Week 1, on Week 2, on Week 3, on Week 4, on Week 5, on Week 6, on Week 7, on Week 8, on Week 9, on Week 10, on Week 11, on Week 12, on Week 13, on Week 14, on Week 15, on Week 16, on Week 17, on Week 18, on Week 19, on Week 20, on Week 21, on Week 22, on Week 23, on Week 24, on Week 25, on Week 26, on Week 27, on Week 28, on Week 29, or on Week 30 of the further (e.g., second) dosing cycle. For example, in some embodiments, the C2D3 is administered on Week 0, on Week 1, on Week 2, on Week 3, on Week 4, on Week 5, on Week 6, on Week 7, on Week 8, on Week 9, on Week 10, on Week 11, on Week 12, on Week 13, on Week 14, on Week 15, on Week 16, on Week 17, on Week 18, on Week 19, on Week 20, on Week 21, on Week 22, on Week 23, on Week 24, on Week 25, on Week 26, on Week 27, on Week 28, on Week 29, or on Week 30 of the further (e.g., second) dosing cycle. In particular embodiments, the method includes administering the C2D1, the C2D2, and the C2D3 on or about Weeks 4, 12, and 20, respectively, of the further dosing cycle.

In some embodiments, the doses of the further (e.g., second) dosing cycle are administered to the subject every eight weeks (q8w). In some embodiments, each dose of the further (e.g., second) dosing cycle is between about 30 μg/kg to about 90 μg/kg (e.g., about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, or about 90 μg/kg). In some embodiments, each dose of the further (e.g., second) dosing cycle is about 60 μg/kg.

In some embodiments of any of the preceding aspects, the subject is not administered the IL-22 Fc fusion protein for a time period between any two dosing cycles (e.g., the subject is not administered the IL-22 Fc fusion protein for a time period between a first dosing cycle and a further (e.g., second) dosing cycle). In some embodiments, the subject is not administered the IL-22 Fc fusion protein for a time period of about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, three months, four months, five months, six months, seven months, eight months, nine months, ten months, eleven months, twelve months, two years, three years, four years, five years, or longer between any two dosing cycles (e.g., between a first dosing cycle and a further (e.g., second) dosing cycle).

For example, in some embodiments, the first dose of the further (e.g., second) dosing cycle is administered to the subject about 6 weeks to about 10 weeks after the last dose of the preceding (e.g., first) dosing cycle. In some embodiments, the first dose of the further (e.g., second) dosing cycle is administered to the subject about 7 weeks to about 9 weeks after the last dose of the preceding (e.g., first) dosing cycle. In some embodiments, the first dose of the further (e.g., second) dosing cycle is administered to the subject about 8 weeks after the last dose of the preceding (e.g., first) dosing cycle.

In another aspect, the invention features a method of treating a subject having an IBD comprising a dosing regimen, the dosing regimen including administering to the subject an IL-22 Fc fusion protein every four weeks (q4w) until the subject has a clinical remission. In some embodiments, each dose of the dosing regimen is between about 1 μg/kg to about 500 μg/kg, between about 1 μg/kg to about 450 μg/kg, between about 1 μg/kg to about 400 μg/kg, between about 1 μg/kg to about 350 μg/kg, between about 1 μg/kg to about 300 μg/kg, between about 1 μg/kg to about 250 μg/kg, between about 1 μg/kg to about 200 μg/kg, between about 1 μg/kg to about 150 μg/kg, between about 1 μg/kg to about 135 μg/kg, between about 1 μg/kg to about 100 μg/kg, between about 1 μg/kg to about 90 μg/kg, between about 1 μg/kg to about 75 μg/kg, between about 1 μg/kg to about 50 μg/kg, between about 1 μg/kg to about 25 μg/kg, between about 25 μg/kg to about 500 μg/kg, between about 25 μg/kg to about 450 μg/kg, between about 25 μg/kg to about 400 μg/kg, between about 25 μg/kg to about 350 μg/kg, between about 25 μg/kg to about 300 μg/kg, between about 25 μg/kg to about 250 μg/kg, between about 25 μg/kg to about 200 μg/kg, between about 25 μg/kg to about 150 μg/kg, between about 25 μg/kg to about 135 μg/kg, between about 25 μg/kg to about 100 μg/kg, between about 25 μg/kg to about 75 μg/kg, between about 25 μg/kg to about 50 μg/kg, between about 50 μg/kg to about 500 μg/kg, between about 50 μg/kg to about 450 μg/kg, between about 50 μg/kg to about 400 μg/kg, between about 50 μg/kg to about 350 μg/kg, between about 50 μg/kg to about 300 μg/kg, between about 50 μg/kg to about 250 μg/kg, between about 50 μg/kg to about 200 μg/kg, between about 50 μg/kg to about 150 μg/kg, between about 50 μg/kg to about 135 μg/kg, between about 50 μg/kg to about 100 μg/kg, between about 50 μg/kg to about 75 μg/kg, between about 75 μg/kg to about 500 μg/kg, between about 75 μg/kg to about 450 μg/kg, between about 75 μg/kg to about 400 μg/kg, between about 75 μg/kg to about 350 μg/kg, between about 75 μg/kg to about 300 μg/kg, between about 75 μg/kg to about 250 μg/kg, between about 75 μg/kg to about 200 μg/kg, between about 75 μg/kg to about 150 μg/kg, between about 75 μg/kg to about 135 μg/kg, between about 75 μg/kg to about 100 μg/kg, between about 80 μg/kg to about 500 μg/kg, between about 80 μg/kg to about 450 μg/kg, between about 80 μg/kg to about 400 μg/kg, between about 80 μg/kg to about 350 μg/kg, between about 80 μg/kg to about 300 μg/kg, between about 80 μg/kg to about 250 μg/kg, between about 80 μg/kg to about 200 μg/kg, between about 80 μg/kg to about 150 μg/kg, between about 80 μg/kg to about 135 μg/kg, between about 80 μg/kg to about 100 μg/kg, between about 90 μg/kg to about 500 μg/kg, between about 90 μg/kg to about 450 μg/kg, between about 90 μg/kg to about 400 μg/kg, between about 90 μg/kg to about 350 μg/kg, between about 90 μg/kg to about 300 μg/kg, between about 90 μg/kg to about 250 μg/kg, between about 90 μg/kg to about 200 μg/kg, between about 90 μg/kg to about 150 μg/kg, between about 90 μg/kg to about 135 μg/kg, between about 90 μg/kg to about 100 μg/kg, between about 100 μg/kg to about 500 μg/kg, between about 100 μg/kg to about 450 μg/kg, between about 100 μg/kg to about 400 μg/kg, between about 100 μg/kg to about 350 μg/kg, between about 100 μg/kg to about 300 μg/kg, between about 100 μg/kg to about 250 μg/kg, between about 100 μg/kg to about 200 μg/kg, between about 100 μg/kg to about 150 μg/kg, between about 100 μg/kg to about 135 μg/kg, between about 125 μg/kg to about 500 μg/kg, between about 125 μg/kg to about 450 μg/kg, between about 125 μg/kg to about 400 μg/kg, between about 125 μg/kg to about 350 μg/kg, between about 125 μg/kg to about 300 μg/kg, between about 125 μg/kg to about 250 μg/kg, between about 125 μg/kg to about 200 μg/kg, between about 125 μg/kg to about 150 μg/kg, between about 125 μg/kg to about 135 μg/kg, between about 135 μg/kg to about 500 μg/kg, between about 135 μg/kg to about 450 μg/kg, between about 135 μg/kg to about 400 μg/kg, between about 135 μg/kg to about 350 μg/kg, between about 135 μg/kg to about 300 μg/kg, between about 135 μg/kg to about 250 μg/kg, between about 135 μg/kg to about 200 μg/kg, between about 135 μg/kg to about 150 μg/kg, between about 150 μg/kg to about 500 μg/kg, between about 150 μg/kg to about 450 μg/kg, between about 150 μg/kg to about 400 μg/kg, between about 150 μg/kg to about 350 μg/kg, between about 150 μg/kg to about 300 μg/kg, between about 150 μg/kg to about 250 μg/kg, between about 150 μg/kg to about 200 μg/kg, between about 200 μg/kg to about 500 μg/kg, between about 200 μg/kg to about 450 μg/kg, between about 200 μg/kg to about 400 μg/kg, between about 200 μg/kg to about 350 μg/kg, between about 200 μg/kg to about 300 μg/kg, between about 200 μg/kg to about 250 μg/kg, between about 250 μg/kg to about 500 μg/kg, between about 250 μg/kg to about 450 μg/kg, between about 250 μg/kg to about 400 μg/kg, between about 250 μg/kg to about 350 μg/kg, between about 250 μg/kg to about 300 μg/kg, between about 300 μg/kg to about 500 μg/kg, between about 300 μg/kg to about 450 μg/kg, between about 300 μg/kg to about 400 μg/kg, between about 300 μg/kg to about 350 μg/kg, between about 350 μg/kg to about 500 μg/kg, between about 350 μg/kg to about 450 μg/kg, between about 350 μg/kg to about 400 μg/kg, between about 400 μg/kg to about 500 μg/kg, between about 400 μg/kg to about 450 μg/kg, or between about 450 μg/kg to about 500 μg/kg. In some embodiments, each dose of the dosing regimen is between about 15 μg/kg to about 90 μg/kg. In some embodiments, each dose of the dosing regimen is about 30 μg/kg, about 60 μg/kg, or about 90 μg/kg. In some embodiments, each dose of the dosing regimen is about 30 μg/kg. In some embodiments, each dose of the dosing regimen is about 60 μg/kg. In some embodiments, each dose of the dosing regimen is about 90 μg/kg.

In one aspect, the invention provides a method of treating a subject having an IBD comprising administering to the subject an IL-22 Fc fusion protein in a dosing regimen comprising a dosing cycle having a length of about 10 weeks, wherein the dosing cycle comprises a first dose (C1D1) and a second dose (C1D2) of the IL-22 Fc fusion protein, wherein the C1D1 and the C1D2 are each about 90 μg/kg, and wherein the C1D1 and C1D2 are administered to the subject on or about Weeks 0 and 6, respectively, of the dosing cycle.

In another aspect, the invention provides a method of treating a subject having an IBD comprising administering to the subject an IL-22 Fc fusion protein in a dosing regimen comprising a dosing cycle having a length of about 10 weeks, wherein the dosing cycle comprises a first dose (C1D1), a second dose (C1D2), and a third dose (C1D3) of the IL-22 Fc fusion protein, wherein the C1D1, the Ci D2, and the C1D3 are each about 30 μg/kg, and wherein the C1D1, the Ci D2, and the C1D3 are administered to the subject on or about Weeks 0, 4, and 8, respectively, of the dosing cycle. In some embodiments, the IL-22 Fc fusion protein comprises the amino acid sequence of SEQ ID NO:8, SEQ ID NO:10, or SEQ ID NO:16. In some embodiments, the IL-22 Fc fusion protein comprises the amino acid sequence of SEQ ID NO:8. In some embodiments, the IL-22 Fc fusion protein consists of the amino acid sequence of SEQ ID NO:8.

In yet another aspect, the invention provides a method of treating a subject having an IBD comprising administering to the subject an IL-22 Fc fusion protein in a dosing regimen comprising a dosing cycle having a length of about 10 weeks, wherein the dosing cycle comprises a first dose (C1D1), a second dose (C1D2), and a third dose (C1D3) of the IL-22 Fc fusion protein, wherein the C1D1, the Ci D2, and the C1D3 are each about 60 μg/kg, and wherein the C1D1, the Ci D2, and the C1D3 are administered to the subject on or about Weeks 0, 4, and 8, respectively, of the dosing cycle. In some embodiments, the IL-22 Fc fusion protein comprises the amino acid sequence of SEQ ID NO:8, SEQ ID NO:10, or SEQ ID NO:16. In some embodiments, the IL-22 Fc fusion protein comprises the amino acid sequence of SEQ ID NO:8. In some embodiments, the IL-22 Fc fusion protein consists of the amino acid sequence of SEQ ID NO:8.

In another aspect, the invention features a method of treating a subject having an IBD comprising administering to the subject an IL-22 Fc fusion protein in a dosing regimen comprising a dosing cycle having a length of about eight weeks, wherein the dosing cycle comprises a first dose (C1D1), a second dose (C1D2), and a third dose (C1D3) of the IL-22 Fc fusion protein, wherein the C1D1, the Ci D2, and the C1D3 are each about 90 μg/kg, and wherein the C1D1, the Ci D2, and the C1D3 are administered to the subject on or about Weeks 0, 4, and 8, respectively, of the dosing cycle. In some embodiments, the IL-22 Fc fusion protein comprises the amino acid sequence of SEQ ID NO:8, SEQ ID NO:10, or SEQ ID NO:16. In some embodiments, the IL-22 Fc fusion protein comprises the amino acid sequence of SEQ ID NO:8. In some embodiments, the IL-22 Fc fusion protein consists of the amino acid sequence of SEQ ID NO:8.

In a still further aspect, the invention provides a method of treating a subject having an IBD comprising administering to the subject an IL-22 Fc fusion protein in a dosing regimen comprising a dosing cycle having a length of about 10 weeks, wherein the dosing cycle comprises a first dose (C1D1), a second dose (C1D2), a third dose (C1D3), a fourth dose (C1D4), a fifth dose (C1D5), and a sixth dose (C1D6) of the IL-22 Fc fusion protein, wherein the C1D1, C1D2, the C1D3, the C1D4, the C1D5, and the C1D6 are each about 60 μg/kg, and wherein the C1D1, the C1D2, the C1D3, the C1D4, the C1D5, and the C1D6 are administered to the subject on or about Weeks 0, 2, 4, 6, 8, and 10, respectively, of the dosing cycle. In some embodiments, the IL-22 Fc fusion protein comprises the amino acid sequence of SEQ ID NO:8, SEQ ID NO:10, or SEQ ID NO:16. In some embodiments, the IL-22 Fc fusion protein comprises the amino acid sequence of SEQ ID NO:8. In some embodiments, the IL-22 Fc fusion protein consists of the amino acid sequence of SEQ ID NO:8.

In another aspect, the invention provides a method of treating a subject having an IBD comprising administering to the subject an IL-22 Fc fusion protein in a dosage regimen comprising at least a first dosing cycle and a second dosing cycle, wherein: (a) the first dosing cycle has a length of about 10 weeks and comprises a first dose (C1D1) and a second dose (C1D2) of the IL-22 Fc fusion protein, wherein the C1D1 and the C1D2 are each about 90 μg/kg, and wherein the C1D1 and the C1D2 are administered to the subject on or about Weeks 0 and 6, respectively, of the first dosing cycle; and (b) the second dosing cycle has a length of about 20 weeks and comprises a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are administered on or about Weeks 4, 12, and 20, respectively, of the second dosing cycle. In some embodiments, the IL-22 Fc fusion protein comprises the amino acid sequence of SEQ ID NO:8, SEQ ID NO:10, or SEQ ID NO:16. In some embodiments, the IL-22 Fc fusion protein comprises the amino acid sequence of SEQ ID NO:8. In some embodiments, the IL-22 Fc fusion protein consists of the amino acid sequence of SEQ ID NO:8.

In another aspect, the invention features a method of treating a subject having an IBD comprising administering to the subject an IL-22 Fc fusion protein in a dosage regimen comprising at least a first dosing cycle and a second dosing cycle, wherein: (a) the first dosing cycle has a length of about 8 weeks and comprises a first dose (C1D1), a second dose (C1D2), and a third dose (C1D3) of the IL-22 Fc fusion protein, wherein the C1D1, the C1D2, and the C1D3 are each about 90 μg/kg, and wherein the C1D1, the C1D2, and the C1D2 are administered to the subject on or about Weeks 0, 4, and 8, respectively, of the first dosing cycle; and (b) the second dosing cycle has a length of about 20 weeks and comprises a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are administered on or about Weeks 4, 12, and 20, respectively, of the second dosing cycle. In some embodiments, the IL-22 Fc fusion protein comprises the amino acid sequence of SEQ ID NO:8, SEQ ID NO:10, or SEQ ID NO:16. In some embodiments, the IL-22 Fc fusion protein comprises the amino acid sequence of SEQ ID NO:8. In some embodiments, the IL-22 Fc fusion protein consists of the amino acid sequence of SEQ ID NO:8.

In yet another aspect, the invention provides a method of treating a subject having an IBD comprising administering to the subject an IL-22 Fc fusion protein in a dosage regimen comprising at least a first dosing cycle and a second dosing cycle, wherein: (a) the first dosing cycle has a length of about 10 weeks and comprises a first dose (C1D1), a second dose (C1D2), and a third dose (C1D3) of the IL-22 Fc fusion protein, wherein the C1D1, the C1D2, and the C1D3 are each about 30 μg/kg, and wherein the C1D1, the C1D2, and the C1D2 are administered to the subject on or about Weeks 0, 4, and 8, respectively, of the first dosing cycle; and (b) the second dosing cycle has a length of about 20 weeks and comprises a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are administered on or about Weeks 4, 12, and 20, respectively, of the second dosing cycle. In some embodiments, the IL-22 Fc fusion protein comprises the amino acid sequence of SEQ ID NO:8, SEQ ID NO:10, or SEQ ID NO:16. In some embodiments, the IL-22 Fc fusion protein comprises the amino acid sequence of SEQ ID NO:8. In some embodiments, the IL-22 Fc fusion protein consists of the amino acid sequence of SEQ ID NO:8.

In a further aspect, the invention provides a method of treating a subject having an IBD comprising administering to the subject an IL-22 Fc fusion protein in a dosage regimen comprising at least a first dosing cycle and a second dosing cycle, wherein: (a) the first dosing cycle has a length of about 10 weeks and comprises a first dose (C1D1), a second dose (C1D2), and a third dose (C1D3) of the IL-22 Fc fusion protein, wherein the C1D1, the C1D2, and the C1D3 are each about 60 μg/kg, and wherein the C1D1, the C1D2, and the C1D2 are administered to the subject on or about Weeks 0, 4, and 8, respectively, of the first dosing cycle; and (b) the second dosing cycle has a length of about 20 weeks and comprises a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are administered on or about Weeks 4, 12, and 20, respectively, of the second dosing cycle. In some embodiments, the IL-22 Fc fusion protein comprises the amino acid sequence of SEQ ID NO:8, SEQ ID NO:10, or SEQ ID NO:16. In some embodiments, the IL-22 Fc fusion protein comprises the amino acid sequence of SEQ ID NO:8. In some embodiments, the IL-22 Fc fusion protein consists of the amino acid sequence of SEQ ID NO:8.

In yet a still further aspect, the invention provides a method of treating a subject having an IBD comprising administering to the subject an IL-22 Fc fusion protein in a dosage regimen comprising at least a first dosing cycle and a second dosing cycle, wherein: (a) the first dosing cycle has a length of about 10 weeks and comprises a first dose (C1D1), a second dose (C1D2), a third dose (C1D3), a fourth dose (C1D4), a fifth dose (C1D5), and a sixth dose (C1D6) of the IL-22 Fc fusion protein, wherein the C1D1, the C1D2, the C1D3, the C1D4, the C1D5, and the C1D6 are each about 60 μg/kg, and wherein the C1D1, the C1D2, the C1D3, the C1D4, the C1D5, and the C1D6 are administered to the subject on or about Weeks 0, 2, 4, 6, 8, and 10, respectively, of the dosing cycle; and (b) the second dosing cycle has a length of about 20 weeks and comprises a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are administered on or about Weeks 4, 12, and 20, respectively, of the second dosing cycle. In some embodiments, the IL-22 Fc fusion protein comprises the amino acid sequence of SEQ ID NO:8, SEQ ID NO:10, or SEQ ID NO:16. In some embodiments, the IL-22 Fc fusion protein comprises the amino acid sequence of SEQ ID NO:8. In some embodiments, the IL-22 Fc fusion protein consists of the amino acid sequence of SEQ ID NO:8.

In another aspect, provided herein is a method of treating a subject having an IBD comprising administering to the subject an IL-22 Fc fusion protein in a dosing regimen comprising a first dosing cycle having a length of about 8 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 30 μg/kg, and wherein the C1D1, the C1D2, and the C1D3 are administered to the subject on or about Weeks 1, 4, and 8, respectively, of the dosing cycle. The C1D1 may be administered on Day 0 of Week 1. In some embodiments, the IBD is UC. In other embodiments, the IBD is Crohn's disease.

In another aspect, provided herein is a method of treating a subject having an IBD comprising administering to the subject an IL-22 Fc fusion protein in a dosing regimen comprising a first dosing cycle having a length of about 8 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 60 μg/kg, and wherein the C1D1, the C1D2, and the C1D3 are administered to the subject on or about Weeks 1, 4, and 8, respectively, of the dosing cycle. The C1D1 may be administered on Day 0 of Week 1. In some embodiments, the IBD is UC. In other embodiments, the IBD is Crohn's disease.

In another aspect, provided herein is a method of treating a subject having an IBD comprising administering to the subject an IL-22 Fc fusion protein in a dosing regimen comprising a first dosing cycle having a length of about 8 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 90 μg/kg, and wherein the C1D1, the C1D2, and the C1D3 are administered to the subject on or about Weeks 1, 4, and 8, respectively, of the dosing cycle. The C1D1 may be administered on Day 0 of Week 1. In some embodiments, the IBD is UC. In other embodiments, the IBD is Crohn's disease.

In another aspect, provided herein is an IL-22 Fc fusion protein for use in a method of treating a subject having an IBD, wherein the IL-22 Fc fusion protein is for administration to the subject in a dosing regimen comprising a first dosing cycle having a length of about 8 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 30 μg/kg, and wherein the C1D1, the C1D2, and the C1D3 are administered to the subject on or about Weeks 1, 4, and 8, respectively, of the dosing cycle. In some embodiments, the C1D1 is administered on Day 0 of Week 1. In some embodiments, the IBD is UC. In other embodiments, the IBD is Crohn's disease.

In another aspect, provided herein is an IL-22 Fc fusion protein for use in a method of treating a subject having an IBD, wherein the IL-22 Fc fusion protein is for administration to the subject in a dosing regimen comprising a first dosing cycle having a length of about 8 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 60 μg/kg, and wherein the C1D1, the C1D2, and the C1D3 are administered to the subject on or about Weeks 1, 4, and 8, respectively, of the dosing cycle. In some embodiments, the C1D1 is administered on Day 0 of Week 1. In some embodiments, the IBD is UC. In other embodiments, the IBD is Crohn's disease.

In another aspect, provided herein is an IL-22 Fc fusion protein for use in a method of treating a subject having an IBD, wherein the IL-22 Fc fusion protein is for administration to the subject in a dosing regimen comprising a first dosing cycle having a length of about 8 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 90 μg/kg, and wherein the C1D1, the C1D2, and the C1D3 are administered to the subject on or about Weeks 1, 4, and 8, respectively, of the dosing cycle. In some embodiments, the C1D1 is administered on Day 0 of Week 1. In some embodiments, the IBD is UC. In other embodiments, the IBD is Crohn's disease.

In another aspect, provided herein is a method of treating a subject having an IBD comprising administering to the subject a pharmaceutical composition comprising an IL-22 Fc fusion protein in a dosing regimen comprising a first dosing cycle having a length of about 8 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 30 μg/kg, wherein the C1D1, the C1D2, and the C1D3 are administered to the subject on or about Weeks 1, 4, and 8, respectively, of the dosing cycle, and wherein the pharmaceutical composition has an average sialic acid content in the range of 8 to 12 moles of sialic acid per mole of the IL-22 Fc fusion protein. The C1D1 may be administered on Day 0 of Week 1. In some embodiments, the IBD is UC. In other embodiments, the IBD is Crohn's disease. In some embodiments, the pharmaceutical composition has an average sialic acid content in the range of 8 to 9 moles of sialic acid per mole of the IL-22 Fc fusion protein.

In another aspect, provided herein is a method of treating a subject having an IBD comprising administering to the subject a pharmaceutical composition comprising an IL-22 Fc fusion protein in a dosing regimen comprising a first dosing cycle having a length of about 8 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 60 μg/kg, and wherein the C1D1, the C1D2, and the C1D3 are administered to the subject on or about Weeks 1, 4, and 8, respectively, of the dosing cycle, and wherein the pharmaceutical composition has an average sialic acid content in the range of 8 to 12 moles of sialic acid per mole of the IL-22 Fc fusion protein. The C1D1 may be administered on Day 0 of Week 1. In some embodiments, the IBD is UC. In other embodiments, the IBD is Crohn's disease. In some embodiments, the pharmaceutical composition has an average sialic acid content in the range of 8 to 9 moles of sialic acid per mole of the IL-22 Fc fusion protein.

In another aspect, provided herein is a method of treating a subject having an IBD comprising administering to the subject a pharmaceutical composition comprising an IL-22 Fc fusion protein in a dosing regimen comprising a first dosing cycle having a length of about 8 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 90 μg/kg, wherein the C1D1, the C1D2, and the C1D3 are administered to the subject on or about Weeks 1, 4, and 8, respectively, of the dosing cycle, and wherein the pharmaceutical composition has an average sialic acid content in the range of 8 to 12 moles of sialic acid per mole of the IL-22 Fc fusion protein. The C1D1 may be administered on Day 0 of Week 1. In some embodiments, the IBD is UC. In other embodiments, the IBD is Crohn's disease. In some embodiments, the pharmaceutical composition has an average sialic acid content in the range of 8 to 9 moles of sialic acid per mole of the IL-22 Fc fusion protein.

In another aspect, provided herein is a pharmaceutical composition comprising an IL-22 Fc fusion protein for use in a method of treating a subject having an IBD, the pharmaceutical composition having an average sialic acid content in the range of 8 to 12 moles of sialic acid per mole of the IL-22 Fc fusion protein, wherein the pharmaceutical composition is for administration to the subject in a dosing regimen comprising a first dosing cycle having a length of about 8 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 30 μg/kg, and wherein the C1D1, the C1D2, and the C1D3 are administered to the subject on or about Weeks 1, 4, and 8, respectively, of the dosing cycle. In some embodiments, the C1D1 is administered on Day 0 of Week 1. In some embodiments, the IBD is UC. In other embodiments, the IBD is Crohn's disease. In some embodiments, the pharmaceutical composition has an average sialic acid content in the range of 8 to 9 moles of sialic acid per mole of the IL-22 Fc fusion protein.

In another aspect, provided herein is a pharmaceutical composition comprising an IL-22 Fc fusion protein for use in a method of treating a subject having an IBD, the pharmaceutical composition having an average sialic acid content in the range of 8 to 12 moles of sialic acid per mole of the IL-22 Fc fusion protein, wherein the pharmaceutical composition is for administration to the subject in a dosing regimen comprising a first dosing cycle having a length of about 8 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 60 μg/kg, and wherein the C1D1, the C1D2, and the C1D3 are administered to the subject on or about Weeks 1, 4, and 8, respectively, of the dosing cycle. In some embodiments, the C1D1 is administered on Day 0 of Week 1. In some embodiments, the IBD is UC. In other embodiments, the IBD is Crohn's disease. In some embodiments, the pharmaceutical composition has an average sialic acid content in the range of 8 to 9 moles of sialic acid per mole of the IL-22 Fc fusion protein.

In another aspect, provided herein is a pharmaceutical composition comprising an IL-22 Fc fusion protein for use in a method of treating a subject having an IBD, the pharmaceutical composition having an average sialic acid content in the range of 8 to 12 moles of sialic acid per mole of the IL-22 Fc fusion protein, wherein the pharmaceutical composition is for administration to the subject in a dosing regimen comprising a first dosing cycle having a length of about 8 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 90 μg/kg, and wherein the C1D1, the C1D2, and the C1D3 are administered to the subject on or about Weeks 1, 4, and 8, respectively, of the dosing cycle. In some embodiments, the C1D1 is administered on Day 0 of Week 1. In some embodiments, the IBD is UC. In other embodiments, the IBD is Crohn's disease. In some embodiments, the pharmaceutical composition has an average sialic acid content in the range of 8 to 9 moles of sialic acid per mole of the IL-22 Fc fusion protein.

In some embodiments of any of the preceding methods, the dosing regimen further comprises a further (e.g., a second) dosing cycle. In some embodiments, the length of the further (e.g., second) dosing cycle is between about 10 weeks and about 40 weeks. In some embodiments, the length of the further (e.g., second) dosing cycle is between about 15 weeks and about 25 weeks. In some embodiments, the length of the further (e.g., second) dosing cycle is about 20 weeks. In some embodiments, the further (e.g., second) dosing cycle continues indefinitely or until clinical remission. In some embodiments, the further (e.g., second) dosing cycle is stopped following the clinical remission, and then restarted following a relapse of the IBD.

In some embodiments of any of the preceding methods, the doses of the further (e.g., second) dosing cycle are administered to the subject every week (q1w), every two weeks (q2w), every four weeks (q4w), every six weeks (q6w), every eight weeks (q8w), every ten weeks (q10w), or every twelve weeks (q12w). In some embodiments, the doses of the further (e.g., second) dosing cycle are administered to the subject every eight weeks (q8w). In some embodiments, each dose of the further (e.g., second) dosing cycle is between about 30 μg/kg to about 90 μg/kg. In some embodiments, each dose of the further (e.g., second) dosing cycle is about 60 μg/kg.

In some embodiments of any of the preceding methods, the further (e.g., second) dosing cycle comprises a first dose (C2D1), a second dose (C2D2), and a third dose (C2D3) of the IL-22 Fc fusion protein. In some embodiments, the C2D1, the C2D2, and the C2D3 are each between about 30 μg/kg to about 90 μg/kg. In some embodiments, the C2D1, the C2D2, and the C2D3 are each between about 50 μg/kg to about 70 μg/kg. In some embodiments, the C2D1, the C2D2, and the C2D3 are each about 60 μg/kg. In some embodiments, the method comprises administering the C2D1, the C2D2, and the C2D3 on or about Weeks 4, 12, and 20, respectively, of the further (e.g., second) dosing cycle.

In some embodiments of any of the preceding methods, the first dose of the second dosing cycle is administered to the subject about 6 weeks to about 10 weeks after the last dose of the first dosing cycle.

In another aspect, provided herein is a method of treating a subject having an IBD comprising administering to the subject an IL-22 Fc fusion protein in a dosage regimen comprising at least a first dosing cycle and a second dosing cycle, wherein: (a) the first dosing cycle has a length of about 8 weeks and comprises a first dose (C1D1), a second dose (C1D2), and a third dose (C1D3) of the IL-22 Fc fusion protein, wherein the C1D1, the C1D2, and the C1D3 are each about 30 μg/kg, and wherein the C1D1, the C1D2, and the C1D2 are administered to the subject on or about Weeks 1, 4, and 8, respectively, of the first dosing cycle; and (b) the second dosing cycle continues indefinitely or until clinical remission, and comprises administering about 60 μg/kg of the IL-22 Fc fusion protein to the subject every 8 weeks (q8w), wherein the first dose of the second dosing cycle is administered to the subject about 8 weeks after the last dose of the first dosing cycle. The C1D1 may be administered on Day 0 of Week 1. In some embodiments, the IBD is UC. In other embodiments, the IBD is Crohn's disease.

In another aspect, provided herein is a method of treating a subject having an IBD comprising administering to the subject an IL-22 Fc fusion protein in a dosage regimen comprising at least a first dosing cycle and a second dosing cycle, wherein: (a) the first dosing cycle has a length of about 8 weeks and comprises a first dose (C1D1), a second dose (C1D2), and a third dose (C1D3) of the IL-22 Fc fusion protein, wherein the C1D1, the C1D2, and the C1D3 are each about 60 μg/kg, and wherein the C1D1, the C1D2, and the C1D2 are administered to the subject on or about Weeks 1, 4, and 8, respectively, of the first dosing cycle; and (b) the second dosing cycle continues indefinitely or until clinical remission, and comprises administering about 60 μg/kg of the IL-22 Fc fusion protein to the subject every 8 weeks (q8w), wherein the first dose of the second dosing cycle is administered to the subject about 8 weeks after the last dose of the first dosing cycle. The C1D1 may be administered on Day 0 of Week 1. In some embodiments, the IBD is UC. In other embodiments, the IBD is Crohn's disease.

In another aspect, provided herein is a method of treating a subject having an IBD comprising administering to the subject an IL-22 Fc fusion protein in a dosage regimen comprising at least a first dosing cycle and a second dosing cycle, wherein: (a) the first dosing cycle has a length of about 8 weeks and comprises a first dose (C1D1), a second dose (C1D2), and a third dose (C1D3) of the IL-22 Fc fusion protein, wherein the C1D1, the C1D2, and the C1D3 are each about 90 μg/kg, and wherein the C1D1, the C1D2, and the C1D2 are administered to the subject on or about Weeks 1, 4, and 8, respectively, of the first dosing cycle; and (b) the second dosing cycle continues indefinitely or until clinical remission, and comprises administering about 60 μg/kg of the IL-22 Fc fusion protein to the subject every 8 weeks (q8w), wherein the first dose of the second dosing cycle is administered to the subject about 8 weeks after the last dose of the first dosing cycle. The C1D1 may be administered on Day 0 of Week 1. In some embodiments, the IBD is UC. In other embodiments, the IBD is Crohn's disease.

In another aspect, provided herein is an IL-22 Fc fusion protein for use in a method of treating a subject having an IBD, wherein the IL-22 Fc fusion protein is for administration to the subject in a dosing regimen comprising at least a first dosing cycle and a second dosing cycle, wherein: (a) the first dosing cycle has a length of about 8 weeks and comprises a first dose (C1D1), a second dose (C1D2), and a third dose (C1D3) of the IL-22 Fc fusion protein, wherein the C1D1, the C1D2, and the C1D3 are each about 30 μg/kg, and wherein the C1D1, the C1D2, and the C1D2 are administered to the subject on or about Weeks 1, 4, and 8, respectively, of the first dosing cycle; and (b) the second dosing cycle continues indefinitely or until clinical remission, and comprises administering about 60 μg/kg of the IL-22 Fc fusion protein to the subject every 8 weeks (q8w), wherein the first dose of the second dosing cycle is administered to the subject about 8 weeks after the last dose of the first dosing cycle. In some embodiments, the C1D1 is administered on Day 0 of Week 1. In some embodiments, the IBD is UC. In other embodiments, the IBD is Crohn's disease.

In another aspect, provided herein is an IL-22 Fc fusion protein for use in a method of treating a subject having an IBD, wherein the IL-22 Fc fusion protein is for administration to the subject in a dosing regimen comprising at least a first dosing cycle and a second dosing cycle, wherein: (a) the first dosing cycle has a length of about 8 weeks and comprises a first dose (C1D1), a second dose (C1D2), and a third dose (C1D3) of the IL-22 Fc fusion protein, wherein the C1D1, the C1D2, and the C1D3 are each about 60 μg/kg, and wherein the C1D1, the C1D2, and the C1D2 are administered to the subject on or about Weeks 1, 4, and 8, respectively, of the first dosing cycle; and (b) the second dosing cycle continues indefinitely or until clinical remission, and comprises administering about 60 μg/kg of the IL-22 Fc fusion protein to the subject every 8 weeks (q8w), wherein the first dose of the second dosing cycle is administered to the subject about 8 weeks after the last dose of the first dosing cycle. In some embodiments, the C1D1 is administered on Day 0 of Week 1. In some embodiments, the IBD is UC. In other embodiments, the IBD is Crohn's disease.

In another aspect, provided herein is an IL-22 Fc fusion protein for use in a method of treating a subject having an IBD, wherein the IL-22 Fc fusion protein is for administration to the subject in a dosing regimen comprising at least a first dosing cycle and a second dosing cycle, wherein: (a) the first dosing cycle has a length of about 8 weeks and comprises a first dose (C1D1), a second dose (C1D2), and a third dose (C1D3) of the IL-22 Fc fusion protein, wherein the C1D1, the C1D2, and the C1D3 are each about 90 μg/kg, and wherein the C1D1, the C1D2, and the C1D2 are administered to the subject on or about Weeks 1, 4, and 8, respectively, of the first dosing cycle; and (b) the second dosing cycle continues indefinitely or until clinical remission, and comprises administering about 60 μg/kg of the IL-22 Fc fusion protein to the subject every 8 weeks (q8w), wherein the first dose of the second dosing cycle is administered to the subject about 8 weeks after the last dose of the first dosing cycle. In some embodiments, the C1D1 is administered on Day 0 of Week 1. In some embodiments, the IBD is UC. In other embodiments, the IBD is Crohn's disease.

In another aspect, provided herein is a method of treating a subject having an IBD comprising administering to the subject a pharmaceutical composition comprising an IL-22 Fc fusion protein in a dosage regimen comprising at least a first dosing cycle and a second dosing cycle, the pharmaceutical composition having an average sialic acid content in the range of 8 to 12 moles of sialic acid per mole of the IL-22 Fc fusion protein, wherein: (a) the first dosing cycle has a length of about 8 weeks and comprises a first dose (C1D1), a second dose (C1D2), and a third dose (C1D3) of the IL-22 Fc fusion protein, wherein the C1D1, the C1D2, and the C1D3 are each about 30 μg/kg, and wherein the C1D1, the C1D2, and the C1D2 are administered to the subject on or about Weeks 1, 4, and 8, respectively, of the first dosing cycle; and (b) the second dosing cycle continues indefinitely or until clinical remission, and comprises administering about 60 μg/kg of the IL-22 Fc fusion protein to the subject every 8 weeks (q8w), wherein the first dose of the second dosing cycle is administered to the subject about 8 weeks after the last dose of the first dosing cycle. The C1D1 may be administered on Day 0 of Week 1. In some embodiments, the IBD is UC. In other embodiments, the IBD is Crohn's disease. In some embodiments, the pharmaceutical composition has an average sialic acid content in the range of 8 to 9 moles of sialic acid per mole of the IL-22 Fc fusion protein.

In another aspect, provided herein is a method of treating a subject having an IBD comprising administering to the subject a pharmaceutical composition comprising an IL-22 Fc fusion protein in a dosage regimen comprising at least a first dosing cycle and a second dosing cycle, the pharmaceutical composition having an average sialic acid content in the range of 8 to 12 moles of sialic acid per mole of the IL-22 Fc fusion protein, wherein: (a) the first dosing cycle has a length of about 8 weeks and comprises a first dose (C1D1), a second dose (C1D2), and a third dose (C1D3) of the IL-22 Fc fusion protein, wherein the C1D1, the C1D2, and the C1D3 are each about 60 μg/kg, and wherein the C1D1, the C1D2, and the C1D2 are administered to the subject on or about Weeks 1, 4, and 8, respectively, of the first dosing cycle; and (b) the second dosing cycle continues indefinitely or until clinical remission, and comprises administering about 60 μg/kg of the IL-22 Fc fusion protein to the subject every 8 weeks (q8w), wherein the first dose of the second dosing cycle is administered to the subject about 8 weeks after the last dose of the first dosing cycle. The C1D1 may be administered on Day 0 of Week 1. In some embodiments, the IBD is UC. In other embodiments, the IBD is Crohn's disease. In some embodiments, the pharmaceutical composition has an average sialic acid content in the range of 8 to 9 moles of sialic acid per mole of the IL-22 Fc fusion protein.

In another aspect, provided herein is a method of treating a subject having an IBD comprising administering to the subject a pharmaceutical composition comprising an IL-22 Fc fusion protein in a dosage regimen comprising at least a first dosing cycle and a second dosing cycle, the pharmaceutical composition having an average sialic acid content in the range of 8 to 12 moles of sialic acid per mole of the IL-22 Fc fusion protein, wherein: (a) the first dosing cycle has a length of about 8 weeks and comprises a first dose (C1D1), a second dose (C1D2), and a third dose (C1D3) of the IL-22 Fc fusion protein, wherein the C1D1, the C1D2, and the C1D3 are each about 90 μg/kg, and wherein the C1D1, the C1D2, and the C1D2 are administered to the subject on or about Weeks 1, 4, and 8, respectively, of the first dosing cycle; and (b) the second dosing cycle continues indefinitely or until clinical remission, and comprises administering about 60 μg/kg of the IL-22 Fc fusion protein to the subject every 8 weeks (q8w), wherein the first dose of the second dosing cycle is administered to the subject about 8 weeks after the last dose of the first dosing cycle. The C1D1 may be administered on Day 0 of Week 1. In some embodiments, the IBD is UC. In other embodiments, the IBD is Crohn's disease. In some embodiments, the pharmaceutical composition has an average sialic acid content in the range of 8 to 9 moles of sialic acid per mole of the IL-22 Fc fusion protein.

In another aspect, provided herein is a pharmaceutical composition comprising an IL-22 Fc fusion protein for use in a method of treating a subject having an IBD, the pharmaceutical composition having an average sialic acid content in the range of 8 to 12 moles of sialic acid per mole of the IL-22 Fc fusion protein, wherein the pharmaceutical composition is for administration to the subject in a dosing regimen comprising at least a first dosing cycle and a second dosing cycle, wherein: (a) the first dosing cycle has a length of about 8 weeks and comprises a first dose (C1D1), a second dose (C1D2), and a third dose (C1D3) of the IL-22 Fc fusion protein, wherein the C1D1, the C1D2, and the C1D3 are each about 30 μg/kg, and wherein the C1D1, the C1D2, and the C1D2 are administered to the subject on or about Weeks 1, 4, and 8, respectively, of the first dosing cycle; and (b) the second dosing cycle continues indefinitely or until clinical remission, and comprises administering about 60 μg/kg of the IL-22 Fc fusion protein to the subject every 8 weeks (q8w), wherein the first dose of the second dosing cycle is administered to the subject about 8 weeks after the last dose of the first dosing cycle. In some embodiments, the C1D1 is administered on Day 0 of Week 1. In some embodiments, the IBD is UC. In other embodiments, the IBD is Crohn's disease. In some embodiments, the pharmaceutical composition has an average sialic acid content in the range of 8 to 9 moles of sialic acid per mole of the IL-22 Fc fusion protein.

In another aspect, provided herein is a pharmaceutical composition comprising an IL-22 Fc fusion protein for use in a method of treating a subject having an IBD, the pharmaceutical composition having an average sialic acid content in the range of 8 to 12 moles of sialic acid per mole of the IL-22 Fc fusion protein, wherein the pharmaceutical composition is for administration to the subject in a dosing regimen comprising at least a first dosing cycle and a second dosing cycle, wherein: (a) the first dosing cycle has a length of about 8 weeks and comprises a first dose (C1D1), a second dose (C1D2), and a third dose (C1D3) of the IL-22 Fc fusion protein, wherein the C1D1, the C1D2, and the C1D3 are each about 60 μg/kg, and wherein the C1D1, the C1D2, and the C1D2 are administered to the subject on or about Weeks 1, 4, and 8, respectively, of the first dosing cycle; and (b) the second dosing cycle continues indefinitely or until clinical remission, and comprises administering about 60 μg/kg of the IL-22 Fc fusion protein to the subject every 8 weeks (q8w), wherein the first dose of the second dosing cycle is administered to the subject about 8 weeks after the last dose of the first dosing cycle. In some embodiments, the C1D1 is administered on Day 0 of Week 1. In some embodiments, the IBD is UC. In other embodiments, the IBD is Crohn's disease. In some embodiments, the pharmaceutical composition has an average sialic acid content in the range of 8 to 9 moles of sialic acid per mole of the IL-22 Fc fusion protein.

In another aspect, provided herein is a pharmaceutical composition comprising an IL-22 Fc fusion protein for use in a method of treating a subject having an IBD, the pharmaceutical composition having an average sialic acid content in the range of 8 to 12 moles of sialic acid per mole of the IL-22 Fc fusion protein, wherein the pharmaceutical composition is for administration to the subject in a dosing regimen comprising at least a first dosing cycle and a second dosing cycle, wherein: (a) the first dosing cycle has a length of about 8 weeks and comprises a first dose (C1D1), a second dose (C1D2), and a third dose (C1D3) of the IL-22 Fc fusion protein, wherein the C1D1, the C1D2, and the C1D3 are each about 90 μg/kg, and wherein the C1D1, the C1D2, and the C1D2 are administered to the subject on or about Weeks 1, 4, and 8, respectively, of the first dosing cycle; and (b) the second dosing cycle continues indefinitely or until clinical remission, and comprises administering about 60 μg/kg of the IL-22 Fc fusion protein to the subject every 8 weeks (q8w), wherein the first dose of the second dosing cycle is administered to the subject about 8 weeks after the last dose of the first dosing cycle. In some embodiments, the C1D1 is administered on Day 0 of Week 1. In some embodiments, the IBD is UC. In other embodiments, the IBD is Crohn's disease. In some embodiments, the pharmaceutical composition has an average sialic acid content in the range of 8 to 9 moles of sialic acid per mole of the IL-22 Fc fusion protein.

In any of the preceding methods, the IL-22 Fc fusion protein may be included in a pharmaceutical composition, e.g., a pharmaceutical composition comprising an IL-22 Fc fusion protein and a pharmaceutically acceptable carrier.

In any of the preceding methods, the pharmaceutical composition may have an average sialic acid content of 8 moles of sialic acid per mole of the IL-22 Fc fusion protein. In other embodiments of any of the preceding methods, the pharmaceutical composition may have an average sialic acid content of 9 moles of sialic acid per mole of the IL-22 Fc fusion protein. In some embodiments, the sialic acid comprises N-acetylneuraminic acid (NANA). In some embodiments, the pharmaceutical composition has an average NGNA content of less than 1 mole of NGNA per mole of the IL-22 Fc fusion protein.

In any of the preceding methods, the IL-22 polypeptide may be N-glycosylated. In some embodiments, the IL-22 polypeptide is glycosylated at one or more locations corresponding to amino acid residues Asn21, Asn35, Asn64, and/or Asn143 of SEQ ID NO: 4. In some embodiments, the IL-22 Fc fusion protein comprises a glycosylated IL-22 polypeptide linked to an Fc region by a linker, wherein the IL-22 polypeptide is glycosylated at one or more locations corresponding to amino acid residues Asn21, Asn35, Asn64, and/or Asn143 of SEQ ID NO: 4, and wherein: (a) the percent N-glycosylation site occupancy at residue Asn21 is in the range of 70 to 90; (b) the percent N-glycosylation site occupancy at residue Asn35 is in the range of 90 to 100; (c) the percent N-glycosylation site occupancy at residue Asn64 is in the range of 90 to 100; and/or (d) the percent N-glycosylation site occupancy at residue Asn143 is in the range of 25 to 35.

In any of the preceding methods, the pharmaceutical composition may be a liquid composition.

In any of the preceding methods, (i) the IL-22 Fc fusion protein may have a maximum observed concentration (Cₘₐₓ) of about 8,000 ng/mL to about 19,000 ng; (ii) the IL-22 Fc fusion protein may have an area under the serum concentration-time curve from time 0 to the last measureable time point (AUCₗₐₛₜ) of about 7,000 day·ng/mL to about 25,000 day·ng/mL; and/or (iii) the IL-22 Fc fusion protein may have a clearance (CL) of about 40 mL/kg/day to about 140 mL/kg/day. In some embodiments, the Cₘₐₓ, AUCₗₐₛₜ, and/or CL is assessed following intravenous administration of about 1,000 μg/kg of the IL-22 Fc fusion protein to a CD1 mouse.

In any of the preceding methods, the IL-22 polypeptide may comprise N-glycans having monoantennary, biantennary, triantennary, and/or tetraantennary structure. In some embodiments: (i) about 0.1% to about 2% of the N-glycans have monoantennary structure; (ii) about 10% to about 25% of the N-glycans have biantennary structure; (iii) about 25% to about 40% of the N-glycans have triantennary structure; and/or (iv) about 30% to about 51% of the N-glycans have tetraantennary structure. In some embodiments: (i) 0.1% to 2% of the N-glycans have monoantennary structure; (ii) 10% to 25% of the N-glycans have biantennary structure; (iii) 25% to 40% of the N-glycans have triantennary structure; and/or (iv) 30% to 51% of the N-glycans have tetraantennary structure.

In any of the preceding methods, the IL-22 Fc fusion protein may comprise N-glycans comprising zero, one, two, three, or four galactose moieties. In some embodiments: (i) about 9% to about 32% of the N-glycans comprise zero galactose moieties; (ii) about 10% to about 20% of the N-glycans comprise one galactose moiety; (iii) about 8% to about 25% of the N-glycans comprise two galactose moieties; (iv) about 12% to about 25% of the N-glycans comprise three galactose moieties; and/or (v) about 12% to about 30% of the N-glycans comprise four galactose moieties. In some embodiments: (i) 9% to 32% of the N-glycans comprise zero galactose moieties; (ii) 10% to 20% of the N-glycans comprise one galactose moiety; (iii) 8% to 25% of the N-glycans comprise two galactose moieties; (iv) 12% to 25% of the N-glycans comprise three galactose moieties; and/or (v) 12% to 30% of the N-glycans comprise four galactose moieties.

In any of the preceding methods, the IL-22 Fc fusion protein may comprise N-glycans comprising zero, one, two, three, or four sialic acid moieties. In some embodiments: (i) about 12% to about 35% of the N-glycans comprise zero sialic acid moieties; (ii) about 10% to about 30% of the N-glycans comprise one sialic acid moiety; (iii) about 10% to about 30% of the N-glycans comprise two sialic acid moieties; (iv) about 10% to about 30% of the N-glycans comprise three sialic acid moieties; and/or (v) about 1% to about 20% of the N-glycans comprise four sialic acid moieties. In some embodiments: (i) 12% to 35% of the N-glycans comprise zero sialic acid moieties; (ii) 10% to 30% of the N-glycans comprise one sialic acid moiety; (iii) 10% to 30% of the N-glycans comprise two sialic acid moieties; (iv) 10% to 30% of the N-glycans comprise three sialic acid moieties; and/or (v) 1% to 20% of the N-glycans comprise four sialic acid moieties.

In any of the preceding methods, (i) the IL-22 polypeptide may comprise about 0% to about 10% N-glycans comprising a terminal mannose moiety; and/or (ii) the IL-22 polypeptide may comprise about 30% to about 55% N-glycans comprising a terminal N-acetylglucosamine (GlcNAc) moiety. In some embodiments, (i) the IL-22 polypeptide comprises 0% to 10% N-glycans comprising a terminal mannose moiety; and/or (ii) the IL-22 polypeptide comprises 30% to 55% N-glycans comprising a terminal GlcNAc moiety. In some embodiments, the IL-22 polypeptide comprises 0% to 10% N-glycans comprising a terminal mannose moiety. In some embodiments, the IL-22 polypeptide comprises 30% to 55% N-glycans comprising a terminal GlcNAc moiety.

In any of the preceding methods, the N-glycans may comprise one, two, three, or four terminal GlcNAc moieties. In some embodiments: (i) about 1% to about 20% of the N-glycans comprise one terminal GlcNAc moiety; (ii) about 1% to about 20% of the N-glycans comprise two terminal GlcNAc moieties; (iii) about 5% to about 25% of the N-glycans comprise three terminal GlcNAc moieties; and/or (iv) about 0% to about 15% of the N-glycans comprise four terminal GlcNAc moieties. In some embodiments: (i) 1% to 20% of the N-glycans comprise one terminal GlcNAc moiety; (ii) 1% to 20% of the N-glycans comprise two terminal GlcNAc moieties; (iii) 5% to 25% of the N-glycans comprise three terminal GlcNAc moieties; and/or (iv) 0% to 15% of the N-glycans comprise four terminal GlcNAc moieties.

In any of the preceding methods, (i) the IL-22 polypeptide may comprise about 20% to about 45% N-glycans comprising a terminal galactose (Gal) moiety; and/or (ii) the N-glycans comprise one, two, or three terminal Gal moieties. In some embodiments, (i) the IL-22 polypeptide comprises 20% to 45% N-glycans comprising a terminal Gal moiety; and/or (ii) the N-glycans comprise one, two, or three terminal Gal moieties.

In any of the preceding methods, (i) about 15% to about 30% of the N-glycans may comprise one terminal Gal moiety; (ii) about 1% to about 15% of the N-glycans may comprise two terminal Gal moieties; and/or (iii) about 0.1% to about 6% of the N-glycans may comprise three terminal Gal moieties. In some embodiments: (i) 15% to 30% of the N-glycans comprise one terminal Gal moiety; (ii) 1% to 15% of the N-glycans comprise two terminal Gal moieties; and/or (iii) 0.1% to 6% of the N-glycans comprise three terminal Gal moieties.

In any of the preceding methods, (i) the IL-22 polypeptide may comprise N-glycans comprises galactose N-acetylglucosamine (LacNAc) repeats; (ii) the IL-22 polypeptide may comprise N-glycans comprising fucosylated N-glycans; and/or (iii) the IL-22 polypeptide may comprise N-glycans comprising afucosylated N-glycans.

Any suitable concentration of the IL-22 Fc fusion protein may be used. For example, in some embodiments, the concentration of the IL-22 Fc fusion protein is about 0.5 mg/mL to about 20 mg/mL. In some embodiments, the concentration of the IL-22 Fc fusion protein is about 0.5 mg/mL to about 5 mg/mL. In some embodiments, the concentration of the IL-22 Fc fusion protein is about 1 mg/mL. In some embodiments, the concentration of the IL-22 Fc fusion protein is about 8 mg/mL to about 12 mg/mL. In some embodiments, the concentration of the IL-22 Fc fusion protein is about 10 mg/mL.

In any of the preceding methods, the IL-22 Fc fusion may be produced from a production culture having a volume of at least about 500 L. In some embodiments of any of the preceding aspects, the IL-22 Fc fusion protein has been produced from a production culture having a volume of about 500 L to about 5,000 L. In some embodiments, the IL-22 Fc fusion protein has been produced from a production culture having a volume of about 1,000 L to about 3,000 L. In some embodiments the IL-22 Fc fusion protein has been produced from a production culture having a volume of about 1,500 L to about 2,500 L. In some embodiments, the IL-22 Fc fusion protein has been produced from a production culture having a volume of about 2000 L.

In any of the preceding methods, the treating can ameliorate one or more symptoms of the IBD. For example, in some embodiments, the one or more symptoms of IBD include stool frequency, rectal bleeding, or mucosal appearance. In some embodiments, mucosal appearance comprises erythema, decreased or absent vascular pattern, friability, erosions, spontaneous bleeding, and/or ulceration.

In any of the preceding methods, the treating can result in a clinical remission. In some embodiments, the treating results in a clinical remission within about 1 week, about 2 weeks, about 3 weeks, about 4 weeks, about 5 weeks, about 6 weeks, about 7 weeks, about 9 weeks, about 10 weeks, about 11 weeks, about 12 weeks, about 14 weeks, about 16 weeks, about 18 weeks, about 20 weeks, about 22 weeks, about 24 weeks, about 26 weeks, about 28 weeks, about 30 weeks, about 32 weeks, about 34 weeks, about 36 weeks, about 38 weeks, about 40 weeks, about 42 weeks, about 44 weeks, about 46 weeks, about 48 weeks, about 50 weeks, about 52 weeks, about 54 weeks, about 56 weeks, about 58 weeks, about 60 weeks, about 62 weeks, about 64 weeks, about 66 weeks, about 68 weeks, about 70 weeks, about 72 weeks, about 74 weeks, about 76 weeks, about 78 weeks, or about 80 weeks from the first dose. In some embodiments, the treating results in a clinical remission within about 10 weeks from the first dose. In some embodiments, the treating results in a clinical remission within about 6 weeks from the first dose. In some embodiments, the treating results in a clinical remission at about 6 weeks from the first dose and at about 10 weeks from the first dose.

In some embodiments of any of the preceding methods, the clinical remission is a modified Mayo Clinic Score (MCS) of less than or equal to about 2 (e.g., about 0, about 1, or about 2) and a Mayo rectal bleeding subscore of 0 and other Mayo subscores of less than or equal to about 1 (e.g., about 0 or about 1).

In some embodiments of any of the preceding methods, the clinical remission is a sustained remission. For example, in some embodiments, the sustained remission is a clinical remission at about 10 weeks, about 15 weeks, about 20 weeks, about 25 weeks, about 30 weeks, about 35 weeks, about 40 weeks, about 45 weeks, about 50 weeks, about 52 weeks, about 55 weeks, about 60 weeks, about 65 weeks, about 70 weeks, about 72 weeks, about 75 weeks, about 80 weeks, about 85 weeks, about 90 weeks, about 95 weeks, about 100 weeks, about 102 weeks, about 105 weeks, or about 110 weeks from the first dose. In some embodiments, the sustained remission is a clinical remission at about ten weeks from the first dose and at about 30 weeks from the first dose. In some embodiments, the sustained remission has a length of at least about 30 weeks, or at least about 7, about 8, about 9, about 10, about 11, or about 12 months.

In any of the preceding methods, the treating can result in a clinical response. In some embodiments, the clinical response comprises a decrease in the subject's mMCS score relative to a baseline mMCS score. In some embodiments, the decrease in the subject's mMCS score is a decrease of at least about 1 point or higher relative to the baseline mMCS score, e.g., about 1 point or higher, about 2 points or higher, about 3 points or higher, about 4 points or higher, about 5 points or higher, about 6 points or higher, about 7 points or higher, about 8 points or higher, or about 9 points relative to the baseline mMCS score. In some embodiments, the decrease in the subject's mMCS score is a decrease of at least about 3 points or higher relative to the baseline mMCS score. In some embodiments, the clinical response comprises a decrease in the subject's Mayo rectal bleeding subscore relative to a baseline Mayo rectal bleeding subscore or a Mayo rectal bleeding subscore of 0 or 1. In some embodiments, a decrease in the subject's Mayo rectal bleeding subscore is a decrease of about 1 point or higher relative to the baseline Mayo rectal bleeding subscore, e.g., about 1 point or higher, about 2 points or higher, or about 3 points relative to the baseline Mayo rectal bleeding subscore.

In any of the preceding methods, the clinical response may be present within about 1 week, about 2 weeks, about 3 weeks, about 4 weeks, about 5 weeks, about 6 weeks, about 7 weeks, about 9 weeks, about 10 weeks, about 11 weeks, about 12 weeks, about 14 weeks, about 16 weeks, about 18 weeks, about 20 weeks, about 22 weeks, about 24 weeks, about 26 weeks, about 28 weeks, about 30 weeks, about 32 weeks, about 34 weeks, about 36 weeks, about 38 weeks, about 40 weeks, about 42 weeks, about 44 weeks, about 46 weeks, about 48 weeks, about 50 weeks, about 52 weeks, about 54 weeks, about 56 weeks, about 58 weeks, about 60 weeks, about 62 weeks, about 64 weeks, about 66 weeks, about 68 weeks, about 70 weeks, about 72 weeks, about 74 weeks, about 76 weeks, about 78 weeks, or about 80 weeks from the first dose. In some embodiments, the treating results in a clinical response within about 30 weeks from the first dose. In some embodiments, the treating results in a clinical response within about 10 weeks from the first dose. In some embodiments, the treating results in a clinical response within about 6 weeks from the first dose. In some embodiments, the treating results in a clinical response at about 6 weeks, about 10 weeks, and about 30 weeks from the first dose.

In any of the preceding methods, the treating can result in endoscopic healing. In some embodiments, the endoscopic healing is a Mayo endoscopic subscore of less than or equal to about 2 (e.g., about 0, about 1, or about 2). In some embodiments, the endoscopic healing is a Mayo endoscopic subscore of less than or equal to about 1. In some embodiments, the endoscopic healing may be present within about 1 week, about 2 weeks, about 3 weeks, about 4 weeks, about 5 weeks, about 6 weeks, about 7 weeks, about 9 weeks, about 10 weeks, about 11 weeks, about 12 weeks, about 14 weeks, about 16 weeks, about 18 weeks, about 20 weeks, about 22 weeks, about 24 weeks, about 26 weeks, about 28 weeks, about 30 weeks, about 32 weeks, about 34 weeks, about 36 weeks, about 38 weeks, about 40 weeks, about 42 weeks, about 44 weeks, about 46 weeks, about 48 weeks, about 50 weeks, about 52 weeks, about 54 weeks, about 56 weeks, about 58 weeks, about 60 weeks, about 62 weeks, about 64 weeks, about 66 weeks, about 68 weeks, about 70 weeks, about 72 weeks, about 74 weeks, about 76 weeks, about 78 weeks, or about 80 weeks from the first dose. In some embodiments, the treating results in endoscopic healing within about 30 weeks from the first dose. In some embodiments, the treating results in endoscopic healing within about 10 weeks from the first dose. In some embodiments, the treating results in endoscopic healing within about 6 weeks from the first dose. In some embodiments, the treating results in endoscopic healing at about 6 weeks, about 10 weeks, and about 30 weeks from the first dose.

In any of the preceding methods, the treating can result in an endoscopic remission. In some embodiments, the endoscopic remission is a Mayo endoscopic subscore of zero (0). In some embodiments, the endoscopic remission may be present within about 1 week, about 2 weeks, about 3 weeks, about 4 weeks, about 5 weeks, about 6 weeks, about 7 weeks, about 9 weeks, about 10 weeks, about 11 weeks, about 12 weeks, about 14 weeks, about 16 weeks, about 18 weeks, about 20 weeks, about 22 weeks, about 24 weeks, about 26 weeks, about 28 weeks, about 30 weeks, about 32 weeks, about 34 weeks, about 36 weeks, about 38 weeks, about 40 weeks, about 42 weeks, about 44 weeks, about 46 weeks, about 48 weeks, about 50 weeks, about 52 weeks, about 54 weeks, about 56 weeks, about 58 weeks, about 60 weeks, about 62 weeks, about 64 weeks, about 66 weeks, about 68 weeks, about 70 weeks, about 72 weeks, about 74 weeks, about 76 weeks, about 78 weeks, or about 80 weeks from the first dose. In some embodiments, the treating results in an endoscopic remission within about 30 weeks from the first dose. In some embodiments, the treating results in an endoscopic remission within about 10 weeks from the first dose. In some embodiments, the treating results in an endoscopic remission within about 6 weeks from the first dose. In some embodiments, the treating results in an endoscopic remission at about 6 weeks, about 10 weeks, and about 30 weeks from the first dose.

In any of the preceding methods, the treating can result in a change from baseline in the subject's bowel movement signs and symptoms as assessed by the Ulcerative Colitis Patient-Reported Outcome Signs and Symptoms (UC-PRO/SS) score. In some embodiments, the change from baseline in the subject's bowel movement signs and symptoms as assessed by the UC-PRO/SS score may be present within about 1 week, about 2 weeks, about 3 weeks, about 4 weeks, about 5 weeks, about 6 weeks, about 7 weeks, about 9 weeks, about 10 weeks, about 11 weeks, about 12 weeks, about 14 weeks, about 16 weeks, about 18 weeks, about 20 weeks, about 22 weeks, about 24 weeks, about 26 weeks, about 28 weeks, about 30 weeks, about 32 weeks, about 34 weeks, about 36 weeks, about 38 weeks, about 40 weeks, about 42 weeks, about 44 weeks, about 46 weeks, about 48 weeks, about 50 weeks, about 52 weeks, about 54 weeks, about 56 weeks, about 58 weeks, about 60 weeks, about 62 weeks, about 64 weeks, about 66 weeks, about 68 weeks, about 70 weeks, about 72 weeks, about 74 weeks, about 76 weeks, about 78 weeks, or about 80 weeks from the first dose. In some embodiments, the treating results in the change from baseline in the subject's bowel movement signs and symptoms as assessed by the UC-PRO/SS score within about 30 weeks from the first dose. In some embodiments, the treating results in the change from baseline in the subject's bowel movement signs and symptoms as assessed by the UC-PRO/SS score within about 10 weeks from the first dose. In some embodiments, the treating results in the change from baseline in the subject's bowel movement signs and symptoms as assessed by the UC-PRO/SS score within about 6 weeks from the first dose. In some embodiments, the treating results in the change from baseline in the subject's bowel movement signs and symptoms as assessed by the UC-PRO/SS score at about 6 weeks, about 10 weeks, and about 30 weeks from the first dose.

In any of the preceding methods, the treating can result in a change from baseline in the subject's abdominal signs and symptoms as assessed by the Ulcerative Colitis Patient-Reported Outcome Signs and Symptoms (UC-PRO/SS) score. In some embodiments, the change from baseline in the subject's abdominal signs and symptoms as assessed by the UC-PRO/SS score may be present within about 1 week, about 2 weeks, about 3 weeks, about 4 weeks, about 5 weeks, about 6 weeks, about 7 weeks, about 9 weeks, about 10 weeks, about 11 weeks, about 12 weeks, about 14 weeks, about 16 weeks, about 18 weeks, about 20 weeks, about 22 weeks, about 24 weeks, about 26 weeks, about 28 weeks, about 30 weeks, about 32 weeks, about 34 weeks, about 36 weeks, about 38 weeks, about 40 weeks, about 42 weeks, about 44 weeks, about 46 weeks, about 48 weeks, about 50 weeks, about 52 weeks, about 54 weeks, about 56 weeks, about 58 weeks, about 60 weeks, about 62 weeks, about 64 weeks, about 66 weeks, about 68 weeks, about 70 weeks, about 72 weeks, about 74 weeks, about 76 weeks, about 78 weeks, or about 80 weeks from the first dose. In some embodiments, the treating results in the change from baseline in the subject's abdominal signs and symptoms as assessed by the UC-PRO/SS score within about 30 weeks from the first dose. In some embodiments, the treating results in the change from baseline in the subject's abdominal signs and symptoms as assessed by the UC-PRO/SS score within about 10 weeks from the first dose. In some embodiments, the treating results in the change from baseline in the subject's abdominal signs and symptoms as assessed by the UC-PRO/SS score within about 6 weeks from the first dose. In some embodiments, the treating results in the change from baseline in the subject's abdominal signs and symptoms as assessed by the UC-PRO/SS score at about 6 weeks, about 10 weeks, and about 30 weeks from the first dose.

In any of the preceding methods, the treating can result in a change from baseline in the subject's patient-reported health-related quality of life (QOL) as assessed by an Inflammatory Bowel Disease Questionnaire (IBDQ) score. In some embodiments, the change from baseline in the subject's patient-reported health-related QOL as assessed by the IBDQ score may be present within about 1 week, about 2 weeks, about 3 weeks, about 4 weeks, about 5 weeks, about 6 weeks, about 7 weeks, about 9 weeks, about 10 weeks, about 11 weeks, about 12 weeks, about 14 weeks, about 16 weeks, about 18 weeks, about 20 weeks, about 22 weeks, about 24 weeks, about 26 weeks, about 28 weeks, about 30 weeks, about 32 weeks, about 34 weeks, about 36 weeks, about 38 weeks, about 40 weeks, about 42 weeks, about 44 weeks, about 46 weeks, about 48 weeks, about 50 weeks, about 52 weeks, about 54 weeks, about 56 weeks, about 58 weeks, about 60 weeks, about 62 weeks, about 64 weeks, about 66 weeks, about 68 weeks, about 70 weeks, about 72 weeks, about 74 weeks, about 76 weeks, about 78 weeks, or about 80 weeks from the first dose. In some embodiments, the treating results in the change from baseline in the subject's patient-reported health-related QOL as assessed by the IBDQ score within about 30 weeks from the first dose. In some embodiments, the treating results in the change from baseline in the subject's patient-reported health-related QOL as assessed by the IBDQ score within about 10 weeks from the first dose. In some embodiments, the treating results in the change from baseline in the subject's patient-reported health-related QOL as assessed by the IBDQ score within about 6 weeks from the first dose. In some embodiments, the treating results in the change from baseline in the subject's patient-reported health-related QOL as assessed by the IBDQ score at about 6 weeks, about 10 weeks, and about 30 weeks from the first dose.

In any of the preceding methods, the treating can result in mucosal healing. In some embodiments, the mucosal healing is endoscopic healing and histological remission of less than or equal to about 6, as assessed by Robarts Histological Index. In some embodiments, the mucosal healing may be present within about 1 week, about 2 weeks, about 3 weeks, about 4 weeks, about 5 weeks, about 6 weeks, about 7 weeks, about 9 weeks, about 10 weeks, about 11 weeks, about 12 weeks, about 14 weeks, about 16 weeks, about 18 weeks, about 20 weeks, about 22 weeks, about 24 weeks, about 26 weeks, about 28 weeks, about 30 weeks, about 32 weeks, about 34 weeks, about 36 weeks, about 38 weeks, about 40 weeks, about 42 weeks, about 44 weeks, about 46 weeks, about 48 weeks, about 50 weeks, about 52 weeks, about 54 weeks, about 56 weeks, about 58 weeks, about 60 weeks, about 62 weeks, about 64 weeks, about 66 weeks, about 68 weeks, about 70 weeks, about 72 weeks, about 74 weeks, about 76 weeks, about 78 weeks, or about 80 weeks from the first dose. In some embodiments, the mucosal healing is present within about 30 weeks from the first dose. In some embodiments, the mucosal healing is present within about 10 weeks from the first dose. In some embodiments, the mucosal healing is present within about 6 weeks from the first dose. In some embodiments, the mucosal healing is present at about 6 weeks, about 10 weeks, and about 30 weeks from the first dose.

In any of the preceding methods, the treating can result in a change from baseline in the subject's UC Endoscopic Index of Severity. In some embodiments, the change from baseline in the subject's UC Endoscopic Index of Severity may be present within about 1 week, about 2 weeks, about 3 weeks, about 4 weeks, about 5 weeks, about 6 weeks, about 7 weeks, about 9 weeks, about 10 weeks, about 11 weeks, about 12 weeks, about 14 weeks, about 16 weeks, about 18 weeks, about 20 weeks, about 22 weeks, about 24 weeks, about 26 weeks, about 28 weeks, about 30 weeks, about 32 weeks, about 34 weeks, about 36 weeks, about 38 weeks, about 40 weeks, about 42 weeks, about 44 weeks, about 46 weeks, about 48 weeks, about 50 weeks, about 52 weeks, about 54 weeks, about 56 weeks, about 58 weeks, about 60 weeks, about 62 weeks, about 64 weeks, about 66 weeks, about 68 weeks, about 70 weeks, about 72 weeks, about 74 weeks, about 76 weeks, about 78 weeks, or about 80 weeks from the first dose. In some embodiments, the treating results in the change from baseline in the subject's UC Endoscopic Index of Severity within about 30 weeks from the first dose. In some embodiments, the treating results in the change from baseline in the subject's UC Endoscopic Index of Severity within about 10 weeks from the first dose. In some embodiments, the treating results in the change from baseline in the subject's UC Endoscopic Index of Severity within about 6 weeks from the first dose. In some embodiments, the treating results in the change from baseline in the subject's UC Endoscopic Index of Severity at about 6 weeks, about 10 weeks, and about 30 weeks from the first dose.

In any of the preceding methods, the treating can result in histological healing. In some embodiments, the histological healing is a Nancy score of 0 or 1 as assessed by the Nancy Histological Index. In some embodiments, the histological healing may be present within about 1 week, about 2 weeks, about 3 weeks, about 4 weeks, about 5 weeks, about 6 weeks, about 7 weeks, about 9 weeks, about 10 weeks, about 11 weeks, about 12 weeks, about 14 weeks, about 16 weeks, about 18 weeks, about 20 weeks, about 22 weeks, about 24 weeks, about 26 weeks, about 28 weeks, about 30 weeks, about 32 weeks, about 34 weeks, about 36 weeks, about 38 weeks, about 40 weeks, about 42 weeks, about 44 weeks, about 46 weeks, about 48 weeks, about 50 weeks, about 52 weeks, about 54 weeks, about 56 weeks, about 58 weeks, about 60 weeks, about 62 weeks, about 64 weeks, about 66 weeks, about 68 weeks, about 70 weeks, about 72 weeks, about 74 weeks, about 76 weeks, about 78 weeks, or about 80 weeks from the first dose. In some embodiments, the treating results in the histological healing within about 30 weeks from the first dose. In some embodiments, the treating results in the histological healing within about 10 weeks from the first dose. In some embodiments, the treating results in the histological healing within about 6 weeks from the first dose. In some embodiments, the treating results in the histological healing at about 6 weeks, about 10 weeks, and about 30 weeks from the first dose.

In any of the preceding methods, the IBD can be UC or Crohn's disease. In some embodiments, the IBD is UC. In some embodiments, the UC is moderate to severe UC. In some embodiments, the moderate to severe UC is defined as a mMCS of 5-9 with an endoscopic subscore of about 2 or higher, a rectal bleeding subscore of about 1 or higher, and a stool frequency subscore of about 1 or higher prior to the treating. In some embodiments, the subject has UC a minimum of about 20 cm from the anal verge as determined by baseline endoscopy. In other embodiments, the IBD is Crohn's disease. In some embodiments, the subject has left-sided colitis, extensive colitis, or pancolitis prior to the treating. In some embodiments of any of the preceding aspects, the amelioration of one or more symptoms of IBD, clinical remission, and/or clinical response is maintained at least one month (e.g., at least one month, at least two months, at least three months, at least four months, at least five months, at least six months, at least seven months, at least eight months, at least nine months, at least ten months, at least eleven months, at least twelve months, or longer) after the end of treatment. In some embodiments, the amelioration of symptoms, clinical remission, and/or clinical response is maintained at least three months after the end of treatment.

In some embodiments of any of the preceding methods, the subject has had an inadequate response, loss of response, or intolerance to prior immunosuppressant treatment. In some embodiments, the prior immunosuppressant treatment is treatment with an aminosalicylate, an immunomodulatory agent, a tumor necrosis factor (TNF) antagonist, and/or a corticosteroid. In some embodiments, the subject has had persistent signs or symptoms of active disease despite treatment with at least one 12-week regimen of azathioprine (AZA), mercaptopurine (6-MP), and/or methotrexate (MTX) within five years prior to the treating. In some embodiments, the subject has a history of intolerance to AZA, 6-MP, or MTX within five years prior to the treating. In some embodiments, the subject has had persistent signs or symptoms of active disease despite treatment with at least two induction doses of infliximab, adalimumab, or golimumab within five years prior to the treating. In some embodiments, the subject has had recurrence of signs or symptoms of active disease during maintenance after initial response to induction therapy with infliximab, adalimumab, or golimumab. In some embodiments, the subject has had intolerance to a TNF antagonist. In some embodiments, (i) the subject has had persistent signs or symptoms of active disease despite treatment with at least one 4-week induction regimen that included 30 mg/day of oral prednisone (or equivalent) for at least 2 weeks or 30 mg/day of IV prednisone (or equivalent) for at least 1 week within five years prior to the treating; (ii) the subject has had two failed attempts to taper corticosteroids below 10 mg/day of oral prednisone (or equivalent); or (iii) the subject has a history of intolerance to corticosteroids within five years prior to the treating.

In any of the preceding methods, the IL-22 Fc fusion protein can be any IL-22 Fc fusion protein described in Subsection 1 below.

In some embodiments of any of the preceding methods, the IL-22 Fc fusion protein is administered to the patient as a monotherapy. In other embodiments, the IL-22 Fc fusion protein is administered to the subject as a combination therapy. In some embodiments, the IL-22 Fc fusion protein is administered to the subject concurrently with an additional therapeutic agent. In other embodiments, the IL-22 Fc fusion protein is administered to the subject prior to the administration of an additional therapeutic agent. In other embodiments, the IL-22 Fc fusion protein is administered to the subject following the administration of an additional therapeutic agent.

In some embodiments of any of the preceding methods, the IL-22 Fc fusion protein is administered in combination with an additional IBD therapy selected from an aminosalicylate, an immunomodulatory agent, a tumor necrosis factor (TNF) antagonist, an anti-integrin agent, a mucosal addressin cell adhesion molecule (MAdCAM) antagonist, an IL-23 antagonist, an IL-12 antagonist, an IL-12/IL-23 antagonist, an antibiotic, or a corticosteroid. In some embodiments, the additional IBD therapy is an aminosalicylate. In some embodiments, the aminosalicylate comprises 5-aminosalicylic acid (5-ASA). In some embodiments, the additional IBD therapy is an immunomodulatory agent (e.g., azathioprine, mercaptopurine, cyclosporine, tacrolimus, sirolimus, mycophenolic acid, or methotrexate). In some embodiments, the additional IBD therapy is a TNF antagonist. In some embodiments, the TNF antagonist is an anti-TNF antibody (e.g., infliximab, adalimumab, golimumab, certolizumab pegol, a fragment thereof, or a derivative thereof) or a soluble TNF receptor (e.g., etanercept, a fragment thereof, or a derivative thereof). In some embodiments, the additional IBD therapy is an anti-integrin agent. In some embodiments, the anti-integrin agent is an anti-integrin antibody (e.g., an anti-α4-integrin antibody (e.g., natalizumab, vedolizumab, a fragment thereof, or a derivative thereof). In some embodiments, the MAdCAM antagonist is an anti-MAdCAM antibody (e.g. PF-00547659 or SHP647). In some embodiments the IL-23 antagonist is an anti-IL-23 antibody (e.g., briakizumab, guselkumab, risankizumab, tilorakizumab, or ustekinumab). In some embodiments, the IL-12 antagonist is an anti-IL-12 antibody (e.g., ABT-874/J695). In some embodiments, the IL-12/IL-23 antagonist is an anti-IL-12/IL-23 antibody (e.g., ustekinumab or briakinumab).

In any of the preceding methods, the IL-22 Fc fusion protein of the invention (and any additional therapeutic agent) can be administered by any suitable means, including parenteral, intrapulmonary, topical and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein. In some embodiments, the administering is intravenous, e.g., by intravenous infusion or injection. In other embodiments, the administering is by subcutaneous administration, e.g., injection.

In another aspect, a composition (e.g., an IL-22 Fc fusion protein or a pharmaceutical composition thereof) for use as a medicament is provided. In further aspects, a composition (e.g., an IL-22 Fc fusion protein or a pharmaceutical composition thereof) for use in treating IBD, including UC and CD, is provided. In certain embodiments, a composition (e.g., an IL-22 Fc fusion protein or a pharmaceutical composition thereof) for use in a method of treatment is provided. In certain embodiments, the invention provides a composition (e.g., an IL-22 Fc fusion protein or a pharmaceutical composition thereof) for use in a method of treating an subject having UC or CD comprising administering to the an effective amount of the IL-22 Fc fusion protein. In one such embodiment, the method further comprises administering to the subject an effective amount of at least one additional therapeutic agent. In further embodiments, the invention provides a composition (e.g., an IL-22 Fc fusion protein or a pharmaceutical composition thereof) for use in enhancing epithelial proliferation, differentiation and/or migration. In certain particular embodiments, the epithelial tissue is intestinal epithelial tissue. In certain embodiments, the invention provides a composition (e.g., an IL-22 Fc fusion protein or a pharmaceutical composition thereof) for use in a method of enhancing epithelial proliferation, differentiation, and/or migration in a subject comprising administering to the subject an effective amount of the composition to enhance epithelial proliferation, differentiation, and/or migration.

In a further aspect, the invention provides for the use of a composition (e.g., an IL-22 Fc fusion protein or a pharmaceutical composition thereof) in the manufacture or preparation of a medicament. In one embodiment, the medicament is for treatment of IBD and wound healing. In a further embodiment, the medicament is for use in a method of treating IBD and wound healing comprising administering to a subject having IBD an effective amount of the medicament. In one such embodiment, the method further comprises administering to the subject an effective amount of at least one additional therapeutic agent. In a further embodiment, the medicament is for suppressing inflammatory response in the gut epithelial cells. In a further embodiment, the medicament is for use in a method of enhancing epithelial proliferation, differentiation and/or migration in a subject comprising administering to the individual an amount effective of the medicament to enhance epithelial proliferation, differentiation, and/or migration.

In a further aspect, the invention provides a method for treating IBD, including UC and CD. In one embodiment, the method comprises administering to a subject having IBD an effective amount of a composition (e.g., an IL-22 Fc fusion protein or a pharmaceutical composition thereof). In one such embodiment, the method further comprises administering to the subject an effective amount of at least one additional therapeutic agent, as described below.

In a further aspect, the invention provides a method for enhancing epithelial proliferation, differentiation and/or migration in a subject. In one embodiment, the method comprises administering to the subject an effective amount of a composition (e.g., an IL-22 Fc fusion protein or a pharmaceutical composition thereof) to enhance epithelial proliferation, differentiation, and/or migration.

In any of the preceding methods, uses, and compositions, the subject may be a human.

Graft Versus Host Disease (GVHD)

Graft versus host disease (GVHD) is the most frequent and potentially fatal complication of an allogeneic hematopoietic progenitor cell transplantation. It appears when immunocompetent T cells from donor origin recognize antigens from recipient origin as foreign. The immune response activates donor T cells and destroys recipient tissues. The clinical picture of this immune response is called acute and chronic GVHD. Acute GVHD (aGVHD) is the main fatal complication during the first months after allogeneic hematopoietic progenitor cell transplantation, while chronic GVHD (cGVHD) accounts for a significant long-term fraction of the mortality, morbidity and reduced quality of life of patients.

aGVHD is a common and life-threatening complication of allogeneic hematopoietic stem cell transplantation (allo-HSCT) with a high unmet need for effective, non-immunosuppressive therapies for prevention and treatment. aGVHD can be summarized in three stages: initial tissue damage (including the gastrointestinal (GI) tract) from the conditioning regimen (myeloablative versus non-myeloablative) that activates the host antigen-presenting cells (APCs), followed by APCs activating donor T cells that finally lead to destruction of host tissue, including the skin, GI tract and liver. Risk factors for developing aGVHD include degree of human leukocyte antigen (HLA) mismatch between donor and recipient, relatedness of donor and recipient, female donor-male recipient, use of peripheral blood stem cell grafts, and intensity of conditioning regimen.

Diagnosis of aGVHD depends on the clinical, laboratory and biopsy assessment of target organs. aGVHD severity can be categorized as Grade I-IV based on the Glucksberg scale (Glucksberg H, et al. Transplantation 1974; 18(4):295-304), depending on the degree of skin, GI and/or liver involvement, with Grade IV representing the most severe disease. The Mount Sinai Acute GVHD International Consortium (MAGIC) updated the clinical staging criteria of aGVHD to allow a more standardized approach to aGVHD grading (Harris et al. Biol Blood Marrow Transplant 2016; 22:4-10).

Signs and symptoms associated with aGVHD include rash, dermatitis, hepatitis, jaundice, abdominal pain, and diarrhea. aGVHD most commonly involves the skin and GI tract, with the skin being the most frequent and usually the earliest clinical manifestation. aGVHD with GI involvement (GI or intestinal GVHD) is the most difficult to treat and associated with the highest rates of GVHD-related morbidity and mortality.

Prevention is an integral component to the management of patients undergoing allo-HSCT. To date, no pharmacologic therapies have been approved for the prevention of aGVHD. Although there is no universal prophylaxis regimen for aGVHD, the majority of centers use a combination of a calcineurin inhibitor (e.g., cyclosporine or tacrolimus) and methotrexate. Additional prophylaxis agents include sirolimus, mycophenolate mofetil (MMF), anti-thymocyte thymoglobulin (ATG), and post-transplant cyclophosphamide. Due to their immunosuppressive nature, current prophylaxis agents can cause increased serious infections, delayed hematologic recovery, and reduce graft versus tumor effects leading to an increased rate of relapse. Despite the use of prophylaxis, Grade II-IV aGVHD develops in approximately 35%-50% of patients after allo-HSCT, with approximately 15% developing severe aGVHD (Grades III-IV). Subsequently, approximately 59%-85% of patients who develop Grade II-IV aGVHD will develop chronic GVHD. Thus, there is a significant unmet medical need for non-immunosuppressive effective therapies to prevent aGVHD and the significant long-term morbidity and mortality associated with the disease in patients undergoing allo-HSCT.

Novel treatment modalities are also needed for cGVHD. Patients who have an increased risk of developing cGVHD are those who have received stem cells/bone marrow from an HLA (human leukocyte antigen) mismatched related donor or from an HLA matched unrelated donor, patients that may have already experienced acute GVHD, and older recipients. Chronic GVHD can appear at any time after allogenic transplant or several years after the transplant. Chronic GVHD can occur in the skin, liver, eyes, mouth, lungs, gastrointestinal tract, neuromuscular system, or genitourinary tract.

Chronic GVHD presents with the following key clinical manifestations: mucocutaneous, myofascial, pulmonary, and “other,” affecting essentially any organ system in the body. Characteristic features may include chronic inflammatory changes that can be relatively acellular involving ocular, oral, esophageal, skin, joint and fascial, and genital tissues. Progression to clinically significant fibrosis involving multiple organs in the integumentary, musculoskeletal, aerodigestive, gastrointestinal, cardiorespiratory, reproductive, and peripheral nervous systems occurs in severely affected individuals. Rare but severe clinical presentations of chronic GVHD also can include polyserositis (with pericardial and pleural effusions) or polymyositis with severe muscle weakness and elevated muscle enzyme levels. For a review of the signs and symptoms of cGVHD, as well as current treatment modalities, see K. R. Cooke et al. Biol Blood Marrow Transplant 23 (2017) 211-234.

Thus, in some embodiments, the invention provides methods, dosing regimens, and dosing cycles for treating or preventing GVHD in a subject. For example, any of the methods, dosing regimens, and/or dosing cycles described above or herein can be used in a method of treating or preventing GVHD. In some embodiments, the GVHD is chronic GVHD or acute GVHD. In particular embodiments, the GVHD is acute GVHD. In some embodiments, the GVHD is intestinal GVHD. In other embodiments, the GVHD is skin GVHD or liver GVHD. Such methods can provide a prophylactic effect against the development of, or a therapeutic effect against the progression of, clinical and/or histological and/or biochemical and/or pathological indicia (including both symptoms and signs) of GVHD. Administration of an IL-22 Fc fusion protein or composition thereof according to the methods described herein may reduce one or more symptoms of GVHD, including pain, rashes, skin thickness, yellow skin or eyes, mouth dryness or ulcers, taste abnormalities, dry eyes, infections, or weight loss.

In one aspect, the invention provides a method of treating or preventing GVHD in a subject that includes administering to the subject an IL-22 Fc fusion protein in a dosing regimen comprising a dosing cycle, wherein the dosing cycle comprises between about one and about twenty doses (e.g., about one, about two, about three, about four, about five, about six, about seven, about eight, about nine, about ten, about eleven, about twelve, about thirteen, about fourteen, about fifteen, about sixteen, about seventeen, about eighteen, about nineteen, or about twenty doses), and wherein a total of about 1 μg/kg to about 2000 μg/kg of the IL-22 Fc fusion protein is administered to the subject in the dosing cycle.

In one aspect, the invention provides an IL-22 Fc fusion protein for use in a method of treating or preventing graft versus host disease (GVHD) in a subject, wherein the IL-22 Fc fusion protein is for administration to the subject in a dosing regimen comprising a dosing cycle, wherein the dosing cycle comprises between about one and about twenty doses (e.g., about one, about two, about three, about four, about five, about six, about seven, about eight, about nine, about ten, about eleven, about twelve, about thirteen, about fourteen, about fifteen, about sixteen, about seventeen, about eighteen, about nineteen, or about twenty doses), and wherein a total of about 1 μg/kg to about 2000 μg/kg of the IL-22 Fc fusion protein is administered to the subject in the dosing cycle.

For example, provided herein is a method of treating or preventing GVHD in a subject that includes administering to the subject an IL-22 Fc fusion protein in a dosing regimen comprising a dosing cycle, wherein the dosing cycle comprises between two and ten doses, and wherein a total of about 60 μg/kg to about 900 μg/kg of the IL-22 Fc fusion protein is administered to the subject in the dosing cycle. In some embodiments, the first dose of the dosing cycle is administered to the subject about 3 (±2) days prior to allogeneic hematopoietic stem cell transplantation (allo-HSCT). In some embodiments, the second dose is administered on or about Day 11 following the allo-HSCT. In some embodiments, a total of about 480 μg/kg of the IL-22 Fc fusion protein is administered to the subject in the dosing cycle. In some embodiments, the dosing cycle comprises a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein. In some embodiments, the dosing cycle consists of a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein. In some embodiments, the C1D1, the C1D2, the C1D3, the C1D4, the C1D5, the C1D6, the C1D7, and the C1D8 are each between about 15 μg/kg to about 90 μg/kg. In some embodiments, the C1D1, the C1D2, the C1D3, the C1D4, the C1D5, the C1D6, the C1D7, and the C1D8 are each about 60 μg/kg. In some embodiments, the length of the dosing cycle is between about 2 weeks and about 20 weeks. In some embodiments, the length of the dosing cycle is about 96 days.

In another aspect, the invention features an IL-22 Fc fusion protein for use in a method of treating or preventing graft versus host disease (GVHD) in a subject, wherein the IL-22 Fc fusion protein is for administration to the subject in a dosing regimen comprising a dosing cycle, wherein the dosing cycle comprises between two and ten doses, and wherein a total of about 60 μg/kg to about 900 μg/kg of the IL-22 Fc fusion protein is administered to the subject in the dosing cycle. In some embodiments, the first dose of the dosing cycle is administered to the subject about 3 (±2) days prior to allogeneic hematopoietic stem cell transplantation (allo-HSCT). In some embodiments, the second dose is administered on or about Day 11 following the allo-HSCT. In some embodiments, a total of about 480 μg/kg of the IL-22 Fc fusion protein is administered to the subject in the dosing cycle. In some embodiments, the dosing cycle comprises a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein. In some embodiments, the dosing cycle consists of a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein. In some embodiments, the C1D1, the C1D2, the C1D3, the C1D4, the C1D5, the C1D6, the C1D7, and the C1D8 are each between about 15 μg/kg to about 90 μg/kg. In some embodiments, the C1D1, the C1D2, the C1D3, the C1D4, the C1D5, the C1D6, the C1D7, and the C1D8 are each about 60 μg/kg. In some embodiments, the length of the dosing cycle is between about 2 weeks and about 20 weeks. In some embodiments, the length of the dosing cycle is about 96 days.

In another aspect, provided herein is the use of an IL-22 Fc fusion protein in the preparation of a medicament for use in a method of treating or preventing graft versus host disease (GVHD) in a subject, wherein the IL-22 Fc fusion protein is for administration to the subject in a dosing regimen comprising a dosing cycle, wherein the dosing cycle comprises between two and ten doses, and wherein a total of about 60 μg/kg to about 900 μg/kg of the IL-22 Fc fusion protein is administered to the subject in the dosing cycle. In some embodiments, the first dose of the dosing cycle is administered to the subject about 3 (±2) days prior to allogeneic hematopoietic stem cell transplantation (allo-HSCT). In some embodiments, the second dose is administered on or about Day 11 following the allo-HSCT. In some embodiments, a total of about 480 μg/kg of the IL-22 Fc fusion protein is administered to the subject in the dosing cycle. In some embodiments, the dosing cycle comprises a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein. In some embodiments, the dosing cycle consists of a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein. In some embodiments, the C1D1, the C1D2, the C1D3, the C1D4, the C1D5, the C1D6, the C1D7, and the C1D8 are each between about 15 μg/kg to about 90 μg/kg. In some embodiments, the C1D1, the C1D2, the C1D3, the C1D4, the C1D5, the C1D6, the C1D7, and the C1D8 are each about 60 μg/kg. In some embodiments, the length of the dosing cycle is between about 2 weeks and about 20 weeks. In some embodiments, the length of the dosing cycle is about 96 days.

In some embodiments, the dose(s) are administered to the subject every week (q1w), every two weeks (q2w), every three weeks (q3w), every four weeks, (q4w), every five weeks (q5w), every six weeks (q6w), every seven weeks (q7w), every eight weeks (q8w), every nine weeks (q9w), every ten weeks (q10w), every 12 weeks (q12w), every fourteen weeks (q14w), every sixteen weeks (q16w), every eighteen weeks (q18w), or every twenty weeks (q20w). For example, in some embodiments, the doses are administered to the subject every week (q1w), every two weeks (q2w), every four weeks (q4w), or every six weeks (q6w). In particular embodiments, the doses are administered to the subject every two weeks (q2W).

In some embodiments, a total of about 720 μg/kg of the IL-22 Fc fusion protein is administered to the subject in the dosing cycle (e.g., about 90 μg/kg q2w in a ninety-six day dosing cycle). In some embodiments, a total of about 480 μg/kg of the IL-22 Fc fusion protein is administered to the subject in the dosing cycle (e.g., about 60 μg/kg q2w in a ninety-six day dosing cycle). In some embodiments, a total of about 240 μg/kg of the IL-22 Fc fusion protein is administered to the subject in the dosing cycle (e.g., about 30 μg/kg q2w in a ninety-six day dosing cycle).

For example, in some embodiments of any of the preceding methods, the dosing cycle includes about one to about twenty doses, about one to about nineteen doses, about one to about eighteen doses, about one to about seventeen doses, about one to about sixteen doses, about one to about fifteen doses, about one to about fourteen doses, about one to about thirteen doses, about one to about twelve doses, about one to about eleven doses, about one to about ten doses, about one to about nine doses, about one to about eight doses, about one to about seven doses, about one to about six doses, about one to about five doses, about one to about four doses, about one to about three doses, about one to about two doses, about two to about twenty doses, about two to about nineteen doses, about two to about eighteen doses, about two to about seventeen doses, about two to about sixteen doses, about two to about fifteen doses, about two to about fourteen doses, about two to about thirteen doses, about two to about twelve doses, about two to about eleven doses, about two to about ten doses, about two to about nine doses, about two to about eight doses, about two to about seven doses, about two to about six doses, about two to about five doses, about two to about four doses, about two to about three doses, about three to about twenty doses, about three to about nineteen doses, about three to about eighteen doses, about three to about seventeen doses, about three to about sixteen doses, about three to about fifteen doses, about three to about fourteen doses, about three to about thirteen doses, about three to about twelve doses, about three to about eleven doses, about three to about ten doses, about three to about nine doses, about three to about eight doses, about three to about seven doses, about three to about six doses, about three to about five doses, about three to about four doses, about four to about twenty doses, about four to about nineteen doses, about four to about eighteen doses, about four to about seventeen doses, about four to about sixteen doses, about four to about fifteen doses, about four to about fourteen doses, about four to about thirteen doses, about four to about twelve doses, about four to about eleven doses, about four to about ten doses, about four to about nine doses, about four to about eight doses, about four to about seven doses, about four to about six doses, about four to about five doses, about five to about twenty doses, about five to about nineteen doses, about five to about eighteen doses, about five to about seventeen doses, about five to about sixteen doses, about five to about fifteen doses, about five to about fourteen doses, about five to about thirteen doses, about five to about twelve doses, about five to about eleven doses, about five to about ten doses, about five to about nine doses, about five to about eight doses, about five to about seven doses, about five to about six doses, about six to about twenty doses, about six to about nineteen doses, about six to about eighteen doses, about six to about seventeen doses, about six to about sixteen doses, about six to about fifteen doses, about six to about fourteen doses, about six to about thirteen doses, about six to about twelve doses, about six to about eleven doses, about six to about ten doses, about six to about nine doses, about six to about eight doses, about six to about seven doses, about seven to about twenty doses, about seven to about nineteen doses, about seven to about eighteen doses, about seven to about seventeen doses, about seven to about sixteen doses, about seven to about fifteen doses, about seven to about fourteen doses, about seven to about thirteen doses, about seven to about twelve doses, about seven to about eleven doses, about seven to about ten doses, about seven to about nine doses, about seven to about eight doses, about eight to about twenty doses, about eight to about nineteen doses, about eight to about eighteen doses, about eight to about seventeen doses, about eight to about sixteen doses, about eight to about fifteen doses, about eight to about fourteen doses, about eight to about thirteen doses, about eight to about twelve doses, about eight to about eleven doses, about eight to about ten doses, about eight to about nine doses, about nine to about twenty doses, about nine to about nineteen doses, about nine to about eighteen doses, about nine to about seventeen doses, about nine to about sixteen doses, about nine to about fifteen doses, about nine to about fourteen doses, about nine to about thirteen doses, about nine to about twelve doses, about nine to about eleven doses, about nine to about ten doses, about ten to about twenty doses, about ten to about nineteen doses, about ten to about eighteen doses, about ten to about seventeen doses, about ten to about sixteen doses, about ten to about fifteen doses, about ten to about fourteen doses, about ten to about thirteen doses, about ten to about twelve doses, about ten to about eleven doses, about eleven to about twenty doses, about eleven to about nineteen doses, about eleven to about eighteen doses, about eleven to about seventeen doses, about eleven to about sixteen doses, about eleven to about fifteen doses, about eleven to about fourteen doses, about eleven to about thirteen doses, about eleven to about twelve doses, about twelve to about twenty doses, about twelve to about nineteen doses, about twelve to about eighteen doses, about twelve to about seventeen doses, about twelve to about sixteen doses, about twelve to about fifteen doses, about twelve to about fourteen doses, about twelve to about thirteen doses, about thirteen to about twenty doses, about thirteen to about nineteen doses, about thirteen to about eighteen doses, about thirteen to about seventeen doses, about thirteen to about sixteen doses, about thirteen to about fifteen doses, about thirteen to about fourteen doses, about fourteen to about twenty doses, about fourteen to about nineteen doses, about fourteen to about eighteen doses, about fourteen to about seventeen doses, about fourteen to about sixteen doses, about fourteen to about fifteen doses, about fifteen to about twenty doses, about fifteen to about nineteen doses, about fifteen to about eighteen doses, about fifteen to about seventeen doses, about fifteen to about sixteen doses, about sixteen to about twenty doses, about sixteen to about nineteen doses, about sixteen to about eighteen doses, about sixteen to about seventeen doses, about seventeen to about twenty doses, about seventeen to about nineteen doses, about seventeen to about eighteen doses, about eighteen to about twenty doses, about eighteen to about nineteen doses, or about nineteen to about twenty doses.

For example, in some embodiments of any of the preceding methods, a total of about 1 μg/kg to about 2000 μg/kg of the IL-22 Fc fusion protein is administered to the subject in the dosing cycle, e.g., about 1 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 110 μg/kg, about 120 μg/kg, about 130 μg/kg, about 140 μg/kg, about 150 μg/kg, about 160 μg/kg, about 170 μg/kg, about 180 μg/kg, about 190 μg/kg, about 200 μg/kg, about 210 μg/kg, about 220 μg/kg, about 230 μg/kg, about 240 μg/kg, about 250 μg/kg, about 260 μg/kg, about 270 μg/kg, about 280 μg/kg, about 290 μg/kg, about 300 μg/kg, about 310 μg/kg, about 320 μg/kg, about 330 μg/kg, about 340 μg/kg, about 350 μg/kg, about 360 μg/kg, about 370 μg/kg, about 380 μg/kg, about 390 μg/kg, about 400 μg/kg, about 410 μg/kg, about 420 μg/kg, about 430 μg/kg, about 440 μg/kg, about 450 μg/kg, about 460 μg/kg, about 470 μg/kg, about 480 μg/kg, about 490 μg/kg, about 500 μg/kg, about 510 μg/kg, about 520 μg/kg, about 530 μg/kg, about 540 μg/kg, about 550 μg/kg, about 560 μg/kg, about 570 μg/kg, about 580 μg/kg, about 590 μg/kg, about 600 μg/kg, about 610 μg/kg, about 620 μg/kg, about 630 μg/kg, about 640 μg/kg, about 650 μg/kg, about 660 μg/kg, about 670 μg/kg, about 680 μg/kg, about 690 μg/kg, about 700 μg/kg, about 710 μg/kg, about 720 μg/kg, about 730 μg/kg, about 740 μg/kg, about 750 μg/kg, about 760 μg/kg, about 770 μg/kg, about 780 μg/kg, about 790 μg/kg, about 800 μg/kg, about 810 μg/kg, about 820 μg/kg, about 830 μg/kg, about 840 μg/kg, about 850 μg/kg, about 860 μg/kg, about 870 μg/kg, about 880 μg/kg, about 890 μg/kg, about 900 μg/kg, about 910 μg/kg, about 920 μg/kg, about 930 μg/kg, about 940 μg/kg, about 950 μg/kg, about 960 μg/kg, about 970 μg/kg, about 980 μg/kg, about 990 μg/kg, about 1000 μg/kg, about 1010 μg/kg, about 1020 μg/kg, about 1030 μg/kg, about 1040 μg/kg, about 1050 μg/kg, about 1060 μg/kg, about 1070 μg/kg, about 1080 μg/kg, about 1090 μg/kg, about 1100 μg/kg, about 1110 μg/kg, about 1120 μg/kg, about 1130 μg/kg, about 1140 μg/kg, about 1150 μg/kg, about 1160 μg/kg, about 1170 μg/kg, about 1180 μg/kg, about 1190 μg/kg, about 1200 μg/kg, about 1210 μg/kg, about 1220 μg/kg, about 1230 μg/kg, about 1240 μg/kg, about 1250 μg/kg, about 1260 μg/kg, about 1270 μg/kg, about 1280 μg/kg, about 1290 μg/kg, about 1300 μg/kg, about 1310 μg/kg, about 1320 μg/kg, about 1330 μg/kg, about 1340 μg/kg, about 1350 μg/kg, about 1360 μg/kg, about 1370 μg/kg, about 1380 μg/kg, about 1390 μg/kg, about 1400 μg/kg, about 1410 μg/kg, about 1420 μg/kg, about 1430 μg/kg, about 1440 μg/kg, about 1450 μg/kg, about 1460 μg/kg, about 1470 μg/kg, about 1480 μg/kg, about 1490 μg/kg, about 1500 μg/kg, about 1510 μg/kg, about 1520 μg/kg, about 1530 μg/kg, about 1540 μg/kg, about 1550 μg/kg, about 1560 μg/kg, about 1570 μg/kg, about 1580 μg/kg, about 1590 μg/kg, about 1600 μg/kg, about 1610 μg/kg, about 1620 μg/kg, about 1630 μg/kg, about 1640 μg/kg, about 1650 μg/kg, about 1660 μg/kg, about 1670 μg/kg, about 1680 μg/kg, about 1690 μg/kg, about 1700 μg/kg, about 1710 μg/kg, about 1720 μg/kg, about 1730 μg/kg, about 1740 μg/kg, about 1750 μg/kg, about 1760 μg/kg, about 1770 μg/kg, about 1780 μg/kg, about 1790 μg/kg, about 1800 μg/kg, about 1810 μg/kg, about 1820 μg/kg, about 1830 μg/kg, about 1840 μg/kg, about 1850 μg/kg, about 1860 μg/kg, about 1870 μg/kg, about 1880 μg/kg, about 1890 μg/kg, about 1900 μg/kg, about 1910 μg/kg, about 1920 μg/kg, about 1930 μg/kg, about 1940 μg/kg, about 1950 μg/kg, about 1960 μg/kg, about 1970 μg/kg, about 1980 μg/kg, about 1990 μg/kg, or about 2000 μg/kg.

For example, in some embodiments of any of the preceding methods, a total of about 1 μg/kg to about 2000 μg/kg, about 1 μg/kg to about 1900 μg/kg, about 1 μg/kg to about 1800 μg/kg, about 1 μg/kg to about 1700 μg/kg, about 1 μg/kg to about 1600 μg/kg, about 1 μg/kg to about 1500 μg/kg, about 1 μg/kg to about 1400 μg/kg, about 1 μg/kg to about 1300 μg/kg, about 1 μg/kg to about 1200 μg/kg, about 1 μg/kg to about 1100 μg/kg, about 1 μg/kg to about 1000 μg/kg, about 1 μg/kg to about 900 μg/kg, about 1 μg/kg to about 800 μg/kg, about 1 μg/kg to about 750 μg/kg, about 1 μg/kg to about 700 μg/kg, about 1 μg/kg to about 650 μg/kg, about 1 μg/kg to about 600 μg/kg, about 1 μg/kg to about 550 μg/kg, about 1 μg/kg to about 500 μg/kg, about 1 μg/kg to about 450 μg/kg, about 1 μg/kg to about 400 μg/kg, about 1 μg/kg to about 350 μg/kg, about 1 μg/kg to about 300 μg/kg, about 1 μg/kg to about 250 μg/kg, about 1 μg/kg to about 200 μg/kg, about 1 μg/kg to about 150 μg/kg, about 1 μg/kg to about 100 μg/kg, about 1 μg/kg to about 50 μg/kg, 50 μg/kg to about 2000 μg/kg, about 50 μg/kg to about 1900 μg/kg, about 50 μg/kg to about 1800 μg/kg, about 50 μg/kg to about 1700 μg/kg, about 50 μg/kg to about 1600 μg/kg, about 50 μg/kg to about 1500 μg/kg, about 50 μg/kg to about 1400 μg/kg, about 50 μg/kg to about 1300 μg/kg, about 50 μg/kg to about 1200 μg/kg, about 50 μg/kg to about 1100 μg/kg, about 50 μg/kg to about 1000 μg/kg, about 50 μg/kg to about 900 μg/kg, about 50 μg/kg to about 800 μg/kg, about 50 μg/kg to about 750 μg/kg, about 50 μg/kg to about 700 μg/kg, about 50 μg/kg to about 650 μg/kg, about 50 μg/kg to about 600 μg/kg, about 50 μg/kg to about 550 μg/kg, about 50 μg/kg to about 500 μg/kg, about 50 μg/kg to about 450 μg/kg, about 50 μg/kg to about 400 μg/kg, about 50 μg/kg to about 350 μg/kg, about 50 μg/kg to about 300 μg/kg, about 50 μg/kg to about 250 μg/kg, about 50 μg/kg to about 200 μg/kg, about 50 μg/kg to about 150 μg/kg, about 50 μg/kg to about 100 μg/kg, about 100 μg/kg to about 2000 μg/kg, about 100 μg/kg to about 1900 μg/kg, about 100 μg/kg to about 1800 μg/kg, about 100 μg/kg to about 1700 μg/kg, about 100 μg/kg to about 1600 μg/kg, about 100 μg/kg to about 1500 μg/kg, about 100 μg/kg to about 1400 μg/kg, about 100 μg/kg to about 1300 μg/kg, about 100 μg/kg to about 1200 μg/kg, about 100 μg/kg to about 1100 μg/kg, about 100 μg/kg to about 1000 μg/kg, about 100 μg/kg to about 900 μg/kg, about 100 μg/kg to about 800 μg/kg, about 100 μg/kg to about 750 μg/kg, about 100 μg/kg to about 700 μg/kg, about 100 μg/kg to about 650 μg/kg, about 100 μg/kg to about 600 μg/kg, about 100 μg/kg to about 550 μg/kg, about 100 μg/kg to about 500 μg/kg, about 100 μg/kg to about 450 μg/kg, about 100 μg/kg to about 400 μg/kg, about 100 μg/kg to about 350 μg/kg, about 100 μg/kg to about 300 μg/kg, about 100 μg/kg to about 250 μg/kg, about 100 μg/kg to about 200 μg/kg, about 100 μg/kg to about 150 μg/kg, about 150 μg/kg to about 2000 μg/kg, about 150 μg/kg to about 1900 μg/kg, about 150 μg/kg to about 1800 μg/kg, about 150 μg/kg to about 1700 μg/kg, about 150 μg/kg to about 1600 μg/kg, about 150 μg/kg to about 1500 μg/kg, about 150 μg/kg to about 1400 μg/kg, about 150 μg/kg to about 1300 μg/kg, about 150 μg/kg to about 1200 μg/kg, about 150 μg/kg to about 1100 μg/kg, about 150 μg/kg to about 1000 μg/kg, about 150 μg/kg to about 900 μg/kg, about 150 μg/kg to about 800 μg/kg, about 150 μg/kg to about 750 μg/kg, about 150 μg/kg to about 700 μg/kg, about 150 μg/kg to about 650 μg/kg, about 150 μg/kg to about 600 μg/kg, about 150 μg/kg to about 550 μg/kg, about 150 μg/kg to about 500 μg/kg, about 150 μg/kg to about 450 μg/kg, about 150 μg/kg to about 400 μg/kg, about 150 μg/kg to about 350 μg/kg, about 150 μg/kg to about 300 μg/kg, about 150 μg/kg to about 250 μg/kg, about 150 μg/kg to about 200 μg/kg, about 200 μg/kg to about 2000 μg/kg, about 200 μg/kg to about 1900 μg/kg, about 200 μg/kg to about 1800 μg/kg, about 200 μg/kg to about 1700 μg/kg, about 200 μg/kg to about 1600 μg/kg, about 200 μg/kg to about 1500 μg/kg, about 200 μg/kg to about 1400 μg/kg, about 200 μg/kg to about 1300 μg/kg, about 200 μg/kg to about 1200 μg/kg, about 200 μg/kg to about 1100 μg/kg, about 200 μg/kg to about 1000 μg/kg, about 200 μg/kg to about 900 μg/kg, about 200 μg/kg to about 800 μg/kg, about 200 μg/kg to about 750 μg/kg, about 200 μg/kg to about 700 μg/kg, about 200 μg/kg to about 650 μg/kg, about 200 μg/kg to about 600 μg/kg, about 200 μg/kg to about 550 μg/kg, about 200 μg/kg to about 500 μg/kg, about 200 μg/kg to about 450 μg/kg, about 200 μg/kg to about 400 μg/kg, about 200 μg/kg to about 350 μg/kg, about 200 μg/kg to about 300 μg/kg, about 200 μg/kg to about 250 μg/kg, about 250 μg/kg to about 2000 μg/kg, about 250 μg/kg to about 1900 μg/kg, about 250 μg/kg to about 1800 μg/kg, about 250 μg/kg to about 1700 μg/kg, about 250 μg/kg to about 1600 μg/kg, about 250 μg/kg to about 1500 μg/kg, about 250 μg/kg to about 1400 μg/kg, about 250 μg/kg to about 1300 μg/kg, about 250 μg/kg to about 1200 μg/kg, about 250 μg/kg to about 1100 μg/kg, about 250 μg/kg to about 1000 μg/kg, about 250 μg/kg to about 900 μg/kg, about 250 μg/kg to about 800 μg/kg, about 250 μg/kg to about 750 μg/kg, about 250 μg/kg to about 700 μg/kg, about 250 μg/kg to about 650 μg/kg, about 250 μg/kg to about 600 μg/kg, about 250 μg/kg to about 550 μg/kg, about 250 μg/kg to about 500 μg/kg, about 250 μg/kg to about 450 μg/kg, about 250 μg/kg to about 400 μg/kg, about 250 μg/kg to about 350 μg/kg, about 250 μg/kg to about 300 μg/kg, about 300 μg/kg to about 2000 μg/kg, about 300 μg/kg to about 1900 μg/kg, about 300 μg/kg to about 1800 μg/kg, about 300 μg/kg to about 1700 μg/kg, about 300 μg/kg to about 1600 μg/kg, about 300 μg/kg to about 1500 μg/kg, about 300 μg/kg to about 1400 μg/kg, about 300 μg/kg to about 1300 μg/kg, about 300 μg/kg to about 1200 μg/kg, about 300 μg/kg to about 1100 μg/kg, about 300 μg/kg to about 1000 μg/kg, about 300 μg/kg to about 900 μg/kg, about 300 μg/kg to about 800 μg/kg, about 300 μg/kg to about 750 μg/kg, about 300 μg/kg to about 700 μg/kg, about 300 μg/kg to about 650 μg/kg, about 300 μg/kg to about 600 μg/kg, about 300 μg/kg to about 550 μg/kg, about 300 μg/kg to about 500 μg/kg, about 300 μg/kg to about 450 μg/kg, about 300 μg/kg to about 400 μg/kg, about 300 μg/kg to about 350 μg/kg, about 350 μg/kg to about 2000 μg/kg, about 350 μg/kg to about 1900 μg/kg, about 350 μg/kg to about 1800 μg/kg, about 350 μg/kg to about 1700 μg/kg, about 350 μg/kg to about 1600 μg/kg, about 350 μg/kg to about 1500 μg/kg, about 350 μg/kg to about 1400 μg/kg, about 350 μg/kg to about 1300 μg/kg, about 350 μg/kg to about 1200 μg/kg, about 350 μg/kg to about 1100 μg/kg, about 350 μg/kg to about 1000 μg/kg, about 350 μg/kg to about 900 μg/kg, about 350 μg/kg to about 800 μg/kg, about 350 μg/kg to about 750 μg/kg, about 350 μg/kg to about 700 μg/kg, about 350 μg/kg to about 650 μg/kg, about 350 μg/kg to about 600 μg/kg, about 350 μg/kg to about 550 μg/kg, about 350 μg/kg to about 500 μg/kg, about 350 μg/kg to about 450 μg/kg, about 350 μg/kg to about 400 μg/kg, about 400 μg/kg to about 2000 μg/kg, about 400 μg/kg to about 1900 μg/kg, about 400 μg/kg to about 1800 μg/kg, about 400 μg/kg to about 1700 μg/kg, about 400 μg/kg to about 1600 μg/kg, about 400 μg/kg to about 1500 μg/kg, about 400 μg/kg to about 1400 μg/kg, about 400 μg/kg to about 1300 μg/kg, about 400 μg/kg to about 1200 μg/kg, about 400 μg/kg to about 1100 μg/kg, about 400 μg/kg to about 1000 μg/kg, about 400 μg/kg to about 900 μg/kg, about 400 μg/kg to about 800 μg/kg, about 400 μg/kg to about 750 μg/kg, about 400 μg/kg to about 700 μg/kg, about 400 μg/kg to about 650 μg/kg, about 400 μg/kg to about 600 μg/kg, about 400 μg/kg to about 550 μg/kg, about 400 μg/kg to about 500 μg/kg, about 400 μg/kg to about 450 μg/kg, about 450 μg/kg to about 2000 μg/kg, about 450 μg/kg to about 1900 μg/kg, about 450 μg/kg to about 1800 μg/kg, about 450 μg/kg to about 1700 μg/kg, about 450 μg/kg to about 1600 μg/kg, about 450 μg/kg to about 1500 μg/kg, about 450 μg/kg to about 1400 μg/kg, about 450 μg/kg to about 1300 μg/kg, about 450 μg/kg to about 1200 μg/kg, about 450 μg/kg to about 1100 μg/kg, about 450 μg/kg to about 1000 μg/kg, about 450 μg/kg to about 900 μg/kg, about 450 μg/kg to about 800 μg/kg, about 450 μg/kg to about 750 μg/kg, about 450 μg/kg to about 700 μg/kg, about 450 μg/kg to about 650 μg/kg, about 450 μg/kg to about 600 μg/kg, about 450 μg/kg to about 550 μg/kg, about 450 μg/kg to about 500 μg/kg, about 500 μg/kg to about 2000 μg/kg, about 500 μg/kg to about 1900 μg/kg, about 500 μg/kg to about 1800 μg/kg, about 500 μg/kg to about 1700 μg/kg, about 500 μg/kg to about 1600 μg/kg, about 500 μg/kg to about 1500 μg/kg, about 500 μg/kg to about 1400 μg/kg, about 500 μg/kg to about 1300 μg/kg, about 500 μg/kg to about 1200 μg/kg, about 500 μg/kg to about 1100 μg/kg, about 500 μg/kg to about 1000 μg/kg, about 500 μg/kg to about 900 μg/kg, about 500 μg/kg to about 800 μg/kg, about 500 μg/kg to about 750 μg/kg, about 500 μg/kg to about 700 μg/kg, about 500 μg/kg to about 650 μg/kg, about 500 μg/kg to about 600 μg/kg, about 500 μg/kg to about 550 μg/kg, about 550 μg/kg to about 2000 μg/kg, about 550 μg/kg to about 1900 μg/kg, about 550 μg/kg to about 1800 μg/kg, about 550 μg/kg to about 1700 μg/kg, about 550 μg/kg to about 1600 μg/kg, about 550 μg/kg to about 1500 μg/kg, about 550 μg/kg to about 1400 μg/kg, about 550 μg/kg to about 1300 μg/kg, about 550 μg/kg to about 1200 μg/kg, about 550 μg/kg to about 1100 μg/kg, about 550 μg/kg to about 1000 μg/kg, about 550 μg/kg to about 900 μg/kg, about 550 μg/kg to about 800 μg/kg, about 550 μg/kg to about 750 μg/kg, about 550 μg/kg to about 700 μg/kg, about 550 μg/kg to about 650 μg/kg, about 550 μg/kg to about 600 μg/kg, about 600 μg/kg to about 2000 μg/kg, about 600 μg/kg to about 1900 μg/kg, about 600 μg/kg to about 1800 μg/kg, about 600 μg/kg to about 1700 μg/kg, about 600 μg/kg to about 1600 μg/kg, about 600 μg/kg to about 1500 μg/kg, about 600 μg/kg to about 1400 μg/kg, about 600 μg/kg to about 1300 μg/kg, about 600 μg/kg to about 1200 μg/kg, about 600 μg/kg to about 1100 μg/kg, about 600 μg/kg to about 1000 μg/kg, about 600 μg/kg to about 900 μg/kg, about 600 μg/kg to about 800 μg/kg, about 600 μg/kg to about 750 μg/kg, about 600 μg/kg to about 700 μg/kg, about 600 μg/kg to about 650 μg/kg, about 650 μg/kg to about 2000 μg/kg, about 650 μg/kg to about 1900 μg/kg, about 650 μg/kg to about 1800 μg/kg, about 650 μg/kg to about 1700 μg/kg, about 650 μg/kg to about 1600 μg/kg, about 650 μg/kg to about 1500 μg/kg, about 650 μg/kg to about 1400 μg/kg, about 650 μg/kg to about 1300 μg/kg, about 650 μg/kg to about 1200 μg/kg, about 650 μg/kg to about 1100 μg/kg, about 650 μg/kg to about 1000 μg/kg, about 650 μg/kg to about 900 μg/kg, about 650 μg/kg to about 800 μg/kg, about 650 μg/kg to about 750 μg/kg, about 650 μg/kg to about 700 μg/kg, about 700 μg/kg to about 2000 μg/kg, about 700 μg/kg to about 1900 μg/kg, about 700 μg/kg to about 1800 μg/kg, about 700 μg/kg to about 1700 μg/kg, about 700 μg/kg to about 1600 μg/kg, about 700 μg/kg to about 1500 μg/kg, about 700 μg/kg to about 1400 μg/kg, about 700 μg/kg to about 1300 μg/kg, about 700 μg/kg to about 1200 μg/kg, about 700 μg/kg to about 1100 μg/kg, about 700 μg/kg to about 1000 μg/kg, about 700 μg/kg to about 900 μg/kg, about 700 μg/kg to about 800 μg/kg, about 700 μg/kg to about 750 μg/kg, about 750 μg/kg to about 2000 μg/kg, about 750 μg/kg to about 1900 μg/kg, about 750 μg/kg to about 1800 μg/kg, about 750 μg/kg to about 1700 μg/kg, about 750 μg/kg to about 1600 μg/kg, about 750 μg/kg to about 1500 μg/kg, about 750 μg/kg to about 1400 μg/kg, about 750 μg/kg to about 1300 μg/kg, about 750 μg/kg to about 1200 μg/kg, about 750 μg/kg to about 1100 μg/kg, about 750 μg/kg to about 1000 μg/kg, about 750 μg/kg to about 900 μg/kg, about 750 μg/kg to about 800 μg/kg, about 800 μg/kg to about 2000 μg/kg, about 800 μg/kg to about 1900 μg/kg, about 800 μg/kg to about 1800 μg/kg, about 800 μg/kg to about 1700 μg/kg, about 800 μg/kg to about 1600 μg/kg, about 800 μg/kg to about 1500 μg/kg, about 800 μg/kg to about 1400 μg/kg, about 800 μg/kg to about 1300 μg/kg, about 800 μg/kg to about 1200 μg/kg, about 800 μg/kg to about 1100 μg/kg, about 800 μg/kg to about 1000 μg/kg, about 800 μg/kg to about 900 μg/kg, about 850 μg/kg to about 2000 μg/kg, about 850 μg/kg to about 1900 μg/kg, about 850 μg/kg to about 1800 μg/kg, about 850 μg/kg to about 1700 μg/kg, about 850 μg/kg to about 1600 μg/kg, about 850 μg/kg to about 1500 μg/kg, about 850 μg/kg to about 1400 μg/kg, about 850 μg/kg to about 1300 μg/kg, about 850 μg/kg to about 1200 μg/kg, about 850 μg/kg to about 1100 μg/kg, about 850 μg/kg to about 1000 μg/kg, about 850 μg/kg to about 900 μg/kg, about 900 μg/kg to about 2000 μg/kg, about 900 μg/kg to about 1900 μg/kg, about 900 μg/kg to about 1800 μg/kg, about 900 μg/kg to about 1700 μg/kg, about 900 μg/kg to about 1600 μg/kg, about 900 μg/kg to about 1500 μg/kg, about 900 μg/kg to about 1400 μg/kg, about 900 μg/kg to about 1300 μg/kg, about 900 μg/kg to about 1200 μg/kg, about 900 μg/kg to about 1100 μg/kg, about 900 μg/kg to about 1000 μg/kg, about 950 μg/kg to about 2000 μg/kg, about 950 μg/kg to about 1900 μg/kg, about 950 μg/kg to about 1800 μg/kg, about 950 μg/kg to about 1700 μg/kg, about 950 μg/kg to about 1600 μg/kg, about 950 μg/kg to about 1500 μg/kg, about 950 μg/kg to about 1400 μg/kg, about 950 μg/kg to about 1300 μg/kg, about 950 μg/kg to about 1200 μg/kg, about 950 μg/kg to about 1100 μg/kg, about 950 μg/kg to about 1000 μg/kg, about 1000 μg/kg to about 2000 μg/kg, about 1000 μg/kg to about 1900 μg/kg, about 1000 μg/kg to about 1800 μg/kg, about 1000 μg/kg to about 1700 μg/kg, about 1000 μg/kg to about 1600 μg/kg, about 1000 μg/kg to about 1500 μg/kg, about 1000 μg/kg to about 1400 μg/kg, about 1000 μg/kg to about 1300 μg/kg, about 1000 μg/kg to about 1200 μg/kg, about 1000 μg/kg to about 1100 μg/kg, about 1100 μg/kg to about 2000 μg/kg, about 1100 μg/kg to about 1900 μg/kg, about 1100 μg/kg to about 1800 μg/kg, about 1100 μg/kg to about 1700 μg/kg, about 1100 μg/kg to about 1600 μg/kg, about 1100 μg/kg to about 1500 μg/kg, about 1100 μg/kg to about 1400 μg/kg, about 1100 μg/kg to about 1300 μg/kg, about 1100 μg/kg to about 1200 μg/kg, about 1200 μg/kg to about 2000 μg/kg, about 1200 μg/kg to about 1900 μg/kg, about 1200 μg/kg to about 1800 μg/kg, about 1200 μg/kg to about 1700 μg/kg, about 1200 μg/kg to about 1600 μg/kg, about 1200 μg/kg to about 1500 μg/kg, about 1200 μg/kg to about 1400 μg/kg, about 1200 μg/kg to about 1300 μg/kg, about 1300 μg/kg to about 2000 μg/kg, about 1300 μg/kg to about 1900 μg/kg, about 1300 μg/kg to about 1800 μg/kg, about 1300 μg/kg to about 1700 μg/kg, about 1300 μg/kg to about 1600 μg/kg, about 1300 μg/kg to about 1500 μg/kg, about 1300 μg/kg to about 1400 μg/kg, about 1400 μg/kg to about 2000 μg/kg, about 1400 μg/kg to about 1900 μg/kg, about 1400 μg/kg to about 1800 μg/kg, about 1400 μg/kg to about 1700 μg/kg, about 1400 μg/kg to about 1600 μg/kg, about 1400 μg/kg to about 1500 μg/kg, about 1500 μg/kg to about 2000 μg/kg, about 1500 μg/kg to about 1900 μg/kg, about 1500 μg/kg to about 1800 μg/kg, about 1500 μg/kg to about 1700 μg/kg, about 1500 μg/kg to about 1600 μg/kg, about 1600 μg/kg to about 2000 μg/kg, about 1600 μg/kg to about 1900 μg/kg, about 1600 μg/kg to about 1800 μg/kg, about 1600 μg/kg to about 1700 μg/kg, about 1700 μg/kg to about 2000 μg/kg, about 1700 μg/kg to about 1900 μg/kg, about 1700 μg/kg to about 1800 μg/kg, about 1800 μg/kg to about 2000 μg/kg, about 1800 μg/kg to about 1900 μg/kg, or about 1900 μg/kg to about 2000 μg/kg of the IL-22 Fc fusion protein is administered to the subject in the dosing cycle.

For example, in some embodiments, the invention provides a method of treating or preventing GVHD in a subject comprising administering to the subject an IL-22 Fc fusion protein in a dosing regimen comprising a dosing cycle, wherein the dosing cycle comprises between two and six doses, and wherein a total of about 30 μg/kg to about 720 μg/kg of the IL-22 Fc fusion protein is administered to the subject in the dosing cycle. In some embodiments, a total of about 30 μg/kg to about 540 μg/kg of the IL-22 Fc fusion protein is administered to the subject in the dosing cycle.

In some embodiments of any of the preceding methods, the length of the dosing cycle is between about 1 week and about 30 weeks, e.g., about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, or about 30 weeks. In some embodiments, the length of the dosing cycle is about 96 days.

For example, in some embodiments, the length of the dosing cycle is between about 1 week and about 30 weeks, between about 1 week about 25 weeks, between about 1 week and about 20 weeks, between about 1 week and about 15 weeks, between about 1 week and about 11 weeks, between about 1 week and about 10 weeks, between about 1 week and about 9 weeks, between about 1 week and about 8 weeks, between about 1 week and about 7 weeks, between about 1 week and about 6 weeks, between about 1 week and about 5 weeks, between about 1 week and about 4 weeks, between about 1 week and about 3 weeks, between about 1 week and about 2 weeks, about 2 weeks and about 30 weeks, between about 2 weeks about 25 weeks, between about 2 weeks and about 20 weeks, between about 2 weeks and about 15 weeks, between about 2 weeks and about 11 weeks, between about 2 weeks and about 10 weeks, between about 2 weeks and about 9 weeks, between about 2 weeks and about 8 weeks, between about 2 weeks and about 7 weeks, between about 2 weeks and about 6 weeks, between about 2 weeks and about 5 weeks, between about 2 weeks and about 4 weeks, between about 2 weeks and about 3 weeks, between about 3 weeks and about 30 weeks, between about 3 weeks about 25 weeks, between about 3 weeks and about 20 weeks, between about 3 weeks and about 15 weeks, between about 3 weeks and about 11 weeks, between about 3 weeks and about 10 weeks, between about 3 weeks and about 9 weeks, between about 3 weeks and about 8 weeks, between about 3 weeks and about 7 weeks, between about 3 weeks and about 6 weeks, between about 3 weeks and about 5 weeks, between about 3 weeks and about 4 weeks, between about 4 weeks and about 30 weeks, between about 4 weeks about 25 weeks, between about 4 weeks and about 20 weeks, between about 4 weeks and about 15 weeks, between about 4 weeks and about 11 weeks, between about 4 weeks and about 10 weeks, between about 4 weeks and about 9 weeks, between about 4 weeks and about 8 weeks, between about 4 weeks and about 7 weeks, between about 4 weeks and about 6 weeks, between about 4 weeks and about 5 weeks, between about 5 weeks and about 30 weeks, between about 5 weeks about 25 weeks, between about 5 weeks and about 20 weeks, between about 5 weeks and about 15 weeks, between about 5 weeks and about 11 weeks, between about 5 weeks and about 10 weeks, between about 5 weeks and about 9 weeks, between about 5 weeks and about 8 weeks, between about 5 weeks and about 7 weeks, between about 5 weeks and about 6 weeks, between about 6 weeks and about 30 weeks, between about 6 weeks about 25 weeks, between about 6 weeks and about 20 weeks, between about 6 weeks and about 15 weeks, between about 6 weeks and about 11 weeks, between about 6 weeks and about 10 weeks, between about 6 weeks and about 9 weeks, between about 6 weeks and about 8 weeks, between about 6 weeks and about 7 weeks, between about 7 weeks and about 30 weeks, between about 7 weeks about 25 weeks, between about 7 weeks and about 20 weeks, between about 7 weeks and about 15 weeks, between about 7 weeks and about 11 weeks, between about 7 weeks and about 10 weeks, between about 7 weeks and about 9 weeks, between about 7 weeks and about 8 weeks, between about 8 weeks and about 30 weeks, between about 8 weeks about 25 weeks, between about 8 weeks and about 20 weeks, between about 8 weeks and about 15 weeks, between about 8 weeks and about 11 weeks, between about 8 weeks and about 10 weeks, between about 8 weeks and about 9 weeks, between about 9 weeks and about 30 weeks, between about 9 weeks about 25 weeks, between about 9 weeks and about 20 weeks, between about 9 weeks and about 15 weeks, between about 9 weeks and about 11 weeks, between about 9 weeks and about 10 weeks, between about 10 weeks and about 30 weeks, between about 10 weeks about 25 weeks, between about 10 weeks and about 20 weeks, between about 10 weeks and about 15 weeks, between about 10 weeks and about 11 weeks, between about 11 weeks and about 30 weeks, between about 11 weeks about 25 weeks, between about 11 weeks and about 20 weeks, between about 11 weeks and about 15 weeks, between about 12 weeks and about 30 weeks, between about 12 weeks about 25 weeks, between about 12 weeks and about 20 weeks, between about 12 weeks and about 15 weeks, between about 13 weeks and about 30 weeks, between about 13 weeks about 25 weeks, between about 13 weeks and about 20 weeks, between about 13 weeks and about 15 weeks, between about 14 weeks and about 30 weeks, between about 14 weeks about 25 weeks, between about 14 weeks and about 20 weeks, between about 14 weeks and about 15 weeks, between about 15 weeks and about 30 weeks, between about 15 weeks about 25 weeks, between about 15 weeks and about 20 weeks, between about 20 weeks and about 30 weeks, between about 20 weeks about 25 weeks, or between about 25 weeks and about 30 weeks. In some embodiments, the length of the dosing cycle is between 5 weeks and 15 weeks. In some embodiments, the length of the dosing cycle is between 8 weeks and 12 weeks. In particular embodiments, the length of the dosing cycle is about 8 weeks. In other particular embodiments, the length of the dosing cycle is about 10 weeks. In yet other particular embodiments, the length of the dosing cycle is about 11 weeks. In yet other particular embodiments, the length of the dosing cycle is about 12 weeks. In yet other particular embodiments, the length of the dosing cycle is about 13 weeks. In yet other particular embodiments, the length of the dosing cycle is about 14 weeks. In yet other particular embodiments, the length of the dosing cycle is about 15 weeks.

In any of the methods described herein, the first dose of the dosing cycle may be administered to the subject prior to allogeneic hematopoietic stem cell transplantation (allo-HSCT). The first dose of the dosing cycle may be administered at any suitable time prior to the allo-HSCT. For example, the first dose of the dosing cycle may be administered about 0.5 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 2 months, about 3 months, about 4 months, about 5 months, about 6 months, about 7 months, about 8 months, about 9 months, about 10 months, about 11 months, or about a year prior to allo-HSCT. In some embodiments, the first dose of the dosing cycle is administered to the subject about 3 (±2) days prior to allo-HSCT.

In dosing cycles that contain at least a first dose and a second dose, the second dose may be administered to the subject at any suitable time. In some embodiments, the second dose is administered to the subject on or about Day 1, Day 2, Day 3, Day 4, Day 5, Day 6, Day 7, Day 8, Day 9, Day 10, Day 11, Day 12, Day 13, Day 14, Day 15, Day 16, Day 17, Day 18, Day 19, Day 20, Day 21, Day 22, Day 23, Day 24, Day 25, Day 26, Day 27, Day 28, Day 29, Day 30, Day 31 following the allo-HSCT. For example, in some embodiments, the second dose is administered on or about Day 11 following the allo-HSCT.

In some embodiments of any of the preceding methods, the dosing cycle comprises a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein. In some embodiments, consists of a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein. In some embodiments, the C1D1, the C1D2, the C1D3, the C1D4, the C1D5, the C1D6, the C1D7, and the C1D8 are each between about 15 μg/kg to about 90 μg/kg. In some embodiments, the C1D1, the C1D2, the C1D3, the C1D4, the C1D5, the C1D6, the C1D7, and the C1D8 are each about 60 μg/kg.

In another example, provided herein is a method of treating or preventing GVHD in a subject comprising a dosing regimen, the dosing regimen comprising administering to the subject an IL-22 Fc fusion protein every two weeks (q2w).

In another example, provided herein is an IL-22 Fc fusion protein for use in a method of treating or preventing GVHD in a subject, wherein the IL-22 Fc fusion protein is for administration to the subject in a dosing regimen, the dosing regimen comprising administering to the subject an IL-22 Fc fusion protein every two weeks (q2w).

In another example, provided herein is an IL-22 Fc fusion protein for use in the preparation of a medicament for use in a method of treating or preventing GVHD in a subject, wherein the IL-22 Fc fusion protein is for administration to the subject in a dosing regimen, the dosing regimen comprising administering to the subject an IL-22 Fc fusion protein every two weeks (q2w).

In another example, provided herein is a method of treating GVHD in a subject comprising a dosing regimen, the dosing regimen comprising administering to the subject an IL-22 Fc fusion protein every two weeks (q2w) until the subject has a clinical remission of the GVHD.

In another example, provided herein is an IL-22 Fc fusion protein for use in a method of treating GVHD in a subject, wherein the IL-22 Fc fusion protein is for administration to the subject in a dosing regimen, the dosing regimen comprising administering to the subject an IL-22 Fc fusion protein every two weeks (q2w) until the subject has a clinical remission of the GVHD.

In another example, provided herein is an IL-22 Fc fusion protein for use in the preparation of a medicament for use in a method of treating GVHD in a subject, wherein the IL-22 Fc fusion protein is for administration to the subject in a dosing regimen, the dosing regimen comprising administering to the subject an IL-22 Fc fusion protein every two weeks (q2w) until the subject has a clinical remission of the GVHD.

In any of the preceding methods, the GVHD may be acute GVHD or chronic GVHD. In particular embodiments, the GVHD is acute GVHD.

Any of the preceding methods may be a method of preventing GVHD (e.g., a method of preventing acute GVHD, including acute intestinal GVHD).

In another example, provided herein is a method of preventing acute GVHD in a subject comprising administering to the subject an IL-22 Fc fusion protein in a dosing regimen comprising a dosing cycle having a length of about 96 days, wherein the dosing cycle comprises a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein, wherein the C1D1, the C1D2, the C1D3, the C1D4, the C1D5, the C1D6, the C1D7, and the C1D8 are each about 30 μg/kg, wherein the C1D1 is administered to the subject about 3 (±2) days prior to allo-HSCT, the C1D2 is administered about eleven days after the allo-HSCT, and the C1D3, C1D4, C1D5, C1D6, C1D7, and C1D8 are administered to the subject every two weeks (q2w) following administration of the C1D2.

In another example, provided herein is a method of preventing acute GVHD in a subject comprising administering to the subject an IL-22 Fc fusion protein in a dosing regimen comprising a dosing cycle having a length of about 96 days, wherein the dosing cycle comprises a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein, wherein the C1D1, the C1D2, the C1D3, the C1D4, the C1D5, the C1D6, the C1D7, and the C1D8 are each about 60 μg/kg, wherein the C1D1 is administered to the subject about 3 (±2) days prior to allo-HSCT, the C1D2 is administered about eleven days after the allo-HSCT, and the C1D3, C1D4, C1D5, C1D6, C1D7, and C1D8 are administered to the subject every two weeks (q2w) following administration of the C1D2.

In another example, provided herein is a method of preventing acute GVHD in a subject comprising administering to the subject an IL-22 Fc fusion protein in a dosing regimen comprising a dosing cycle having a length of about 96 days, wherein the dosing cycle comprises a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein, wherein the C1D1, the C1D2, the C1D3, the C1D4, the C1D5, the C1D6, the C1D7, and the C1D8 are each about 90 μg/kg, wherein the C1D1 is administered to the subject about 3 (±2) days prior to allo-HSCT, the C1D2 is administered about eleven days after the allo-HSCT, and the C1D3, C1D4, C1D5, C1D6, C1D7, and C1D8 are administered to the subject every two weeks (q2w) following administration of the C1D2. In some embodiments, the pharmaceutical composition has an average sialic acid content in the range of 8 to 9 moles of sialic acid per mole of the IL-22 Fc fusion protein.

In another example, provided herein is an IL-22 Fc fusion protein for use in a method of treating or preventing GVHD in a subject, wherein the IL-22 Fc fusion protein is for administration to the subject in a dosing regimen comprising a dosing cycle having a length of about 96 days, wherein the dosing cycle comprises a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein, wherein the C1D1, the C1D2, the C1D3, the C1D4, the C1D5, the C1D6, the C1D7, and the C1D8 are each about 30 μg/kg, wherein the C1D1 is administered to the subject about 3 (±2) days prior to allo-HSCT, the C1D2 is administered about eleven days after the allo-HSCT, and the C1D3, C1D4, C1D5, C1D6, C1D7, and C1D8 are administered to the subject every two weeks (q2w) following administration of the C1D2. In some embodiments, the pharmaceutical composition has an average sialic acid content in the range of 8 to 9 moles of sialic acid per mole of the IL-22 Fc fusion protein.

In another example, provided herein is an IL-22 Fc fusion protein for use in a method of treating or preventing GVHD in a subject, wherein the IL-22 Fc fusion protein is for administration to the subject in a dosing regimen comprising a dosing cycle having a length of about 96 days, wherein the dosing cycle comprises a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein, wherein the C1D1, the C1D2, the C1D3, the C1D4, the C1D5, the C1D6, the C1D7, and the C1D8 are each about 60 μg/kg, wherein the C1D1 is administered to the subject about 3 (±2) days prior to allo-HSCT, the C1D2 is administered about eleven days after the allo-HSCT, and the C1D3, C1D4, C1D5, C1D6, C1D7, and C1D8 are administered to the subject every two weeks (q2w) following administration of the C1D2. In some embodiments, the pharmaceutical composition has an average sialic acid content in the range of 8 to 9 moles of sialic acid per mole of the IL-22 Fc fusion protein.

In another example, provided herein is provided herein is an IL-22 Fc fusion protein for use in a method of treating or preventing GVHD in a subject, wherein the IL-22 Fc fusion protein is for administration to the subject in a dosing regimen comprising a dosing cycle having a length of about 96 days, wherein the dosing cycle comprises a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein, wherein the C1D1, the C1D2, the C1D3, the C1D4, the C1D5, the C1D6, the C1D7, and the C1D8 are each about 90 μg/kg, wherein the C1D1 is administered to the subject about 3 (±2) days prior to allo-HSCT, the C1D2 is administered about eleven days after the allo-HSCT, and the C1D3, C1D4, C1D5, C1D6, C1D7, and C1D8 are administered to the subject every two weeks (q2w) following administration of the C1D2. In some embodiments, the pharmaceutical composition has an average sialic acid content in the range of 8 to 9 moles of sialic acid per mole of the IL-22 Fc fusion protein.

In another example, provided herein is a method of preventing acute GVHD in a subject comprising administering to the subject a pharmaceutical composition comprising an IL-22 Fc fusion protein in a dosing regimen comprising a dosing cycle having a length of about 96 days, the pharmaceutical composition having an average sialic acid content in the range of 8 to 12 moles of sialic acid per mole of the IL-22 Fc fusion protein, wherein the dosing cycle comprises a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein, wherein the C1D1, the C1D2, the C1D3, the C1D4, the C1D5, the C1D6, the C1D7, and the C1D8 are each about 30 μg/kg, wherein the C1D1 is administered to the subject about 3 (±2) days prior to allo-HSCT, the C1D2 is administered about eleven days after the allo-HSCT, and the C1D3, C1D4, C1D5, C1D6, C1D7, and C1D8 are administered to the subject every two weeks (q2w) following administration of the C1D2. In some embodiments, the pharmaceutical composition has an average sialic acid content in the range of 8 to 9 moles of sialic acid per mole of the IL-22 Fc fusion protein.

In another example, provided herein is a method of preventing acute GVHD in a subject comprising administering to the subject a pharmaceutical composition comprising an IL-22 Fc fusion protein in a dosing regimen comprising a dosing cycle having a length of about 96 days, the pharmaceutical composition having an average sialic acid content in the range of 8 to 12 moles of sialic acid per mole of the IL-22 Fc fusion protein, wherein the dosing cycle comprises a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein, wherein the C1D1, the C1D2, the C1D3, the C1D4, the C1D5, the C1D6, the C1D7, and the C1D8 are each about 60 μg/kg, wherein the C1D1 is administered to the subject about 3 (±2) days prior to allo-HSCT, the C1D2 is administered about eleven days after the allo-HSCT, and the C1D3, C1D4, C1D5, C1D6, C1D7, and C1D8 are administered to the subject every two weeks (q2w) following administration of the C1D2. In some embodiments, the pharmaceutical composition has an average sialic acid content in the range of 8 to 9 moles of sialic acid per mole of the IL-22 Fc fusion protein.

In another example, provided herein is a method of preventing acute GVHD in a subject comprising administering to the subject a pharmaceutical composition comprising an IL-22 Fc fusion protein in a dosing regimen comprising a dosing cycle having a length of about 96 days, the pharmaceutical composition having an average sialic acid content in the range of 8 to 12 moles of sialic acid per mole of the IL-22 Fc fusion protein, wherein the dosing cycle comprises a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein, wherein the C1D1, the C1D2, the C1D3, the C1D4, the C1D5, the C1D6, the C1D7, and the C1D8 are each about 90 μg/kg, wherein the C1D1 is administered to the subject about 3 (±2) days prior to allo-HSCT, the C1D2 is administered about eleven days after the allo-HSCT, and the C1D3, C1D4, C1D5, C1D6, C1D7, and C1D8 are administered to the subject every two weeks (q2w) following administration of the C1D2. In some embodiments, the pharmaceutical composition has an average sialic acid content in the range of 8 to 9 moles of sialic acid per mole of the IL-22 Fc fusion protein.

In another example, provided herein is a pharmaceutical composition comprising an IL-22 Fc fusion protein for use in a method of treating or preventing GVHD in a subject, the pharmaceutical composition having an average sialic acid content in the range of 8 to 12 moles of sialic acid per mole of the IL-22 Fc fusion protein, wherein the IL-22 Fc fusion protein is for administration to the subject in a dosing regimen comprising a dosing cycle having a length of about 96 days, wherein the dosing cycle comprises a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein, wherein the C1D1, the C1D2, the C1D3, the C1D4, the C1D5, the C1D6, the C1D7, and the C1D8 are each about 30 μg/kg, wherein the C1D1 is administered to the subject about 3 (±2) days prior to allo-HSCT, the C1D2 is administered about eleven days after the allo-HSCT, and the C1D3, C1D4, C1D5, C1D6, C1D7, and C1D8 are administered to the subject every two weeks (q2w) following administration of the C1D2. In some embodiments, the pharmaceutical composition has an average sialic acid content in the range of 8 to 9 moles of sialic acid per mole of the IL-22 Fc fusion protein.

In another example, provided herein is a pharmaceutical composition comprising an IL-22 Fc fusion protein for use in a method of treating or preventing GVHD in a subject, the pharmaceutical composition having an average sialic acid content in the range of 8 to 12 moles of sialic acid per mole of the IL-22 Fc fusion protein, wherein the IL-22 Fc fusion protein is for administration to the subject in a dosing regimen comprising a dosing cycle having a length of about 96 days, wherein the dosing cycle comprises a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein, wherein the C1D1, the C1D2, the C1D3, the C1D4, the C1D5, the C1D6, the C1D7, and the C1D8 are each about 60 μg/kg, wherein the C1D1 is administered to the subject about 3 (±2) days prior to allo-HSCT, the C1D2 is administered about eleven days after the allo-HSCT, and the C1D3, C1D4, C1D5, C1D6, C1D7, and C1D8 are administered to the subject every two weeks (q2w) following administration of the C1D2. In some embodiments, the pharmaceutical composition has an average sialic acid content in the range of 8 to 9 moles of sialic acid per mole of the IL-22 Fc fusion protein.

In another example, provided herein is provided herein is a pharmaceutical composition comprising an IL-22 Fc fusion protein for use in a method of treating or preventing GVHD in a subject, the pharmaceutical composition having an average sialic acid content in the range of 8 to 12 moles of sialic acid per mole of the IL-22 Fc fusion protein, wherein the IL-22 Fc fusion protein is for administration to the subject in a dosing regimen comprising a dosing cycle having a length of about 96 days, wherein the dosing cycle comprises a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein, wherein the C1D1, the C1D2, the C1D3, the C1D4, the C1D5, the C1D6, the C1D7, and the C1D8 are each about 90 μg/kg, wherein the C1D1 is administered to the subject about 3 (±2) days prior to allo-HSCT, the C1D2 is administered about eleven days after the allo-HSCT, and the C1D3, C1D4, C1D5, C1D6, C1D7, and C1D8 are administered to the subject every two weeks (q2w) following administration of the C1D2. In some embodiments, the pharmaceutical composition has an average sialic acid content in the range of 8 to 9 moles of sialic acid per mole of the IL-22 Fc fusion protein.

In any of the preceding methods, the IL-22 Fc fusion protein may be included in a pharmaceutical composition, e.g., a pharmaceutical composition comprising an IL-22 Fc fusion protein and a pharmaceutically acceptable carrier.

In any of the preceding methods, the pharmaceutical composition may have an average sialic acid content of 8 moles of sialic acid per mole of the IL-22 Fc fusion protein. In other embodiments of any of the preceding methods, the pharmaceutical composition may have an average sialic acid content of 9 moles of sialic acid per mole of the IL-22 Fc fusion protein. In some embodiments, the sialic acid comprises N-acetylneuraminic acid (NANA). In some embodiments, the pharmaceutical composition has an average NGNA content of less than 1 mole of NGNA per mole of the IL-22 Fc fusion protein.

In any of the preceding methods, the IL-22 polypeptide may be N-glycosylated. In some embodiments, the IL-22 polypeptide is glycosylated at one or more locations corresponding to amino acid residues Asn21, Asn35, Asn64, and/or Asn143 of SEQ ID NO: 4. In some embodiments, the IL-22 Fc fusion protein comprises a glycosylated IL-22 polypeptide linked to an Fc region by a linker, wherein the IL-22 polypeptide is glycosylated at one or more locations corresponding to amino acid residues Asn21, Asn35, Asn64, and/or Asn143 of SEQ ID NO: 4, and wherein: (a) the percent N-glycosylation site occupancy at residue Asn21 is in the range of 70 to 90; (b) the percent N-glycosylation site occupancy at residue Asn35 is in the range of 90 to 100; (c) the percent N-glycosylation site occupancy at residue Asn64 is in the range of 90 to 100; and/or (d) the percent N-glycosylation site occupancy at residue Asn143 is in the range of 25 to 35.

In any of the preceding methods, the pharmaceutical composition may be a liquid composition.

In any of the preceding methods, (i) the IL-22 Fc fusion protein may have a maximum observed concentration (Cₘₐₓ) of about 8,000 ng/mL to about 19,000 ng; (ii) the IL-22 Fc fusion protein may have an area under the serum concentration-time curve from time 0 to the last measureable time point (AUCₗₐₛₜ) of about 7,000 day·ng/mL to about 25,000 day·ng/mL; and/or (iii) the IL-22 Fc fusion protein may have a clearance (CL) of about 40 mL/kg/day to about 140 mL/kg/day. In some embodiments, the Cₘₐₓ, AUCₗₐₛₜ, and/or CL is assessed following intravenous administration of about 1,000 μg/kg of the IL-22 Fc fusion protein to a CD1 mouse.

In any of the preceding methods, the IL-22 polypeptide may comprise N-glycans having monoantennary, biantennary, triantennary, and/or tetraantennary structure. In some embodiments: (i) about 0.1% to about 2% of the N-glycans have monoantennary structure; (ii) about 10% to about 25% of the N-glycans have biantennary structure; (iii) about 25% to about 40% of the N-glycans have triantennary structure; and/or (iv) about 30% to about 51% of the N-glycans have tetraantennary structure. In some embodiments: (i) 0.1% to 2% of the N-glycans have monoantennary structure; (ii) 10% to 25% of the N-glycans have biantennary structure; (iii) 25% to 40% of the N-glycans have triantennary structure; and/or (iv) 30% to 51% of the N-glycans have tetraantennary structure.

In any of the preceding methods, the IL-22 Fc fusion protein may comprise N-glycans comprising zero, one, two, three, or four galactose moieties. In some embodiments: (i) about 9% to about 32% of the N-glycans comprise zero galactose moieties; (ii) about 10% to about 20% of the N-glycans comprise one galactose moiety; (iii) about 8% to about 25% of the N-glycans comprise two galactose moieties; (iv) about 12% to about 25% of the N-glycans comprise three galactose moieties; and/or (v) about 12% to about 30% of the N-glycans comprise four galactose moieties. In some embodiments: (i) 9% to 32% of the N-glycans comprise zero galactose moieties; (ii) 10% to 20% of the N-glycans comprise one galactose moiety; (iii) 8% to 25% of the N-glycans comprise two galactose moieties; (iv) 12% to 25% of the N-glycans comprise three galactose moieties; and/or (v) 12% to 30% of the N-glycans comprise four galactose moieties.

In any of the preceding methods, the IL-22 Fc fusion protein may comprise N-glycans comprising zero, one, two, three, or four sialic acid moieties. In some embodiments: (i) about 12% to about 35% of the N-glycans comprise zero sialic acid moieties; (ii) about 10% to about 30% of the N-glycans comprise one sialic acid moiety; (iii) about 10% to about 30% of the N-glycans comprise two sialic acid moieties; (iv) about 10% to about 30% of the N-glycans comprise three sialic acid moieties; and/or (v) about 1% to about 20% of the N-glycans comprise four sialic acid moieties. In some embodiments: (i) 12% to 35% of the N-glycans comprise zero sialic acid moieties; (ii) 10% to 30% of the N-glycans comprise one sialic acid moiety; (iii) 10% to 30% of the N-glycans comprise two sialic acid moieties; (iv) 10% to 30% of the N-glycans comprise three sialic acid moieties; and/or (v) 1% to 20% of the N-glycans comprise four sialic acid moieties.

In any of the preceding methods, (i) the IL-22 polypeptide may comprise about 0% to about 10% N-glycans comprising a terminal mannose moiety; and/or (ii) the IL-22 polypeptide may comprise about 30% to about 55% N-glycans comprising a terminal N-acetylglucosamine (GlcNAc) moiety. In some embodiments, (i) the IL-22 polypeptide comprises 0% to 10% N-glycans comprising a terminal mannose moiety; and/or (ii) the IL-22 polypeptide comprises 30% to 55% N-glycans comprising a terminal GlcNAc moiety. In some embodiments, the IL-22 polypeptide comprises 0% to 10% N-glycans comprising a terminal mannose moiety. In some embodiments, the IL-22 polypeptide comprises 30% to 55% N-glycans comprising a terminal GlcNAc moiety.

In any of the preceding methods, the N-glycans may comprise one, two, three, or four terminal GlcNAc moieties. In some embodiments: (i) about 1% to about 20% of the N-glycans comprise one terminal GlcNAc moiety; (ii) about 1% to about 20% of the N-glycans comprise two terminal GlcNAc moieties; (iii) about 5% to about 25% of the N-glycans comprise three terminal GlcNAc moieties; and/or (iv) about 0% to about 15% of the N-glycans comprise four terminal GlcNAc moieties. In some embodiments: (i) 1% to 20% of the N-glycans comprise one terminal GlcNAc moiety; (ii) 1% to 20% of the N-glycans comprise two terminal GlcNAc moieties; (iii) 5% to 25% of the N-glycans comprise three terminal GlcNAc moieties; and/or (iv) 0% to 15% of the N-glycans comprise four terminal GlcNAc moieties.

In any of the preceding methods, (i) the IL-22 polypeptide may comprise about 20% to about 45% N-glycans comprising a terminal galactose (Gal) moiety; and/or (ii) the N-glycans comprise one, two, or three terminal Gal moieties. In some embodiments, (i) the IL-22 polypeptide comprises 20% to 45% N-glycans comprising a terminal Gal moiety; and/or (ii) the N-glycans comprise one, two, or three terminal Gal moieties.

In any of the preceding methods, (i) about 15% to about 30% of the N-glycans may comprise one terminal Gal moiety; (ii) about 1% to about 15% of the N-glycans may comprise two terminal Gal moieties; and/or (iii) about 0.1% to about 6% of the N-glycans may comprise three terminal Gal moieties. In some embodiments: (i) 15% to 30% of the N-glycans comprise one terminal Gal moiety; (ii) 1% to 15% of the N-glycans comprise two terminal Gal moieties; and/or (iii) 0.1% to 6% of the N-glycans comprise three terminal Gal moieties.

In any of the preceding methods, (i) the IL-22 polypeptide may comprise N-glycans comprises galactose N-acetylglucosamine (LacNAc) repeats; (ii) the IL-22 polypeptide may comprise N-glycans comprising fucosylated N-glycans; and/or (iii) the IL-22 polypeptide may comprise N-glycans comprising afucosylated N-glycans.

Any suitable concentration of the IL-22 Fc fusion protein may be used. For example, in some embodiments, the concentration of the IL-22 Fc fusion protein is about 0.5 mg/mL to about 20 mg/mL. In some embodiments, the concentration of the IL-22 Fc fusion protein is about 0.5 mg/mL to about 5 mg/mL. In some embodiments, the concentration of the IL-22 Fc fusion protein is about 1 mg/mL. In some embodiments, the concentration of the IL-22 Fc fusion protein is about 8 mg/mL to about 12 mg/mL. In some embodiments, the concentration of the IL-22 Fc fusion protein is about 10 mg/mL.

In any of the preceding methods, the IL-22 Fc fusion may be produced from a production culture having a volume of at least about 500 L. In some embodiments of any of the preceding aspects, the IL-22 Fc fusion protein has been produced from a production culture having a volume of about 500 L to about 5,000 L. In some embodiments, the IL-22 Fc fusion protein has been produced from a production culture having a volume of about 1,000 L to about 3,000 L. In some embodiments the IL-22 Fc fusion protein has been produced from a production culture having a volume of about 1,500 L to about 2,500 L. In some embodiments, the IL-22 Fc fusion protein has been produced from a production culture having a volume of about 2000 L.

In any of the preceding methods, in some embodiments, the dosing regimen further includes one or more further dosing cycles. For example, in some embodiments, the dosing regimen further includes one, two, three, four, five, six, seven, eight, nine, ten, eleven, twelve, thirteen, fourteen, fifteen, sixteen, seventeen, eighteen, nineteen, or twenty further dosing cycles. In particular embodiments, the dosing regimen further comprises a second dosing cycle.

In some embodiments, the dose(s) in the further (e.g., second) dosing cycle(s) are administered to the subject every week (q1w), every two weeks (q2w), every three weeks (q3w), every four weeks, (q4w), every five weeks (q5w), every six weeks (q6w), every seven weeks (q7w), every eight weeks (q8w), every nine weeks (q9w), every ten weeks (q10w), every 12 weeks (q12w), every fourteen weeks (q14w), every sixteen weeks (q16w), every eighteen weeks (q18w), or every twenty weeks (q20w).

In some embodiments of any of the preceding methods, the length of the further (e.g., second) dosing cycle is between about 5 weeks and about 80 weeks, e.g., about 5 weeks, about 6 weeks, about 7 weeks, about 9 weeks, about 10 weeks, about 11 weeks, about 12 weeks, about 14 weeks, about 16 weeks, about 18 weeks, about 20 weeks, about 22 weeks, about 24 weeks, about 26 weeks, about 28 weeks, about 30 weeks, about 32 weeks, about 34 weeks, about 36 weeks, about 38 weeks, about 40 weeks, about 42 weeks, about 44 weeks, about 46 weeks, about 48 weeks, about 50 weeks, about 52 weeks, about 54 weeks, about 56 weeks, about 58 weeks, about 60 weeks, about 62 weeks, about 64 weeks, about 66 weeks, about 68 weeks, about 70 weeks, about 72 weeks, about 74 weeks, about 76 weeks, about 78 weeks, or about 80 weeks.

For example, in some embodiments of any of the preceding methods, the length of the further (e.g., second) dosing cycle is between about 5 weeks and about 80 weeks, between about 5 weeks and about 75 weeks, between about 5 weeks and about 70 weeks, between about 5 weeks and about 65 weeks, between about 5 weeks and about 60 weeks, between about 5 weeks and about 55 weeks, between about 5 weeks and about 50 weeks, between about 5 weeks and about 45 weeks, between about 5 weeks and about 40 weeks, between about 5 weeks and about 35 weeks, between about 5 weeks and about 30 weeks, between about 5 weeks and about 25 weeks, between about 5 weeks and about 20 weeks, between about 5 weeks and about 15 weeks, between about 5 weeks and about 10 weeks, between about 10 weeks and about 80 weeks, between about 10 weeks and about 75 weeks, between about 10 weeks and about 70 weeks, between about 10 weeks and about 65 weeks, between about 10 weeks and about 60 weeks, between about 10 weeks and about 55 weeks, between about 10 weeks and about 50 weeks, between about 10 weeks and about 45 weeks, between about 10 weeks and about 40 weeks, between about 10 weeks and about 35 weeks, between about 10 weeks and about 30 weeks, between about 10 weeks and about 25 weeks, between about 10 weeks and about 20 weeks, between about 10 weeks and about 15 weeks, between about 15 weeks and about 80 weeks, between about 15 weeks and about 75 weeks, between about 15 weeks and about 70 weeks, between about 15 weeks and about 65 weeks, between about 15 weeks and about 60 weeks, between about 15 weeks and about 55 weeks, between about 15 weeks and about 50 weeks, between about 15 weeks and about 45 weeks, between about 15 weeks and about 40 weeks, between about 15 weeks and about 35 weeks, between about 15 weeks and about 30 weeks, between about 15 weeks and about 25 weeks, between about 15 weeks and about 20 weeks, between about 20 weeks and about 80 weeks, between about 20 weeks and about 75 weeks, between about 20 weeks and about 70 weeks, between about 20 weeks and about 65 weeks, between about 20 weeks and about 60 weeks, between about 20 weeks and about 55 weeks, between about 20 weeks and about 50 weeks, between about 20 weeks and about 45 weeks, between about 20 weeks and about 40 weeks, between about 20 weeks and about 35 weeks, between about 20 weeks and about 30 weeks, between about 20 weeks and about 25 weeks, between about 25 weeks and about 80 weeks, between about 25 weeks and about 75 weeks, between about 25 weeks and about 70 weeks, between about 25 weeks and about 65 weeks, between about 25 weeks and about 60 weeks, between about 25 weeks and about 55 weeks, between about 25 weeks and about 50 weeks, between about 25 weeks and about 45 weeks, between about 25 weeks and about 40 weeks, between about 25 weeks and about 35 weeks, between about 25 weeks and about 30 weeks, between about 30 weeks and about 80 weeks, between about 30 weeks and about 75 weeks, between about 30 weeks and about 70 weeks, between about 30 weeks and about 65 weeks, between about 30 weeks and about 60 weeks, between about 30 weeks and about 55 weeks, between about 30 weeks and about 50 weeks, between about 30 weeks and about 45 weeks, between about 30 weeks and about 40 weeks, between about 30 weeks and about 35 weeks, between about 35 weeks and about 80 weeks, between about 35 weeks and about 75 weeks, between about 35 weeks and about 70 weeks, between about 35 weeks and about 65 weeks, between about 35 weeks and about 60 weeks, between about 35 weeks and about 55 weeks, between about 35 weeks and about 50 weeks, between about 35 weeks and about 45 weeks, between about 35 weeks and about 40 weeks, between about 40 weeks and about 80 weeks, between about 40 weeks and about 75 weeks, between about 40 weeks and about 70 weeks, between about 40 weeks and about 65 weeks, between about 40 weeks and about 60 weeks, between about 40 weeks and about 55 weeks, between about 40 weeks and about 50 weeks, between about 40 weeks and about 45 weeks, between about 45 weeks and about 80 weeks, between about 45 weeks and about 75 weeks, between about 45 weeks and about 70 weeks, between about 45 weeks and about 65 weeks, between about 45 weeks and about 60 weeks, between about 45 weeks and about 55 weeks, between about 45 weeks and about 50 weeks, between about 50 weeks and about 80 weeks, between about 50 weeks and about 75 weeks, between about 50 weeks and about 70 weeks, between about 50 weeks and about 65 weeks, between about 50 weeks and about 60 weeks, between about 50 weeks and about 55 weeks, between about 55 weeks and about 80 weeks, between about 55 weeks and about 75 weeks, between about 55 weeks and about 70 weeks, between about 55 weeks and about 65 weeks, between about 55 weeks and about 60 weeks, between about 60 weeks and about 80 weeks, between about 60 weeks and about 75 weeks, between about 60 weeks and about 70 weeks, between about 60 weeks and about 65 weeks, between about 65 weeks and about 80 weeks, between about 65 weeks and about 75 weeks, between about 65 weeks and about 70 weeks, between about 70 weeks and about 80 weeks, between about 70 weeks and about 75 weeks, or between about 75 weeks and about 80 weeks. In some embodiments, the length of the further (e.g., second) dosing cycle is between about 10 weeks and about 40 weeks. In some embodiments, the length of the further (e.g., second) dosing cycle is between about 15 weeks and about 25 weeks. In particular embodiments, the length of the further (e.g., second) dosing cycle is about 20 weeks.

The methods, dosing regimens, and dosing cycles may be used to treat GVHD in any organ, including the intestines, skin, and the liver. In some embodiments, the GVHD is intestinal GVHD.

Any of the methods described herein may prevent Grade II-IV acute GVHD, for example, as assessed by the MAGIC GVHD Target Organ Staging (see, e.g., Harris et al. Biol. Blood Marrow Transplant. 22(1):4-10, 2016). In some embodiments, the methods, dosing regimens, and dosing cycles prevents Grade II-IV acute GVHD, as assessed by the MAGIC GVHD Target Organ Staging, at Day 100 after the allo-HSCT.

Any of the methods described herein may reduce the incidence of Stage 1, Stage 2, Stage 3, or Stage 4 acute GVHD of the skin, gut, and/or liver, for example, as assessed by the MAGIC GVHD Target Organ Staging. In some embodiments, the method may reduce the incidence of Stage 1, Stage 2, Stage 3, or Stage 4 acute GVHD of the skin, gut, and/or liver, for example, as assessed by the MAGIC GVHD Target Organ Staging, at Day 100 after the allo-HSCT.

Any of the methods described herein may reduce the incidence of Grade I, Grade II, Grade III, or Grade IV acute GVHD, for example, as assessed by the MAGIC GVHD Target Organ Staging. In some embodiments, the method may reduce the incidence of Grade I, Grade II, Grade III, or Grade IV acute GVHD, as assessed by the MAGIC GVHD Target Organ Staging, at Day 100 after the allo-HSCT.

Any of the methods described herein may (i) improve the gastrointestinal (GI) acute GVHD-free survival rate of the subject; (ii) improve the overall survival of the subject; (iii) improve the relapse-free survival rate of the subject, and/or (iv) reduce the incidence of chronic GVHD in the subject. The GI acute GVHD survival rate may be defined as the proportion of subjects with absence of Grade II-IV GI acute GVHD at post-transplant Day 180. The overall survival may be defined as the proportion of subjects who have not experienced death from any cause at post-transplant Day 180 or Day 365. The relapse-free survival rate may be defined as the proportion of subjects who have not experienced relapse of primary disease or death, whichever occurs first, at post-transplant Day 180 or Day 365. The incidence of chronic GVHD may be assessed according to the National Institute of Health cGVHD score at post-transplant Day 365 (see, e.g., Jagasia et al. Biol. Blood Marrow Transplant. 21(3):389-401.el, 2015).

The IL-22 Fc fusion proteins or compositions thereof can be administered in combination with an additional GVHD therapy, including, for example, immunosuppressive agents (e.g., cyclosporine, mycophenolate mofetil (MMF), or tacrolimus), mTOR inhibitors (e.g., sirolimus or everolimus)), chemotherapy agents (e.g., imatinib, pentostatin, methotrexate, or thalidomide), TNF antagonists (e.g., etanercept), steroids (e.g., prednisolone, methylprednisolone, topical steroids, or steroid eye drops), light treatment (e.g., extracorporeal photopheresis), hydroxychloroquine, anti-fibrotic agents (e.g., halofuginone), monoclonal antibodies (e.g., alemtuzumab, infliximab, or rituximab), or combinations thereof. In some embodiments, the additional GVHD therapy is an immunosuppressive agent (e.g., cyclosporine or tacrolimus). In some embodiments, the additional GVHD therapy is standard of care for acute GVHD prophylaxis (e.g., calcineurin (CN) inhibitor (e.g., cyclosporine or tacrolimus)+methotrexate or mycophenolate mofetil (MMF)). Any suitable standard of care aGVHD prophylaxis may be used (see, e.g., Gatza et al. Int. J. Hematol. Oncol. 4(3):113-126, 2015, which is incorporated herein by reference in its entirety).

In any of the preceding methods, uses, and compositions, the subject may be a human.

Other Indications

It is to be understood that any of the methods, dosing regimens, and/or dosing cycles described above can be used in a method of treating hidradenitis suppurativa, COPD, nonalcoholic fatty acid liver disease (e.g., NASH), or other IL-22 associated diseases.

In any of the preceding methods, uses, and compositions, the subject may be a human.

1. Exemplary IL-22 Fc Fusion Proteins for Use in the Methods

Any suitable IL-22 Fc fusion protein can be used in the methods, uses, articles of manufacture, and kits described herein. In general, the IL-22 Fc fusion proteins include an IL-22 polypeptide linked to an Fc region by a linker. Any of the IL-22 Fc fusion proteins described in U.S. Pat. No. 9,815,880, which is incorporated by reference herein in its entirety, may be used in the methods and uses described herein. In some embodiments of any of the preceding IL-22 Fc fusion proteins, the Fc region is not glycosylated. In some embodiments, the amino acid residue at position 297 as in the EU index of the Fc region is Gly. In some embodiments, the amino acid residue at position 297 as in the EU index of the Fc region is Ala. In some embodiments, the amino acid residue at position 299 as in the EU index of the Fc region is Ala, Gly, or Val. In some embodiments, the Fc region comprises the CH2 and CH3 domain of IgG1 or IgG4. In some embodiments, the Fc region comprises the CH2 and CH3 domain of IgG4.

In some embodiments of any of the preceding methods, the IL-22 Fc fusion protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence selected from the group consisting of SEQ ID NO:8, SEQ ID NO:10, SEQ ID NO:12, SEQ ID NO:14, and SEQ ID NO:16. In some embodiments, the IL-22 Fc fusion protein comprises an amino acid sequence having at least 96% sequence identity to the amino acid sequence of SEQ ID NO:8. In some embodiments, the IL-22 Fc fusion protein comprises an amino acid sequence having at least 97% sequence identity to the amino acid sequence of SEQ ID NO:8. In some embodiments, the IL-22 Fc fusion protein comprises an amino acid sequence having at least 98% sequence identity to the amino acid sequence of SEQ ID NO:8. In some embodiments, the IL-22 Fc fusion protein comprises an amino acid sequence having at least 99% sequence identity to the amino acid sequence of SEQ ID NO:8. In some embodiments, the IL-22 Fc fusion protein comprises the amino acid sequence of SEQ ID NO:8, SEQ ID NO:10, or SEQ ID NO:16. In some embodiments, the IL-22 Fc fusion protein comprises the amino acid sequence of SEQ ID NO:8. In some embodiments, the IL-22 Fc fusion protein consists of the amino acid sequence of SEQ ID NO:8. In some embodiments, the IL-22 Fc fusion protein comprises the amino acid sequence of SEQ ID NO:10. In some embodiments, the IL-22 Fc fusion protein consists of the amino acid sequence of SEQ ID NO:10. In some embodiments, the IL-22 Fc fusion protein comprises the amino acid sequence of SEQ ID NO:16. In some embodiments, the IL-22 Fc fusion protein consists of the amino acid sequence of SEQ ID NO:16. In some embodiments, the Fc region is not N-glycosylated.

Any of the preceding IL-22 Fc fusion proteins can be a dimeric IL-22 Fc fusion protein. In other embodiments, any of the preceding IL-22 Fc fusion proteins can be a monomeric IL-22 Fc fusion protein.

Any of the preceding IL-22 Fc fusion proteins can include a human IL-22 polypeptide. In some embodiments, the amino acid sequence of SEQ ID NO:4.

Any suitable linker can be used in the IL-22 Fc fusion proteins described herein. In some embodiments, the linker comprises the amino acid sequence RVESKYGPP (SEQ ID NO: 44). In some embodiments, the linker consists of the amino acid sequence RVESKYGPP (SEQ ID NO: 44).

In some embodiments, any of the IL-22 Fc fusion proteins described herein binds to IL-22 receptor. In some embodiments, the IL-22 receptor is human IL-22 receptor. In some embodiments, the IL-22 Fc fusion protein binds to IL-22RA1 and/or IL-10R2. In some embodiments, the IL-22 Fc fusion protein binds to IL-22RA1.

In some embodiments, any of the preceding IL-22 Fc fusion proteins is produced by the method comprising the step of culturing a host cell capable of expressing the IL-22 Fc fusion protein under conditions suitable for expression of the IL-22 Fc fusion protein. In some embodiments, the method further comprises the step of obtaining the IL-22 Fc fusion protein from the cell culture or culture medium. In some embodiments, the host cell is a CHO cell.

In certain embodiments, any of the IL-22 Fc fusion proteins described herein binds to and induces IL-22 receptor activity or signaling and/or is an agonist of IL-22 receptor activity.

In another aspect, an IL-22 Fc fusion protein provided herein comprises a polypeptide having at least 90%, 91%, 92%, 93%, 94%, 95%, 96%, 97%, 98%, 99%, or 100% sequence identity to the amino acid sequence of SEQ ID NO:4. In other embodiments, the IL-22 Fc fusion protein comprises a polypeptide having at least 90%, 91%, 92%, 93%, 94%, 95%, 96%, 97%, 98%, or 99% sequence identity contains substitutions (e.g., conservative substitutions), insertions, or deletions relative to the reference sequence, but an IL-22 Fc fusion protein comprising that sequence retains the ability to bind to IL-22 receptor. In certain embodiments, a total of 1 to 10 amino acids have been substituted, inserted, and/or deleted in SEQ ID NOs:8, 10, 12, 14, 16, 24, or 26. In certain embodiments, substitutions, insertions, or deletions occur in regions outside the IL-22 (i.e., in the Fc). In some embodiments, the substitutions, insertions, or deletions can be in the linker, the hinge, the CH2 domain, the CH3 domain of the IL-22 Fc fusion protein In certain particular embodiments, the C-terminus Lys residue of Fc is deleted. In certain other embodiments, the C-terminus Gly and Lys residues of Fc are both deleted.

In some embodiments, the IL-22 Fc fusion proteins or compositions thereof (e.g., pharmaceutical compositions) described in International Patent Application No. PCT/US2019/015277, which is incorporated herein by reference in its entirety, may be used in the methods, dosing regimens, and dosing cycles described herein. Without intending to be bound by any one particular theory or mechanism of action, in some embodiments, it is preferred for the IL-22 Fc fusion protein to have an average sialic content in the range of 8 to 12 moles (e.g., about 8, about 9, about 10, about 11, or about 12 moles) of sialic acid per mole of the IL-22 Fc fusion protein such that both the potency and pharmacokinetic properties of the IL-22 Fc fusion proteins are within the desired range (e.g., as described in detail in International Patent Application No. PCT/US2019/015277). This discovery was made in part in connection with identifying certain properties of the molecule that are affected by the manufacturing process and that impact the activity and PK/PD properties of the molecule. For example, such IL-22 Fc-containing compositions having overall low glycosylation (including, but not limited to, e.g., IL-22 Fc fusion proteins and compositions thereof with an average sialic acid content of less than about 8 moles of sialic acid per mole of IL-22 Fc fusion protein), as described in International Patent Application No. PCT/US2019/015277, have undesirably fast clearance in vivo. Further, high glycosylation of those compositions (including, but not limited to, e.g., IL-22 Fc fusion proteins and compositions thereof having greater than about 12 moles of sialic acid per mole of IL-22 Fc fusion protein) can have undesirable binding properties to the IL-22 receptor. Thus, in certain aspects, a solution to the identified problems was to identify a range of average sialic acid content for the IL-22 Fc fusion proteins and compositions thereof which have both suitable clearance rates as well as suitable binding activity. In a specific embodiment, a particularly preferred range of average sialic acid content for the IL-22 Fc fusion proteins and compositions thereof for use in the methods described herein (e.g. methods for treating IBD, e.g., UC or CD, or treating or preventing GVHD, e.g., acute GVHD or chronic GVHD) is 8 to 9 moles of sialic acid per mole of IL-22 Fc fusion protein.

For example, in some embodiments, the composition has an average sialic acid content in the range of 8 to 12 moles (e.g., about 8, about 9, about 10, about 11, or about 12 moles) of sialic acid per mole of the IL-22 Fc fusion protein. In some embodiments, the IL-22 polypeptide is N-glycosylated. In some embodiments, the IL-22 polypeptide is glycosylated at one or more locations corresponding to amino acid residues Asn21, Asn35, Asn64, and/or Asn143 of SEQ ID NO: 4. In some embodiments, the IL-22 Fc fusion protein comprises a glycosylated IL-22 polypeptide linked to an Fc region by a linker, wherein the IL-22 polypeptide is glycosylated at one or more locations corresponding to amino acid residues Asn21, Asn35, Asn64, and/or Asn143 of SEQ ID NO: 4, and wherein: (a) the percent N-glycosylation site occupancy at residue Asn21 is in the range of 70 to 90; (b) the percent N-glycosylation site occupancy at residue Asn35 is in the range of 90 to 100; (c) the percent N-glycosylation site occupancy at residue Asn64 is in the range of 90 to 100; and/or (d) the percent N-glycosylation site occupancy at residue Asn143 is in the range of 25 to 35.

In some embodiments of any of the preceding aspects, the composition has an average sialic acid content in the range of 8 to 9 moles of sialic acid per mole of the IL-22 Fc fusion protein. In some embodiments, the composition has an average sialic acid content of 8 or 9 moles of sialic acid per mole of the IL-22 Fc fusion protein. In some embodiments, the composition has an average sialic acid content of 8 moles of sialic acid per mole of the IL-22 Fc fusion protein. In other embodiments, the composition has an average sialic acid content of 9 moles of sialic acid per mole of the IL-22 Fc fusion protein.

In any of the compositions described herein, the sialic acid may be N-acetylneuraminic acid (NANA).

Any of the compositions may have an average NGNA content of less than 1 mole of NGNA per mole of the IL-22 Fc fusion protein.

In some embodiments of any of the preceding aspects, the composition has an average N-glycolylneuraminic acid (NGNA) content of less than 1 mole of NGNA per mole of the IL-22 Fc fusion protein.

In some embodiments of any of the preceding aspects, the composition is a liquid composition. In some embodiments of any of the preceding aspects: (i) the IL-22 Fc fusion protein has a maximum observed concentration (Cₘₐₓ) of about 8,000 ng/mL to about 19,000 ng; (ii) the IL-22 Fc fusion protein has an area under the serum concentration-time curve from time 0 to the last measureable time point (AUCₗₐₛₜ) of about 7,000 day·ng/mL to about 25,000 day·ng/mL; and/or (iii) the IL-22 Fc fusion protein has a clearance (CL) of about 40 mL/kg/day to about 140 mL/kg/day. In some embodiments, the Cₘₐₓ, AUCₗₐₛₜ, and/or CL is assessed following intravenous administration of about 1,000 μg/kg of the IL-22 Fc fusion protein to a CD1 mouse.

In any of the compositions, the IL-22 polypeptide may include N-glycans having monoantennary, biantennary, triantennary, and/or tetraantennary structure. In some embodiments: (i) about 0.1% to about 2% of the N-glycans have monoantennary structure; (ii) about 10% to about 25% of the N-glycans have biantennary structure; (iii) about 25% to about 40% of the N-glycans have triantennary structure; and/or (iv) about 30% to about 51% of the N-glycans have tetraantennary structure. In some embodiments: (i) 0.1% to 2% of the N-glycans have monoantennary structure; (ii) 10% to 25% of the N-glycans have biantennary structure; (iii) 25% to 40% of the N-glycans have triantennary structure; and/or (iv) 30% to 51% of the N-glycans have tetraantennary structure.

In any of the compositions, the IL-22 Fc fusion protein may include N-glycans including zero, one, two, three, or four galactose moieties. In some embodiments: (i) about 9% to about 32% of the N-glycans include zero galactose moieties; (ii) about 10% to about 20% of the N-glycans include one galactose moiety; (iii) about 8% to about 25% of the N-glycans include two galactose moieties; (iv) about 12% to about 25% of the N-glycans include three galactose moieties; and/or (v) about 12% to about 30% of the N-glycans include four galactose moieties. In some embodiments: (i) 9% to 32% of the N-glycans include zero galactose moieties; (ii) 10% to 20% of the N-glycans include one galactose moiety; (iii) 8% to 25% of the N-glycans include two galactose moieties; (iv) 12% to 25% of the N-glycans include three galactose moieties; and/or (v) 12% to 30% of the N-glycans include four galactose moieties.

In any of the compositions, the IL-22 Fc fusion protein may include N-glycans including zero, one, two, three, or four sialic acid moieties. In some embodiments: (i) about 12% to about 35% of the N-glycans include zero sialic acid moieties; (ii) about 10% to about 30% of the N-glycans include one sialic acid moiety; (iii) about 10% to about 30% of the N-glycans include two sialic acid moieties; (iv) about 10% to about 30% of the N-glycans include three sialic acid moieties; and/or (v) about 1% to about 20% of the N-glycans include four sialic acid moieties. In some embodiments: (i) 12% to 35% of the N-glycans include zero sialic acid moieties; (ii) 10% to 30% of the N-glycans include one sialic acid moiety; (iii) 10% to 30% of the N-glycans include two sialic acid moieties; (iv) 10% to 30% of the N-glycans include three sialic acid moieties; and/or (v) 1% to 20% of the N-glycans include four sialic acid moieties.

In any of the compositions, (i) the IL-22 polypeptide may include about 0% to about 10% N-glycans including a terminal mannose moiety; and/or (ii) the IL-22 polypeptide includes about 30% to about 55% N-glycans including a terminal N-acetylglucosamine (GlcNAc) moiety. In some embodiments, (i) the IL-22 polypeptide includes 0% to 10% N-glycans including a terminal mannose moiety; and/or (ii) the IL-22 polypeptide includes 30% to 55% N-glycans including a terminal GlcNAc moiety. In some embodiments, the IL-22 polypeptide includes 0% to 10% N-glycans including a terminal mannose moiety. In some embodiments, the IL-22 polypeptide includes 30% to 55% N-glycans including a terminal GlcNAc moiety.

In any of the compositions, the N-glycans may include one, two, three, or four terminal GlcNAc moieties. In some embodiments: (i) about 1% to about 20% of the N-glycans include one terminal GlcNAc moiety; (ii) about 1% to about 20% of the N-glycans include two terminal GlcNAc moieties; (iii) about 5% to about 25% of the N-glycans include three terminal GlcNAc moieties; and/or (iv) about 0% to about 15% of the N-glycans include four terminal GlcNAc moieties. In some embodiments: (i) 1% to 20% of the N-glycans include one terminal GlcNAc moiety; (ii) 1% to 20% of the N-glycans include two terminal GlcNAc moieties; (iii) 5% to 25% of the N-glycans include three terminal GlcNAc moieties; and/or (iv) 0% to 15% of the N-glycans include four terminal GlcNAc moieties.

In any of the compositions, (i) the IL-22 polypeptide may include about 20% to about 45% N-glycans including a terminal galactose (Gal) moiety; and/or (ii) the N-glycans include one, two, or three terminal Gal moieties. In some embodiments, (i) the IL-22 polypeptide includes 20% to 45% N-glycans including a terminal Gal moiety; and/or (ii) the N-glycans include one, two, or three terminal Gal moieties.

In any of the compositions: (i) about 15% to about 30% of the N-glycans may include one terminal Gal moiety; (ii) about 1% to about 15% of the N-glycans may include two terminal Gal moieties; and/or (iii) about 0.1% to about 6% of the N-glycans may include three terminal Gal moieties. In some embodiments: (i) 15% to 30% of the N-glycans include one terminal Gal moiety; (ii) 1% to 15% of the N-glycans include two terminal Gal moieties; and/or (iii) 0.1% to 6% of the N-glycans include three terminal Gal moieties.

In any of the compositions: (i) the IL-22 polypeptide may include N-glycans including galactose N-acetylglucosamine (LacNAc) repeats; (ii) the IL-22 polypeptide may include N-glycans including fucosylated N-glycans; and/or (iii) the IL-22 polypeptide may include N-glycans including afucosylated N-glycans.

Any suitable concentration of the IL-22 Fc fusion protein may be used. For example, in some embodiments, the concentration of the IL-22 Fc fusion protein may be about 0.5 mg/mL to about 20 mg/mL. In some embodiments, the concentration of the IL-22 Fc fusion protein is about 0.5 mg/mL to about 5 mg/mL. In some embodiments, the concentration of the IL-22 Fc fusion protein is about 1 mg/mL. In some embodiments, the concentration of the IL-22 Fc fusion protein is about 8 mg/mL to about 12 mg/mL. In some embodiments, the concentration of the IL-22 Fc fusion protein is about 10 mg/mL.

The IL-22 Fc fusion proteins described herein may be produced from a production culture having a volume of at least about 500 L. In some embodiments of any of the preceding aspects, the IL-22 Fc fusion protein has been produced from a production culture having a volume of about 500 L to about 5,000 L. In some embodiments, the IL-22 Fc fusion protein has been produced from a production culture having a volume of about 1,000 L to about 3,000 L. In some embodiments the IL-22 Fc fusion protein has been produced from a production culture having a volume of about 1,500 L to about 2,500 L. In some embodiments, the IL-22 Fc fusion protein has been produced from a production culture having a volume of about 2000 L.

In certain embodiments, IL-22 Fc fusion proteins variants having one or more amino acid substitutions are provided. Conservative substitutions are shown in Table 2 under the heading of “preferred substitutions.” More substantial changes are provided in Table 2 under the heading of “exemplary substitutions,” and as further described below in reference to amino acid side chain classes. Amino acid substitutions may be introduced into the IL-22 Fc fusion protein and the products screened for a desired activity, e.g., retained/improved IL-22 receptor binding, decreased immunogenicity, or improved IL-22 receptor signaling.

TABLE 2

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

A useful method for identification of residues or regions of a protein that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the protein with its binding partner is affected. Further substitutions may be introduced at the amino acid locations demonstrating functional sensitivity to the initial substitutions. Alternatively, or additionally, a crystal structure of a protein complex (e.g., a cytokine-receptor complex) can be used to identify contact points between a protein and its binding partner. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues.

Provided herein are nucleic acids encoding IL-22 Fc fusion proteins. In some embodiments, the nucleic acid encodes the IL-22 Fc fusion protein comprising the amino acid sequence of SEQ ID NO:8, SEQ ID NO:10, SEQ ID NO:12, SEQ ID NO:14, SEQ ID NO:24 or SEQ ID NO:26, preferably SEQ ID NO:8, SEQ ID NO:10, or SEQ ID NO:16, more preferably SEQ ID NO:8. In certain other embodiments, the nucleic acid comprises the polynucleotide sequence of SEQ ID NO:7, SEQ ID NO:9, SEQ ID NO:11, SEQ ID NO:13, SEQ ID NO:23 or SEQ ID NO:25. In certain particular embodiments, the nucleic acid comprises the polynucleotide sequence of SEQ ID NO:7 or SEQ ID NO:11, preferably SEQ ID NO:7. In certain embodiments, the isolated nucleic acid comprises a polynucleotide sequence that is at least 80%, 85%, 86%, 87%, 88%, 89%, 90%, 91%, 92%, 93%, 94%, 95%, 96%, 97%, 98%, 99% or 100% sequence identity to the polynucleotide sequence of SEQ ID NO:7, SEQ ID NO:9, SEQ ID NO:11, SEQ ID NO:13; SEQ ID NO:23 or SEQ ID NO:25. In certain embodiments, the isolated nucleic acid comprises a polynucleotide sequence that is at least 80%, 85%, 86%, 87%, 88%, 89%, 90%, 91%, 92%, 93%, 94%, 95%, 96%, 97%, 98%, 99% or 100% sequence identity to the polynucleotide sequence of SEQ ID NO:7, SEQ ID NO:9, SEQ ID NO:11, SEQ ID NO:13; SEQ ID NO:23 or SEQ ID NO:25, wherein the isolated nucleic acid is capable of encoding an IL-22 Fc fusion protein that is capable of binding to IL-22R and/or triggering IL-22R activity and wherein the Fc region of the IL-22 Fc fusion protein is not glycosylated. In certain embodiments, the isolated nucleic acid comprises a polynucleotide sequence that is at least 80%, 85%, 86%, 87%, 88%, 89%, 90%, 91%, 92%, 93%, 94%, 95%, 96%, 97%, 98%, 99% or 100% sequence identity to the polynucleotide sequence of SEQ ID NO:7, SEQ ID NO:9, SEQ ID NO:11, SEQ ID NO:13; SEQ ID NO:23 or SEQ ID NO:25, wherein the isolated nucleic acid is capable of encoding an IL-22 Fc fusion protein comprising the amino acid sequence of SEQ ID NO:8, 10, 12, or 14. In related aspects, the invention provides vectors comprising the nucleic acid described above, and a host cell comprising the vector. In certain embodiments, the host cell is a prokaryotic cell or eukaryotic cell. In certain particular embodiments, the host cell is a prokaryotic cell, including without limitation, an E. coli cell. In certain other embodiments, the host cell is a eukaryotic cell, including without limitation, a CHO cell. In certain embodiments, the host cell comprises a vector comprising a nucleic acid encoding the IL-22 Fc fusion protein comprising the amino acid sequence of SEQ ID NO:8.

a) Glycosylation Variants

In certain embodiments, an IL-22 Fc fusion protein described herein is altered to increase or decrease the extent to which the fusion protein or a portion thereof (e.g., the Fc portion of the fusion protein) is glycosylated. Addition or deletion of glycosylation sites to a protein may be conveniently accomplished by altering the amino acid sequence such that one or more glycosylation sites is created or removed.

Where the fusion protein comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody or the Fc region of an antibody may be made in order to create Fc variants with certain improved properties.

The amount of fucose attached to the CH2 domain of the Fc region can be determined by calculating the average amount of fucose within the sugar chain at Asn297, relative to the sum of all glycostructures attached to Asn 297 or N297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108; US 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); U.S. Pat. Appl. No. US 2003/0157108 A1; and WO 2004/056312 A1, especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878; U.S. Pat. No. 6,602,684; and US 2005/0123546. Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

b) Fc Region Variants

In certain embodiments, one or more amino acid modifications may be introduced into the Fc region of an Fc fusion protein provided herein, thereby generating an Fc region variant. The Fc region variant may comprise a human Fc region sequence (e.g., a human IgG1, IgG2, IgG3, or IgG4 Fc region) comprising an amino acid modification (e.g., a substitution) at one or more amino acid positions. For example, the hinge may include a Ser to Pro substitution, for example, as shown in the bolded and underlined Pro residue in the amino acid sequence of CPPCP (SEQ ID NO:31). Such a Ser to Pro substitution may increase the stability of the molecule.

In certain embodiments, the invention contemplates an Fc variant that possesses some but not all effector functions, which make it a desirable candidate for applications in which the half-life of the antibody or a fusion protein comprising an Fc region in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody or Fc lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch et al., Annu. Rev. Immunol. 9:457-492 (1991). Non-limiting examples of in vitro assays to assess ADCC activity of a molecule of interest is described in U.S. Pat. No. 5,500,362 (see, e.g. Hellstrom et al., Proc. Nat'l Acad. Sci. USA 83:7059-7063 (1986) and Hellstrom et al., Proc. Nat'l Acad. Sci. USA 82:1499-1502 (1985); U.S. Pat. No. 5,821,337 (see Bruggemann et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CYTOTOX 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody or Fc is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg et al., Blood 101:1045-1052 (2003); and Cragg et al., Blood 103:2738-2743 (2004)). FcRn binding and in vivo clearance/half-life determinations can also be performed using methods known in the art (see, e.g., Petkova et al., Intl. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody or Fc variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an IL-22 Fc fusion protein comprises an Fc variant with one or more amino acid substitutions which reduce ADCC, e.g., substitution at position 297 of the Fc region to remove the N-glycosylation site and yet retain FcRn binding activity (EU numbering of residues).

In some embodiments, alterations are made in the Fc region that result in diminished C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

c) Cysteine Engineered Variants

In certain embodiments, it may be desirable to create cysteine engineered Fc fusion protein, in which one or more residues of the Fc region of an antibody are substituted with cysteine residues. In particular embodiments, the substituted residues occur at accessible sites of the Fc. By substituting those residues with cysteine, reactive thiol groups are thereby positioned at accessible sites of the Fc and may be used to conjugate the Fc to other moieties, such as drug moieties or linker-drug moieties, to create an immunoconjugate, as described further herein. For example, S400 (EU numbering) of the heavy chain Fc region can be substituted with Cys. See, e.g., U.S. Pat. No. 7,521,541.

2. Exemplary IL-22 Polypeptides

Any suitable IL-22 polypeptide can be included in the IL-22 Fc fusion proteins used in the methods, uses, articles of manufacture, and kits described herein. For example, in any of the IL-22 Fc fusion proteins described herein, the IL-22 polypeptide can include a polypeptide comprising an amino acid sequence comprising SEQ ID NO:71 (human IL-22 with the endogenous IL-22 leader sequence), or a polypeptide comprising an amino acid sequence that has at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity) with SEQ ID NO:71. In certain embodiments, the IL-22 polypeptide comprises an amino acid sequence comprising SEQ ID NO:4 (human IL-22 without a leader sequence) or a polypeptide comprising an amino acid sequence that has at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity) with SEQ ID NO:4. In certain embodiments, the IL-22 polypeptide comprises an amino acid sequence comprising SEQ ID NO:4.

The preparation of native IL-22 molecules, along with their nucleic acid and polypeptide sequences, can be achieved through methods known to those of ordinary skill in the art. For example, IL-22 polypeptides can be produced by culturing cells transformed or transfected with a vector containing IL-22 nucleic acid. It is, of course, contemplated that alternative methods, which are well known in the art, can be employed to prepare IL-22. For instance, the IL-22 sequence, or portions thereof, can be produced by direct peptide synthesis using solid-phase techniques (see, e.g., Stewart et al., 1969, Solid-Phase Peptide Synthesis, W.H. Freeman Co., San Francisco, Calif. (1969); Merrifield, J. Am. Chem. Soc., 1963, 85:2149-2154). In vitro protein synthesis can be performed using manual techniques or by automation. Automated synthesis can be accomplished, for instance, using an Applied Biosystems Peptide Synthesizer (Foster City, Calif.) using manufacturer's instructions. Various portions of IL-22 can be chemically synthesized separately and combined using chemical or enzymatic methods to produce the full-length IL-22.

IL-22 variants can be prepared by introducing appropriate nucleotide changes into the DNA encoding a native sequence IL-22 polypeptide, or by synthesis of the desired IL-22 polypeptide. Those skilled in the art will appreciate that amino acid changes can alter post-translational processes of IL-22, such as changing the number or position of glycosylation sites or altering the membrane anchoring characteristics.

Variations in the native sequence IL-22 polypeptides described herein can be made, for example, using any of the techniques and guidelines for conservative and non-conservative mutations set forth, for instance, in U.S. Pat. No. 5,364,934. Variations can be a substitution, deletion, or insertion of one or more codons encoding a native sequence or variant IL-22 that results in a change in its amino acid sequence as compared with a corresponding native sequence or variant IL-22. Optionally the variation is by substitution of at least one amino acid with any other amino acid in one or more of the domains of a native sequence IL-22 polypeptide. Guidance in determining which amino acid residue can be inserted, substituted or deleted without adversely affecting the desired activity can be found by comparing the sequence of the IL-22 with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions can optionally be in the range of 1 to 5 amino acids. The variation allowed can be determined by systematically making insertions, deletions or substitutions of amino acids in the sequence and testing the resulting variants for activity, for example, in the in vitro assay described in the Examples below.

In particular embodiments, conservative substitutions of interest are shown in Table 2 under the heading of preferred substitutions. If such substitutions result in a change in biological activity, then more substantial changes, denominated exemplary substitutions in Table 2, or as further described below in reference to amino acid classes, are introduced and the products screened.

Another type of covalent modification of the IL-22 polypeptides included within the scope of this invention comprises altering the native glycosylation pattern of the polypeptides. “Altering the native glycosylation pattern” is intended for purposes herein to mean deleting one or more carbohydrate moieties found in native sequence IL-22, and/or adding one or more glycosylation sites that are not present in the native sequence IL-22, and/or alteration of the ratio and/or composition of the sugar residues attached to the glycosylation site(s).

Glycosylation of polypeptides is typically either N-linked or O-linked. Addition of glycosylation sites to the IL-22 polypeptide can be accomplished by altering the amino acid sequence. The alteration can be made, for example, by the addition of, or substitution by, one or more serine or threonine residues to the native sequence IL-22 (for N-linked glycosylation sites), or the addition of a recognition sequence for O-linked glycosylation. The IL-22 amino acid sequence can optionally be altered through changes at the DNA level, particularly by mutating the DNA encoding the IL-22 polypeptide at preselected bases such that codons are generated that will translate into the desired amino acids.

Another means of increasing the number of carbohydrate moieties on the IL-22 polypeptide is by chemical or enzymatic coupling of glycosides to the polypeptide. Such methods are described in the art, e.g., in WO 87/05330 and in Aplin et al., CRC Crit. Rev. Biochem., pp. 259-306 (1981).

Removal of carbohydrate moieties present on an IL-22 polypeptide can be accomplished chemically or enzymatically or by mutational substitution of codons encoding for amino acid residues that serve as targets for glycosylation. Chemical deglycosylation techniques are known in the art and described, for instance, by Hakimuddin et al., Arch. Biochem. Biophys. 259:52 (1987) and by Edge et al., Anal. Biochem. 118:131 (1981). Enzymatic cleavage of carbohydrate moieties on polypeptides can be achieved by the use of a variety of endo- and exo-glycosidases as described by Thotakura et al., Meth. Enzymol. 138:350 (1987).

The variations can be made using methods known in the art such as oligonucleotide-mediated (site-directed) mutagenesis, alanine scanning, and PCR mutagenesis. Site-directed mutagenesis (Carter et al., 1986, Nucl. Acids Res. 13:4331; Zoller et al., 1987, Nucl. Acids Res. 10:6487), cassette mutagenesis (Wells et al., 1985, Gene 34:315), restriction selection mutagenesis (Wells et al., 1986, Philos. Trans. R. Soc. London A 317:415), or other known techniques can be performed on the cloned DNA to produce the IL-22 variant DNA.

Fragments of an IL-22 polypeptide are also provided herein. Such fragments can be truncated at the N-terminus or C-terminus, or can lack internal residues, for example, when compared with a full length native protein. Certain fragments lack amino acid residues that are not essential for a desired biological activity of an IL-22 polypeptide of the present invention. Accordingly, in certain embodiments, a fragment of an IL-22 polypeptide is biologically active. In certain embodiments, a fragment of full length IL-22 lacks the N-terminal signal peptide sequence.

Covalent modifications of native sequence and variant IL-22 polypeptides are included within the scope of this invention. One type of covalent modification includes reacting targeted amino acid residues of IL-22 with an organic derivatizing agent that is capable of reacting with selected side chains or the N- or C-terminal residues of the IL-22 polypeptide. Derivatization with bifunctional agents is useful, for instance, for crosslinking IL-22 to a water-insoluble support matrix or surface, for example, for use in the method for purifying anti-IL-22 antibodies. Commonly used crosslinking agents include, e.g., 1,1-bis(diazo-acetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidyl-propionate), bifunctional maleimides such as bis-N-maleimido-1,8-octane, and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the a-amino groups of lysine, arginine, and histidine side chains (T. E. Creighton, 1983, Proteins: Structure and Molecular Properties, W. H. Freeman & Co., San Francisco, pp. 79-86i), acetylation of the N-terminal amine, and amidation of any C-terminal carboxyl group.

Another type of covalent modification of IL-22 comprises linking the IL-22 polypeptide to one of a variety of nonproteinaceous polymers, e.g., polyethylene glycol, polypropylene glycol, or polyoxyalkylenes, for example in the manner set forth in U.S. Pat. Nos. 4,640,835; 4,496,689; 4,301,144; 4,670,417; 4,791,192; or 4,179,337. The native sequence and variant IL-22 can also be modified in a way to form a chimeric molecule comprising IL-22, including fragments of IL-22, fused to another, heterologous polypeptide or amino acid sequence.

In one embodiment, such a chimeric molecule comprises a fusion of IL-22 with a tag polypeptide which provides an epitope to which an anti-tag antibody can selectively bind. The epitope tag is generally placed at the amino- or carboxyl-terminus of the IL-22 polypeptide. The presence of such epitope-tagged forms of the IL-22 polypeptide can be detected using an antibody against the tag polypeptide. Also, provision of the epitope tag enables the IL-22 polypeptide to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 (Field et al., 1988, Mol. Cell. Biol., 8:2159-2165); the c-myc tag and the 8F9, 3C7, 6E10, G4, and 9E10 antibodies thereto (Evan et al., 1985, Mol. Cell. Biol. 5:3610-3616); and the Herpes Simplex virus glycoprotein D (gD) tag and its antibody (Paborsky et al., 1990, Protein Engineering 3(6):547-553). Other tag polypeptides include the Flag-peptide (Hopp et al., 1988, BioTechnology 6:1204-1210); the KT3 epitope peptide (Martin et al., 1992, Science 255:192-194); a tubulin epitope peptide (Skinner et al., 1991, J. Biol. Chem. 266:15163-15166); and the T7 gene 10 protein peptide tag (Lutz-Freyermuth et al., 1990, Proc. Natl. Acad. Sci. USA, 87:6393-6397).

In another embodiment, the chimeric molecule can comprise a fusion of the IL-22 polypeptide or a fragment thereof with an immunoglobulin or a particular region of an immunoglobulin. For a bivalent form of the chimeric molecule, such a fusion can be to the Fc region of an IgG molecule. These fusion polypeptides are antibody-like molecules which combine the binding specificity of a heterologous protein (an “adhesin”) with the effector functions of immunoglobulin constant domains, and are often referred to as immunoadhesins. Structurally, the immunoadhesins comprise a fusion of an amino acid sequence of IL-22, or a variant thereof, and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin can be obtained from any immunoglobulin, such as IgG1, IgG2, IgG3, or IgG4 subtypes, IgA (including IgA₁ and IgA₂), IgE, IgD, or IgM. In certain embodiments, the IL-22 Fc fusion protein exhibits modified effector activities.

The IL-22 polypeptide, or a fragment thereof, can be fused, for example, to an immunoglobulin heavy chain constant region sequence to produce an IL-22-Ig fusion protein (e.g., IL-22 Fc fusion protein). The IL-22 polypeptide can be human or murine IL-22. The immunoglobulin heavy chain constant region sequence can be human or murine immunoglobulin heavy chain constant region

### B. Methods of Making IL-22 Fc Fusion Proteins for Use in the Methods

The IL-22 Fc fusion proteins used in the methods, uses, articles of manufacture, and kits described herein can be prepared by any suitable method, e.g., culturing cells transformed or transfected with a vector containing a nucleic acid encoding an IL-22 Fc fusion protein, a fragment, or a variant thereof. In some embodiments, the IL-22 Fc fusion proteins are produced by a method described in International Patent Application No. PCT/US2019/015277, which is incorporated herein by reference in its entirety. Host cells comprising any such vector are also provided. Any suitable host cell can be used, e.g., mammalian cells (e.g., CHO cells), E. coli, or yeast. Processes for producing any of the herein described IL-22 Fc fusion proteins are further provided and, in general, involve culturing host cells under conditions suitable for expression of the desired IL-22 Fc fusion protein and recovering, and optionally purifying, the desired IL-22 Fc fusion protein from the cell culture.

Host cells are transfected or transformed with expression or cloning vectors described herein for IL-22 polypeptide production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: A Practical Approach, M. Butler, ed. (IRL Press, 1991) and Sambrook et al., supra.

Methods of transfection are known to the ordinarily skilled artisan, for example, by CaPO₄ and electroporation. Depending on the host cell used, transformation is performed using standard techniques appropriate to such cells. The calcium treatment employing calcium chloride, as described in Sambrook et al., supra, or electroporation is generally used for prokaryotes or other cells that contain substantial cell-wall barriers. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transformations have been described in U.S. Pat. No. 4,399,216. Transformations into yeast are typically carried out according to the method of Van Solingen et al., J. Bact, 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, can also be used. For various techniques for transforming mammalian cells, see Keown et al., Methods in Enzymology, 185:527-537 (1990) and Mansour et al., Nature, 336:348-352 (1988).

Recombinantly expressed polypeptides of the present invention can be recovered from culture medium or from host cell lysates. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of a polypeptide of the present invention. Various methods of protein purification can be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular polypeptide produced.

Alternative methods, which are well known in the art, can be employed to prepare a polypeptide of the present invention. For example, a sequence encoding a polypeptide or portion thereof, can be produced by direct peptide synthesis using solid-phase techniques (see, e.g., Stewart et al., 1969, Solid-Phase Peptide Synthesis, W.H. Freeman Co., San Francisco, Calif.; Merrifield, J. 1963, Am. Chem. Soc., 85:2149-2154. In vitro protein synthesis can be performed using manual techniques or by automation. Automated synthesis can be accomplished, for instance, using an Applied Biosystems Peptide Synthesizer (Foster City, Calif.) using manufacturer's instructions. Various portions of a polypeptide of the present invention or portion thereof can be chemically synthesized separately and combined using chemical or enzymatic methods to produce the full-length polypeptide or portion thereof.

In other embodiments, the invention provides chimeric molecules comprising any of the herein described polypeptides fused to a heterologous polypeptide or amino acid sequence. Examples of such chimeric molecules include, but are not limited to, any of the herein described polypeptides fused to an epitope tag sequence or an Fc region of an immunoglobulin.

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635).

In addition to prokaryotes, eukaryotic microbes such as filamentous fungi or yeast are suitable cloning or expression hosts for IL-22-encoding vectors. Saccharomyces cerevisiae is a commonly used lower eukaryotic host microorganism.

Suitable host cells for the expression of glycosylated IL-22 are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 cells transformed by SV40 (COS-7, ATCC CRL 1651); human embryonic kidney cells (293 or 293 cells subcloned for growth in suspension culture, Graham et al., J. Gen Virol., 36:59 (1977)); Chinese hamster ovary cells/-DHFR (CHO, Urlaub and Chasin, Proc. Natl. Acad. Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor cells (MMT 060562, ATCC CCL51). The selection of the appropriate host cell is deemed to be within the skill in the art.

The nucleic acid (e.g., cDNA or genomic DNA) encoding IL-22 can be inserted into a replicable vector for cloning (amplification of the DNA) or for expression. Various vectors are publicly available. The vector can, for example, be in the form of a plasmid, cosmid, viral particle, or phage. The appropriate nucleic acid sequence can be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The IL-22 polypeptides can be produced recombinantly not only directly, but also as a fusion polypeptide with a heterologous polypeptide, which can be a signal sequence or other polypeptide having a specific cleavage site at the N-terminus of the mature protein or polypeptide, as well as an IL-22 Fc fusion protein. In general, the signal sequence can be a component of the vector, or it can be a part of the IL-22 DNA that is inserted into the vector. The signal sequence can be a prokaryotic signal sequence selected, for example, from the group of the alkaline phosphatase, penicillinase, 1 pp, or heat-stable enterotoxin II leaders. For yeast secretion the signal sequence can be, e.g., the yeast invertase leader, alpha factor leader (including Saccharomyces and Kluyveromyces alpha-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences can be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2: plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells is one that enables the identification of cells competent to take up the IL-22 nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 (see, e.g., Stinchcomb et al., Nature, 282:39(1979); Kingsman et al., Gene, 7:141 (1979); Tschemper et al., Gene, 10:157 (1980)). The trp1 gene provides a selection marker for a mutant strain of yeast lacking the ability to grow in tryptophan, for example, ATCC No. 44076 or PEP4-1 (Jones, Genetics, 85:12 (1977)).

Expression and cloning vectors usually contain a promoter operably linked to the IL-22 nucleic acid sequence to direct mRNA synthesis. Promoters recognized by a variety of potential host cells are well known. Promoters suitable for use with prokaryotic hosts include the quadrature-lactamase and lactose promoter systems (see, e.g., Chang et al., Nature, 275:615 (1978); Goeddel et al., Nature, 281:544 (1979)), alkaline phosphatase, a tryptophan (trp) promoter system (see, e.g., Goeddel, Nucleic Acids Res., 8:4057 (1980); EP 36,776), and hybrid promoters such as the tac promoter (see, e.g., deBoer et al., Proc. Natl. Acad. Sci. USA, 80:21-25 (1983)). Promoters for use in bacterial systems also will contain a Shine-Dalgarno (S.D.) sequence operably linked to the DNA encoding IL-22.

Examples of suitable promoter sequences for use with yeast hosts include the promoters for 3-phosphoglycerate kinase (see, e.g., Hitzeman et al., J. Biol. Chem, 255:2073 (1980)) or other glycolytic enzymes (see, e.g., Hess et al., J. Adv. Enzyme Reg., 7:149 (1968); Holland, Biochemistry, 17:4900 (1978)), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

IL-22 transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the IL-22 polypeptides by higher eukaryotes can be increased by inserting an enhancer sequence into the vector. Enhancers are cis-acting elements of DNA, usually about from 10 to 300 bp, which act on a promoter to increase its transcription. Many enhancer sequences are now known from mammalian genes (globin, elastase, albumin, a-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer can be spliced into the vector at a position 5′ or 3′ to the IL-22 coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding IL-22.

Still other methods, vectors, and host cells suitable for adaptation to the synthesis of IL-22 in recombinant vertebrate cell culture are described in Gething et al., Nature, 293:620-625 (1981); Mantei et al., Nature, 281:4046 (1979); EP 117,060; and EP 117,058.

Gene amplification and/or expression can be measured in a sample directly, for example, by conventional Southern blotting, Northern blotting to quantitate the transcription of mRNA (see, e.g., Thomas, Proc. Natl. Acad. Sci. USA, 77:5201-5205 (1980)), dot blotting (DNA analysis), or in situ hybridization, using an appropriately labeled probe, based on the sequences provided herein. Alternatively, antibodies can be employed that can recognize specific duplexes, including DNA duplexes, RNA duplexes, and DNA-RNA hybrid duplexes or DNA-protein duplexes. The antibodies in turn can be labeled and the assay can be carried out where the duplex is bound to a surface, so that upon the formation of duplex on the surface, the presence of antibody bound to the duplex can be detected.

Gene expression, alternatively, can be measured by immunological methods, such as immunohistochemical staining of cells or tissue sections and assay of cell culture or body fluids, to quantitate directly the expression of gene product. Antibodies useful for immunohistochemical staining and/or assay of sample fluids can be either monoclonal or polyclonal, and can be prepared in any mammal. Conveniently, the antibodies can be prepared against a native sequence IL-22 polypeptide or against a synthetic peptide based on the DNA sequences provided herein or against exogenous sequence fused to IL-22 DNA and encoding a specific antibody epitope.

IL-22 Fc fusion proteins can be recovered from culture medium or from host cell lysates. If membrane-bound, it can be released from the membrane using a suitable detergent solution (e.g. TRITON® X-100) or by enzymatic cleavage. Cells employed in expression of IL-22 can be disrupted by various physical or chemical means, such as freeze-thaw cycling, sonication, mechanical disruption, or cell lysing agents.

It may be desired to purify IL-22 Fc fusion proteins from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the IL-22 polypeptide. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular IL-22 produced. The above-described general methods can be applied to the preparation of IL-2 Fc fusion protein as well.

Similarly, IL-22 Fc fusion proteins may be produced using recombinant methods and compositions, as described in, e.g., Molecular Cloning: A Laboratory Manual (Sambrook, et al., 1989, Cold Spring Harbor Laboratory Press) and PCR Protocols: A Guide to Methods and Applications (Innis, et al. 1990. Academic Press, San Diego, Calif.). In one embodiment, isolated nucleic acid encoding IL-22 Fc fusion proteins described herein is provided. In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with) a vector comprising a nucleic acid that encodes an amino acid sequence comprising the IL-22 Fc fusion protein. In certain embodiment, the vector is an expression vector. In one embodiment, the host cell is eukaryotic, e.g. a Chinese Hamster Ovary (CHO) cell or lymphoid cell (e.g., Y0, NS0, Sp20 cell). In one embodiment, a method of making an IL-22 Fc fusion protein is provided, wherein the method comprises culturing a host cell comprising a nucleic acid encoding the IL-22 Fc fusion protein, as provided above, under conditions suitable for expression of the Fc fusion protein, and optionally recovering the Fc fusion protein from the host cell (or host cell culture medium).

For recombinant production of an IL-22 Fc fusion protein, nucleic acid encoding an Fc fusion protein, e.g., as described herein, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the fusion protein). In certain embodiments, when preparing the IL-22 Fc fusion proteins, nucleic acid encoding the IL-22 polypeptide or a fragment thereof can be ligated to nucleic acid encoding an immunoglobulin constant domain sequence at specified location on the constant domain to result in an Fc fusion at the C-terminus of IL-22; however N-terminal fusions are also possible.

As an example of constructing an IL-22 Fc fusion protein, the DNA encoding IL-22 is cleaved by a restriction enzyme at or proximal to the 3′ end of the DNA encoding IL-22 and at a point at or near the DNA encoding the N-terminal end of the mature polypeptide (where use of a different leader is contemplated) or at or proximal to the N-terminal coding region for IL-22 full-length protein (where a native signal is employed). This DNA fragment then is readily inserted into DNA encoding an immunoglobulin light or heavy chain constant region and, if necessary, tailored by deletional mutagenesis. Preferably, this is a human immunoglobulin when the fusion protein is intended for in vivo therapy for humans.

In some embodiments, the IL-22-immunoglobulin chimeras are assembled as monomers, hetero- or homo-multimer, or as dimers or tetramers. Generally, these assembled immunoglobulins will have known unit structures as represented by the following diagrams. A basic four chain structural unit is the form in which IgG, IgD, and IgE exist. A four chain unit is repeated in the higher molecular weight immunoglobulins; IgM generally exists as a pentamer of, basic four-chain units held together by disulfide bonds. IgA globulin, and occasionally IgG globulin, may also exist in a multimeric form in serum. In the case of multimers, each four chain unit may be the same or different. See also Capon et al. U.S. Pat. No. 5,116,964, incorporated herein by reference in its entirety.

DNA encoding immunoglobulin light or heavy chain constant regions is known or readily available from cDNA libraries or is synthesized. See for example, Adams et al., Biochemistry 19:2711-2719 (1980); Gough et al., Biochemistry 19:2702-2710 (1980); Dolby et al; P.N.A.S. USA, 77:6027-6031 (1980); Rice et al P.N.A.S USA 79:7862-7865 (1982); Falkner et al; Nature 298:286-288 (1982); and Morrison et al; Ann. Rev. Immunol. 2:239-256 (1984). DNA sequence encoding human IL-22 with the endogenous leader sequence is provided herein (SEQ ID NO:70). DNA sequences encoding other desired binding partners which are known or readily available from cDNA libraries are suitable in the practice of this invention.

DNA encoding an IL-22 Fc fusion protein of this invention is transfected into a host cell for expression. If multimers are desired then the host cell is transformed with DNA encoding each chain that will make up the multimer, with the host cell optimally being selected to be capable of assembling the chains of the multimers in the desired fashion. If the host cell is producing an immunoglobulin prior to transfection then one needs only transfect with the binding partner fused to light or to heavy chain to produce a heteroantibody. The aforementioned immunoglobulins having one or more arms bearing the binding partner domain and one or more arms bearing companion variable regions result in dual specificity for the binding partner ligand and for an antigen or therapeutic moiety. Multiply cotransformed cells are used with the above-described recombinant methods to produce polypeptides having multiple specificities such as the heterotetrameric immunoglobulins discussed above.

Although the presence of an immunoglobulin light chain is not required in the immunoadhesins of the present invention, an immunoglobulin light chain might be present either covalently associated to an IL-22-immunoglobulin heavy chain fusion polypeptide. In this case, DNA encoding an immunoglobulin light chain is typically co-expressed with the DNA encoding the IL-22-immunoglobulin heavy chain fusion protein. Upon secretion, the hybrid heavy chain and the light chain will be covalently associated to provide an immunoglobulin-like structure comprising two disulfide-linked immunoglobulin heavy chain-light chain pairs. Methods suitable for the preparation of such structures are, for example, disclosed in U.S. Pat. No. 4,816,567 issued Mar. 28, 1989.Suitable host cells for cloning or expression of target protein-encoding vectors include prokaryotic or eukaryotic cells described herein. For example, IL-22 Fc fusion protein may be produced in bacteria, in particular when glycosylation and Fc effector function are not needed or are detrimental. For expression of polypeptides in bacteria, see, e.g., U.S. Pat. Nos. 5,648,237, 5,789,199, and 5,840,523. See also Charlton, Methods in Molecular Biology, Vol. 248 (B. K. C. Lo, ed., Humana Press, Totowa, N.J., 2003), pp. 245-254, describing expression of antibody fragments in E. coli. After expression, the Fc fusion protein may be isolated from the bacterial cell paste in a soluble fraction and can be further purified. As exemplified in the example section, further purification methods include without limitation purification using a Protein A column.

In addition to prokaryotes, eukaryotic microbes such as filamentous fungi or yeast are suitable cloning or expression hosts, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated proteins are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177; 6,040,498; 6,420,548; 7,125,978; and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### C. Assays

The compositions (e.g., IL-22 Fc fusion proteins, or pharmaceutical compositions thereof) for use in the methods, uses, articles of manufacture, and kits described herein may be identified, screened for, or characterized for their physical/chemical properties and/or biological activities by various assays known in the art.

1. Binding Assays and Other Assays

In one aspect, an IL-22 Fc fusion protein is tested for its receptor binding activity, e.g., by known methods such as ELISA, western blotting analysis, cell surface binding by Scatchard, surface plasmon resonance. In another aspect, competition assays may be used to identify an antibody that competes with the IL-22 Fc fusion protein for binding to the IL-22 receptor. In a further aspect, an IL-22 Fc fusion protein of the invention can be used for detecting the presence or amount of IL-22 receptor or IL22-Binding Protein (soluble receptor) present in a biological sample. In a further aspect, an IL-22 Fc fusion protein of the invention can be used for detecting the presence or amount of IL-22 receptor present in a biological sample. In certain embodiments, the biological sample is first blocked with a non-specific isotype control antibody to saturate any Fc receptors in the sample. Exemplary assays are described in Example 1 of International Patent Application No. International Patent Application No. PCT/US2019/015268, which is incorporated herein by reference in its entirety.

2. Activity Assays

In one aspect, assays are provided for identifying biological activity of a composition (e.g., an IL-22 Fc fusion protein or a pharmaceutical composition thereof). Biological activity of an IL-22 polypeptide or IL-22 Fc fusion protein in a composition (e.g., a pharmaceutical composition) may include, e.g., binding to IL-22 receptor, stimulating IL-22 signaling, and inducing STAT3, or REG3 (also known as PAP or HIP/PAP (hepatocarcinoma-intestine-pancrease/pancreatic associated protein) expression. Further, in the case of a cardiovascular disease or condition, the biological activity may include affecting the formation of atherosclerotic plaques, in particular to inhibit formation of atherosclerotic plaque formation. Inhibition of plaque formation can be assessed by any suitable imaging method known to those of ordinary skill in the art.

3. Stability Assays

In one aspect, assays are provided for determining the stability of a composition (e.g., an IL-22 Fc fusion protein or a pharmaceutical composition thereof). For example, a composition (e.g., a pharmaceutical composition) can be evaluated qualitatively and/or quantitatively in a variety of different ways, including evaluation of aggregate formation (for example, using size exclusion chromatography, by measuring turbidity, and/or by visual inspection); evaluation of ROS formation (for example, by using a light stress assay or an 2,2′-azobis(2-amidinopropane) dihydrochloride (AAPH) stress assay); oxidation of specific amino acid residues of the protein (for example, a Met residue of an IL-22 Fc fusion protein); by assessing charge heterogeneity using cation exchange chromatography, image capillary isoelectric focusing (icIEF) or capillary zone electrophoresis; amino-terminal or carboxy-terminal sequence analysis; mass spectrometric analysis; SDS-PAGE analysis to compare reduced and intact polypeptides (e.g., IL-22 Fc fusion proteins); peptide map (for example, tryptic or LYS-C) analysis; evaluating biological activity or target binding function of the protein (e.g., binding of an IL-22 Fc fusion protein to an IL-22 receptor); and the like. Instability may involve any one or more of: aggregation, deamidation (e.g., Asn deamidation), oxidation (e.g., Met oxidation and/or Trp oxidation), isomerization (e.g., Asp isomerization), clipping/hydrolysis/fragmentation (e.g., hinge region fragmentation), succinimide formation, unpaired cysteine(s), N-terminal extension, C-terminal processing, glycosylation differences, and the like. Exemplary assays are described in Example 1 and Example 3 of U.S. Provisional Patent Application No. 62/622,704, which is incorporated herein by reference in its entirety.

### D. Conjugates for Use in the Methods

In any of the methods, uses, articles of manufacture, and kits described herein, the IL-22 Fc fusion protein may be a conjugate comprising an IL-22 Fc fusion protein described herein conjugated to one or more agents for detection, formulation, half-life extension, mitigating immunogenicity, or tissue penetration. Exemplary types of conjugation include, without limitation, PEGylation and conjugation to radioactive isotopes.

In another embodiment, a conjugate comprises an IL-22 Fc fusion protein as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹², and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese, or iron.

### E. Additional Therapeutic Methods and Uses

The invention provides additional therapeutic methods and uses described below.

Any of the methods, dosage regimens, and dosing cycles described herein can be used for treating cardiovascular diseases and conditions, metabolic syndrome, acute endotoxemia and sepsis, skin disorders (e.g., hidradenitis suppurativa), COPD, nonalcoholic fatty acid liver disease (e.g., NASH), and diabetes. For the prevention, treatment or reduction in the severity of a given disease or condition, the appropriate dosage of a composition of the invention will depend on the type of disease or condition to be treated, as defined above, the severity and course of the disease or condition, whether the agent is administered for preventive or therapeutic purposes, previous therapy, the subjects clinical history and response to the compound, and the discretion of the attending physician. The compound is suitably administered to the subject at one time or over a series of treatments. Preferably, it is desirable to determine the dose-response curve and the pharmaceutical composition of the invention first in vitro, and then in useful animal models prior to testing in humans. Any of the dosing regimens and dosing cycles described herein can be used for treatment of treating cardiovascular diseases and conditions, metabolic syndrome, acute endotoxemia and sepsis, and diabetes.

In one aspect, the present invention provides methods of treatment for a cardiovascular disease or disorder, metabolic syndrome, acute endotoxemia and sepsis, a skin disorder (e.g., hidradenitis suppurativa), and an insulin-related disorder. In one embodiment, the method comprises administering to a subject in need a therapeutically effective amount of a composition (e.g., an IL-22 Fc fusion protein or a pharmaceutical composition thereof). In another aspect, the invention provides a method for the delaying or slowing down of the progression of a cardiovascular disease or disorder, metabolic syndrome, a skin disorder (e.g., hidradenitis suppurativa), and an insulin-related disorder. In one embodiment, the method comprises administering to subject diagnosed with the disease, condition, or disorder, an effective amount of a composition (e.g., an IL-22 Fc fusion protein or a pharmaceutical composition thereof). In another aspect, the invention provides a method for preventing indicia of a cardiovascular disease or disorder, and an insulin-related disorder. In one embodiment, the method comprises administering an effective amount of a composition (e.g., an IL-22 Fc fusion protein or a pharmaceutical composition thereof) to a subject at risk of the disease, condition, or disorder, wherein the composition is effective against the development of indicia of the disease, condition, or disorder.

1. Cardiovascular Diseases and Conditions

In one aspect, the invention provides methods in which IL-22 Fc fusion proteins provide a therapeutic, preventative, or prophylactic effect against the development of, or the progression of, clinical and/or histological and/or biochemical and/or pathological indicia (including both symptoms and signs) of cardiovascular diseases or conditions in a subject. Any of the methods, dosing regimens, and dosing cycles described herein can be used in a method of treating a cardiovascular disease or condition in a subject. In one embodiment, the disease or condition is atherosclerosis. In one embodiment, the indicia include atherosclerotic plaque formation and/or vascular inflammation. In another embodiment, the subject is at risk for cardiovascular disease. In general, a subject at risk will previously have had a cardiovascular disease or condition as described herein, or will have a genetic predisposition for a cardiovascular disease or condition.

The efficacy of the treatment of cardiovascular diseases and conditions can be measured by various assessments commonly used in evaluating cardiovascular diseases. For example, cardiovascular health can be assessed. Cardiovascular health can be evaluated by, but not limited to, e.g., blood tests (e.g., total cholesterol, LDL-C, HDL-C, triglyceride, C-reactive protein, fibrinogen, homocysteine, fasting insulin, ferritin, lipoprotein, and LPS), blood pressure, auscultation, electrocardiogram, cardiac stress testing, cardiac imaging (e.g., coronary catheterization, echocardiogram, intravascular ultrasound, positron emission tomography, computed tomography angiography, and magnetic resonance imaging).

2. Metabolic Syndrome

In one aspect, the methods, dosing regimens, or dosing cycles described herein provide a therapeutic, preventative, or prophylactic effect against the development of, or the progression of, clinical and/or histological and/or biochemical and/or pathological indicia (including both symptoms and signs) of metabolic syndrome (or metabolic disorder or disease) in a subject. In one or more embodiment, the subject is at risk for metabolic syndrome.

The efficacy of the treatment of metabolic syndrome can be measured by various assessments commonly used in evaluating metabolic syndrome. For example, obesity can be measured. As a further example, hyperglycemia, dyslipidemia, insulin resistance, chronic adipose tissue inflammation, and/or hypertension can be measured. Reduction in in levels of one or more of C-reactive protein, IL-6, LPS, and plasminogen activator inhibitor 1 can be measured. These measurements can be performed by any methods well known in the art.

3. Insulin-Related Disorders

Any of the methods, dosing regimens, and dosing cycles described herein can be used in a method of treating an insulin-related disorder in a subject. For insulin-related disorders, the term “treatment” refers to both therapeutic treatment and prophylactic or preventative measures for the disorder, wherein the object is to prevent or slow down (lessen) the targeted pathologic condition or disorder. Those in need of treatment include those already with an insulin-related disorder as well as those prone to have such a disorder or those in whom the disorder is to be prevented.

In one aspect, the methods provide a preventative or prophylactic effect against the development of, or the progression of, clinical and/or histological and/or biochemical and/or pathological indicia (including both symptoms and signs) of an insulin-related disorder in a subject. In one embodiment, the disorder is Type I diabetes, Type II diabetes, or gestational diabetes. In one embodiment, the pathology or pathological indicia include one or more of: little or no insulin production by the pancreas (e.g., islet cells), insulin resistance, and hyperglycemia. In another embodiment, the subject is at risk for an insulin-related disorder. In general, a subject at risk has a genetic predisposition for an insulin-related disorder, has been exposed to a virus that triggers autoimmune destruction of islet cells (e.g., Epstein-Barr virus, coxsackievirus, mumps virus or cytomegalovirus), is obese, is pre-diabetic (higher than normal blood sugar levels), or has gestational diabetes.

The efficacy of the treatment of an insulin-related disorder can be measured by various assessments commonly used in evaluating such disorders. For example, both Type I and Type II diabetes can be evaluated with one or more of the following: a glycated hemoglobin test (A1C), a regular blood sugar test, and a fasting blood sugar test. Type I can also be evaluated by testing for autoantibodies in the blood and/or ketones in the urine. Type II can also be evaluated by testing for oral glucose tolerance.

4. Acute Endotoxemia and Sepsis

Any of the methods, dosing regimens, and dosing cycles described herein can be used in a method of treating an acute endotoxemia or sepsis in a subject. In one aspect, the methods provide a therapeutic, preventative or prophylactic effect against the development of, or the progression of, clinical and/or histological and/or biochemical and/or pathological indicia (including both symptoms and signs) of acute endotoxemia, sepsis, or both, in a subject. In one or more embodiment, the subject is at risk for acute endotoxemia, sepsis, or both.

The efficacy of the treatment of acute endotoxemia, sepsis, or both can be measured by various assessments commonly used in evaluating acute endotoxemia, sepsis, or both. For example, reduction in levels of LPS or inflammatory markers can be measured. These measurements can be performed by any methods well known in the art.

5. Wound Healing

Any of the methods, dosing regimens, and dosing cycles described herein can be used in a method of treating a wound in a subject. There are a variety of ways to measure wound healing. Often images are taken to calculate linear dimensions, perimeter and area. The NIH has a free program, Image J, which allows measurement of wound areas from an image. The final healing prognosis can be extrapolated from initial healing rates based on the migration of the periphery towards the center. This is done using a number of mathematical equations, the most common of which is a modified Gilman's equation. In addition to visual inspection, wound healing measurement can also be aided by spectroscopic methods or MRI. See e.g., Dargaville et al., Biosensors Bioelectronics, 2013, 41:30-42, Tan et al., 2007, British J. Radiol. 80:939-48. If healing is slow/inadequate, biopsies of the wound edges may be taken to rule out or determine infection and malignancy. In certain embodiments, the acceleration or improvement of wound healing can be assessed by comparing wound closure in IL-22-treated and control wounds. In certain embodiments, the acceleration or improvement of wound healing is at least 20%, 30%, 40%, 50%, 60%, 70%, 80%, or 90% faster or better than the control.

In certain aspect, the invention provides methods for promoting/accelerating/improving healing of a wound with or without active infection, microbial contamination or colonization in the wound. The methods can be used for treating infected wounds or promoting/accelerating/improving infected wound healing. In certain embodiments, the methods can be used for treating wounds, or promoting/accelerating/improving wound healing, in the presence of infection. In some embodiments, the methods can be used for treating wounds or promoting/accelerating/improving wound healing in the presence of microbial contamination or colonization with risk for infection. In further embodiments, the patient in need of wound healing treatment can be a diabetic patient. Accordingly, in some embodiments, the wound is a diabetic wound, for example, diabetic foot ulcer. In some further embodiments, the wound is an infected diabetic wound, for example, infected diabetic foot ulcer.

6. Skin Disorders

Any of the methods, dosing regimens, and dosing cycles described herein can be used in a method of treating a skin disorder (e.g., hidradenitis suppurativa) in a subject. In one aspect, the methods provide a therapeutic, preventative or prophylactic effect against the development of, or the progression of, clinical and/or histological and/or biochemical and/or pathological indicia (including both symptoms and signs) of a skin disorder (e.g., hidradenitis suppurativa), in a subject. In one or more embodiment, the subject is at risk for a skin disorder (e.g., hidradenitis suppurativa).

The efficacy of the treatment of a skin disorder (e.g., hidradenitis suppurativa) can be measured by various assessments commonly used in evaluating such skin disorders. For example, reduction in the number or severity of pimples, abcesses, sores, lesions, boils, lumps, and/or scarring can be measured. These measurements can be performed by any methods well known in the art.

7. Combination Therapies

In the methods, uses, articles of manufacture, and kits described herein, a composition (e.g., an IL-22 Fc fusion protein or a pharmaceutical composition thereof) can be used either alone or in combination with other agents in a therapy. For instance, a composition (e.g., an IL-22 Fc fusion protein or a pharmaceutical composition thereof) described herein may be co-administered with at least one additional therapeutic agent. In certain embodiments, an additional therapeutic agent is an immune suppressant that reduces the inflammatory response, including, without limitation, methotrexate, a TNF inhibitor, a TNF antagonist, mesalazine, steroid, dexamethasone, azathioprine, and a combination thereof. Suitable additional therapeutic agents that reduce an inflammatory response include, without limitation, 5-aminosalicylic acid (5-ASA), mercaptopurine (also called 6-mercaptopurine or 6-MP), or combination thereof. In certain embodiments, the composition (e.g., an IL-22 Fc fusion protein or a pharmaceutical composition thereof) may be co-administered with one or more additional therapeutic agents that reduce an inflammatory response (for example, 5-ASA, 6-MP, or a TNF antagonist) for the treatment of IBD. In certain other embodiments, the composition (e.g., an IL-22 Fc fusion protein or a pharmaceutical composition thereof) may be co-administered with an integrin antagonist such as etrolizumab for the treatment of IBD. In one embodiment, the composition (e.g., an IL-22 Fc fusion protein or a pharmaceutical composition thereof) is used in combination with an IL-22 agonist.

For accelerating chronic wound healing, such as for the treatment of diabetic foot ulcer, the administration of a composition (e.g., an IL-22 Fc fusion protein or a pharmaceutical composition thereof) can be combined with one or more additional wound healing agents. Suitable additional wound healing agents include, without limitation, growth factors (e.g., EGF, FGF, IGF, PDGF, TGF, and VEGF), nerve growth factor (NGF), angiogenesis factors (e.g., HGF, TNF-α, angiogenin, IL-8, angiopoietins 1 and 2, Tie-2, integrin α5, matrix metalloproteinases, nitric oxide, and COX-2), members of the platelet derived growth factor (PDGF) family (e.g., PDGF-A, PDGF-B, PDGF-C, and PDGF-D), members of the insulin growth factor (IGF) family (e.g., IGF-I and IGF-II), members of the transforming growth factor (TGF) family (e.g., TGF-α and TGF-β), and anabolic oxygen (vacuum therapy). In certain embodiments, the composition (e.g., an IL-22 Fc fusion protein or a pharmaceutical composition thereof) can be co-administered with one or more additional wound healing agents described herein and/or one or more antibacterial agents or antibiotics suitable for use in topical administration. See, e.g., WO 2006/138468, which is incorporated herein by reference in its entirety. In such embodiments, the antibiotic can be a sulfur antibiotic, including, without limitation, silver sulfadiazine, i.e., silvadeen. The co-administered one or more additional agents can be administered concurrently, alternatively, or sequentially with the composition (e.g., an IL-22 Fc fusion protein or a pharmaceutical composition thereof).

In further exemplary embodiments, if the target is prevention or treatment of cardiovascular diseases or conditions or metabolic syndrome, the administration of a composition (e.g., an IL-22 Fc fusion protein or a pharmaceutical composition thereof) can be combined with or supplement the administration of the cholesterol-lowering agents such as statins (e.g., lovastatin, rosuvastatin, fluvastatin, atorvastatin, pravastatin, and simvastatin), bile acid binding resins (colestipol, cholestyramine sucrose, and colesevelam), ezetimibe, or a ezetimibe-simvastatin combination; anti-platelet agents such as cyclooxygenase inhibitors (e.g., aspirin), adenosine diphosphate (ADP) receptor inhibitors (e.g., clopidogrel, prasugrel, ticagrelor, and ticlopidine), phosphodiesterase inhibitors (e.g., cilostazol), glycoprotein IIB/IIIA inhibitors (e.g., abciximab, eptifibatide, and tirofiban), adenosine reuptake inhibitors (e.g., dipyridamole), thromboxane inhibitors (e.g., thromboxane synthase inhibitors, thromboxane receptor antagonists, and terutroban); beta blockers such as alprenolol, bucindolol, carteolol, carvedilol, labetalol, nadolol, oxprenolol, penbutolol, pindolol, propranolol, sotalol, timolol, eucommia bark, acebutolol, atenolol, betaxolol, bisoprolol, celiprolol, esmolol, metoprolol, nebivolol, butaxamine, ICI-118,551, and SR 59230A; angiotensin-converting enzyme (ACE) inhibitors such as captopril, zofenopril, dicarboxylate-containing agents (e.g., enalapril, ramipril, quinapril, perindopril, lisinopril, benazepril, imidapril, and zofenopril), phosphonate-containing agents (e.g., fosinopril), casokinins, lactokinins, lactotripeptides (e.g., Val-Pro-Pro, and Ile-Pro-Pro produced by the probiotic Lactobacillus helveticus or derived from casein); calcium channel blockers such as dihydropyridines (e.g., amlodipine, aranidipine, azelnidipine, barnidipine, benidipine, cilnidipine, clevidipine, isradipine, efonidipine, felodipine, lacidipine, lercanidipine, manidipine, nicardipine, nifedipine, nilvadipine, nimodipine, nisoldipine, nitrendipine, and pranidipine), phenylalkylamine (e.g., verapamil), benzothiazepines (e.g., diltiazem), mibefradil, bepridil, fluspirilene, and fendiline; diuretics such as high ceiling loop diuretics (e.g., furosemide, ethacrynic acid, torsemide and bumetanide), thiazides (e.g., hydrochlorothiazide acid), carbonic anhydrase inhibitors (e.g., acetazolamide and methazolamide), potassium-sparing diuretics (e.g., aldosterone antagonists: spironolactone, and epithelial sodium channel blockers: amiloride and triamterene), and calcium-sparing diuretics, and pharmaceutically acceptable salts, acids or derivatives of any of the above.

For insulin-related disorders or metabolic syndrome, the administration of a composition (e.g., an IL-22 Fc fusion protein or a pharmaceutical composition thereof) can be combined with or supplement the administration of various therapeutic agents. In the case of Type I diabetes (insulin-dependent diabetes mellitus or IDDM), the IL-22 Fc fusion protein described herein can be combined with one or more of regular insulin replacement therapy (including rapid-acting and long-acting insulin), immunosuppression treatment, islet transplantation and stem cell therapy. In one embodiment, the regular insulin replacement therapy includes, without limitation, regular insulin (e.g., HUMULIN R®, NOVOLIN R®), insulin isophane (e.g., HUMULIN N®, NOVOLIN N®), insulin lispro (e.g., HUMALOG®), insulin aspart (e.g., NOVOLOG®), insulin glargine (e.g., LANTUS®), and insulin detemir (e.g., LEVEMIR®). In other embodiments, the insulin replacement therapy further includes pramlintide (SYMLIN®).

In the case of Type II diabetes (non-insulin dependent diabetes mellitus or NIDDM) or metabolic syndrome, the composition (e.g., a pharmaceutical composition that includes an IL-22 Fc fusion protein) described herein can be combined with one or more of insulin replacement therapy (as discussed above), an agent to lower glucose production by the liver, an agent to stimulate pancreatic production and release of insulin, an agent that blocks enzymatic break down of carbohydrates, or an agent that increases insulin sensitivity. In one embodiment, the agent to lower glucose production is metformin (e.g., GLUCOPHAGE® and GLUMETZA®). In another embodiment, the agent to stimulate pancreatic production and release of insulin is glipizide (e.g., GLUCOTROL® and GLUCOTROL XL®), glyburide (e.g., DIABETA® and GLYNASE®) or glimepiride (e.g., AMARYL®). In one other embodiment, the agent that blocks enzymatic break down of carbohydrates or increases insulin sensitivity is pioglitazone (e.g., Actos). In another embodiment, the IL-22 Fc fusion protein can be combined with one of the following replacements for metformin: sitagliptin (e.g., JANUVIA®), saxagliptin (e.g., ONGLYZA®), repaglinide (e.g., PRANDIN®) and nateglinide (e.g., STARLIX®), exenatide (e.g., BYETTA®) and liraglutide (e.g., VICTOZA®). In another embodiment, the composition (e.g., a pharmaceutical composition that includes an IL-22 Fc fusion protein) can be combined with an oral hypoglycemic agent, e.g., sulfonylureas.

In the case of gestational diabetes or metabolic syndrome, the composition (e.g., an IL-22 Fc fusion protein or a pharmaceutical composition thereof) described herein can be combined with an oral blood sugar control medication. In one embodiment, the medication is glyburide.

The combination therapy can provide “synergy” and prove “synergistic,” i.e., the effect achieved when the active ingredients used together is greater than the sum of the effects that results from using the compounds separately. A synergistic effect can be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds are administered or delivered sequentially, e.g. by different injections in separate syringes. In general, during alternation therapy, an effective dosage of each active ingredient is administered sequentially, i.e., serially, whereas in combination therapy, effective dosages of two or more active ingredients are administered together.

Such combination therapies noted above encompass combined administration (where two or more therapeutic agents are included in the same or separate formulations), and separate administration, in which case, administration of composition (e.g., an IL-22 Fc fusion protein or a pharmaceutical composition thereof) can occur prior to, simultaneously, and/or following, administration of the additional therapeutic agent or agents. In one embodiment, administration of the composition (e.g., an IL-22 Fc fusion protein or a pharmaceutical composition thereof) and administration of an additional therapeutic agent occur within about one month, or within about one, two or three weeks, or within about one, two, three, four, five, or six days, of each other.

A composition (e.g., an IL-22 Fc fusion protein or a pharmaceutical composition thereof) described herein (and any additional therapeutic agent) can be administered by any suitable means, including parenteral, intrapulmonary, topical and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

A composition (e.g., an IL-22 Fc fusion protein or a pharmaceutical composition thereof) described herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The composition (e.g., an IL-22 Fc fusion protein or a pharmaceutical composition thereof) need not be, but is optionally formulated with one or more agents currently used to prevent or treat the disorder in question. The effective amount of such other agents depends on the amount of the fusion protein present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of a composition (e.g., an IL-22 Fc fusion protein or a pharmaceutical composition thereof) of the invention (when used alone or in combination with one or more other additional therapeutic agents) will depend on the type of disease to be treated, the type of Fc region, the severity and course of the disease, whether the fusion protein is administered for preventive or therapeutic purposes, previous therapy, the patients clinical history and response to the IL-22 Fc fusion protein, and the discretion of the attending physician. The composition (e.g., an IL-22 Fc fusion protein or a pharmaceutical composition thereof) is suitably administered to the patient at one time or over a series of treatments. Depending on the type and severity of the disease, about 1 μg/kg to 15 mg/kg (e.g., 0.1 mg/kg-10 mg/kg) or about 0.1 μg/kg to 1.5 mg/kg (e.g., 0.01 mg/kg-1 mg/kg) of the IL-22 Fc fusion protein can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the IL-22 Fc fusion protein would be in the range from about 0.05 mg/kg to about 10 mg/kg. Certain other dosages include the range from about 0.01 mg/kg to about 10 mg/kg, about 0.02 mg/kg to about 10 mg/kg, and about 0.05 mg/kg to about 10 mg/kg. Thus, one or more doses of about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 2.0 mg/kg, 3.0 mg/kg, 4.0 mg/kg, 5 mg/kg, 6 mg/kg, 7 mg/kg, 8 mg/kg, 9 mg/kg or 10 mg/kg (or any combination thereof) may be administered to the patient. For topical wound healing, one or more doses of about 0.001 mg/cm² to about 10 mg/cm² wound area, about 0.05 mg/cm² to about 5 mg/cm² wound area, about 0.01 mg/cm² to about 1 mg/cm² wound area, about 0.05 mg/cm² to about 0.5 mg/cm² wound area, about 0.01 mg/cm² to about 0.5 mg/cm² wound area, about 0.05 mg/cm² to about 0.2 mg/cm² wound area, or about 0.1 mg/cm² to about 0.5 mg/cm² wound area (or any combination thereof) may be administered to the patient. In certain embodiments, one or more doses of about 0.01 mg/cm², 0.02 mg/cm², 0.03 mg/cm², 0.04 mg/cm², 0.05 mg/cm², 0.06 mg/cm², 0.07 mg/cm², 0.08 mg/cm², 0.09 mg/cm², 0.1 mg/cm², 0.15 mg/cm², 0.2 mg/cm², 0.25 mg/cm², 0.3 mg/cm², 0.4 mg/cm², or 0.5 mg/cm² wound area may be administered to the patient. Such doses may be administered intermittently, e.g., every week or every three weeks (e.g., such that the patient receives from about two to about twenty, or e.g., about six doses of the IL-22 Fc fusion protein). An initial higher loading dose, followed by one or more lower doses may be administered. However, other dosage regimens may be useful. The progress of this therapy is easily monitored by conventional techniques and assays.

It is understood that any of the above formulations or therapeutic methods may be carried out using conjugate of the invention in place of or in addition to an IL-22 Fc fusion protein.

### F. Pharmaceutical Formulations

The invention also provides compositions (e.g., pharmaceutical compositions) that include IL-22 Fc fusion proteins for use in the methods, uses, articles of manufacture, and kits described herein. Any of the IL-22 Fc fusion proteins described herein can be used in the compositions. In some embodiments, any of the pharmaceutical compositions described in International Patent Application No. PCT/US2019/015268 may be used in the methods, uses, articles of manufacture, and kits described herein.

Pharmaceutical formulations can be prepared using standard methods known in the art by mixing the active ingredient having the desired degree of purity with one or more optional pharmaceutically acceptable carriers (see, e.g., Remington's Pharmaceutical Sciences 16th edition, Osol, A. Ed. (1980) and Remington's Pharmaceutical Sciences 20th edition, ed. A. Gennaro, 2000, Lippincott, Williams & Wilkins, Philadelphia, Pa.), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Optionally, the formulation contains a pharmaceutically acceptable salt, preferably sodium chloride, and preferably at about physiological concentrations.

Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, benzalkonium chloride and propylparaben are preferred preservatives. Optionally, the formulations of the invention can include a pharmaceutically acceptable surfactant, e.g., at a concentration of 0.005 to 0.02%.

The formulation herein can also contain more than one active compound as necessary for the particular indication being treated, preferably those with complementary activities that do not adversely affect each other. Such molecules are suitably present in combination in amounts that are effective for the purpose intended.

Exemplary lyophilized formulations are described in U.S. Pat. No. 6,267,958. Aqueous formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. For example, it may be desirable to further provide a steroid, TNF antagonist or other anti-inflammatory therapeutics. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the IL-22 Fc fusion protein, which matrices are in the form of shaped articles, e.g., films or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and .gamma. ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S-S bond formation through thio-disulfide interchange, stabilization may be achieved by modifying sulfhydryl residues, lyophilizing from acidic solutions, controlling moisture content, using appropriate additives, and developing specific polymer matrix compositions.

A pharmaceutical composition for topical administration can be formulated, for example, in the form of a topical gel. See e.g., U.S. Pat. Nos. 4,717,717; 5,130,298; 5,427,778; 5,457,093; 5,705,485; 6,331,309; and WO2006/138,468. In certain embodiments, the composition can be formulated in the presence of cellulose derivatives. In certain other embodiments, the topical formulation can be reconstituted from lyophilized formulation with sufficient buffer or diluent before administration. In certain embodiments, IL-22 polypeptide or IL-22 Fc fusion protein is formulated for topical administration to a subject having a defect in epithelial wound healing. In certain particular embodiments, the epithelial wound healing occurs in the skin. In certain other particular embodiments, the subject is a human having a defect in wound healing. In certain other embodiments, the topical formulation comprising an IL-22 Fc fusion protein of the invention can be used to improve wound healing after internal or external surgical incisions.

In one embodiment of the invention, an IL-22 polypeptide or IL-22 Fc fusion protein for use in accelerating, promoting or improving wound healing is in a formulation of a topical gel, e.g., in a pre-filled syringe or container, or alternatively, the compound of the invention can be mixed with a gel matrix right before topical administration to a patient. In certain embodiments, an additional therapeutic agent is also administered topically, either concurrently or sequentially. Other routes of administration can also be optionally used, e.g., administered by any suitable means, including but not limited to, parenteral, subcutaneous, intraperitoneal, intrapulmonary, intracerobrospinal, subcutaneous, intra-articular, intrasynovial, intrathecal, oral, and intranasal administration. Parenteral infusions include intramuscular, intravenous, intraarterial, intraperitoneal, or subcutaneous administration.

Typically for wound healing, an IL-22 Fc fusion protein is formulated for site-specific delivery. When applied topically, the IL-22 Fc fusion protein is suitably combined with other ingredients, such as carriers and/or adjuvants. There are no limitations on the nature of such other ingredients, except that they must be pharmaceutically acceptable and efficacious for their intended administration, and cannot degrade the activity of the active ingredients of the composition. Examples of suitable vehicles include ointments, creams, gels, sprays, or suspensions, with or without purified collagen. The compositions also may be impregnated into sterile dressings, transdermal patches, plasters, and bandages, optionally in liquid or semi-liquid form. An oxidized regenerated cellulose/collagen matrices can also be used, e.g., PROMOGRAN Matrix Wound Dressing or PROMOGRAN PRISMA MATRIX.

For obtaining a gel formulation, the IL-22 polypeptide or IL-22 Fc fusion protein formulated in a liquid composition may be mixed with an effective amount of a water-soluble polysaccharide or synthetic polymer to form a gel (e.g., a gelling agent) such as polyethylene glycol to form a formulation of the proper viscosity to be applied topically. The polysaccharide or gelling agent that may be used includes, for example, cellulose derivatives such as etherified cellulose derivatives, including alkyl celluloses, hydroxyalkyl celluloses, and alkylhydroxyalkyl celluloses, for example, methylcellulose, hydroxyethyl cellulose, carboxymethyl cellulose, hydroxypropyl methylcellulose, and hydroxypropyl cellulose; Sodium carboxymethyl cellulose; POE-POP block polymers: poloxamer USP in various grades; Hyaluronic acid; Polyacrylic acid such as carbopol 940; starch and fractionated starch; agar; alginic acid and alginates; gum Arabic; pullullan; agarose; carrageenan; dextrans; dextrin; fructans; inulin; mannans; xylans; arabinans; chitosans; glycogens; glucans; and synthetic biopolymers; as well as gums such as xanthan gum; guar gum; locust bean gum; gum Arabic; tragacanth gum; and karaya gum; and derivatives, combinations and mixtures thereof. In one embodiment of the invention, the gelling agent herein is one that is, e.g., inert to biological systems, nontoxic, simple to prepare, and/or not too runny or viscous, and will not destabilize the IL-22 polypeptide or IL-22 Fc fusion held within it.

In certain embodiments of the invention, the polysaccharide is an etherified cellulose derivative, in another embodiment one that is well defined, purified, and listed in USP, e.g., methylcellulose and the hydroxyalkyl cellulose derivatives, such as hydroxypropyl cellulose, hydroxyethyl cellulose, and hydroxypropyl methylcellulose (all referred to as cellulosic agents). In some embodiments, the polysaccharide is hydroxyethyl methylcellulose or hydroxypropyl methylcellulose.

The polyethylene glycol useful for gelling is typically a mixture of low and high molecular weight polyethylene glycols to obtain the proper viscosity. For example, a mixture of a polyethylene glycol of molecular weight 400-600 with one of molecular weight 1500 would be effective for this purpose when mixed in the proper ratio to obtain a paste.

The term “water soluble” as applied to the polysaccharides and polyethylene glycols is meant to include colloidal solutions and dispersions. In general, the solubility of the cellulose derivatives is determined by the degree of substitution of ether groups, and the stabilizing derivatives useful herein should have a sufficient quantity of such ether groups per anhydroglucose unit in the cellulose chain to render the derivatives water soluble. A degree of ether substitution of at least 0.35 ether groups per anhydroglucose unit is generally sufficient. Additionally, the cellulose derivatives may be in the form of alkali metal salts, for example, the Li, Na, K, or Cs salts.

In certain embodiments, methylcellulose is employed in the gel, for example, it comprises about 1-5%, or about 1%, about 2%, about 3%, about 4% or about 5%, of the gel and the IL-22 Fc fusion protein is present in an amount of about 50-2000 μg, 100-2000 μg, or 100-1000 μg per ml of gel. In certain embodiments, the effective amount of IL-22 Fc fusion protein for wound healing by topical administration can be about 25 μg to about 500 μg, about 50 μg to about 300 μg, about 100 μg to about 250 μg, about 50 μg to about 250 μg, about 50 μg to about 150 μg, about 75 μg, about 100 μg, about 125 μg, about 150 μg, about 175 μg, about 200 μg, about 225 μg, about 250 μg, about 300 μg, or about 350 μg, per cm² wound area.

The formulations to be used for in vivo administration are generally sterile. Sterility may be readily accomplished, e.g., by filtration through sterile filtration membranes.

The compounds of the invention for prevention or treatment of a cardiovascular disease or condition, metabolic syndrome, acute endotoxemia or sepsis, or diabetes are typically administered by intravenous injection.

Other methods of administration can also be used, which includes but is not limited to, topical, parenteral, as intravenous, subcutaneous, intraperitoneal, intrapulmonary, intranasal, ocular, intraocular, intravitreal, intralesional, intracerobrospinal, intra-articular, intrasynovial, intrathecal, oral, or inhalation administration. Parenteral infusions include intramuscular, intravenous, intraarterial, intraperitoneal, or subcutaneous administration. In addition, the compounds described herein are administered to a human subject, in accord with known methods, such as intravenous administration as a bolus or by continuous infusion over a period of time.

### G. Articles of Manufacture and Kits

In another aspect of the invention, an article of manufacture or kit containing materials useful for the methods and uses described herein is provided. The article of manufacture may include any of the compositions (e.g., IL-22 Fc fusion proteins or compositions thereof (e.g., pharmaceutical compositions)) provided herein. The articles of manufacture and kits may include a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). In some embodiments, at least one active agent in the composition is an IL-22 Fc fusion protein. The label or package insert indicates that the composition is used for treating the condition of choice. In some embodiments, the article of manufacture or the containers are protected from light. The articles of manufacture can include any of the compositions (e.g., pharmaceutical compositions) described herein.

The invention provides a kit including any of the IL-22 Fc fusion proteins described herein and instructions to administer the IL-22 Fc fusion protein to a subject suffering from or at risk of an IL-22 associated disease such as IBD (e.g., UC (e.g., moderate to severe UC) or Crohn's disease), GVHD, hidradenitis suppurativa, COPD, or nonalcoholic fatty acid liver disease (e.g., NASH) in accordance with any of the methods described herein.

For example, the invention provides a kit including an IL-22 Fc fusion protein and instructions to administer the IL-22 Fc fusion protein to a subject having an IBD in a method including administering to the subject an IL-22 Fc fusion protein in a dosing regimen including at least a first dosing cycle, wherein the dosing cycle includes between two and ten doses, and wherein a total of about 60 μg/kg to about 900 μg/kg of the IL-22 Fc fusion protein is administered to the subject in the first dosing cycle. In some embodiments, a total of about 90 μg/kg, about 180 μg/kg, about 270 μg/kg, about 360 μg/kg, or about 540 μg/kg of the IL-22 Fc fusion protein is administered to the subject in the first dosing cycle. In some embodiments, the length of the dosing cycle is between about 5 weeks and about 15 weeks. In some embodiments, the length of the dosing cycle is between 8 weeks and 12 weeks. In some embodiments, the length of the dosing cycle is about 8 weeks.

In another example, the invention provides a kit including an IL-22 Fc fusion protein and instructions to administer the IL-22 Fc fusion protein to a subject having an IBD in a method including administering to the subject an IL-22 Fc fusion protein in a dosing regimen including a dosing cycle, wherein the dosing cycle includes between two and six doses, and wherein a total of about 30 μg/kg to about 720 μg/kg of the IL-22 Fc fusion protein is administered to the subject in the dosing cycle. In some embodiments, the length of the dosing cycle is between about 5 weeks and about 15 weeks. In some embodiments, the length of the dosing cycle is between 8 weeks and 12 weeks. In some embodiments, the length of the dosing cycle is about 10 weeks.

In some embodiments of any of the preceding kits, the dose(s) are to be administered to the subject every week (q1w), every two weeks (q2w), every three weeks (q3w), every four weeks, (q4w), every five weeks (q5w), every six weeks (q6w), every seven weeks (q7w), every eight weeks (q8w), every nine weeks (q9w), every ten weeks (q10w), every 12 weeks (q12w), every fourteen weeks (q14w), every sixteen weeks (q16w), every eighteen weeks (q18w), or every twenty weeks (q20w). For example, in some embodiments, the doses are to be administered to the subject every week (q1w), every two weeks (q2w), every four weeks (q4w), or every six weeks (q6w). In some embodiments, a total of about 540 μg/kg of the IL-22 Fc fusion protein is to be administered to the subject in the dosing cycle (e.g., about 54 μg/kg q1w, about 90 μg/kg q2w, about 180 μg/kg q4w, or about 270 μg/kg q6w in a ten-week dosing cycle). In some embodiments, a total of about 360 μg/kg of the IL-22 Fc fusion protein is to be administered to the subject in the dosing cycle (e.g., about 36 μg/kg q1w, about 60 μg/kg q2w, about 120 μg/kg q4w, or about 180 μg/kg q6w in a ten-week dosing cycle). In some embodiments, a total of about 180 μg/kg of the IL-22 Fc fusion protein is to be administered to the subject in the dosing cycle (e.g., about 18 μg/kg q1w, about 30 μg/kg q2w, about 60 μg/kg q4w, or about 90 μg/kg q6w in a ten-week dosing cycle). In some embodiments, a total of about 90 μg/kg of the IL-22 Fc fusion protein is to be administered to the subject in the dosing cycle (e.g., about 9 μg/kg q1w, about 15 μg/kg q2w, about 30 μg/kg q4w, or about 45 μg/kg q6w in a ten-week dosing cycle).

In some embodiments of any of the preceding kits, the dosing cycle (e.g., the first dosing cycle) includes a first dose (C1D1) and a second dose (C1D2) of the IL-22 Fc fusion protein. In some embodiments, the dosing cycle consists of the C1D1 and the C1D2. In some embodiments, the C1D1 and the C1D2 are each between about 45 μg/kg to about 135 μg/kg. In some embodiments, the C1D1 and the C1D2 are each about 90 μg/kg. In some embodiments, the method includes administering to the subject the C1D1 and the C1D2 on or about Weeks 0 and 6, respectively, of the dosing cycle.

In other embodiments of any of the preceding kits, the dosing cycle (e.g., the first dosing cycle) includes a first dose (C1D1), a second dose (C1D2), and a third dose (C1D3) of the IL-22 Fc fusion protein. In some embodiments, the dosing cycle consists of the C1D1, the C1D2, and the C1D3. In some embodiments, the C1D1, the C1D2, and the C1D3 are each between about 15 μg/kg to about 90 μg/kg. In some embodiments, the C1D1, the C1D2, and the C1D3 are each between about 20 μg/kg to about 40 μg/kg. In some embodiments, the C1D1, the C1D2, and the C1D3 are each about 30 μg/kg. In some embodiments, the C1D1, the C1D2, and the C1D3 are each between about 50 μg/kg to about 70 μg/kg. In some embodiments, the C1D1, the C1D2, and the C1D3 are each about 60 μg/kg. In some embodiments, the C1D3 are each about 90 μg/kg. In some embodiments, the method includes administering the C1D1, the C1D2, and the C1D3 on or about Weeks 0, 4, and 8, respectively, of the dosing cycle (e.g., the first dosing cycle). In other embodiments, the method includes administering the C1D1, the C1D2, and the C1D3 on or about Weeks 1, 4, and 8, respectively, of the dosing cycle (e.g., the first dosing cycle).

In still other embodiments of any of the preceding kits, the dosing cycle (e.g., the first dosing cycle)includes a first dose (C1D1), a second dose (C1D2), a third dose (C1D3), a fourth dose (C1D4), a fifth dose (C1D5), and a sixth dose (C1D6) of the IL-22 Fc fusion protein. In some embodiments, the dosing cycle consists of the C1D1, the C1D2, the C1D3, the C1D4, the C1D5, and the C1D6. In some embodiments, the C1D1, the C1D2, the C1D3, the C1D4, the C1D5, and the C1D6 are each between about 30 μg/kg to about 90 μg/kg. In some embodiments, the C1D1, the C1D2, the C1D3, the C1D4, the C1D5, and the C1D6 are each between about 50 μg/kg to about 70 μg/kg. In some embodiments, the C1D1, the C1D2, the C1D3, the C1D4, the C1D5, and the C1D6 are each about 60 μg/kg. In some embodiments, the method includes administering the C1D1, the C1D2, the C1D3, the C1D4, the C1D5, and the C1D6 on or about Weeks 0, 2, 4, 6, 8, and 10, respectively, of the dosing cycle.

In some embodiments of any of the preceding kits, the dosing regimen further includes a further (e.g., a second) dosing cycle. In some embodiments, the length of the further dosing cycle is between about 10 weeks and about 40 weeks. In some embodiments, the length of the further dosing cycle is between about 15 weeks and about 25 weeks. In some embodiments, the length of the further dosing cycle is about 20 weeks. In other embodiments, the second dosing cycle continues indefinitely or until clinical remission. In some embodiments, the further (e.g., second) dosing cycle is stopped following the clinical remission, and then restarted following a relapse of the IBD. In some embodiments, the further dosing cycle includes a first dose (C2D1), a second dose (C2D2), and a third dose (C2D3) of the IL-22 Fc fusion protein. In some embodiments, the C2D1, the C2D2, and the C2D3 are each between about 30 μg/kg to about 90 μg/kg. In some embodiments, the C2D1, the C2D2, and the C2D3 are each between about 50 μg/kg to about 70 μg/kg. In some embodiments, the C2D1, the C2D2, and the C2D3 are each about 60 μg/kg. In some embodiments, the method includes administering the C2D1, the C2D2, and the C2D3 on or about Weeks 4, 12, and 20, respectively, of the further dosing cycle.

In some embodiments of any of the preceding kits, the doses of the further dosing cycle are administered to the subject every eight weeks (q8w). In some embodiments, each dose of the further dosing cycle is between about 30 μg/kg to about 90 μg/kg (e.g., about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, or about 90 μg/kg). In some embodiments, each dose of the further dosing cycle is about 60 μg/kg.

In some embodiments of any of the preceding kits, the subject is not administered the IL-22 Fc fusion protein for a time period between any two dosing cycles (e.g., the subject is not administered the IL-22 Fc fusion protein for a time period between a first dosing cycle and a further (e.g., second) dosing cycle). In some embodiments, the subject is not administered the IL-22 Fc fusion protein for a time period of about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, three months, four months, five months, six months, seven months, eight months, nine months, ten months, eleven months, twelve months, two years, three years, four years, five years, or longer between any two dosing cycles (e.g., between a first dosing cycle and a further (e.g., second) dosing cycle)).

For example, in some embodiments, the first dose of the further (e.g., second) dosing cycle is administered to the subject about 6 weeks to about 10 weeks after the last dose of the preceding (e.g., first) dosing cycle. In some embodiments, the first dose of the further (e.g., second) dosing cycle is administered to the subject about 7 weeks to about 9 weeks after the last dose of the preceding (e.g., first) dosing cycle. In some embodiments, the first dose of the further (e.g., second) dosing cycle is administered to the subject about 8 weeks after the last dose of the preceding (e.g., first) dosing cycle.

In another example, the invention provides a kit including an IL-22 Fc fusion protein and instructions to administer the IL-22 Fc fusion protein to a subject having an IBD in a method including administering to the subject an IL-22 Fc fusion protein in a dosing regimen, the dosing regimen comprising administering to the subject an IL-22 Fc fusion protein every four weeks (q4w) until the subject has a clinical remission of the IBD. In yet another example, the invention provides a kit including an IL-22 Fc fusion protein and instructions to administer the IL-22 Fc fusion protein to a subject having an inflammatory bowel disease (IBD) in a method including administering to the subject an IL-22 Fc fusion protein in a dosing regimen comprising a dosing cycle having a length of about 8 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 30 μg/kg, and wherein the C1D1, the C1D2, and the C1D3 are administered to the subject on or about Weeks 1, 4, and 8, respectively, of the dosing cycle. In some embodiments, the IBD is UC. In other embodiments, the IBD is Crohn's disease.

In another example, the invention provides a kit including an IL-22 Fc fusion protein and instructions to administer the IL-22 Fc fusion protein to a subject having an inflammatory bowel disease (IBD) in a method including administering to the subject an IL-22 Fc fusion protein in a dosing regimen comprising a dosing cycle having a length of about 8 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 60 μg/kg, and wherein the C1D1, the C1D2, and the C1D3 are administered to the subject on or about Weeks 1, 4, and 8, respectively, of the dosing cycle. In some embodiments, the IBD is UC. In other embodiments, the IBD is Crohn's disease.

In a further example, the invention provides a kit including an IL-22 Fc fusion protein and instructions to administer the IL-22 Fc fusion protein to a subject having an inflammatory bowel disease (IBD) in a method including administering to the subject an IL-22 Fc fusion protein in a dosing regimen comprising a dosing cycle having a length of about 8 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 90 μg/kg, and wherein the C1D1, the C1D2, and the C1D3 are administered to the subject on or about Weeks 1, 4, and 8, respectively, of the dosing cycle. In some embodiments, the IBD is UC. In other embodiments, the IBD is Crohn's disease.

In yet another example, the invention provides a kit including an IL-22 Fc fusion protein and instructions to administer the IL-22 Fc fusion protein to a subject having an inflammatory bowel disease (IBD) in a method including administering to the subject an IL-22 Fc fusion protein in a dosage regimen comprising at least a first dosing cycle and a second dosing cycle, wherein: (a) the first dosing cycle has a length of about 8 weeks and comprises a first dose (C1D1), a second dose (C1D2), and a third dose (C1D3) of the IL-22 Fc fusion protein, wherein the C1D1, the C1D2, and the C1D3 are each about 30 μg/kg, about 60 μg/kg, or about 90 μg/kg, and wherein the C1D1, the C1D2, and the C1D2 are administered to the subject on or about Weeks 1, 4, and 8, respectively, of the first dosing cycle; and (b) the second dosing cycle continues indefinitely or until clinical remission, and comprises administering about 60 μg/kg of the IL-22 Fc fusion protein to the subject every 8 weeks (q8w), wherein the first dose of the second dosing cycle is administered to the subject about 8 weeks after the last dose of the first dosing cycle. In some embodiments, the IBD is UC. In other embodiments, the IBD is Crohn's disease. For example, the invention provides a kit including an IL-22 Fc fusion protein and instructions to administer the IL-22 Fc fusion protein to a subject having an inflammatory bowel disease (IBD) in a method including administering to the subject an IL-22 Fc fusion protein in a dosing regimen including a dosing cycle having a length of about 10 weeks, wherein the dosing cycle includes a first dose (C1D1) and a second dose (C1D2) of the IL-22 Fc fusion protein, wherein the C1D1 and the C1D2 are each about 90 μg/kg, and wherein the C1D1 and C1D2 are administered to the subject on or about Weeks 0 and 6, respectively, of the dosing cycle.

In another example, the invention provides a kit including an IL-22 Fc fusion protein and instructions to administer the IL-22 Fc fusion protein to a subject having an IBD in a method including administering to the subject an IL-22 Fc fusion protein in a dosing regimen including a dosing cycle having a length of about 10 weeks, wherein the dosing cycle includes a first dose (C1D1), a second dose (C1D2), and a third dose (C1D3) of the IL-22 Fc fusion protein, wherein the C1D1, the C1D2, and the C1D3 are each about 30 μg/kg, and wherein the C1D1, the C1D2, and the C1D3 are administered to the subject on or about Weeks 0, 4, and 8, respectively, of the dosing cycle.

In yet another example, the invention provides a kit including an IL-22 Fc fusion protein and instructions to administer the IL-22 Fc fusion protein to a subject having an IBD in a method including administering to the subject an IL-22 Fc fusion protein in a dosing regimen including a dosing cycle having a length of about 10 weeks, wherein the dosing cycle includes a first dose (C1D1), a second dose (C1D2), and a third dose (C1D3) of the IL-22 Fc fusion protein, wherein the C1D1, the C1D2, and the C1D3 are each about 60 μg/kg, and wherein the C1D1, the C1D2, and the C1D3 are administered to the subject on or about Weeks 0, 4, and 8, respectively, of the dosing cycle.

In a further example, the invention provides a kit including an IL-22 Fc fusion protein and instructions to administer the IL-22 Fc fusion protein to a subject having an IBD in a method including administering to the subject an IL-22 Fc fusion protein in a dosing regimen including a dosing cycle having a length of about 10 weeks, wherein the dosing cycle includes a first dose (C1D1), a second dose (C1D2), a third dose (C1D3), a fourth dose (C1D4), a fifth dose (C1D5), and a sixth dose (C1D6) of the

IL-22 Fc fusion protein, wherein the C1D1, C1D2, the C1D3, the C1D4, the C1D5, and the C1D6 are each about 60 μg/kg, and wherein the C1D1, the C1D2, the C1D3, the C1D4, the C1D5, and the C1D6 are administered to the subject on or about Weeks 0, 2, 4, 6, 8, and 10, respectively, of the dosing cycle.

In a still further example, the invention provides a kit including an IL-22 Fc fusion protein and instructions to administer the IL-22 Fc fusion protein to a subject having an IBD in a method including administering to the subject an IL-22 Fc fusion protein in a dosage regimen including at least a first dosing cycle and a second dosing cycle, wherein: (a) the first dosing cycle has a length of about 10 weeks and includes a first dose (C1D1) and a second dose (C1D2) of the IL-22 Fc fusion protein, wherein the C1D1 and the C1D2 are each about 90 μg/kg, and wherein the C1D1 and the C1D2 are administered to the subject on or about Weeks 0 and 6, respectively, of the first dosing cycle; and (b) the second dosing cycle has a length of about 20 weeks and includes a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are administered on or about Weeks 4, 12, and 20, respectively, of the second dosing cycle.

In a still further example, the invention provides a kit including an IL-22 Fc fusion protein and instructions to administer the IL-22 Fc fusion protein to a subject having an IBD in a method including administering to the subject an IL-22 Fc fusion protein in a dosage regimen including at least a first dosing cycle and a second dosing cycle, wherein: (a) the first dosing cycle has a length of about 10 weeks and includes a first dose (C1D1), a second dose (C1D2), and a third dose (C1D3) of the IL-22 Fc fusion protein, wherein the C1D1, the C1D2, and the C1D3 are each about 30 μg/kg, and wherein the C1D1, the C1D2, and the C1D2 are administered to the subject on or about Weeks 0, 4, and 8, respectively, of the first dosing cycle; and (b) the second dosing cycle has a length of about 20 weeks and includes a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are administered on or about Weeks 4, 12, and 20, respectively, of the second dosing cycle.

In yet another example, the invention provides a kit including an IL-22 Fc fusion protein and instructions to administer the IL-22 Fc fusion protein to a subject having an IBD in a method including administering to the subject an IL-22 Fc fusion protein in a dosage regimen including at least a first dosing cycle and a second dosing cycle, wherein: (a) the first dosing cycle has a length of about 10 weeks and includes a first dose (C1D1), a second dose (C1D2), and a third dose (C1D3) of the IL-22 Fc fusion protein, wherein the C1D1, the C1D2, and the C1D3 are each about 60 μg/kg, and wherein the C1D1, the C1D2, and the C1D2 are administered to the subject on or about Weeks 0, 4, and 8, respectively, of the first dosing cycle; and (b) the second dosing cycle has a length of about 20 weeks and includes a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are administered on or about Weeks 4, 12, and 20, respectively, of the second dosing cycle.

In yet another example, the invention provides a kit including an IL-22 Fc fusion protein and instructions to administer the IL-22 Fc fusion protein to a subject having an IBD in a method including administering to the subject an IL-22 Fc fusion protein in a dosage regimen including at least a first dosing cycle and a second dosing cycle, wherein: (a) the first dosing cycle has a length of about 10 weeks and includes a first dose (C1D1), a second dose (C1D2), a third dose (C1D3), a fourth dose (C1D4), a fifth dose (C1D5), and a sixth dose (C1D6) of the IL-22 Fc fusion protein, wherein the C1D1, the C1D2, the C1D3, the C1D4, the C1D5, and the C1D6 are each about 60 μg/kg, and wherein the C1D1, the C1D2, the C1D3, the C1D4, the C1D5, and the C1D6 are administered to the subject on or about Weeks 0, 2, 4, 5, 8, and 10, respectively, of the dosing cycle; and (b) the second dosing cycle has a length of about 20 weeks and includes a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are administered on or about Weeks 4, 12, and 20, respectively, of the second dosing cycle.

In another example, provided herein is a kit including an IL-22 Fc fusion protein and instructions to use the IL-22 Fc fusion protein in a method of treating or preventing graft versus host disease (GVHD) in the subject that includes administering to the subject the IL-22 Fc fusion protein in a dosing regimen comprising a dosing cycle, wherein the dosing cycle comprises between two and ten doses, and wherein a total of about 60 μg/kg to about 900 μg/kg of the IL-22 Fc fusion protein is administered to the subject in the dosing cycle.

In another example, provided herein is a kit including an IL-22 Fc fusion protein and instructions to use the IL-22 Fc fusion protein in a method of treating or preventing graft versus host disease (GVHD) in the subject that includes administering to the subject an IL-22 Fc fusion protein in a dosing regimen comprising a dosing cycle having a length of about 96 days, wherein the dosing cycle comprises a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein, wherein the C1D1, the C1D2, the C1D3, the C1D4, the C1D5, the C1D6, the C1D7, and the C1D8 are each about 60 μg/kg, wherein the C1D1 is administered to the subject about 3 (±2) days prior to allo-HSCT, the C1D2 is administered about eleven days after the allo-HSCT, and the C1D3, C1D4, C1D5, C1D6, C1D7, and C1D8 are administered to the subject every two weeks (q2w) following administration of the C1D2.

In any of the preceding methods, the GVHD may be acute or chronic GVHD. In particular embodiments, the GVHD is acute GVHD. In particular embodiments, the kit includes instructions for a method of preventing GVHD. In some embodiments, the GVHD is intestinal GVHD.

It is understood that any of the above articles of manufacture may include a conjugate of the invention in place of or in addition to an IL-22 Fc fusion protein.

### EXAMPLES

The following are examples of methods and compositions of the invention. It is understood that various other embodiments may be practiced, given the general description provided above, and the examples are not intended to limit the scope of the claims.

### Example 1: Safety, Pharmacokinetics, and Pharmacodynamics of IL-22 Fc Fusion Protein as a Therapy for Epithelial Injury in Healthy Volunteers

In this phase 1, first-in-human study, IL-22 Fc fusion protein was administered to healthy volunteers as a single dose to characterize the safety, tolerability, and pharmacokinetics (PK) of intravenous (IV) and subcutaneous (SC) administration. In addition, the effect on various serum PD biomarkers was assessed to demonstrate evidence of target engagement.

### A. Methods

### Trial Design

This trial (EUDRACT: 2014-002252-10) was a randomized, observer-blinded, placebo-controlled, single-dose-escalation study in healthy volunteers at a single phase 1 unit in Belgium. Initially, subjects were randomly allocated into the first IV dose cohort (1 μg/kg). The safety monitoring committee (SMC) made dose-escalation decisions for subsequent cohorts after review of the cumulative clinical and safety data of each cohort (FIG. 1). No subject could be included in more than one cohort. Subjects were followed for 57 days to monitor safety, pharmacokinetics, and pharmacodynamic biomarkers.

### Screening, Randomization, Blinding, and Dose Escalation

Screening occurred within 28 days before randomization. Subjects were randomized using SAS software (Version 9.3; SAS Institute, Inc., Cary, N.C.) and assigned a unique identification number from the master randomization list by the site pharmacist. All subjects and study site personnel were blinded to treatment assignment throughout the study. Subjects in IV cohorts were confined for 3 days (Day −1 through Day 2) and subjects in SC cohorts were confined for 5 days (Day −1 through Day 4). Single doses were administered on Day 1 and follow-up assessments occurred through Day 57, corresponding to the predicted time for five half-lives. After the last subject in the 10 μg/kg (IV) cohort completed Day 15 assessments, PK samples were analyzed for the first four IV cohorts to inform dose-escalation decisions. Dose-escalation decisions were made according to the protocol in consultation with the sponsor's safety monitoring committee, the site investigator, and independent experts as needed.

### Participants

Participants were healthy volunteers, aged 18-50 years, with a body mass index (BMI) of 18-32 kg/m², a weight of 40-120 kg, and unremarkable medical history, 12-lead electrocardiogram (ECG), and vital signs. Concomitant medications and over-the-counter supplements were generally not allowed unless deemed acceptable by both the investigator and sponsor. Key exclusion criteria related to on-target skin effects and potential tumor promotion included: a history of psoriasis, psoriatic arthritis, or atopic dermatitis requiring treatment within the past year; any eczematous skin disorders requiring treatment within the past year; rosacea, or any other inflammatory skin disorders; a history of cancer; or a known family history of gastrointestinal and/or colon cancer defined as one first-degree relative or two second-degree relatives.

### Safety Outcome Measures

The study examined the incidence, nature, and severity of adverse events (AEs), serious AEs (SAEs), dose-limiting adverse events, and deaths. The site investigator monitored and recorded changes in vital signs, physical findings, clinical laboratory results, and ECGs. Severity of skin effects was assessed using visual analog scales for pruritus, erythema, and hypermelanosis; tolerability was assessed on 0 to 10 severity scales where 10 represented the worst itch possible or unbearable distress (Reich et al. J. Immunol. 185:5531-5538, 2010). The investigator also evaluated the location and total body surface involvement of each event. Some subjects were referred to the study dermatologist for diagnosis and treatment. In some subjects, areas of affected skin were biopsied using standard dermatologic technique, and a local laboratory fixed and stained tissues using standard methods.

### Pharmacokinetic Outcome Measures

Serum PK samples were collected predose, and on Day 1 at 0.5, 4, and 8 hours after the end of infusion, and on Days 2, 3, 5, 8, 11, 15, 22, 29, 43, and 57 or at the early-termination visit. A validated enzyme-linked immunosorbent assay (ELISA) with a lower limit of quantification (LLOQ) of 6 ng/ml was used to quantify serum IL-22 Fc fusion protein concentrations. PK parameters derived from the serum concentration-time profile are defined and listed in Table 8 below. Dose-normalized maximum observed serum concentration (Cmax) and area under the concentration-time curve extrapolated to infinity (AUCinf) were plotted in FIG. 4 and used to evaluate dose proportionality. Bioavailability after SC injection was assessed as described below.

Anti-drug antibody (ADAs) incidence in serum at multiple time points before and after IL-22 Fc fusion protein administration was evaluated using a tiered strategy. Serum samples were initially screened in a validated, bridging enzyme-linked immunosorbent assay (ELISA). Positive samples were confirmed by competitive binding with IL-22 Fc fusion protein and then diluted for use in repeat assays to obtain a value in titer units. Baseline prevalence (the ADA-positive proportion of evaluable subjects at baseline) and post-baseline incidence of ADAs (the proportion of the study population who developed treatment-induced ADAs) to IL-22 Fc fusion protein were listed and summarized. ADA responses and potential correlation of responses to relevant clinical safety and activity endpoints were assessed for all subjects treated during the study. An assay to measure ADAs that neutralized endogenous IL-22 was not available for this study.

### Pharmacodynamic (PD) Outcome Measures

The study measured baseline and after-treatment levels of the circulating biomarkers CRP, REG3A, and SAA, and inflammatory cytokines in serum samples collected at predose and protocol-specified postdose timepoints.

CRP serum concentrations were quantified using the CRP high-sensitivity assay performed at Covance by immunonephelometry using the Siemens BNII Nephelometer.

REG3A serum concentrations were measured by a qualified ELISA using a commercially available kit developed for human from Dynabio (Marseille, France). All samples were run according to manufacturer specifications (LLOQ: 150 μg/mL). Circulating levels of SAA were quantified in serum by a validated ELISA (LLOQ: 0.563 ng/mL) using a commercially available kit recognizing all isoforms of SAA

(Pacific Biomarkers, WA).

To assess levels of inflammatory cytokines in serum, we performed SIMPLEPLEX™ immunoassays (Protein Simple, San Jose, Calif.) to detect IFN-γ, IL-1β, IL-2, and TNF-α and IMPACT Chip platform analysis (Roche Diagnostics GmbH, Penzberg, Germany) to detect IL-6, IL-8, and IL-10. Each multiplex immunoassay was previously qualified for use in sera from both healthy volunteers and inflammatory bowel disease (IBD) patients. Mean cytokine levels (IFN-γ, IL-1β, IL-2, and TNF-α) in healthy volunteer serum for the SIMPLEPLEX™ immunoassays were determined by Protein Simple. Mean cytokine levels, tested in house for IL-6, IL-8, and IL-10, were 2.4 μg/mL, 541 μg/mL, and 22 μg/m L, respectively).

### Study Drug and Dose Selection

IL-22 Fc fusion protein (IL-22Fc) is a fusion protein of human IL-22 linked to the crystallizable fragment (Fc) of human immunoglobulin (Ig) G4 to increase its stability and half-life in vivo. A mutation (N297G) was introduced in the Fc region to minimize potential IgG4 effector function. IL-22 Fc fusion protein (Genentech, Inc., South San Francisco, Calif.) was supplied at 10 mg/mL in 2-mL vials in 10 mM sodium phosphate, 240 mM sucrose, 0.02% (w/v) polysorbate 20, pH 7.1. For IV dosing, IL-22 Fc fusion protein was infused over 1 hour at predefined concentrations per cohort. For SC dosing, IL-22 Fc fusion protein was administered by SC injection into the abdomen.

The first-in-human dose was based on safety factors calculated from the no-adverse-effect level (NOAEL) from an 11-week, repeat-dose toxicity study of IL-22 Fc fusion protein in cynomolgus monkeys.

### Statistical Methods

The sample size for this trial was based on dose-escalation rules and not based on any statistical criteria. Safety data from 4 subjects dosed with active drug were expected to provide adequate information to support dose-escalation decisions. A sufficient number of subjects was screened to ensure approximately 6 subjects in each cohort with 4 subjects dosed with active drug.

Demographic and baseline characteristics including age, sex, race, and weight were summarized using means and standard deviations for continuous variables and proportions for categorical variables, as appropriate. Frequency of TEAEs (number of AEs, number of subjects experiencing an AE, and percentage of subjects experiencing an AE) was summarized by treatment group and the Medical Dictionary for Regulatory Activities (Version 17.0) system organ class and preferred term. Statistical summaries were descriptive (e.g., means, standard deviations, and percentiles). Subjects were grouped for analysis according to treatment actually received. Safety analyses included all randomized subjects who received at least one dose of study drug or placebo.

The PK assessment was performed on serum IL-22 Fc fusion protein concentration-time data using standard noncompartmental analysis in Phoenix WinNonlin software (Version 6.4; Certara, Princeton, N.J.). Actual sampling times were used. The following serum PK parameters were determined: maximum observed concentration (Cₘₐₓ), time to reach Cₘₐₓ (Tₘₐₓ), terminal elimination half-life (biz), area under the concentration-time curve up to last measurable time point (AUC₀₋ₜ), area under the concentration-time curve extrapolated to infinity (AUC_{inf}), clearance (CL) or apparent clearance (CL/F) for drug given in SC, volume of distribution (V) or apparent V (V/F) for drug given in SC, and absolute bioavailability (F).

Absolute bioavailability following SC injection was assessed by comparing the AUC_{inf} (or AUC₀₋ₜ if AUC_{inf} could not be calculated) between SC and IV administration at each dose level. Descriptive statistics were calculated for PK parameters grouped by treatment cohort, including the number of observations (n), mean, and standard deviation (SD) or coefficient of variation (CV).

### B. Results

### Study Flow and Participant Characteristics

One hundred fifty-seven subjects were screened, and 68 entered and completed the study; 44 received IV or SC IL-22 Fc fusion protein and 24 received placebo (FIG. 1B; Table 3). The median age was 25 years for the pooled IL-22 Fc fusion protein cohorts and 26 years for the placebo group. All subjects were male, white, and healthy (Table 3).

The first subject was dosed in November 2014 and the last subject had the last safety follow-up visit in December 2015. In general, the planned number of subjects was randomly allocated for each cohort, with the exceptions of Cohorts C and K. Cohort C (3 μg/kg SC) was expanded by 4 subjects (3 IL-22 Fc fusion protein: 1 placebo; FIG. 1B) to obtain additional information on the local skin effects before escalating to the next dose level. Because of moderate dermatological AEs in Cohort J (120 μg/kg IV) that were considered dose limiting, subjects randomly allocated to Cohort L received 90 μg/kg IV instead of 240 μg/kg IV. In Cohort K (120 μg/kg SC), only the sentinel subject was dosed per investigator discretion because this subject experienced AEs similar to those seen in the 90 μg/kg IV cohort, but these AEs were not dose limiting. Subsequently, Cohort M (240 μg/kg SC) was cancelled because Cohort K was not completed (FIG. 1B; see details below).

TABLE 3
Subject Demographics

| | | IL-22 Fc fusion protein IV | | | |
|---|---|---|---|---|---|
| | Pooled | (A) | (B) | (D) | (F) |
| | placebo IV | 1 μg/kg | 3 μg/kg | 10 μg/kg | 30 μg/kg |
| Demographic | (n = 14) | (n = 2) | (n = 4) | (n = 4) | (n = 4) |
| Sex (male), | 14 (100%) | 2 (100%) | 4 (100%) | 4 (100%) | 4 (100%) |
| n (%) | | | | | |
| Race (white), | 14 (100%) | 2 (100%) | 4 (100%) | 4 (100%) | 4 (100%) |
| n (%) | | | | | |
| Age (years), | 33 (9.3) | 35 (17.7) | 22 (4.1) | 39 (12.8) | 29 (7.6) |
| mean (SD) | | | | | |
| Weight (kg), | 78.0 (12.61) | 81.6 (7.35) | 79.2 (10.68) | 74.7 (4.11) | 84.8 (9.82) |
| mean (SD) | | | | | |
| Height (cm), | 179 (7.1) | 180 (4.9) | 177 (6.8) | 174 (6.7) | 185 (7.9) |
| mean (SD) | | | | | |
| BMI (kg/m²), | 24.2 (2.97) | 25.3 (0.85) | 25.6 (4.40) | 24.8 (3.00) | 24.6 (1.28) |
| mean (SD) | | | | | |

| | IL-22 Fc fusion protein IV | | | |
|---|---|---|---|---|
| | (H) | (L) | (J) | IV |
| | 60 μg/kg | 90 μg/kg | 120 μg/kg | total |
| Demographic | (n = 4) | (n = 4) | (n = 4) | (n = 26) |
| Sex (male), | 4 (100%) | 4 (100%) | 4 (100%) | 26 (100%) |
| n (%) | | | | |
| Race (white), | 4 (100%) | 4 (100%) | 4 (100%) | 26 (100%) |
| n (%) | | | | |
| Age (years), | 28 (12.0) | 25 (9.3) | 28 (8.7) | 29 (10.3) |
| mean (SD) | | | | |
| Weight (kg), | 75.0 (3.28) | 77.1 (4.22) | 93.8 (22.76) | 80.8 (11.77) |
| mean (SD) | | | | |
| Height (cm), | 181 (1.9) | 182 (3.4) | 184 (10.6) | 180 (7.1) |
| mean (SD) | | | | |
| BMI (kg/m²), | 23.0 (0.89) | 23.3 (0.74) | 27.4 (4.18) | 24.8 (2.83) |
| mean (SD) | | | | |

IL-22 Fc fusion protein SC

| | Pooled | (C) | (E) | (G) | (I) |
|---|---|---|---|---|---|
| | placebo SC | 3 μg/kg | 10 μg/kg | 30 μg/kg | 60 μg/kg |
| Demographic | (n = 10) | (n = 5) | (n = 4) | (n = 4) | (n = 4) |
| Sex (male), | 10 (100%) | 5 (100%) | 4 (100%) | 4 (100%) | 4 (100%) |
| n (%) | | | | | |
| Race (white), | 10 (100%) | 5 (100%) | 4 (100%) | 4 (100%) | 4 (100%) |
| n (%) | | | | | |
| Ethnicity | — | — | — | — | 1 (25%) |
| (Hispanic or | | | | | |
| Latino), n (%) | | | | | |
| Age (years), | 26 (8.5) | 28 (9.3) | 31 (8.8) | 25 (3.6) | 28 (11.0) |
| mean (SD) | | | | | |
| Weight (kg), | 75.8 (15.63) | 85.2 (12.29) | 79.9 (13.56) | 86.0 (16.97) | 77.0 (9.70) |
| mean (SD) | | | | | |
| Height (cm), | 180 (10.3) | 180 (6.7) | 184 (3.9) | 183 (6.1) | 182 (3.6) |
| mean (SD) | | | | | |
| BMI (kg/m²), | 23.3 (3.60) | 26.3 (3.29) | 23.7 (3.93) | 25.6 (4.07) | 23.2 (2.45) |
| mean (SD) | | | | | |

| | | | Pooled | |
|---|---|---|---|---|
| | IL-22 Fc fusion protein SC | | | IL-22 Fc fusion |
| | (K) | SC | | protein |
| | 120 μg/kg | total | Placebo | (IV and SC) |
| Demographic | (n = 1) | (n = 18) | (n = 24) | (n = 44) |
| Sex (male), | 1 (100%) | 18 (100%) | 24 (100%) | 44 (100%) |
| n (%) | | | | |
| Race (white), | 1 (100%) | 18 (100%) | 24 (100%) | 44 (100%) |
| n (%) | | | | |
| Ethnicity | — | 1 (5.6%) | — | 1 (2.3%) |
| (Hispanic or | | | | |
| Latino), n (%) | | | | |
| Age (years), | 22 (NC) | 28 (8.0) | 30 (9.5) | 28 (9.4) |
| mean (SD) | | | | |
| Weight (kg), | 71.8 (NC) | 81.6 (12.45) | 77.1 (13.66) | 81.1 (11.92) |
| mean (SD) | | | | |
| Height (cm), | 184 (NC) | 182 (4.9) | 179 (8.4) | 181 (6.3) |
| mean (SD) | | | | |
| BMI (kg/m²), | 21.2 (NC) | 24.6 (3.41) | 23.8 (3.21) | 24.7 (3.04) |
| mean (SD) | | | | |

### Safety

Overall, 64 subjects experienced 384 TEAEs during the study (Tables 4-7). No subjects withdrew due to a TEAE, and no deaths occurred. TEAEs occurred in nearly all IL-22 Fc fusion protein-treated subjects: 25 of 26 (96.2%) of IL-22 Fc fusion protein IV subjects and 18 of 18 (100%) SC subjects (Table 4). The majority of TEAEs were mild (97.7% of subjects, 264 events) or moderate (29.5% of subjects, 48 events) (Table 3). Two subjects experienced severe TEAEs (1 treatment-related; Table 7) and nearly all TEAEs resolved by study completion.

The predominant TEAEs that distinguished the IL-22 Fc fusion protein-treated cohorts from placebo were skin effects consistent with IL-22 pharmacology, including dry skin, skin exfoliation, erythema, and pruritus, and injection-site events of erythema, discoloration, and hypersensitivity. Dry lips also occurred frequently with IL-22 Fc fusion protein (Table 4).

AE incidence and the severity of the skin effects increased with increasing dose levels in IV cohorts (Table 4). For example, dry skin events increased in frequency and severity in the IV cohorts at doses of 30 μg/kg and higher (FIGS. 2A-1 and 2A-2). These events generally appeared within a week after treatment and resolved within approximately 2 weeks. Three of 4 subjects in Cohort J (120 μg/kg IV) developed moderate cutaneous adverse reactions that were reversible, but also limited tolerability at that dose level. Two subjects experienced 5 erythema, 2 subjects experienced 2 mild-to-moderate pruritus, and 3 subjects experienced mild-to-moderate skin hyperesthesia at multiple times between Days 9 to 17. The site investigator empirically treated two of these subjects with topical corticosteroids. Consequently, the Cohort L dose level was reduced from 240 μg/kg IV to 90 μg/kg IV, which was adequately tolerated.

Subjects in the SC cohorts all experienced anticipated skin reactions due to the local IL-22 response, including dry skin (Table 4; FIGS. 2A-1, 2A-2, 2B, and 2C). Each IL-22 Fc fusion protein SC-treated subject exhibited a localized, erythematous patch that appeared within 5-10 days after dosing that subsequently became scaly, reached maximal intensity 2-3 weeks post injection (FIG. 2B), and resolved by approximately 60 days. The SC skin reactions were adequately tolerated with occasional mild skin discomfort or pruritus, which was treated with topical emollients. At higher doses 60 μg/kg SC), a few treated subjects experienced dry lips (Table 4), indicating that these SC doses caused reactions remote from the injection site.

Two subjects experienced 4 severe treatment-emergent adverse events (TEAEs) (Table 4). One subject in the 10 μg/kg SC cohort experienced 3 events that were also regarded as SAEs not related to the study treatment: a fall, loss of consciousness, and a head wound requiring sutures. On Day 39, a second subject who received IL-22 Fc fusion protein (120 μg/kg IV) experienced treatment-related acute asthma, which was treated with a corticosteroid injection and resolved within 2 days. This subject had a history consistent with exercise-induced asthma, for which no medical care had been sought. For this event, the subject was not seen in an emergency department and did not receive treatment with bronchodilators.

Nine TEAEs were not resolved by study completion. These TEAEs included toothache, leukocyturia, pyuria, haematuria, conjunctivitis allergy (eye; 2 events), eyelid infection, chlamydia infection, and injection site erythema. Only the injection site erythema event (3 μg/kg SC; Day 11) was considered to be related to the study drug. The event was assessed as mild and was resolving at study completion. All remaining TEAEs were considered resolved and required no further follow-up by study completion.

Nineteen subjects had chemistry test findings that were recorded as TEAEs. Increases in blood creatine phosphokinase (CPK), potassium, aspartate aminotransferase, and glucose measurements were mainly mild, with the exception of 4 moderate CPK elevations; these were preceded by vigorous exercise, and none were related to the study treatment. One subject had mild and clinically significant decreased hematocrit, decreased hemoglobin, and decreased red blood cells that were not related to the study treatment.

TABLE 4
Summary of SAEs, and severity and incidence of notable TEAEs

| | | IL-22 Fc fusion protein IV | | | | | | | |
|---|---|---|---|---|---|---|---|---|---|
| | Pooled | (A) | (B) | (D) | (F) | (H) | (L) | (J) | IV |
| | Placebo | 1 μg/kg | 3 μg/kg | 10 μg/kg | 30 μg/kg | 60 μg/kg | 90 μg/kg | 120 μg/kg | total |
| | IV (n = 14) | (n = 2) | (n = 4) | (n = 4) | (n = 4) | (n = 4) | (n = 4) | (n = 4) | (n = 26) |
| Overall total | 13 (92.9%) | 2 (100.0%) | 4 (100.0%) | 3 (75.0%) | 4 (100.0%) | 4 (100.0%) | 4 (100.0%) | 4 (100.0%) | 25 (96.2%) |
| | [43] | [4] | [10] | [10] | [28] | [30] | [44] | [64] | [190] |
| Subjects | — | — | — | — | — | — | — | — | — |
| with SAEs | | | | | | | | | |
| Severity | | | | | | | | | |
| Mild | 12 (85.7%) | 2 (100.0%) | 4 (100.0%) | 3 (75.0%) | 4 (100.0%) | 4 (100.0%) | 4 (100.0%) | 4 (100.0%) | 25 (96.2%) |
| | [36] | [4] | [10] | [7] | [26] | [27] | [43] | [34] | [151] |
| Moderate | 6 (42.9%) | — | — | 2 (50.0%) | 1 (25.0%) | 2 (50.0%) | 1 (25.0%) | 3 (75.0%) | 9 (34.6%) |
| | [7] | | | [3] | [2] | [3] | [1] | [29] | [38] |
| Severe | | | | | | | | 1 (25.0%) | 1 (3.8%) |
| | | | | | | | | [1] | [1]) |
| System organ class | | | | | | | | | |
| Preferred term | | | | | | | | | |
| Skin and subcutaneous | 2 (14.3%) | | 1 (25.0%) | 1 (25.0%) | 3 (75.0%) | 3 (75.0%) | 4 (100.0%) | 4 (100.0%) | 16 (61.5%) |
| tissue disorders | [4] | | [2] | [1] | [10] | [12] | [28] | [35] | [88] |
| Dry skin | — | — | — | — | 3 (75.0%) | 3 (75.0%) | 4 (100.0%) | 4 (100.0%) | 14 (53.8%) |
| | | | | | [5] | [6] | [5] | [10] | [26] |
| Skin exfoliation | — | — | — | — | 1 (25.0%) | 2 (50.0%) | 4 (100.0%) | 3 (75.0%) | 10 (38.5%) |
| | | | | | [1] | [2] | [11] | [8] | [22] |
| Erythema | 1 (7.1%) | — | — | — | — | 2 (50.0%) | 2 (50.0%) | 3 (75.0%) | 7 (26.9%) |
| | [1] | | | | | [2] | [8] | [12] | [22] |
| Pruritus | 1 (7.1%) | — | — | — | 2 (50.0%) | 1 (25.0%) | 2 (50.0%) | 2 (50.0%) | 7 (26.9%) |
| | [1] | | | | [4] | [1] | [2] | [2] | [9] |
| Pain of skin | — | — | — | — | — | — | 1 (25.0%) | 1 (25.0%) | 2 (7.7%) |
| | | | | | | | [2] | [1] | [3] |
| Eczema asteatotic | — | — | — | — | — | — | — | 2 (50.0%) | 2 (7.7%) |
| | | | | | | | | [2] | [2] |
| Gastrointestinal | 7 (50.0%) | 1 (50.0%) | 2 (50.0%) | — | 3 (75.0%) | 4 (100.0%) | 4 (100.0%) | 4 (100.0%) | 18 (69.2%) |
| disorders | [12] | [1] | [2] | | [11] | [6] | [6] | [5] | [31] |
| Lip dry | 1 (7.1%) | — | — | — | 1 (25.0%) | 3 (75.0%) | 4 (100.0%) | 4 (100.0%) | 12 (46.2%) |
| | [1] | | | | [1] | [3] | [4] | [4] | [12] |
| Diarrhea | 3 (21.4%) | 1 (50.0%) | 2 (50.0%) | — | 1 (25.0%) | — | 1 (25.0%) | — | 5 (19.2%) |
| | [3] | [1] | [2] | | [2] | | [1] | | [6] |
| Flatulence | 2 (14.3%) | — | — | — | 2 (50.0%) | 1 (25.0%) | — | — | 3 (11.5%) |
| | [2] | | | | [2] | [1] | | | [3] |
| Nervous system | 3 (21.4%) | 1 (50.0%) | 2 (50.0%) | 1 (25.0%) | — | — | 2 (50.0%) | 3 (75.0%) | 9 (34.6%) |
| disorders | [6] | [1] | [2] | [1] | | | [2] | [8] | [14] |
| Hyperesthesia | — | — | — | — | — | — | — | 3 (75.0%) | 3 (11.5%) |
| | | | | | | | | [6] | [6] |

| | | IL-22 Fc fusion protein SC | | | | | |
|---|---|---|---|---|---|---|---|
| | Placebo | (C) | (E) | (G) | (I) | (K) | SC |
| | SC | 3 μg/kg | 10 μg/kg | 30 μg/kg | 60 μg/kg | 120 μg/kg | total |
| | (n = 10) | (n =5) | (n = 4) | (n = 4) | (n = 4) | (n = 1) | (n = 18) |
| Overall total | 8 (80.0%) | 5 (100.0%) | 4 (100.0%) | 4 (100.0%) | 4 (100.0%) | 1 (100.0%) | 18 (100.0%) |
| | [25] | [21] | [22] | [25] | [41] | [17] | [126] |
| Subjects with SAEs | — | — | 1 (25.0%) | — | — | — | 1 (5.6%) |
| | | | [3] | | | | [3] |
| Severity | | | | | | | |
| Mild | 6 (60.0%) | 5 (100.0%) | 4 (100.0%) | 4 (100.0%) | 4 (100.0%) | 1 (100.0%) | 18 (100.0%) |
| | [21] | [21] | [19] | [24] | [34] | [15] | [113] |
| Moderate | 3 (30.0%) | — | — | 1 (25.0%) | 2 (50.0%) | 1 (100.0%) | 4 (22.2%) |
| | [4] | | | [1] | [7] | [2] | [10] |
| Severe | — | — | 1 (25.0%) | — | — | — | 1 (5.6%) |
| | | | [3] | | | | [3] |
| System organ class | | | | | | | |
| Preferred term | | | | | | | |
| Skin and subcutaneous | — | 1 (20.0%) | 1 (25.0%) | — | 2 (50.0%) | 1 (100.0%) | 5 (27.8%) |
| tissue disorders | | [3] | [1] | | [12] | [6] | [22] |
| Dry skin | — | 1 (20.0%) | — | — | 2 (50.0%) | 1 (100.0%) | 4 (22.2%) |
| | | [1] | | | [6] | [4] | [11] |
| Skin exfoliation | — | — | — | — | 1 (25.0%) | 1 (100.0%) | 2 (11.1%) |
| | | | | | [2] | [2] | [4] |
| Erythema | — | 1 (20.0%) | — | — | 1 (25.0%) | — | 2 (11.1%) |
| | | [1] | | | [1] | | [2] |
| Pruritus | — | 1 (20.0%) | — | — | 1 (25.0%) | — | 2 (11.1%) |
| | | [1] | | | [2] | | [3] |
| Pain of skin | — | — | — | — | 1 (25.0%) | — | 1 (5.6%) |
| | | | | | [1] | | [1] |
| General disorders and | 2 (20.0%) | 5 (100.0%) | 4 (100.0%) | 4 (100.0%) | 4 (100.0%) | 1 (100.0%) | 18 (100.0%) |
| administration site | [4] | [12] | [14] | [19] | [11] | [7] | [63] |
| conditions | | | | | | | |
| Injection-site | 1 (10.0%) | 5 (100.0%) | 4 (100.0%) | 4 (100.0%) | 4 (100.0%) | 1 (100.0%) | 18 (100.0%) |
| erythema | [1]^{a} | [8] | [7] | [12] | [7] | [3] | [37] |
| Injection-site | 1 (10.0%) | 2 (40.0%) | 4 (100.0%) | 2 (50.0%) | 1 (25.0%) | — | 9 (50.0%) |
| discoloration | [1] | [2] | [4] | [2] | [1] | | [9] |
| Injection-site | — | — | 1 (25.0%) | 2 (50.0%) | 1 (25.0%) | 1 (100.0%) | 5 (27.8%) |
| hypersensitivity | | | [1] | [2] | [1] | [1] | [5] |
| Injection-site | 1 (10.0%) | — | 2 (50.0%) | — | 1 (25.0%) | 1 (100.0%) | 4 (22.2%) |
| exfoliation | [1] | | [2] | | [1] | [1] | [4] |
| Injection-site | 1 (10.0%) | 1 (20.0%) | — | 1 (25.0%) | — | — | 2 (11.1%) |
| hemorrhage | | | | | | | |
| Injection-site pain | — | — | — | 2 (50.0%) | — | — | 2 (11.1%) |
| | | | | [2] | | | [2] |
| Injection-site | — | 1 (20.0%) | — | — | 1 (25.0%) | — | 2 (11.1%) |
| pruritus | | [1] | | | [1] | | [2] |
| Gastrointestinal | 3 (30.0%) | 2 (40.0%) | 1 (25.0%) | | 2 (50.0%) | 1 (100.0%) | 6 (33.3%) |
| disorders | [4] | [4] | [1] | | [8] | [2] | [15] |
| Lip dry | — | — | — | — | 2 (50.0%) | 1 (100.0%) | 3 (16.7%) |
| | | | | | [2] | [1] | [3] |
| Diarrhea | 2 (20.0%) | 1 (20.0%) | — | — | 1 (25.0%) | — | 2 (11.1%) |
| | [2] | [2] | | | [1] | | [3] |
| Flatulence | 1 (10.0%) | — | — | — | 2 (50.0%) | — | 2 (11.1%) |
| | [1] | | | | [2] | | [2] |
| Abdominal pain | — | 2 (40.0%) | — | — | — | — | 2 (11.1%) |
| | | [2] | | | | | [2] |
| Nausea | — | — | 1 (25.0%) | — | 1 (25.0%) | — | 2 (11.1%) |
| | | | [1] | | [1] | | [2] |
| Nervous system | 2 (20.0%) | — | 1 (25.0%) | — | 2 (50.0%) | — | 3 (16.7%) |
| disorders | [2] | | [2] | | [2] | | [4] |
| Hyperesthesia | — | — | — | — | 1 (25.0%) | — | 1 (5.6%) |
| | | | | | [1] | | [1] |

Data are number of subjects (percentage of subjects with adverse events) [number of adverse events].
^{a}One subject in the placebo group received a minimal amount of IL-22 Fc fusion protein instead of placebo due to a pharmacy error. This subject developed a small patch of mild erythema at the injection site, which completely resolved by study end.

TABLE 5
Frequency of TEAEs occurring in ≥2 subjects for any treatment (all causalities)-IV cohorts

| | | IL-22 Fc fusion protein IV | | | | | | | |
|---|---|---|---|---|---|---|---|---|---|
| System organ | Pooled | (A) | (B) | (D) | (F) | (H) | (L) | (J) | IV |
| class | placebo | 1 μg/kg | 3 μg/kg | 10 μg/kg | 30 μg/kg | 60 μg/kg | 90 μg/kg | 120 μg/kg | total |
| Preferred term | IV (n = 14) | (n = 2) | (n = 4) | (n = 4) | (n = 4) | (n = 4) | (n = 4) | (n = 4) | (n = 26) |
| Overall total | 13 (92.9%) | 2 (100.0%) | 4 (100.0%) | 3 (75.0%) | 4 (100.0%) | 4 (100.0%) | 4 (100.0%) | 4 (100.0%) | 25 (96.2%) |
| | [43] | [4] | [10] | [10] | [28] | [30] | [44] | [64] | [190] |
| Skin and subcutaneous | 2 (14.3%) | — | 1 (25.0%) | 1 (25.0%) | 3 (75.0%) | 3 (75.0%) | 4 (100.0%) | 4 (100.0%) | 16 (61.5%) |
| tissue disorders | [4] | | [2] | [1] | [10] | [12] | [28] | [35] | [88] |
| Dry skin | — | — | — | — | 3 (75.0%) | 3 (75.0%) | 4 (100.0%) | 4 (100.0%) | 14 (53.8%) |
| | | | | | [5] | [6] | [5] | [10] | [26] |
| Skin exfoliation | — | — | — | — | 1 (25.0%) | 2 (50.0%) | 4 (100.0%) | 3 (75.0%) | 10 (38.5%) |
| | | | | | [1] | [2] | [11] | [8] | [22] |
| Erythema | 1 (7.1%) | — | — | — | — | 2 (50.0%) | 2 (50.0%) | 3 (75.0%) | 7 (26.9%) |
| | [1] | | | | | [2] | [8] | [12] | [22] |
| Pruritus | 1 (7.1%) | — | — | — | 2 (50.0%) | 1 (25.0%) | 2 (50.0%) | 2 (50.0%) | 7 (26.9%) |
| | [1] | | | | [4] | [1] | [2] | [2] | [9] |
| Pain of skin | — | — | — | — | — | — | 1 (25.0%) | 1 (25.0%) | 2 (7.7%) |
| | | | | | | | [2] | [1] | [3] |
| Eczema asteatotic | — | — | — | — | — | — | — | 2 (50.0%) | 2 (7.7%) |
| | | | | | | | | [2] | [2] |
| Gastrointestinal | 7 (50.0%) | 1 (50.0%) | 2 (50.0%) | — | 3 (75.0%) | 4 (100.0%) | 4 (100.0%) | 4 (100.0%) | 18 (69.2%) |
| disorders | [12] | [1] | [2] | | [11] | [6] | [6] | [5] | [31] |
| Lip dry | 1 (7.1%) | — | — | — | 1 (25.0%) | 3 (75.0%) | 4 (100.0%) | 4 (100.0%) | 12 (46.2%) |
| | [1] | | | | [1] | [3] | [4] | [4] | [12] |
| Diarrhea | 3 (21.4%) | 1 (50.0%) | 2 (50.0%) | — | 1 (25.0%) | — | 1 (25.0%) | — | 5 (19.2%) |
| | [3] | [1] | [2] | | [2] | | [1] | | [6] |
| Flatulence | 2 (14.3%) | — | — | — | 2 (50.0%) | 1 (25.0%) | — | — | 3 (11.5%) |
| | [2] | | | | [2] | [1] | | | [3] |
| Toothache | 2 (14.3%) | — | — | — | 1 (25.0%) | — | — | — | 1 (3.8%) |
| | [2] | | | | [1] | | | | [1] |
| Investigations | 4 (28.6%) | — | 1 (25.0%) | 3 (75.0%) | 3 (75.0%) | 4 (100.0%) | 3 (75.0%) | 4 (100.0%) | 18 (69.2%) |
| | [4] | | [1] | [4] | [3] | [7] | [4] | [6] | [25] |
| Blood creatine | 4 (28.6%) | — | — | 1 (25.0%) | 1 (25.0%) | 2 (50.0%) | 1 (25.0%) | 1 (25.0%) | 6 (23.1%) |
| phosphokinase | [4] | | | [1] | [1] | [3] | [1] | [2] | [8] |
| increased | | | | | | | | | |
| Nervous system | 3 (21.4%) | 1 (50.0%) | 2 (50.0%) | 1 (25.0%) | — | — | 2 (50.0%) | 3 (75.0%) | 9 (34.6%) |
| disorders | [6] | [1] | [2] | [1] | | | [2] | [8] | [14] |
| Hyperesthesia | — | — | — | — | — | — | — | 3 (75.0%) | 3 (11.5%) |
| | | | | | | | | [6] | [6] |
| Headache | 3 (21.4%) | 1 (50.0%) | 1 (25.0%) | 1 (25.0%) | — | — | — | — | 3 (11.5%) |
| | [5] | [1] | [1] | [1] | | | | | [3] |
| Paresthesia | — | — | 1 (25.0%) | — | — | — | 1 (25.0%) | 1 (25.0%) | 3 (11.5%) |
| | | | [1] | | | | [1] | [2] | [4] |
| Infections and | 3 (21.4%) | — | 1 (25.0%) | 3 (75.0%) | 2 (50.0%) | 2 (50.0%) | 2 (50.0%) | 1 (25.0%) | 11 (42.3%) |
| infestations | [3] | | [1] | [3] | [2] | [2] | [2] | [1] | [11] |
| Nasopharyngitis | 2 (14.3%) | — | 1 (25.0%) | 1 (25.0%) | 1 (25.0%) | 1 (25.0%) | 1 (25.0%) | — | 5 (19.2%) |
| | [2] | | [1] | [1] | [1] | [1] | [1] | | [5] |
| Renal and urinary | 4 (28.6%) | 1 (50.0%) | 2 (50.0%) | — | — | 1 (25.0%) | 1 (25.0%) | 1 (25.0%) | 6 (23.1%) |
| disorders | [5] | [1] | [2] | | | [1] | [1] | [1] | [6] |
| Haematuria | 3 (21.4%) | 1 (50.0%) | 2 (50.0%) | — | — | 1 (25.0%) | 1 (25.0%) | 1 (25.0%) | 6 (23.1%) |
| | [3] | [1] | [2] | | | [1] | [1] | [1] | [6] |

Data are no. of subjects (percentage of subjects with adverse events) [number of adverse events].

TABLE 6
Frequency of TEAEs occurring in ≥2 subjects for any treatment (all causalities)-SC cohorts

| | | IL-22 Fc fusion protein SC | | | | | |
|---|---|---|---|---|---|---|---|
| | Placebo | (C) | (E) | (G) | (I) | (K) | SC |
| System organ class | SC | 3 μg/kg | 10 μg/kg | 30 μg/kg | 60 μg/kg | 120 μg/kg | total |
| Preferred term | (n = 10) | (n = 5) | (n = 4) | (n = 4) | (n = 4) | (n = 1) | (n = 18) |
| Overall Total | 8 (80.0%) | 5 (100.0%) | 4 (100.0%) | 4 (100.0%) | 4(100.0%) | 1 (100.0%) | 18 (100.0%) |
| | [25] | [21] | [22] | [25] | [41] | [17] | [126] |
| Skin and subcutaneous | — | 1 (20.0%) | 1 (25.0%) | — | 2 (50.0%) | 1 (100.0%) | 5 (27.8%) |
| tissue disorders | | [3] | [1] | | [12] | [6] | [22] |
| Dry skin | — | 1 (20.0%) | — | — | 2 (50.0%) | 1 (100.0%) | 4 (22.2%) |
| | | [1] | | | [6] | [4] | [11] |
| Skin exfoliation | — | — | — | — | 1 (25.0%) | 1 (100.0%) | 2 (11.1%) |
| | | | | | [2] | [2] | [4] |
| Erythema | — | 1 (20.0%) | — | — | 1 (25.0%) | — | 2 (11.1%) |
| | | [1] | | | [1] | | [2] |
| Pruritus | — | 1 (20.0%) | — | — | 1 (25.0%) | — | 2 (11.1%) |
| | | [1] | | | [2] | | [3] |
| Pain of skin | — | — | — | — | 1 (25.0%) | — | 1 (5.6%) |
| | | | | | [1] | | [1] |
| Eczema asteatotic | — | — | — | — | — | — | — |
| General disorders and | 2 (20.0%) | 5 (100.0%) | 4 (100.0%) | 4 (100.0%) | 4 (100.0%) | 1 (100.0%) | 18 (100.0%) |
| administration site | [4] | [12] | [14] | [19] | [11] | [7] | [63] |
| conditions | | | | | | | |
| Injection-site | 1 (10.0%) | 5 (100.0%) | 4 (100.0%) | 4 (100.0%) | 4 (100.0%) | 1 (100.0%) | 18 (100.0%) |
| erythema | [1]^{a} | [8] | [7] | [12] | [7] | [3] | [37] |
| Injection-site | 1 (10.0%) | 2 (40.0%) | 4 (100.0%) | 2 (50.0%) | 1 (25.0%) | — | 9 (50.0%) |
| discoloration | [1] | [2] | [4] | [2] | [1] | | [9] |
| Injection-site | — | — | 1 (25.0%) | 2 (50.0%) | 1 (25.0%) | 1 (100.0%) | 5 (27.8%) |
| hypersensitivity | | | [1] | [2] | [1] | [1] | [5] |
| Injection-site | 1 (10.0%) | — | 2 (50.0%) | — | 1 (25.0%) | 1 (100.0%) | 4 (22.2%) |
| exfoliation | [1] | | [2] | | [1] | [1] | [4] |
| Injection-site | 1 (10.0%) | 1 (20.0%) | — | 1 (25.0%) | — | — | 2 (11.1%) |
| hemorrhage | [1] | [1] | | [1] | | | [2] |
| Injection-site pain | — | — | — | 2 (50.0%) | — | — | 2 (11.1%) |
| | | | | [2] | | | [2] |
| Injection-site | — | 1 (20.0%) | — | — | 1 (25.0%) | — | 2 (11.1%) |
| pruritus | | [1] | | | [1] | | [2] |
| Gastrointestinal | 3 (30.0%) | 2 (40.0%) | 1 (25.0%) | — | 2 (50.0%) | 1 (100.0%) | 6 (33.3%) |
| disorders | [4] | [4] | [1] | | [8] | [2] | [15] |
| Lip dry | — | — | — | — | 2 (50.0%) | 1 (100.0%) | 3 (16.7%) |
| | | | | | [2] | [1] | [3] |
| Diarrhea | 2 (20.0%) | 1 (20.0%) | — | — | 1 (25.0%) | — | 2 (11.1%) |
| | [2] | [2] | | | [1] | | [3] |
| Flatulence | 1 (10.0%) | — | — | — | 2 (50.0%) | — | 2 (11.1%) |
| | [1] | | | | [2] | | [2] |
| Abdominal pain | — | 2 (40.0%) | — | — | — | — | 2 (11.1%) |
| | | [2] | | | | | [2] |
| Nausea | — | — | 1 (25.0%) | — | 1 (25.0%) | — | 2 (11.1%) |
| | | | [1] | | [1] | | [2] |
| Toothache | — | — | — | — | — | — | — |
| Investigations | 4 (40.0%) | 1 (20.0%) | 2 (50.0%) | — | 3 (75.0%) | — | 6 (33.3%) |
| | [9] | [1] | [2] | | [3] | | [6] |
| Blood creatine | 4 (40.0%) | 1 (20.0%) | 1 (25.0%) | — | — | — | 2 (11.1%) |
| phosphokinase | [4] | [1] | [1] | | | | [2] |
| increased | | | | | | | |
| Nervous system | 2 (20.0%) | — | 1 (25.0%) | — | 2 (50.0%) | — | 3 (16.7%) |
| disorders | [2] | | [2] | | [2] | | [4] |
| Hyperesthesia | — | — | — | — | 1 (25.0%) | — | 1 (5.6%) |
| | | | | | [1] | | [1] |
| Headache | 2 (20.0%) | — | 1 (25.0%) | — | — | — | 1 (5.6%) |
| | [2] | | [1] | | | | [1] |
| Paresthesia | — | — | — | — | — | — | — |
| Infections and | 1 (10.0%) | — | — | 1 (25.0%) | 2 (50.0%) | 1 (100.0%) | 4 (22.2%) |
| infestations | [2] | | | [1] | [2] | [1] | [4] |
| Nasopharyngitis | 1 (10.0%) | — | — | — | 2 (50.0%) | 1 (100.0%) | 3 (16.7%) |
| | [1] | | | | [2] | [1] | [3] |
| Gastroenteritis | — | — | — | — | — | — | — |
| Renal and urinary | 1 (10.0%) | — | — | 1 (25.0%) | 2 (50.0%) | — | 3 (16.7%) |
| disorders | [2] | | | [4] | [2] | | [6] |
| Haematuria | — | — | — | 1 (25.0%) | 2 (50.0%) | — | 3 (16.7%) |
| | | | | [2] | [2] | | [4] |
| Leukocyturia | 1 (10.0%) | — | — | 1 (25.0%) | — | — | 1 (5.6%) |
| | [2] | | | [1] | | | [1] |
| Eye disorders | — | — | — | — | 1 (25.0%) | 1 (100.0%) | 2 (11.1%) |
| | | | | | [1] | [1] | [2] |
| Dry eye | — | — | — | — | 1 (25.0%) | 1 (100.0%) | 2 (11.1%) |
| | | | | | [1] | [1] | [2] |

Data are no. of subjects (percentage of subjects with adverse events) [number of adverse events].
^{a}One subject in the placebo group received a minimal amount of IL-22 Fc fusion protein instead of placebo due to a pharmacy error. This subject developed a small patch of mild erythema at the injection site, which completely resolved by study end.

TABLE 7
Serious AEs, severity of AEs, and frequency of treatment-emergent adverse events occurring in ≥2
subjects for any treatment (all causalities and treatment-related): pooled IV and SC cohorts

| | All Causalities | | | | Treatment-related | | | |
|---|---|---|---|---|---|---|---|---|
| | Pooled | | Pooled IL-22 Fc | | Pooled | | Pooled IL-22 Fc | |
| | placebo | | fusion protein | | placebo | | fusion protein | |
| | (N = 24) | | (N = 44) | | (N = 24) | | (N = 44) | |
| Overall Total | 21 (87.5%) | [68] | 43 (97.7%) | [316] | 11 (45.8%) | [24] | 38 (86.4%) | [243] |
| Subjects with SAEs | — | | 1 (2.3%) | [3] | — | | — | |
| Severity | | | | | | | | |
| Mild | 18 (75.0%) | [57] | 43 (97.7%) | [264] | 11 (45.8%) | [22] | 38 (86.4%) | [199] |
| Moderate | 9 (37.5%) | [11] | 13 (29.5%) | [48] | 2 (8.3%) | [2] | 10 (22.7%) | [43] |
| Severe | — | | 2 (4.5%) | [4] | — | | 1 (2.3%) | [1] |
| System Organ Class | | | | | | | | |
| Preferred Term | | | | | | | | |
| Skin and subcutaneous | 2 (8.3%) | [4] | 21 (47.7%) | [110] | 2 (8.3%) | [3] | 19 (43.2%) | [104] |
| tissue disorders | | | | | | | | |
| Dry skin | — | | 18 (40.9%) | [37] | — | | 17 (38.6%) | [36] |
| Skin exfoliation | — | | 12 (27.3%) | [26] | — | | 12 (27.3%) | [26] |
| Erythema | 1 (4.2%) | [1] | 9 (20.5%) | [24] | 1 (4.2%) | [1] | 8 (18.2%) | [23] |
| Pruritus | 1 (4.2%) | [1] | 9 (20.5%) | [12] | 1 (4.2%) | [1] | 8 (18.2%) | [11] |
| Pain of skin | — | | 3 (6.8%) | [4] | — | | 3 (6.8%) | [4] |
| Eczema asteatotic | — | | 2 (4.5%) | [2] | — | | 2 (4.5%) | [2] |
| General disorders | 3 (12.5%) | [5] | 21 (47.7%) | [66] | 1 (4.2%) | [3] | 19 (43.2%) | [62] |
| and administration | | | | | | | | |
| site conditions | | | | | | | | |
| Injection-site | 1 (4.2%) | [1]^{a} | 18 (40.9%) | [37] | 1 (4.2%) | [1]^{a} | 18 (40.9%) | [37] |
| erythema | | | | | | | | |
| Injection-site | 1 (4.2%) | [1] | 9 (20.5%) | [9] | 1 (4.2%) | [1] | 9 (20.5%) | [9] |
| discoloration | | | | | | | | |
| Injection-site | — | | 5 (11.4%) | [5] | — | | 5 (11.4%) | [5] |
| hypersensitivity | | | | | | | | |
| Injection-site | 1 (4.2%) | [1] | 4 (9.1%) | [4] | 1 (4.2%) | [1] | 4 (9.1%) | [4] |
| exfoliation | | | | | | | | |
| Injection-site | 1 (4.2%) | [1] | 2 (4.5%) | | | | | |
| hemorrhage | | | | | | | | |
| Injection-site pain | — | | 2 (4.5%) | [2] | — | | 2 (4.5%) | [2] |
| Injection-site | — | | 2 (4.5%) | [2] | — | | 2 (4.5%) | [2] |
| pruritus | | | | | | | | |
| Gastrointestinal | 10 (41.7%) | [16] | 24 (54.5%) | [46] | 6 (25.0%) | [12] | 20 (45.5%) | [40] |
| disorders | | | | | | | | |
| Lip dry | 1 (4.2%) | [1] | 15 (34.1%) | [15] | 1 (4.2%) | [1] | 15 (34.1%) | [15] |
| Diarrhea | 5 (20.8%) | [5] | 7 (15.9%) | [9] | 5 (20.8%) | [5] | 6 (13.6%) | [8] |
| Flatulence | 3 (12.5%) | [3] | 5 (11.4%) | [5] | 3 (12.5%) | [3] | 5 (11.4%) | [5] |
| Abdominal pain | 1 (4.2%) | [3] | 3 (6.8%) | [5] | 1 (4.2%) | [3] | 3 (6.8%) | [4] |
| Nausea | — | | 3 (6.8%) | [3] | — | | — | |
| Toothache | 2 (8.3%) | [2] | 1 (2.3%) | [1] | — | | — | |
| Investigations | 8 (33.3%) | [13] | 24 (54.5%) | [31] | — | | 15 (34.1%) | [15] |
| Blood creatine | 8 (33.3%) | [8] | 8 (18.2%) | [10] | — | | — | |
| phosphokinase | | | | | | | | |
| increased | | | | | | | | |
| Nervous system | 5 (20.8%) | [8] | 12 (27.3%) | [18] | 4 (16.7%) | [6] | 9 (20.5%) | [14] |
| disorders | | | | | | | | |
| Hyperesthesia | — | | 4 (9.1%) | [7] | — | | 4 (9.1%) | [7] |
| Headache | 5 (20.8%) | [7] | 4 (9.1%) | [4] | 4 (16.7%) | [6] | 3 (6.8%) | [3] |

| Paresthesia | — | | 3 (6.8%) | [4] | — | 2 (4.5%) | [3] |
|---|---|---|---|---|---|---|---|
| Infections and | 4 (16.7%) | [5] | 15 (34.1%) | [15] | — | — | |
| infestations | | | | | | | |
| Nasopharyngitis | 3 (12.5%) | [3] | 8 (18.2%) | [8] | — | — | |
| Gastroenteritis | 1 (4.2%) | [1] | 2 (4.5%) | [2] | — | — | |
| Renal and urinary | 5 (20.8%) | [7] | 9 (20.5%) | [12] | — | — | |
| disorders | | | | | | | |
| Hematuria | 3 (12.5%) | [3] | 9 (20.5%) | [10] | — | — | |
| Leukocyturia | 2 (8.3%) | [4] | 1 (2.3%) | [1] | — | — | |
| Eye disorders | 1 (4.2%) | [1] | 5 (11.4%) | [5] | — | 4 (9.1%) | [4] |

| | | | | | | |
|---|---|---|---|---|---|---|
| Dry eye | — | 2 (4.5%) | [2] | — | 2 (4.5%) | [2] |

Data are no. of subjects (percentage of subjects with adverse events) [number of adverse events].
^{a}One subject in the placebo group received a minimal amount of IL-22 Fc fusion protein instead of placebo due to a pharmacy error. This subject developed a small patch of mild erythema at the injection site, which completely resolved by study end.

Nine subjects from Cohorts C (3 subjects), D (1 subject), E (4 subjects), and G (1 subject) underwent skin biopsy for further evaluation of dermatologic AEs after consultation with a dermatologist. Histopathologic biopsy findings were similar to those observed in nonclinical studies and demonstrated psoriasiform epidermal hyperplasia with variably increased vascularity in the superficial dermis with discrete, patchy, mild perivascular lymphoplasmacytic infiltrates (FIG. 2C). There was normal proliferation and no atypia.

In Cohort K (120 μg/kg SC), the sentinel subject who received IL-22 Fc fusion protein experienced mild-to-moderate local and systemic dermatological AEs and dry eyes. The investigator determined that these dermatological AEs were not dose limiting but were similar to those observed at 90 μg/kg IV (Cohort L) and thus warranted no further dosing of the cohort. Cohort M (240 μg/kg SC) was cancelled because Cohort K (120 μg/kg SC) was not completed.

Safety data (including blood tests, urinalysis, ECG, vital signs, or body weight) showed no treatment-related, clinically significant abnormalities except for CRP (see details below) and fibrinogen (which was slightly elevated in some subjects). Similar to CRP and consistent with nonclinical results, reversible increases in fibrinogen were only seen in IL-22 Fc fusion protein-treated subjects, were not deemed clinically significant, and were likely due to direct stimulation of fibrinogen production from hepatocytes.

One subject (2.3%) in the lowest SC dose group (3 μg/kg) was negative for ADAs before dosing and through Day 43, but tested positive for ADAs on Day 57 (study completion). Two subjects (10 μg/kg SC; placebo SC) had detectable ADAs at baseline and post dose, but postdose ADA titers did not rise above their respective baseline values. We observed no relationship between the presence of ADAs and either safety or PK parameters in these subjects.

### Pharmacokinetics

PK parameters following single IV or SC dose administrations of IL-22 Fc fusion protein are summarized in Table 8. Pharmacokinetics for IL-22 Fc fusion protein exhibited a biphasic disposition following IV infusion, with an initial rapid distribution phase followed by a slow elimination phase (FIG. 3A). Systemic exposure, based on maximum serum concentration (Cₘₐₓ) and area under the curve extrapolated to infinity (AUC_{inf}), was approximately dose proportional across 10- to 120-μg/kg dose levels (FIGS. 4A and 4B). Average Cₘₐₓ and AUC_{inf} for the highest dose level (120 μg/kg) was 1110 ng/mL and 7280 ng*d/mL, respectively. Mean half-life values ranged from 6.03-11.2 days across the 10- to 120-μg/kg doses. Mean CL values were generally similar across the 10- to 120-μg/kg dose levels, ranging from 1.26-1.77 L/day, indicating dose-proportional PK within this dose range.

TABLE 8
Summary of serum pharmacokinetic parameters of IL-22 Fc fusion protein following single dose administration by IV infusion

| | Dose | | Cₘₐₓ | | | CL or CL/F | V or V/F | AUC₀₋ₜ | AUC_{inf} |
|---|---|---|---|---|---|---|---|---|---|
| Cohort | (μg/kg) | n | (ng/mL) | Tₘₐₓ (d) | t_{1/2}^{c} (d) | (L/d) | (L) | (ng*d/mL) | (ng*d/mL) |
| IV (A) | 1 | 2 | 9.24-10.8^{b} | | NC | NC | NC | 6.90-7.44^{b} | NC |
| IV (B) | 3 | 4 | 26.7 (22.0) | | NC | NC | NC | 58.5 (11.3) | NC |
| IV (D) | 10 | 4 | 98.7 (12.7) | | 6.03 (1.70) | 1.77 (17.0) | 14.9 (17.0) | 360 (22.0) | 419 (21.1) |
| IV (F) | 30 | 4 | 297 (8.4) | | 7.88 (1.07) | 1.52 (13.5) | 17.2 (18.4) | 1530 (7.4) | 1660 (6.2) |
| IV (H) | 60 | 4 | 559 (5.2) | | 11.2 (1.83) | 1.34 (14.8) | 21.4 (8.3) | 3230 (15.5) | 3370 (14.9) |
| IV (L) | 90 | 4 | 840 (20.5) | | 9.48 (0.781) | 1.59 (18.5) | 21.7 (14.4) | 5020 (31.2) | 5130 (30.7) |
| IV (J) | 120 | 4 | 1110 (6.4) | | 9.92 (0.267) | 1.26 (6.8) | 18.0 (5.4) | 7150 (10.6) | 7280 (10.7) |
| SC (C) | 3 | 4 | 8.36 (13.9) | 6.99 (6.97-10.0) | NC | NC | NC | 57.9^{a} | NC |
| SC (E) | 10 | 4 | 22.2 (16.4) | 4.00 (3.99-7.06) | 5.86-8.08^{b} | 2.50-2.88^{b} | 24.3-29.1^{b} | 243 (23.5) | 236-395^{b} |
| SC (G) | 30 | 4 | 54.0 (18.6) | 4.00 (4.00-4.00) | 8.73 (1.07) | 2.89 (17.7) | 36.1 (22.3) | 760 (20.0) | 873 (20.0) |
| SC (I) | 60 | 4 | 137 (48.4) | 4.00 (2.00-7.00) | 10.2 (0.965) | 1.72 (26.4) | 25.1 (20.9) | 2510 (40.4) | 2650 (39.3) |
| SC (K) | 120 | 1 | 335 | 7 | 11.1 | 1.49 | 23.8 | 5620 | 5820 |

Parameters are presented as mean (CV %) except where otherwise indicated. Tₘₐₓ was only available for SC cohorts shown as median (min-max). Parameters CL and V were calculated for IV cohorts, and CL/F and V/F for SC cohorts.
^{a}n = 1; individual values are reported
^{b}n = 2; range is reported
^{c}t_{1/2} is reported as mean (SD)

IL-22 Fc fusion protein, administered subcutaneously, exhibited pharmacokinetics characterized by slow absorption followed by a slow, monophasic decline (FIG. 3B), and systemic exposure, based on Cₘₐₓ and AUC_{inf}, also increased with increasing dose levels (FIGS. 4C and 4D). The observed Cₘₐₓ and AUC_{inf} from the highest dose cohort (120 μg/kg; n=1) was 335 ng/mL and 5820 ng*d/mL, respectively. Tₘₐₓ median values ranged from 4-6.99 days. Half-life values ranged from 5.86-11.1 days and were shorter at 10 μg/kg than at higher dose levels. SC bioavailability, using least squares means ratios (90% confidence intervals [Cl]) comparing the AUC_{inf} following SC injections to those following IV infusions, was 72.9% (45.4, 117.0), 52.7% (43.1, 64.4), and 78.6% (52.9, 116.7) for the 10-, 30-, and 60-μg/kg dose levels, respectively.

### Pharmacodynamic Biomarkers

Because nonclinical studies established REG3A, CRP, and SAA as relevant PD biomarkers of IL-22R signaling, we assessed their serum levels at baseline and after IL-22 Fc fusion protein administration. Serum REG3A levels, normalized to baseline, increased dose-dependently following both IV and SC doses of IL-22 Fc fusion protein (FIGS. 5A, 5B, 6A-1, and 6A-2). In IV cohorts, the mean percent changes from baseline in REG3A levels over time peaked around Day 5 with up to a 300-400% increase for the higher dose cohorts (FIGS. 5A, 5B, 6A-1, and 6A-2). The largest observed percent increase occurred at 90 μg/kg (3/4 subjects had increases of approximately 400% or greater; 4/4 subjects at 120 μg/kg had increases of less than 400%). In the 30-, 60-, and 120-μg/kg SC cohorts, REG3A percent increases peaked later compared to IV injections, on Day 11 (FIG. 5B). Greater inter-patient variability in the percent increases occurred at the lower SC dose levels (3 and 10 μg/kg; FIG. 7), however all SC dose levels showed comparable percent increases at Day 8 compared to Day 15, but all SC dose levels showed comparable percent increases at Day 8 compared to Day 15, except in the 60 μg/kg cohort, which had a steeper decrease after the Day 11 peak (FIG. 5B). The observed REG3A elevations with increasing dose levels returned to near baseline level by study end (Week 8) at higher IL-22 Fc fusion protein doses (60 μg/kg and above). While no changes occurred in the placebo group, REG3A levels increased in all IV and SC IL-22 Fc fusion protein cohorts (FIGS. 5A and 5B), with lower dose levels showing relative increases of ˜30%-45% (1-3 μg/kg IV) and ˜30%-55% (3-10 μg/kg SC) (FIG. 7).

Serum SAA and CRP levels, normalized to baseline, peaked for the 3-120 μg/kg IV cohorts between Days 2 and 3 (FIGS. 5A, 5C, 6B-1, 6-B-2, 6-C-1, and 6C-2). One subject in the 10 μg/kg IV cohort exhibited an additional peak of CRP levels on Day 8, but this that coincided with signs of a viral illness, suggesting that this additional spike was not drug-related (FIGS. 5C, 6B-1, and 6B-2). For the 10-120 μg/kg SC cohorts SAA peak values were lower and generally delayed compared to the IV cohorts, occurring between Day 5 and 8 (FIG. 5B). In the 60- to 120-μg/kg IL-22 Fc fusion protein SC cohorts, dose-dependent increases in CRP peaked generally between Day 2 to Day 8 (FIG. 5B). Levels of SAA and CRP returned to near baseline by Week 2 and Week 4, respectively, across all doses in the IV and SC treatment groups (FIG. 5). Neither the placebo cohort nor the lowest IL-22 Fc fusion protein dose cohorts (1 μg/kg IV and 3 μg/kg SC) showed increases in SAA and CRP (FIGS. 5A-5C, 6A-1, 6A-2, 6B-1, 6B-2, 6C-1, 6C-2, and 7).

Increases in CRP were consistent with results from nonclinical studies and notably, occurred in the absence of signs of inflammation, such as fever, leukocytosis, and vital sign changes. We also examined serum levels of IL-113, IL-2, IL-6, IL-8, IL-10, TNF-α, and IFN-γ, cytokines associated with inflammatory responses (FIGS. 8A and 8B). Importantly, none of the cytokines demonstrated any dose-dependent increases in response to IL-22 Fc fusion protein, corroborating the observations that increases in CRP increases were on-target, dose-dependent effects of IL-22 Fc fusion protein administration. IL-113, IL-8, IL-6, IL-10 and TNF-α levels were in the range of healthy volunteers. All subjects' IFN-γ and IL-2 levels were below the limit of detection, except for one subject in the 10 μg/kg IV cohort with increased IL-2 levels (FIGS. 8A and 8B) on Day 2. At that time, however, this subject exhibited no AEs.

### C. Conclusions and Discussion

In this phase 1a study, single doses of IL-22 Fc fusion protein, administered by IV infusion (1-90 μg/kg) or SC injection (3-60 μg/kg), were adequately tolerated in healthy, male subjects and demonstrated target-related skin effects. Due to moderate, reversible dermatological reactions in three of four subjects, the 120-μg/kg IV dose level was deemed to exceed the maximum tolerated dose in these healthy volunteers. A protocol-defined maximum tolerated dose in the SC cohorts was not determined, but dose escalation was stopped by the investigator in light of AEs consistent with systemic IL-22 exposure and pharmacology. IL-22 Fc fusion protein exhibited a half-life of approximately 1 week, and approximately dose-proportional increases in total exposure following single doses of IV or SC IL-22 Fc fusion protein.

Consistent with results from nonclinical studies, increases in dermatological effects following IL-22 Fc fusion protein treatment at 3 μg/kg IV and above indicated activation of the IL-22 signaling pathway and dose-dependent pharmacological activity. Peak skin AEs occurred approximately 2 weeks after the administration of the drug, an example of hysteresis. Skin biopsies in subjects administered IL-22 Fc fusion protein, particularly those who received IL-22 Fc fusion protein by SC injection, demonstrated epidermal hyperplasia, often with psoriasiform features. This histologic appearance was very similar to that seen in minipigs administered IL-22 Fc fusion protein by SC injection and is consistent with evidence implicating IL-22 in the pathogenesis of psoriasis.

The dose-dependent increases in peripheral PD biomarkers REG3A, SAA, and CRP with higher doses of IL-22 Fc fusion protein further confirmed target engagement and pharmacological activity. These biomarker elevations correlated with IL-22 Fc fusion protein drug levels and peaked at higher concentrations and earlier timepoints following IV compared to SC administration. These increases suggest that both IV and SC IL-22 Fc fusion protein had activity in various epithelial tissues; specifically, CRP and SAA elevations suggest stimulation of hepatocytes, and increases of REG3A indicate stimulation of pancreatic and or intestinal epithelium. Although REG3A percent increases at the 120 μg/kg IV dose were less than those at the 90 μg/kg dose, subject numbers were too low to conclude that REG3A levels had plateaued. SC administration caused pronounced skin effects locally at the injection site (where IL-22 Fc fusion protein tissue concentrations were likely quite high), but we also observed remote effects at higher doses (e.g. dry lips). Together with the PK data from the SC cohorts and the dose-dependent increases in biomarkers, this suggests that SC administration of IL-22 Fc fusion protein in the higher dose cohorts caused systemic effects.

While CRP is used clinically as a diagnostic marker of inflammation, IL-22 Fc fusion protein-induced CRP increases were not accompanied by signs or symptoms of inflammation, including changes in vital signs, laboratory values, or inflammatory cytokines, indicating that IL-22 Fc fusion protein had a direct effect on hepatocytes. The lack of increased inflammatory cytokines was consistent with nonclinical studies in cynomolgus monkeys and with a study showing that CRP infusion in healthy subjects does not induce proinflammatory effects. The temporal pattern of the IL-22 Fc fusion protein-induced CRP elevations was highly reproducible. In this study, a significant deviation from this pattern only occurred in one subject in the context of a concurrent viral illness. Future use of IL-22 Fc fusion protein in diseases associated with elevations in CRP should account for IL-22 Fc fusion protein-induced CRP elevation when monitoring disease activity.

A factor that may influence IL-22 Fc fusion protein activity is IL-22 binding protein (IL-22BP), a soluble form of the IL-22R1 subunit and an important negative regulator of IL-22 signaling. IL-22BP has a higher affinity for IL-22 than the membrane-bound form of the receptor, and its expression levels in multiple tissues and cell types may variably interfere with IL-22 Fc fusion protein signaling. This interference may change IL-22-pathway activity in different tissues or diseases, such as inflammatory bowel disease, or even in individuals. In the PK assay for this study, IL-22BP did not interfere with measurements of IL-22 Fc fusion protein concentrations. Furthermore, circulating levels of IL-22BP are orders of magnitude lower than concentrations of IL-22 Fc fusion protein achieved in this study, so IL-22BP is unlikely to substantially affect systemic levels of IL-22 Fc fusion protein.

Because IL-22 Fc fusion protein induces STAT3 phosphorylation and activation via the IL-22 receptor and subsequent epithelial cell proliferation, IL-22 Fc fusion protein may promote the growth of preexisting epithelial tumors. Previous studies report that IL-22 promotes epithelial tumor progression, including squamous cell and basal cell carcinoma and colorectal cancer. In this study, we saw no evidence of tumor growth promotion, although subjects were excluded if there was a personal or family history of malignancy or preneoplastic lesions.

In this single-ascending-dose study, ADA incidence was low and there were no persistent ADAs. However, repeat administration may increase the chance of ADAs. Because ADAs could not only affect PK and/or IL-22 Fc fusion protein efficacy, but could also neutralize endogenous IL-22, ADAs will be monitored in future studies. Mice lacking IL-22 are normal and healthy, but may be more vulnerable to certain bacterial or fungal infections. IL-22 deficiency in humans is not fully understood, but may predispose patients to certain mucocutaneous and/or opportunistic infections.

In conclusion, this Phase 1a study demonstrated satisfactory safety and pharmacokinetics in addition to providing evidence of IL-22R engagement and dose-dependent pharmacological activity of IL-22 Fc fusion protein in healthy volunteers. Activation of the IL-22 pathway is expected to provide broad therapeutic advantages for diseases involving epithelial damage or dysfunction, for example, colitis, hepatitis, acute kidney injury, pancreatitis, and skin wound healing. IL-22 may also support regeneration in other tissues, such as thymus, and may protect against systemic bacterial infections originating in the lung or intestine. These data support the use of IL-22 Fc fusion protein for inflammatory conditions involving epithelial dysfunction and/or damage, such as IBD (e.g., UC (e.g., moderate to severe UC) and Crohn's disease), hidradenitis suppurativa, COPD, and nonalcoholic fatty acid liver disease (e.g., NASH).

### Example 2: Phase II Study for Safety, Efficacy, and Pharmacokinetics of IL-22 Fc Fusion Protein Compared to Vedolizumab in Patients with Moderate to Severe UC

To evaluate the safety, efficacy, and pharmacokinetics of IL-22 Fc fusion protein compared with vedolizumab in patients with moderate to severe UC, patients are enrolled in a phase II, randomized, parallel-group, double-blind, double-dummy, multicenter study. Specific objectives and corresponding endpoints for the study are outlined in Table 9. Endoscopic scores are based on interpretation by a central reader.

TABLE 9
Exemplary Objectives and Corresponding Endpoints

| | |
|---|---|
| Primary Efficacy Objective | Corresponding Endpoint |
| To evaluate the efficacy of | Clinical remission at Week 10, with clinical remission |
| IL-22 Fc fusion protein | defined as meeting both of the following criteria: |
| compared with vedolizumab | Modified MCS^{a} of ≤2 |
| | Mayo rectal bleeding subscore of 0 and other Mayo |
| | subscores of ≤1 |
| Secondary Efficacy Objective | Corresponding Endpoints |
| To evaluate the efficacy of | Clinical remission at Week 6 |
| IL-22 Fc fusion protein | Sustained remission, defined as clinical remission at |
| compared with vedolizumab | both Week 10 and Week 30 |
| | Clinical response at Weeks 6, 10, and 30 with clinical |
| | response defined as meeting both of the following |
| | criteria: |
| | A ≥3-point decrease from baseline in modified MCS; |
| | and |
| | A ≥1-point decrease from baseline in Mayo rectal |
| | bleeding subscore or a Mayo rectal bleeding |
| | subscore of 0 or 1 |
| | Endoscopic healing at Weeks 6, 10, and 30, with |
| | endoscopic healing defined as a Mayo endoscopic |
| | subscore of ≤1 |
| | Endoscopic remission at Weeks 6, 10, and 30, with |
| | endoscopic remission defined as a Mayo endoscopic |
| | subscore of 0 |
| | Change from baseline in UC bowel movement signs |
| | and symptoms at Week 10 and at Week 30, as |
| | assessed by UC-PRO/SS score |
| | Change from baseline in UC abdominal signs and |
| | symptoms at Week 10 and at Week 30, as assessed by |
| | UC-PRO/SS score |
| | Change from baseline in patient-reported |
| | health-related QOL at Week 10 and at Week 30, as |
| Exploratory Efficacy Objective | Corresponding Endpoints |
| To evaluate the efficacy of | Mucosal healing, defined as endoscopic healing and |
| IL-22 Fc fusion protein | histological remission ≤6 as per Robarts Histological |
| compared with vedolizumab | Index, at Weeks 6, 10, and 30 |
| | Change from baseline in UC Endoscopic Index of |
| | Severity at Weeks 6, 10, and 30 |
| | Histological healing, defined as Nancy score of 0 or 1 as |
| | per Nancy Histological Index, at Weeks 6, 10, and 30 |
| Safety Objective | Corresponding Endpoints |
| To evaluate the safety of | Occurrence and severity of adverse events, with |
| IL-22 Fc fusion protein | severity determined according to NCI CTCAE scale |
| compared with vedolizumab | Change in targeted vital signs, physical findings, and |
| | clinical laboratory test results during and following study |
| | drug administration |
| Pharmacokinetic Objective | Corresponding Endpoint |
| To characterize the | Serum concentration of IL-22 Fc fusion protein at |
| pharmacokinetics of | specified timepoints |
| IL-22 Fc fusion protein | |
| in patients with UC | |
| Exploratory Pharmacokinetic | Corresponding Endpoints |
| Objectives | |
| To evaluate potential | Relationship between serum concentration or PK |
| relationships between | parameters for IL-22 Fc fusion protein and efficacy |
| drug exposure and the | endpoints |
| efficacy and safety | Relationship between serum concentration or PK |
| of IL-22 Fc fusion | parameters for IL-22 Fc fusion protein and safety |
| protein | endpoints |
| To evaluate potential | Relationship between selected covariates and |
| relationships between | serum concentration or PK parameters for IL-22 |
| selected covariates | Fc fusion protein |
| and exposure to IL-22 | |
| Fc fusion protein | |
| Immunogenicity Objective | Corresponding Endpoint |
| To evaluate the immune | Presence of ADAs during the study relative to the |
| response to IL-22 Fc fusion | presence of ADAs at baseline |
| protein | |
| Exploratory Immunogenicity | Corresponding Endpoint |
| Objective | |
| To evaluate potential effects | Relationship between ADA status and efficacy, |
| of ADAs | safety, or PK endpoints |
| Exploratory Biomarker Objective | Corresponding Endpoint |
| To identify biomarkers that are | Relationship between biomarkers in blood, stool, |
| predictive of response to IL-22 Fc | and colonic tissue, and efficacy, safety, PK, |
| fusion protein (i.e., predictive | immunogenicity, or other biomarker endpoints |
| biomarkers), are associated with | |
| progression to a more severe | |
| disease state (i.e., prognostic | |
| biomarkers), are associated with | |
| susceptibility to developing | |
| adverse events (i.e., safety | |
| biomarkers), can provide evidence | |
| of IL-22 Fc fusion protein | |
| activity (i.e., pharmacodynamic | |
| biomarkers), or can increase the | |
| knowledge and understanding of | |
| disease biology | |

ADA, anti-drug antibody; IBDQ, Inflammatory Bowel Disease Questionnaire; MCS, Mayo Clinic Score; NCI CTCAE, National Cancer Institute Common Terminology Criteria for Adverse Events; PD, pharmacodynamic; PK, pharmacokinetic; UC-PRO/SS, Ulcerative Colitis-Patient-Reported Outcome Signs and Symptoms.
Note:
Endoscopic scores will be based on interpretation by a central reader.
^{a}Modified MCS is the composite of three MCS assessments: stool frequency, rectal bleeding, and centrally-read endoscopy

### A. Study Design

The study is a Phase II, randomized, parallel-group, double-blind, double-dummy, multicenter study to evaluate the efficacy, safety, and pharmacokinetics of IL-22 Fc fusion protein compared with vedolizumab in the treatment of moderate to severe UC.

This study includes a screening period of up to 4 weeks, a treatment period consisting of a 10-week phase (Part A) to test the induction of clinical remission and a 20-week phase (Part B) to test the durability of clinical remission, and an 8-week safety follow-up period. Patients enrolled in this study have the opportunity to participate in an Extension Study, if eligible.

Patients are randomly assigned in a 1:1:1:1:1:1:1:1:2 ratio to one of nine treatment arms, as outlined below in Table 10. The indicated therapy is administered on the first day of the indicated week.

TABLE 10
Treatment Arms for Study

| | Part A (Weeks 0-10) | | Part B (Weeks 11-30) | |
|---|---|---|---|---|
| Arm | Active Drug | Placebo | Active Drug | Placebo |
| 1A | IL-22 Fc fusion protein | VDZ placebo IV | IL-22 Fc fusion protein | VDZ placebo IV |
| | 60 μg/kg IV at Weeks | at Weeks 0, 2, 6 | 60 μg/kg IV at Weeks | at Weeks 14, 22, 30 |
| | 0, 2, 4, 6, 8, 10 | | 14, 22, 30 | |
| 1B | IL-22 Fc fusion protein | VDZ placebo IV | — | VDZ placebo IV + |
| | 60 μg/kg IV at Weeks | at Weeks 0, 2, 6 | | IL-22 Fc fusion |
| | 0, 2, 4, 6, 8, 10 | | | protein placebo IV |
| | | | | at Weeks 14, 22, 30 |
| 2A | IL-22 Fc fusion protein | VDZ placebo IV | IL-22 Fc fusion protein | VDZ placebo IV |
| | 60 μg/kg IV at Weeks | at Weeks 0, 2, 6 | 60 μg/kg IV at Weeks | at Weeks 14, 22, 30 |
| | 0, 4, 8 | IL-22 Fc fusion | 14, 22, 30 | |
| | | protein placebo IV | | |
| | | at Weeks 2, 6, 10 | | |
| 2B | IL-22 Fc fusion protein | VDZ placebo IV | — | VDZ placebo IV + |
| | 60 μg/kg IV at Weeks | at Weeks 0, 2, 6 | | IL-22 Fc fusion |
| | 0, 4, 8 | IL-22 Fc fusion | | protein placebo IV |
| | | protein placebo IV | | at Weeks 14, 22, 30 |
| | | at Weeks 2, 6, 10 | | |
| 3A | IL-22 Fc fusion protein | VDZ placebo IV | IL-22 Fc fusion protein | VDZ placebo IV |
| | 30 μg/kg IV at Weeks | at Weeks 0, 2, 6 | 60 μg/kg IV at Weeks | at Weeks 14, 22, 30 |
| | 0, 4, 8 | IL-22 Fc fusion | 14, 22, 30 | |
| | | protein placebo IV | | |
| | | at Weeks 2, 6, 10 | | |
| 3B | IL-22 Fc fusion protein | VDZ placebo IV | — | VDZ placebo IV + |
| | 30 μg/kg IV at Weeks | at Weeks 0, 2, 6 | | IL-22 Fc fusion |
| | 0, 4, 8 | IL-22 Fc fusion | | protein placebo IV |
| | | protein placebo IV | | at Weeks 14, 22, 30 |
| | | at Weeks 2, 6, 10 | | |
| 4A | IL-22 Fc fusion protein | VDZ placebo IV | IL-22 Fc fusion protein | VDZ placebo IV |
| | 90 μg/kg IV at Weeks | at Weeks 0, 2, 6 | 60 μg/kg IV at Weeks | at Weeks 14, 22, 30 |
| | 0, 6 | IL-22 Fc fusion | 14, 22, 30 | |
| | | protein placebo IV | | |
| | | at Weeks 2, 4, 8, 10 | | |
| 4B | IL-22 Fc fusion protein | VDZ placebo IV | — | VDZ placebo IV + |
| | 90 μg/kg IV at Weeks | at Weeks 0, 2, 6 | | IL-22 Fc fusion |
| | 0, 6 | IL-22 Fc fusion | | protein placebo IV |
| | | protein placebo IV | | at Weeks 14, 22, 30 |
| | | at Weeks 2, 4, 8, 10 | | |
| 5 | VDZ 300 mg IV at | IL-22 Fc fusion | VDZ 300 mg IV at | IL-22 Fc fusion |
| | Weeks 0, 2, 6 | protein placebo IV | Weeks 14, 22, 30 | protein placebo IV |
| | | at Weeks 0, 2, 4, | | at Weeks 14, 22, 30 |
| | | 6, 8, 10 | | |

IV, intravenous infusion; VDZ, vedolizumab.

Patient disposition during the study is based on whether patients receive rescue therapy or meet criteria for clinical response or disease flare, as defined below.

(i) Rescue therapy, defined as initiation of any treatment for UC or an increase in the dose of oral 5-aminosalicylic acid (5-ASA) or oral corticosteroids compared with baseline (randomization) Cyclosporine, tacrolimus, sirolimus, mycophenolate mofetil, and anti-integrin agents are not permitted as rescue therapy.

(ii) Clinical response, defined as meeting both of the following criteria:
(a) A 3-point decrease from baseline in modified Mayo Clinic Score (mMCS), defined as the composite of three MCS assessments: stool frequency, rectal bleeding, and centrally-read endoscopy; and
(b) A 1-point decrease from baseline in rectal bleeding subscore or a rectal bleeding subscore of 0 or 1;

(iii) Disease flare, defined as meeting one of the following sets of criteria during Part B:
(a) An increase from Week 10 in partial MCS (pMCS), defined as a composite of three MCS assessments: stool frequency, rectal bleeding, and Physician's Global Assessment, of 3 points, an absolute pMCS of 5, and an endoscopy subscore of 2; or
(b) An absolute pMCS score of 7 and an endoscopy subscore of 2.

### Part A

During Part A, patients receive an IV infusion of either IL-22 Fc fusion protein or IL-22 Fc fusion protein placebo at Weeks 0, 2, 4, 6, 8, and 10. At Weeks 0, 2, and 6, patients also receive an IV infusion of either vedolizumab or vedolizumab placebo (see treatment regimens outlined above).

At Weeks 6 and 10, patients undergo a flexible sigmoidoscopy with biopsy and a full MCS is assessed. The mMCS and pMCS are derived from the MCS.

Patients who receive rescue therapy and patients with persistent or worsening disease for which rescue therapy is indicated, as determined by the investigator, are expected to return to the clinic as soon as possible for a disease evaluation visit, which includes a flexible sigmoidoscopy and MCS evaluation. The disease evaluation visit is expected to occur no later than 1 week after initiation of rescue therapy. Patients who receive rescue therapy discontinue study drug and undergo all scheduled clinic assessments through Week 10, with the exception of the flexible sigmoidoscopy if already performed at a disease evaluation visit. Upon completion of the Week 10 visit, these patients may enroll in the Extension Study, if eligible.

Patients who meet the criteria for clinical response at Week 10 (without use of rescue therapy) continue into Part B. Patients who do not meet the criteria for clinical response discontinue the study drug and may enroll in the Extension Study, if eligible.

Patients who are ineligible for or choose not to enroll in the Extension Study enter the safety follow-up period.

### Part B

During Part B, patients receive IV infusions of IL-22 Fc fusion protein and vedolizumab placebo (Arms 1A, 2A, 3A, and 4A), IL-22 Fc fusion protein placebo and vedolizumab placebo (Arms 1B, 2B, 3B, and 4B), or vedolizumab and IL-22 Fc fusion protein placebo (Arm 5) at Weeks 14, 22, and 30 as per the treatment regimens outlined above.

For patients on concomitant oral corticosteroids at baseline, the corticosteroid dose is tapered until discontinuation, starting at Week 10.

For patients who cannot tolerate the corticosteroid taper without recurrence of UC symptoms or experience symptoms of corticosteroid withdrawal, the corticosteroid dose can be increased. If the corticosteroid dose has not been increased above the baseline level, these patients are expected to re-initiate corticosteroid dose tapering per the above regimen within 2 weeks. Patients who are unable to tolerate a second taper may discontinue study drug and enroll in the Extension Study, if eligible, or may continue in the blinded study, as determined by the investigator. Treatment with corticosteroids above the baseline dose will be considered rescue therapy.

Patients who receive rescue therapy and patients with worsening disease for which rescue therapy is indicated, as determined by the investigator, are expected to return to the clinic as soon as possible for a disease evaluation visit, which includes a flexible sigmoidoscopy and MCS evaluation. The disease evaluation visit is expected to occur no later than 1 week after initiation of rescue therapy. Patients who receive rescue therapy or experience disease flare discontinue study drug and may enroll in the Extension Study, if eligible. Patients who are ineligible for or choose not to enroll in the Extension Study enter the safety follow-up period.

Patients who complete Part B (i.e., through Week 30) enter the safety follow-up period.

### Safety Follow-Up Period

Patients who complete the treatment period (Parts A and B) and patients who discontinue study drug without entering the Extension Study enter the safety follow-up period and undergo assessments at 4 and 8 weeks after their last dose of study drug.

Patients who complete the treatment period (Parts A and B) and the safety follow-up period may enroll in the Extension Study, if eligible.

### Extension Study

Patients who enter the Extension Study at Week 10 or during Part B (i.e., do not complete maintenance treatment) receive treatment with open-label IL-22 Fc fusion protein.

Patients in Arms 1A, 1B, 2A, 2B, 3A, 3B, 4A, and 4B who complete the treatment period (Parts A and B) and safety follow-up period receive blinded vedolizumab placebo during Extension Study GA40209. After the phase II study is unblinded, the extension study is also unblinded and patients in Arms 1A, 1B, 2A, 2B, 3A, 3B, 4A, and 4B discontinue vedolizumab placebo infusions. Patients who experience disease flare at any time during the extension study receive treatment with open-label IL-22 Fc fusion protein.

Patients in Arm 5 who complete the treatment period (Parts A and B) and safety follow-up period receive blinded vedolizumab during the Extension Study. After the phase II study is unblinded, patients in Arm 5 receive open-label vedolizumab in the Extension Study. Patients who experience disease flare at any time during the extension study will discontinue vedolizumab and receive open-label IL-22 Fc fusion protein.

### B. Number of Patients

Approximately 300 patients are enrolled in the study across global investigational sites.

### C. End of Study

The end of this study is defined as the date when the last patient completes the last safety follow-up visit. The end of the study is expected to occur approximately 38 weeks after the last patient initiates treatment.

### D. Length of Study

The total length of the study, from screening of the first patient to the end of the study, is expected to be approximately 36 months.

### E. Investigational Medicinal Products

The investigational medicinal products for this study are IL-22 Fc fusion protein and vedolizumab (active comparator).

### Test Product (Investigational Drug)

IL-22 Fc fusion protein or IL-22 Fc fusion protein placebo is administered by IV infusion. During Part A, patients receive an infusion of IL-22 Fc fusion protein or IL-22 Fc fusion protein placebo according to the following dosing regimen.

Arms 1A and 1B: IL-22 Fc fusion protein 60 μg/kg at Weeks 0, 2, 4, 6, 8, and 10

Arms 2A and 2B: IL-22 Fc fusion protein 60 μg/kg at Weeks 0, 4, and 8; IL-22 Fc fusion protein placebo at Weeks 2, 6, and 10

Arms 3A and 3B: IL-22 Fc fusion protein 30 μg/kg at Weeks 0, 4, and 8; IL-22 Fc fusion protein placebo at Weeks 2, 6, and 10

Arms 4A and 4B: IL-22 Fc fusion protein 90 μg/kg at Weeks 0 and 6; IL-22 Fc fusion protein placebo at Weeks 2, 4, 8, and 10

Arm 5: IL-22 Fc fusion protein placebo at Weeks 0, 2, 4, 6, 8, and 10

During Part B, patients receive an infusion of IL-22 Fc fusion protein or IL-22 Fc fusion protein placebo according to the following dosing regimen.

Arms 1A, 2A, 3A, and 4A: IL-22 Fc fusion protein 60 μg/kg at Weeks 14, 22, and 30

Arms 1B, 2B, 3B, 4B, and 5: IL-22 Fc fusion protein placebo at Weeks 14, 22, and 30

### Comparator

Vedolizumab 300 mg or vedolizumab placebo is administered by IV infusion.

During Parts A and B, patients in Arms 1-4 receive an infusion of vedolizumab placebo at Weeks 0, 2, 6, 14, 22, and 30.

During Parts A and B, patients in Arm 5 receive an infusion of 300 mg vedolizumab at Weeks 0, 2, 6, 14, 22, and 30.

### Example 3: Clinical Trial in Healthy Volunteers and Patients with Moderate to Severe UC

In a Phase 1 clinical trial, healthy volunteers and UC patients were treated with either placebo or one of the following dosing regimens of IL-22 Fc fusion protein (UTTR1147A): 60 μg/kg every four weeks for three administrations (q4w x 3), 60 μg/kg every two weeks for six administrations (q2w x 6), and 90 μg/kg every two weeks for 6 administrations (q2w x 6). In an interim analysis, patients were assessed for clinical response, as well as the other parameters described in Example 1. While no clinical response was observed in UC patients receiving placebo, clinical response and/or clinical remission was observed in a subset of patients receiving study drug at all three dosing regimens, providing an early indication of clinical activity of the study drug. Surprisingly, the observed clinical benefit was maintained at least out to 3 months after the last dose (end of study), indicating that the study drug could have long-lasting benefit.

### Example 4: Multicenter Study to Evaluate the Safety, Efficacy, and Pharmacokinetics of IL-22 Fc Fusion Protein (UTTR1147A) in Combination with Standard of Care in the Prevention of Acute Graft Versus Host Disease (aGVHD) in Patients Undergoing Allogeneic Hematopoietic Stem Cell Transplantation

This study will evaluate the efficacy, safety, and pharmacokinetics of IL-22 Fc fusion protein (UTTR1147A) versus placebo when given in combination with standard of care (SOC) aGVHD prophylaxis to prevent aGVHD in patients undergoing allo-HSCT.

### Study Design

Patients will be randomly assigned in a 1:1 ratio to receive either UTTR1147A 60 μg/kg IV combined with standard of care or UTTR1147A placebo combined with standard of care aGVHD prophylaxis. All patients will receive standard of care treatment consisting of one of the following treatment combinations, as determined by the institution:calcineurin (CN) inhibitor+methotrexate or mycophenolate mofetil (MMF). The first dose of study drug (UTTR1147A or placebo) will be administered 3 (±2) days prior to allo-HSCT, the second dose will be administered on Day 11, and subsequent doses will be administered Q2W through Day 96 for a total of 8 doses of study drug.

The primary efficacy objective for this study is to evaluate the efficacy of UTTR1147A given in combination with standard of care based on the following endpoint:
Incidence of Grade IIIV aGVHD at Post-Transplant Day 100 according to the MAGIC GVHD Target Organ Staging (see, e.g., Harris et al. Biol. Blood Marrow Transplant 22(1):4-10, 2016, which is incorporated herein by reference in its entirety).

The secondary efficacy objective for this study is to evaluate the efficacy of UTTR1147A plus standard of care compared with placebo plus standard of care on the basis of the following endpoints:
Incidence of each of four stages of aGVHD (i.e., Stage 1, 2, 3, or 4) of skin, gut, and liver at Post-Transplant Day 100 according to the MAGIC GVHD Target Organ Staging
Incidence of each of four grades of aGVHD (i.e., Grade I, II, III, or IV) at Post-Transplant Day 100 according to the MAGIC GVHD Target Organ Staging
GI aGVHDfree survival rate at Post-Transplant Day 180, defined as the proportion of patients with absence of Grade IIIV GI aGVHD at Post-Transplant Day 180
Overall survival (OS) rate at Post-Transplant Day 180, defined as the proportion of patients who have not experienced death from any cause at Post-Transplant Day 180% OS rate at Post-Transplant Day 365, defined as the proportion of patients who have not experienced death from any cause at Post-Transplant Day 365
Relapse-free survival rate at Post-Transplant Day 180, defined as the proportion of patients who have not experienced relapse of primary disease or death, whichever occurs first, at Post-Transplant Day 180
Relapse-free survival rate at Post-Transplant Day 365, defined as the proportion of patients who have not experienced relapse of primary disease or death, whichever occurs first, at Post-Transplant Day 365
Incidence of chronic GVHD according to the National Institutes of Health cGVHD score at Post-Transplant Day 365

### Primary Efficacy Endpoint

The primary efficacy endpoint is incidence of Grade II-IV aGVHD at Post-transplant Day 100 Incidence of Grade II-IV GVHD is defined as number of patients diagnosed with Grade II-IV aGVHD, where aGVHD grade is assessed by blinded investigator using MAGIC GVHD Target Organ Staging.

### Secondary Efficacy Endpoints

The secondary efficacy endpoints will be summarized by descriptive statistics based on the following calculations: Proportion of patients with organ-specific aGVHD (i.e. skin, GI tract, or liver) at Post-Transplant Day 100
Proportion of patients with each of four grades of aGVHD (i.e., Grade I, II, III, or IV) at Post-Transplant Day 100
Proportion of patients with absence of Grade IIIV GI aGVHD at Post-Transplant Day 180
Proportion of patients who have not experienced death from any cause at Post-Transplant Day 180
Proportion of patients who have not experienced death from any cause at Post-Transplant Day 365
Proportion of patients who have not experienced relapse of primary disease or death, whichever occurs first, at Post-Transplant Day 180
Proportion of patients who have not experienced relapse of primary disease or death, whichever occurs first, at Post-Transplant Day 365
Proportion of patients with chronic GVHD at Post-Transplant Day 365

### OTHER EMBODIMENTS

Some embodiments of the technology described herein can be defined according to any of the following numbered embodiments:

1. A method of treating a subject having an inflammatory bowel disease (IBD) comprising administering to the subject an IL-22 Fc fusion protein in a dosing regimen comprising a dosing cycle, wherein the dosing cycle comprises between two and six doses, and wherein a total of about 30 μg/kg to about 720 μg/kg of the IL-22 Fc fusion protein is administered to the subject in the dosing cycle.

2. The method of embodiment 1, wherein the doses are administered to the subject every week (q1w), every two weeks (q2w), every four weeks (q4w), or every six weeks (q6w).

3. The method of embodiment 1 or 2, wherein a total of about 540 μg/kg of the IL-22 Fc fusion protein is administered to the subject in the dosing cycle.

4. The method of embodiment 1 or 2, wherein a total of about 360 μg/kg of the IL-22 Fc fusion protein is administered to the subject in the dosing cycle.

5. The method of embodiment 1 or 2, wherein a total of about 180 μg/kg of the IL-22 Fc fusion protein is administered to the subject in the dosing cycle.

6. The method of embodiment 1 or 2, wherein a total of about 90 μg/kg of the IL-22 Fc fusion protein is administered to the subject in the dosing cycle.

7. The method of any one of embodiments 1-6, wherein the length of the dosing cycle is between about 5 weeks and about 15 weeks.

8. The method of embodiment 7, wherein the length of the dosing cycle is between 8 weeks and 12 weeks.

9. The method of embodiment 7, wherein the length of the dosing cycle is about 10 weeks.

10. The method of any one of embodiments 1-9, wherein the dosing cycle comprises a first dose (C1D1) and a second dose (C1D2) of the IL-22 Fc fusion protein.

11. The method of embodiment 10, wherein the dosing cycle consists of the C1D1 and the C1D2.

12. The method of embodiment 10 or 11, wherein the C1D1 and the C1D2 are each between about 30 μg/kg to about 135 μg/kg.

13. The method of embodiment 12, wherein the C1D1 and the C1D2 are each about 90 μg/kg.

14. The method of any one of embodiments 11-13, wherein the method comprises administering to the subject the C1D1 and the C1D2 on or about Weeks 0 and 6, respectively, of the dosing cycle.

15. The method of any one of embodiments 1-10 and 12-14, wherein the dosing cycle comprises a first dose (C1D1), a second dose (C1D2), and a third dose (C1D3) of the IL-22 Fc fusion protein.

16. The method of embodiment 15, wherein the dosing cycle consists of the C1D1, the C1D2, and the C1D3.

17. The method of embodiment 15 or 16, wherein the C1D1, the C1D2, and the C1D3 are each between about 15 μg/kg to about 90 μg/kg.

18. The method of embodiment 17, wherein the C1D1, the C1D2, and the C1D3 are each between about 20 μg/kg to about 40 μg/kg.

19. The method of embodiment 18, wherein the C1D1, the C1D2, and the C1D3 are each about 30 μg/kg.

20. The method of embodiment 17, wherein the C1D1, the C1D2, and the C1D3 are each between about 50 μg/kg to about 70 μg/kg.

21. The method of embodiment 20, wherein the C1D1, the C1D2, and the C1D3 are each about 60 μg/kg.

22. The method of any one of embodiments 15-21, wherein the method comprises administering the C1D1, the C1D2, and the C1D3 on or about Weeks 0, 4, and 8, respectively, of the dosing cycle.

23. The method of any one of embodiments 1-10, 12-15, and 17-22, wherein the dosing cycle comprises a first dose (C1D1), a second dose (C1D2), a third dose (C1D3), a fourth dose (C1D4), a fifth dose (C1D5), and a sixth dose (C1D6) of the IL-22 Fc fusion protein.

24. The method of embodiment 23, wherein the dosing cycle consists of the C1D1, the C1D2, the C1D3, the C1D4, the C1D5, and the C1D6.

25. The method of embodiment 23 or 24, wherein the C1D1, the C1D2, the C1D3, the C1D4, the C1D5, and the C1D6 are each between about 30 μg/kg to about 90 μg/kg.

26. The method of embodiment 25, wherein the C1D1, the C1D2, the C1D3, the C1D4, the C1D5, and the C1D6 are each between about 50 μg/kg to about 70 μg/kg.

27. The method of embodiment 26, wherein the C1D1, the C1D2, the C1D3, the C1D4, the C1D5, and the C1D6 are each about 60 μg/kg.

28. The method of any one of embodiments 23-27, wherein the method comprises administering the C1D1, the C1D2, the C1D3, the C1D4, the C1D5, and the C1D6 on or about Weeks 0, 2, 4, 6, 8, and 10, respectively, of the dosing cycle.

29. The method of any one of embodiments 1-10, 12-15, 17-23, and 25-28, wherein the dosing regimen further comprises a further dosing cycle.

30. The method of embodiment 29, wherein the length of the further dosing cycle is between about 10 weeks and about 40 weeks.

31. The method of embodiment 30, wherein the length of the further dosing cycle is between about 15 weeks and about 25 weeks.

32. The method of embodiment 31, wherein the length of the further dosing cycle is about 20 weeks.

33. The method of any one of embodiments 30-32, wherein the further dosing cycle comprises a first dose (C2D1), a second dose (C2D2), and a third dose (C2D3) of the IL-22 Fc fusion protein.

34. The method of embodiment 33, wherein the C2D1, the C2D2, and the C2D3 are each between about 30 μg/kg to about 90 μg/kg.

35. The method of embodiment 34, wherein the C2D1, the C2D2, and the C2D3 are each between about 50 μg/kg to about 70 μg/kg.

36. The method of embodiment 35, wherein the C2D1, the C2D2, and the C2D3 are each about 60 μg/kg.

37. The method of any one of embodiments 33-36, wherein the method comprises administering the C2D1, the C2D2, and the C2D3 on or about Weeks 4, 12, and 20, respectively, of the further dosing cycle.

38. A method of treating a subject having an IBD comprising administering to the subject an IL-22 Fc fusion protein in a dosing regimen comprising a dosing cycle having a length of about 10 weeks, wherein the dosing cycle comprises a first dose (C1D1) and a second dose (C1D2) of the IL-22 Fc fusion protein, wherein the C1D1 and the C1D2 are each about 90 μg/kg, and wherein the C1D1 and C1D2 are administered to the subject on or about Weeks 0 and 6, respectively, of the dosing cycle.

39. A method of treating a subject having an IBD comprising administering to the subject an IL-22 Fc fusion protein in a dosing regimen comprising a dosing cycle having a length of about 10 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 30 μg/kg, and wherein the C1D1, the C1D2, and the C1D3 are administered to the subject on or about Weeks 0, 4, and 8, respectively, of the dosing cycle.

40. A method of treating a subject having an IBD comprising administering to the subject an IL-22 Fc fusion protein in a dosing regimen comprising a dosing cycle having a length of about 10 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 60 μg/kg, and wherein the C1D1, the C1D2, and the C1D3 are administered to the subject on or about Weeks 0, 4, and 8, respectively, of the dosing cycle.

41. A method of treating a subject having an IBD comprising administering to the subject an IL-22 Fc fusion protein in a dosing regimen comprising a dosing cycle having a length of about 10 weeks, wherein the dosing cycle comprises a first dose (C1D1), a second dose (C1D2), a third dose (C1D3), a fourth dose (C1D4), a fifth dose (C1D5), and a sixth dose (C1D6) of the IL-22 Fc fusion protein, wherein the C1D1, C1D2, the C1D3, the C1D4, the C1D5, and the C1D6 are each about 60 μg/kg, and wherein the C1D1, the C1D2, the C1D3, the C1D4, the C1D5, and the C1D6 are administered to the subject on or about Weeks 0, 2, 4, 6, 8, and 10, respectively, of the dosing cycle.

42. A method of treating a subject having an IBD comprising administering to the subject an IL-22 Fc fusion protein in a dosage regimen comprising at least a first dosing cycle and a second dosing cycle, wherein:

(a) the first dosing cycle has a length of about 10 weeks and comprises a first dose (C1D1) and a second dose (C1D2) of the IL-22 Fc fusion protein, wherein the C1D1 and the C1D2 are each about 90 μg/kg, and wherein the C1D1 and the C1D2 are administered to the subject on or about Weeks 0 and 6, respectively, of the first dosing cycle; and

(b) the second dosing cycle has a length of about 20 weeks and comprises a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are administered on or about Weeks 4, 12, and 20, respectively, of the second dosing cycle.

43. A method of treating a subject having an IBD comprising administering to the subject an IL-22 Fc fusion protein in a dosage regimen comprising at least a first dosing cycle and a second dosing cycle, wherein:

(a) the first dosing cycle has a length of about 10 weeks and comprises a first dose (C1D1), a second dose (C1D2), and a third dose (C1D3) of the IL-22 Fc fusion protein, wherein the C1D1, the C1D2, and the C1D3 are each about 30 μg/kg, and wherein the C1D1, the C1D2, and the C1D2 are administered to the subject on or about Weeks 0, 4, and 8, respectively, of the first dosing cycle; and

(b) the second dosing cycle has a length of about 20 weeks and comprises a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are administered on or about Weeks 4, 12, and 20, respectively, of the second dosing cycle.

44. A method of treating a subject having an IBD comprising administering to the subject an IL-22 Fc fusion protein in a dosage regimen comprising at least a first dosing cycle and a second dosing cycle, wherein:

(a) the first dosing cycle has a length of about 10 weeks and comprises a first dose (C1D1), a second dose (C1D2), and a third dose (C1D3) of the IL-22 Fc fusion protein, wherein the C1D1, the C1D2, and the C1D3 are each about 60 μg/kg, and wherein the C1D1, the C1D2, and the C1D2 are administered to the subject on or about Weeks 0, 4, and 8, respectively, of the first dosing cycle; and

(b) the second dosing cycle has a length of about 20 weeks and comprises a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are administered on or about Weeks 4, 12, and 20, respectively, of the second dosing cycle.

45. A method of treating a subject having an IBD comprising administering to the subject an IL-22 Fc fusion protein in a dosage regimen comprising at least a first dosing cycle and a second dosing cycle, wherein:

(a) the first dosing cycle has a length of about 10 weeks and comprises a first dose (C1D1), a second dose (C1D2), a third dose (C1D3), a fourth dose (C1D4), a fifth dose (C1D5), and a sixth dose (C1D6) of the IL-22 Fc fusion protein, wherein the C1D1, the C1D2, the C1D3, the C1D4, the C1D5, and the C1D6 are each about 60 μg/kg, and wherein the C1D1, the C1D2, the C1D3, the C1D4, the C1D5, and the C1D6 are administered to the subject on or about Weeks 0, 2, 4, 6, 8, and 10, respectively, of the dosing cycle; and

(b) the second dosing cycle has a length of about 20 weeks and comprises a first dose (C2D1), a second dose (C2D2), and a third dose (C2D3) of the IL-22 Fc fusion protein, wherein the C2D1, the C2D2, and the C2D3 are each about 60 μg/kg, and wherein the C2D1, the C2D2, and the C2D3 are administered on or about Weeks 4, 12, and 20, respectively, of the second dosing cycle.

46. The method of any one of embodiments 1-45, wherein the treating ameliorates one or more symptoms of the IBD.

47. The method of embodiment 46, wherein the one or more symptoms of IBD include stool frequency, rectal bleeding, or mucosal appearance.

48. The method of embodiment 47, wherein mucosal appearance comprises erythema, decreased or absent vascular pattern, friability, erosions, spontaneous bleeding, and/or ulceration.

49. The method of any one of embodiments 1-48, wherein the treating results in a clinical remission.

50. The method of embodiment 49, wherein the treating results in a clinical remission within about ten weeks from the first dose.

51. The method of embodiment 50, wherein the treating results in a clinical remission within about six weeks from the first dose.

52. The method of any one of embodiments 49-51, wherein the clinical remission is a modified Mayo Clinic Score (MCS) of less than or equal to about 2 and a Mayo rectal bleeding subscore of 0 and other Mayo subscores of less than or equal to about 1.

53. The method of any one of embodiments 49-52, wherein the clinical remission is a sustained remission.

54. The method of embodiment 53, wherein the sustained remission is a clinical remission at about ten weeks from the first dose and at about 30 weeks from the first dose.

55. The method of any one of embodiments 1-54, wherein the treating results in a clinical response.

56. The method of embodiment 55, wherein the clinical response comprises a decrease in the subject's mMCS score relative to a baseline mMCS score.

57. The method of embodiment 56, wherein the decrease in the subject's mMCS score is a decrease of at least about 1 point or higher relative to the baseline mMCS score.

58. The method of embodiment 57, wherein the decrease in the subject's mMCS score is a decrease of at least about 3 points or higher relative to the baseline mMCS score.

59. The method of any one of embodiments 55-58, wherein the clinical response comprises a decrease in the subject's Mayo rectal bleeding subscore relative to a baseline Mayo rectal bleeding subscore or a Mayo rectal bleeding subscore of 0 or 1.

60. The method of embodiment 59, wherein a decrease in the subject's Mayo rectal bleeding subscore is a decrease of about 1 point or higher relative to the baseline Mayo rectal bleeding subscore.

61. The method of any one of embodiments 55-60, wherein the clinical response is present about 6 weeks after the first dose.

62. The method of any one of embodiments 55-61, wherein the clinical response is present about 10 weeks after the first dose.

63. The method of any one of embodiments 55-62, wherein the clinical response is present about 30 weeks after the first dose.

64. The method of any one of embodiments 1-63, wherein the treating results in endoscopic healing.

65. The method of embodiment 64, wherein the endoscopic healing is a Mayo endoscopic subscore of less than or equal to about 1.

66. The method of any one of embodiments 63-65, wherein the endoscopic healing is present about 6 weeks after the first dose.

67. The method of any one of embodiments 63-66, wherein the endoscopic healing is present about 10 weeks after the first dose.

68. The method of any one of embodiments 63-67, wherein the endoscopic healing is present about 30 weeks after the first dose.

69. The method of any one of embodiments 1-68, wherein the treating results in an endoscopic remission.

70. The method of embodiment 69, wherein the endoscopic remission is a Mayo endoscopic subscore of zero.

71. The method of embodiment 69 or 70, wherein the endoscopic remission is present about 6 weeks after the first dose.

72. The method of any one of embodiments 69-71, wherein the endoscopic remission is present about 10 weeks after the first dose.

73. The method of any one of embodiments 69-72, wherein the endoscopic remission is present about 30 weeks after the first dose.

74. The method of any one of embodiments 1-73, wherein the treating results in a change from baseline in the subject's bowel movement signs and symptoms as assessed by the Ulcerative Colitis Patient-Reported Outcome Signs and Symptoms (UC-PRO/SS) score.

75. The method of embodiment 74, wherein the change from baseline in the subject's bowel movement signs and symptoms as assessed by the UC-PRO/SS score is present about 10 weeks after the first dose.

76. The method of embodiment 75, wherein the change from baseline in the subject's bowel movement signs and symptoms as assessed by the UC-PRO/SS score is present about 30 weeks after the first dose.

77. The method of any one of embodiments 1-76, wherein the treating results in a change from baseline in the subject's abdominal signs and symptoms as assessed by the Ulcerative Colitis Patient-Reported Outcome Signs and Symptoms (UC-PRO/SS) score.

78. The method of embodiment 77, wherein the change from baseline in the subject's abdominal signs and symptoms as assessed by the UC-PRO/SS score is present about 10 weeks after the first dose.

79. The method of embodiment 78, wherein the change from baseline in the subject's abdominal signs and symptoms as assessed by the UC-PRO/SS score is present about 30 weeks after the first dose.

80. The method of any one of embodiments 1-79, wherein the treating results in a change from baseline in the subject's patient-reported health-related quality of life (QOL) as assessed by an Inflammatory Bowel Disease Questionnaire (IBDQ) score.

81. The method of embodiment 80, wherein the change from baseline in the subject's patient-reported health-related QOL as assessed by the IBDQ score is present about 10 weeks after the first dose.

82. The method of embodiment 81, wherein the change from baseline in the subject's patient-reported health-related QOL as assessed by the IBDQ score is present about 30 weeks after the first dose.

83. The method of any one of embodiments 1-82, wherein the treating results in mucosal healing.

84. The method of embodiment 83, wherein the mucosal healing is endoscopic healing and histological remission of less than or equal to about 6, as assessed by Robarts Histological Index.

85. The method of embodiment 83 or 84, wherein the mucosal healing is present about 6 weeks after the first dose.

86. The method of any one of embodiments 83-85, wherein the mucosal healing is present about 10 weeks after the first dose.

87. The method of any one of embodiments 83-86, wherein the mucosal healing is present about 30 weeks after the first dose.

88. The method of any one of embodiments 1-87, wherein the treating results in a change from baseline in the subject's UC Endoscopic Index of Severity.

89. The method of embodiment 87 or 88, wherein the change from baseline in the subject's UC Endoscopic Index of Severity is present about 6 weeks after the first dose.

90. The method of any one of embodiments 87-89, wherein the change from baseline in the subject's UC Endoscopic Index of Severity is present about 10 weeks after the first dose.

91. The method of any one of embodiments 87-90, wherein the change from baseline in the subject's UC Endoscopic Index of Severity is present about 30 weeks after the first dose.

92. The method of any one of embodiments 1-91, wherein the treating results in histological healing.

93. The method of embodiment 92, wherein the histological healing is a Nancy score of 0 or 1 as assessed by the Nancy Histological Index.

94. The method of embodiment 92 or 93, wherein the histological healing is present about 6 weeks after the first dose.

95. The method of any one of embodiments 92-94, wherein the histological healing is present about 10 weeks after the first dose.

96. The method of any one of embodiments 92-95, wherein the histological healing is present about 30 weeks after the first dose.

97. The method of any one of embodiments 1-96, wherein the IBD is ulcerative colitis (UC) or Crohn's disease.

98. The method of embodiment 97, wherein the IBD is UC.

99. The method of embodiment 98, wherein the UC is moderate to severe UC.

100. The method of embodiment 99, wherein the moderate to severe UC is defined as a mMCS of 5-9 with an endoscopic subscore of about 2 or higher, a rectal bleeding subscore of about 1 or higher, and a stool frequency subscore of about 1 or higher prior to the treating.

101. The method of any one of embodiments 97-100, wherein the subject has UC a minimum of about 20 cm from the anal verge as determined by baseline endoscopy.

102. The method of embodiment 97, wherein the IBD is Crohn's disease.

103. The method of any one of embodiments 97-100, wherein the subject has left-sided colitis, extensive colitis, or pancolitis prior to the treating.

104. The method of any one of embodiments 1-103, wherein the subject has had an inadequate response, loss of response, or intolerance to prior immunosuppressant treatment.

105. The method of embodiment 104, wherein the prior immunosuppressant treatment is treatment with an aminosalicylate, an immunomodulatory agent, a tumor necrosis factor (TNF) antagonist, and/or a corticosteroid.

106. The method of embodiment 104 or 105, wherein the subject has had persistent signs or symptoms of active disease despite treatment with at least one 12-week regimen of azathioprine (AZA), mercaptopurine (6-MP), and/or methotrexate (MTX) within five years prior to the treating.

107. The method of any one of embodiments 104-106, wherein the subject has a history of intolerance to AZA, 6-MP, or MTX within five years prior to the treating.

108. The method of any one of embodiments 104-107, wherein the subject has had persistent signs or symptoms of active disease despite treatment with at least two induction doses of infliximab, adalimumab, or golimumab within five years prior to the treating.

109. The method of any one of embodiments 104-108, wherein the subject has had recurrence of signs or symptoms of active disease during maintenance after initial response to induction therapy with infliximab, adalimumab, or golimumab.

110. The method of any one of embodiments 104-109, wherein the subject has had intolerance to a TNF antagonist.

111. The method of any one of embodiments 104-110, wherein:

(i) the subject has had persistent signs or symptoms of active disease despite treatment with at least one 4-week induction regimen that included 30 mg/day of oral prednisone (or equivalent) for at least 2 weeks or 30 mg/day of IV prednisone (or equivalent) for at least 1 week within five years prior to the treating;

(ii) the subject has had two failed attempts to taper corticosteroids below 10 mg/day of oral prednisone (or equivalent); or

(iii) the subject has a history of intolerance to corticosteroids within five years prior to the treating.

112. The method of any one of embodiments 1-111, wherein the IL-22 Fc fusion protein comprises an IL-22 polypeptide linked to an Fc region by a linker.

113. The method of embodiment 112, wherein the IL-22 polypeptide is glycosylated.

114. The method of embodiment 113, wherein the IL-22 polypeptide is N-glycosylated.

115. The method of any one of embodiments 112-114, wherein the Fc region is not glycosylated.

116. The method of embodiment 115, wherein the amino acid residue at position 297 as in the EU index of the Fc region is Gly.

117. The method of embodiment 115, wherein the amino acid residue at position 297 as in the EU index of the Fc region is Ala.

118. The method of any one of embodiments 115-117, wherein the amino acid residue at position 299 as in the EU index of the Fc region is Ala, Gly, or Val.

119. The method of any one of embodiments 112-118, wherein the Fc region comprises the CH2 and CH3 domain of IgG1 or IgG4.

120. The method of embodiment 119, wherein the Fc region comprises the CH2 and CH3 domain of IgG4.

121. The method of any one of embodiments 1-120, wherein the IL-22 Fc fusion protein comprises an amino acid sequence having at least 95% sequence identity to the amino acid sequence of SEQ ID NO:8.

122. The method of embodiment 121, wherein the IL-22 Fc fusion protein comprises an amino acid sequence having at least 96% sequence identity to the amino acid sequence of SEQ ID NO:8.

123. The method of embodiment 122, wherein the IL-22 Fc fusion protein comprises an amino acid sequence having at least 97% sequence identity to the amino acid sequence of SEQ ID NO:8.

124. The method of embodiment 123, wherein the IL-22 Fc fusion protein comprises an amino acid sequence having at least 98% sequence identity to the amino acid sequence of SEQ ID NO:8.

125. The method of embodiment 124, wherein the IL-22 Fc fusion protein comprises an amino acid sequence having at least 99% sequence identity to the amino acid sequence of SEQ ID NO:8.

126. The method of any one of embodiments 1-125, wherein the IL-22 Fc fusion protein comprises the amino acid sequence of SEQ ID NO:8, SEQ ID NO:10, or SEQ ID NO:16.

127. The method of embodiment 126, wherein the IL-22 Fc fusion protein comprises the amino acid sequence of SEQ ID NO:8.

128. The method of embodiment 127, wherein the IL-22 Fc fusion protein consists of the amino acid sequence of SEQ ID NO:8.

129. The method of embodiment 126, wherein the IL-22 Fc fusion protein comprises the amino acid sequence of SEQ ID NO:10.

130. The method of embodiment 129, wherein the IL-22 Fc fusion protein consists of the amino acid sequence of SEQ ID NO:10.

131. The method of embodiment 126, wherein the IL-22 Fc fusion protein comprises the amino acid sequence of SEQ ID NO:16.

132. The method of embodiment 131, wherein the IL-22 Fc fusion protein consists of the amino acid sequence of SEQ ID NO:16.

133. The method of any one of embodiments 112-132, wherein the Fc region is not N-glycosylated.

134. The method of any one of embodiments 1-133, wherein the IL-22 Fc fusion protein is a dimeric IL-22 Fc fusion protein.

135. The method of any one of embodiments 1-133, wherein the IL-22 Fc fusion protein is a monomeric IL-22 Fc fusion protein.

136. The method of any one of embodiments 112-135, wherein the IL-22 polypeptide is a human IL-22 polypeptide.

137. The method of embodiment 136, wherein the IL-22 polypeptide comprises the amino acid sequence of SEQ ID NO:4.

138. The method of any one of embodiments 112-137, wherein the linker comprises the amino acid sequence RVESKYGPP (SEQ ID NO: 44).

139. The method of embodiment 138, wherein the linker consists of the amino acid sequence RVESKYGPP (SEQ ID NO: 44).

140. The method of any one of embodiments 1-139, wherein the IL-22 Fc fusion protein binds to IL-22 receptor.

141. The method of embodiment 140, wherein the IL-22 receptor is human IL-22 receptor.

142. The method of embodiment 140 or 141, wherein the IL-22 Fc fusion protein binds to IL-22RA1 and/or IL-10R2.

143. The method of embodiment 142, wherein the IL-22 Fc fusion protein binds to IL-22RA1.

144. The method of any one of embodiments 1-143, wherein the IL-22 Fc fusion protein is administered to the subject as a monotherapy.

145. The method of any one of embodiments 1-143, wherein the IL-22 Fc fusion protein is administered to the subject as a combination therapy.

146. The method of embodiment 145, wherein the IL-22 Fc fusion protein is administered to the subject concurrently with an additional therapeutic agent.

147. The method of embodiment 145, wherein the IL-22 Fc fusion protein is administered to the subject prior to the administration of an additional therapeutic agent.

148. The method of any one of embodiments 145-147, wherein the IL-22 Fc fusion protein is administered in combination with an additional IBD therapy selected from an aminosalicylate, an immunomodulatory agent, a tumor necrosis factor (TNF) antagonist, an anti-integrin agent, a mucosal addressin cell adhesion molecule (MAdCAM) antagonist, an IL-23 antagonist, an IL-12 antagonist, an IL-12/IL-23 antagonist, an antibiotic, or a corticosteroid.

149. The method of embodiment 148, wherein the additional IBD therapy is an aminosalicylate.

150. The method of embodiment 149, wherein the aminosalicylate comprises 5-aminosalicylic acid (5-ASA).

151. The method of embodiment 148, wherein the additional IBD therapy is an immunomodulatory agent.

152. The method of embodiment 151, wherein the immunomodulatory agent is azathioprine, mercaptopurine, cyclosporine, tacrolimus, sirolimus, mycophenolic acid, or methotrexate.

153. The method of embodiment 148, wherein the additional IBD therapy is a TNF antagonist.

154. The method of embodiment 153, wherein the TNF antagonist is an anti-TNF antibody or a soluble TNF receptor.

155. The method of embodiment 154, wherein the anti-TNF antibody is infliximab, adalimumab, golimumab, or certolizumab pegol.

156. The method of embodiment 154, wherein the soluble TNF receptor is etanercept.

157. The method of embodiment 148, wherein the additional IBD therapy is an anti-integrin agent.

158. The method of embodiment 157, wherein the anti-integrin agent is an anti-integrin antibody.

159. The method of embodiment 158, wherein the anti-integrin antibody is an anti-α4-integrin antibody.

160. The method of embodiment 159, wherein the anti-α4-integrin antibody is natalizumab or vedolizumab.

161. The method of embodiment 148, wherein the MAdCAM antagonist is an anti-MAdCAM antibody.

162. The method of embodiment 161, wherein the anti-MAdCAM antibody is PF-00547659 or SHP647.

163. The method of embodiment 148, wherein the IL-23 antagonist is an anti-IL-23 antibody.

164. The method of embodiment 163, wherein the anti-IL-23 antibody is briakizumab, guselkumab, risankizumab, tilorakizumab, or ustekinumab.

165. The method of embodiment 148, wherein the IL-12 antagonist is an anti-IL-12 antibody.

166. The method of embodiment 165, wherein the anti-IL-12 antibody is ABT-874/J695.

167. The method of embodiment 148, wherein the IL-12/IL-23 antagonist is an anti-IL-12/IL-23 antibody.

168. The method of embodiment 167, wherein the anti-IL-12/IL-23 antibody is ustekinumab or briakinumab.

169. The method of any one of embodiments 1-168, wherein the administering is by intravenous infusion.

170. The method of any one of embodiments 1-168, wherein the administering is by subcutaneous administration.

171. The method of any one of embodiments 1-170, wherein the subject is a human.

172. A kit comprising an IL-22 Fc fusion protein and instructions to administer the IL-22 Fc fusion protein to a subject suffering from an IBD in accordance with the method of any one of embodiments 1-171.

The specification is considered to be sufficient to enable one skilled in the art to practice the invention. Although the foregoing invention has been described in some detail by way of illustration and example for purposes of clarity of understanding, the descriptions and examples should not be construed as limiting the scope of the invention. Indeed, various modifications of the invention in addition to those shown and described herein will become apparent to those skilled in the art from the foregoing description and fall within the scope of the appended claims.

1. A method of treating a subject having an inflammatory bowel disease (IBD) comprising administering to the subject an IL-22 Fc fusion protein in a dosing regimen comprising at least a first dosing cycle, wherein the first dosing cycle comprises between two and ten doses, and wherein a total of about 60 μg/kg to about 900 μg/kg of the IL-22 Fc fusion protein is administered to the subject in the first dosing cycle.

2. The method of claim 1, wherein:
(i) the doses are administered to the subject every week (q1w), every two weeks (q2w), every four weeks (q4w), or every six weeks (q6w);
(ii) a total of about 90 μg/kg, about 180 μg/kg, about 270 μg/kg, about 360 μg/kg, or about 540 μg/kg of the IL-22 Fc fusion protein is administered to the subject in the first dosing cycle;
(iii) the length of the first dosing cycle is between about 5 weeks and about 15 weeks;
(iv) the first dosing cycle comprises a first dose (C1D1), a second dose (C1D2), and a third dose (C1D3) of the IL-22 Fc fusion protein; and/or
(v) the dosing regimen further comprises a second dosing cycle.

3-4. (canceled)

5. The method of claim 2, wherein:
(i) the length of the first dosing cycle is about 8 weeks;
(ii) the C1D1, the C1D2, and the C1D3 are each between about 15 μg/kg to about 90 μg/kg;
(iii) the method comprises administering the C1D1, the C1D2, and the C1D3 on or about Weeks 1, 4, and 8, respectively, of the first dosing cycle;
(iv) the length of the second dosing cycle is between about 10 weeks and about 40 weeks or the second dosing cycle continues indefinitely or until clinical remission;
(v) the doses of the second dosing cycle are administered to the subject every week (q1w), every two weeks (q2w), every four weeks (q4w), every six weeks (q6w), every eight weeks (q8w), every ten weeks (q10w), or every twelve weeks (q12w); and/or
(vi) each dose of the second dosing cycle is between about 30 μg/kg to about 90 μg/kg.

6-8. (canceled)

9. The method of claim 5, wherein:
(i) the C1D1, the C1D2, and the C1D3 are each about 30 μg/kg, about 60 μg/kg, or about 90 μg/kg;
(ii) the second dosing cycle is stopped following the clinical remission, and then restarted following a relapse of the IBD;
(iii) the doses of the second dosing cycle are administered to the subject every eight weeks (q8w);
(iv) each dose of the second dosing cycle is about 60 μg/kg; and/or
(v) the first dose of the second dosing cycle is administered to the subject about 6 weeks to about 10 weeks after the last dose of the first dosing cycle.

10-23. (canceled)

24. A method of treating a subject having an IBD comprising a dosing regimen, the dosing regimen comprising administering to the subject an IL-22 Fc fusion protein every four weeks (q4w) until the subject has a clinical remission of the IBD.

25-26. (canceled)

27. A method of treating a subject having an IBD comprising administering to the subject an IL-22 Fc fusion protein in a dosing regimen comprising a dosing cycle having a length of about 8 weeks, wherein the dosing cycle comprises a first dose (C1D1), a second dose (C1D2), and a third dose (C1D3) of the IL-22 Fc fusion protein, wherein the C1D1, the C1D2, and the C1D3 are each about 30 μg/kg, about 60 μg/kg, or about 90 μg/kg, and wherein the C1D1, the C1D2, and the C1D3 are administered to the subject on or about Weeks 1, 4, and 8, respectively, of the dosing cycle.

28. A method of treating a subject having an IBD comprising administering to the subject an IL-22 Fc fusion protein in a dosage regimen comprising at least a first dosing cycle and a second dosing cycle, wherein:
(a) the first dosing cycle has a length of about 8 weeks and comprises a first dose (C1D1), a second dose (C1D2), and a third dose (C1D3) of the IL-22 Fc fusion protein, wherein the C1D1, the C1D2, and the C1D3 are each about 30 μg/kg, about 60 μg/kg, or about 90 μg/kg, and wherein the C1D1, the C1D2, and the C1D2 are administered to the subject on or about Weeks 1, 4, and 8, respectively, of the first dosing cycle; and
(b) the second dosing cycle continues indefinitely or until clinical remission, and comprises administering about 60 μg/kg of the IL-22 Fc fusion protein to the subject every 8 weeks (q8w), wherein the first dose of the second dosing cycle is administered to the subject about 8 weeks after the last dose of the first dosing cycle.

29. The method of claim 1, wherein:
(a)(i) the treating ameliorates one or more symptoms of the IBD; and/or the treating results in a clinical remission; and/or
(b) the treating results in (i) a clinical response; (ii) endoscopic healing; (iii) endoscopic remission; (iv) a change from baseline in the subject's bowel movement signs and symptoms as assessed by the Ulcerative Colitis Patient-Reported Outcome Signs and Symptoms (UC-PRO/SS) score; (v) a change from baseline in the subject's abdominal signs and symptoms as assessed by the UC-PRO/SS score; (vi) a change from baseline in the subject's patient-reported health-related quality of life (QOL) as assessed by an Inflammatory Bowel Disease Questionnaire (IBDQ) score; (vii) mucosal healing; (viii) a change from baseline in the subject's UC Endoscopic Index of Severity; and/or (ix) histological healing.

30. The method of claim 29, wherein:
(i) the one or more symptoms of IBD include stool frequency, rectal bleeding, or mucosal appearance;
(ii) the clinical remission is a modified Mayo Clinic Score (MCS) of less than or equal to about 2 and a Mayo rectal bleeding subscore of 0 and other Mayo subscores of less than or equal to about 1;
(iii) the clinical remission is a sustained remission;
(iv) the clinical response comprises: (a) a decrease in the subject's mMCS score relative to a baseline mMCS score; or (b) a decrease in the subject's Mayo rectal bleeding subscore relative to a baseline Mayo rectal bleeding subscore or a Mayo rectal bleeding subscore of 0 or 1; and/or
(v) the amelioration of one or more symptoms of IBD, clinical remission, and/or clinical response is maintained at least one month after the end of treatment.

31-36. (canceled)

37. The method of claim 1, wherein:
(i) the IBD is ulcerative colitis (UC) or Crohn's disease; and/or
(ii) the subject has had an inadequate response, loss of response, or intolerance to prior immunosuppressant treatment.

38. (canceled)

39. The method of claim 37, wherein:
(i) the UC is moderate to severe UC; or
(ii) the IBD is Crohn's disease.

40-41. (canceled)

42. A method of treating or preventing graft versus host disease (GVHD) in a subject comprising administering to the subject an IL-22 Fc fusion protein in a dosing regimen comprising a dosing cycle, wherein the dosing cycle comprises between two and ten doses, and wherein a total of about 60 μg/kg to about 900 μg/kg of the IL-22 Fc fusion protein is administered to the subject in the dosing cycle.

43. The method of claim 42, wherein:
(i) the doses are administered to the subject every week (q1w), every two weeks (q2w), every four weeks (q4w), or every six weeks (q6w);
(ii) the first dose of the dosing cycle is administered to the subject about 3 (±2) days prior to allogeneic hematopoietic stem cell transplantation (allo-HSCT);
(iii) the second dose is administered on or about Day 11 following the allo-HSCT;
(iv) a total of about 480 μg/kg of the IL-22 Fc fusion protein is administered to the subject in the dosing cycle;
(v) the dosing cycle comprises a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein; and/or
(vi) the length of the dosing cycle is between about 2 weeks and about 20 weeks.

44-49. (canceled)

50. The method of claim 43, wherein:
(i) the C1D1, the C1D2, the C1D3, the C1D4, the C1D5, the C1D6, the C1D7, and the C1D8 are each between about 15 μg/kg to about 90 μg/kg; and/or
(ii) the length of the dosing cycle is about 96 days.

51. The method of claim 50, wherein the C1D1, the C1D2, the C1D3, the C1D4, the C1D5, the C1D6, the C1D7, and the C1D8 are each about 60 μg/kg.

52-53. (canceled)

54. A method of treating GVHD in a subject comprising a dosing regimen, the dosing regimen comprising administering to the subject an IL-22 Fc fusion protein every two weeks (q2w) until the subject has a clinical remission of the GVHD.

55. The method of claim 42, wherein:
(i) the GVHD is chronic GVHD or acute GVHD;
(ii) the method is a method of preventing GVHD;
(iii) the GVHD is intestinal GVHD;
(iv) the method prevents Grade II-IV acute GVHD;
(v) the method reduces the incidence of Stage 1, Stage 2, Stage 3, or Stage 4 acute GVHD of skin, gut, and liver;
(vi) the method reduces the incidence of Grade I, Grade II, Grade III, or Grade IV acute GVHD; and/or
(vii) the method (a) improves the gastrointestinal (GI) acute GVHD-free survival rate of the subject; (b) improves the overall survival of the subject; (c) improves the relapse-free survival rate of the subject, and/or (d) reduces the incidence of chronic GVHD in the subject.

56-57. (canceled)

58. A method of preventing acute GVHD in a subject comprising administering to the subject an IL-22 Fc fusion protein in a dosing regimen comprising a dosing cycle having a length of about 96 days, wherein the dosing cycle comprises a first dose (C1D1), a second dose (C1D2), a third dose (C1D3), a fourth dose (C1D4), a fifth dose (C1D5), a sixth dose (C1D6), a seventh dose (C1D7), and an eighth dose (C1D8) of the IL-22 Fc fusion protein, wherein the C1D1, the C1D2, the C1D3, the C1D4, the C1D5, the C1D6, the C1D7, and the C1D8 are each about 60 μg/kg, wherein the C1D1 is administered to the subject about 3 (±2) days prior to allo-HSCT, the C1D2 is administered about eleven days after the allo-HSCT, and the C1D3, C1D4, C1D5, C1D6, C1D7, and C1D8 are administered to the subject every two weeks (q2w) following administration of the C1D2.

59-67. (canceled)

68. The method of claim 1, wherein:
(i) the IL-22 Fc fusion protein comprises an IL-22 polypeptide linked to an Fc region by a linker;
(ii) the IL-22 Fc fusion protein comprises an amino acid sequence having at least 95% sequence identity to the amino acid sequence of SEQ ID NO:8;
(iii) the IL-22 Fc fusion protein is a dimeric IL-22 Fc fusion protein or a monomeric IL-22 Fc fusion protein;
(iv) the IL-22 Fc fusion protein binds to IL-22 receptor;
(v) the IL-22 Fc fusion protein is administered to the subject as a monotherapy or as a combination therapy; and/or
(vi) the administering is by intravenous infusion or by subcutaneous administration.

69. The method of claim 68, wherein:
(i) the IL-22 polypeptide is glycosylated and/or the Fc region is not glycosylated;
(ii) the Fc region comprises the CH2 and CH3 domain of IgG1 or IgG4;
(iii) the IL-22 Fc fusion protein comprises or consists of the amino acid sequence of SEQ ID NO:8, SEQ ID NO:10, or SEQ ID NO:16;
(iv) the IL-22 polypeptide is a human IL-22 polypeptide;
(v) the linker comprises or consists of the amino acid sequence RVESKYGPP (SEQ ID NO: 44);
(vi) the IL-22 receptor is human IL-22 receptor;
(vii) the IL-22 Fc fusion protein is administered to the subject in a pharmaceutical composition;
(viii) the IL-22 Fc fusion protein is administered to the subject concurrently with an additional therapeutic agent or prior to the administration of an additional therapeutic agent; and/or
(ix) the IL-22 Fc fusion protein is administered in combination with an additional IBD therapy selected from an aminosalicylate, an immunomodulatory agent, a tumor necrosis factor (TNF) antagonist, an anti-integrin agent, a mucosal addressing cell adhesion molecule (MAdCAM) antagonist, an IL-23 antagonist, an IL-12 antagonist, an IL-12/IL-23 antagonist, an antibiotic, or a corticosteroid.

70. The method of claim 69, wherein:
(a)(i) the amino acid residue at position 297 as in the EU index of the Fc region is Gly or Ala; and/or (ii) the amino acid residue at position 299 as in the EU index of the Fc region is Ala, Gly, or Val; and/or
(b) the pharmaceutical composition has an average sialic acid content in the range of 8 to 12 moles of sialic acid per mole of the IL-22 Fc fusion protein.

71-96. (canceled)

97. A kit comprising an IL-22 Fc fusion protein and instructions to administer the IL-22 Fc fusion protein to a subject suffering from an IBD in accordance with the method of claim 1.

98. A kit comprising an IL-22 Fc fusion protein and instructions to administer the IL-22 Fc fusion protein to a subject suffering from or at risk of GVHD in accordance with the method of claim 42.

99-102. (canceled)
